(19) United States
(12) Patent Application Publication
(10) Pub. No.: US 2026/0131034 A1
(43) Pub. Date: May 14, 2026

(54) RADIOPHARMACEUTICALS TARGETING CARBONIC ANHYDRASE IX PROTEINS

(71) Applicant: NORROY BIOSCIENCE CO., LTD., Wuxi (CN)

(72) Inventors: Peng FANG, Wuxi (CN); Chenglong YAN, Wuxi (CN); Shanyou YU, Wuxi (CN); Wai-si ENG, Wuxi (CN); Si YANG, Wuxi (CN)

(21) Appl. No.: 19/383,270

(22) Filed: Nov. 7, 2025

(30) Foreign Application Priority Data
Nov. 8, 2024 (CN) 2024115952491
Jul. 17, 2025 (CN) 2025109939672
Oct. 31, 2025 (CN) 2025115914371

(51) Int. Cl. A61K 51/08; A61P 35/00; C07K 1/107

(52) U.S. Cl. CPC A61K 51/088; A61P 35/00; C07K 1/1077; A61K 2121/00; A61K 2123/00

(57) ABSTRACT

The present disclosure relates to a radiopharmaceutical targeting carbonic anhydrase IX protein, and in particular to a compound of formula (I), which can effectively target the carbonic anhydrase IX protein, has a fast metabolism rate in the body, high specificity, less uptake by non-target organs, low toxic and side effects on non-target organs, is easy to image, and has high safety, and can be used for diagnosing or treating diseases in which carbonic anhydrase IX protein is overexpressed.

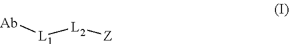

### CROSS REFERENCE TO RELATED APPLICATIONS

The present disclosure claims priority from the Chinese Patent Application No. 202411595249.1 filed on Nov. 8, 2024, the Chinese Patent Application No. 202510993967.2 filed on Jul. 17, 2025, and the Chinese Patent Application No. 202511591437.1 filed on Oct. 31, 2025, all of which are incorporated herein by reference in their entirety.

### TECHNICAL FIELD

The present disclosure relates to a pharmaceutical field, and in particular to radiopharmaceuticals targeting a carbonic anhydrase IX (CAIX) protein.

### BACKGROUND

There are multiple α-carbonic anhydrases (α-CAs) distributed in most organs and tissues of the human body, and in some organs and tissues, only one type of CA is dominant. CAVA/XIV is the dominant CA in the liver, CAIX/XII is the dominant CA in most hypoxic solid tumors, and CAI/II is one of the main protein components of red blood cells in blood. CAIX/XII, which is specifically expressed in tumors, is a transmembrane protein type. CAIX is stably expressed in tumors such as RCC, but not expressed in normal kidney tissue, and only partially weakly expressed in organs and tissues other than the kidney. Therefore, CAIX is a potential excellent target for the diagnosis and treatment of ccRCC. CAIX is also overexpressed in many other types of tumors, such as lung cancer, colorectal cancer, gastric cancer, pancreatic cancer, melanoma, breast cancer, cervical cancer, bladder cancer, ovarian cancer, brain cancer, head and neck cancer, astrocytoma, oral cancer, etc.

CAIX, which is overexpressed in tumors, catalyzes the hydration of carbon dioxide to generate bicarbonate ions and protons, regulating the pH of the cellular environment. The increased acidity of the local microenvironment of the cell can activate the VEGF signaling pathway, promote new angiogenesis, and play an important role in the occurrence and development of tumors. Most inhibitors targeting CA bind to the metal active center of the CAIX extracellular enzyme catalytic domain to block its catalytic activity. The intracellular segment of CAIX can participate in the regulation of glucose metabolism pathway after the phosphorylation of the intracellular segment of CAIX. The structure of CAIX-specific inhibitors is mostly based on the sulfonamide structure, and is ionized, hypoxia-activated structured, glycosylated, and nanoparticle-encapsulated. Labeling of small molecules of CAIX inhibitors with diagnostic and therapeutic nuclides can be used for molecular imaging diagnosis and treatment of tumors.

Currently, the effective treatment for RCC is surgical resection, but the 5-year recurrence rate of localized RCC after surgery is 30% (or about 50%), while advanced or metastatic RCC cannot undergo surgery. The classic effective drug for metastatic RCC is high-dose interleukin-2, but it can only produce sustained complete response in 7-10% of patients. Therefore, for patients with a high risk of recurrence, the development of more sensitive and accurate early diagnosis strategies, as well as effective systemic treatments for advanced and metastatic RCC, is a challenge that needs to be continued to be faced and addressed. The development of better CAIX targeted drugs and CAIX inhibitors for molecular imaging diagnosis and treatment has important scientific research value and broad application prospects.

### SUMMARY

The present disclosure provides a radiopharmaceutical of carbonic anhydrase IX protein, which can be used for diagnosing and or treating diseases in which carbonic anhydrase IX protein is overexpressed.

The radiopharmaceutical targeting carbonic anhydrase IX protein of the present disclosure has the following advantages:
(1) The radiopharmaceutical has fast metabolism in vivo and little toxic and side effects on non-target organs;
(2) The radiopharmaceutical specifically targets a tumor, with little uptake by non-target organs in vivo, which is convenient for imaging and beneficial for clinical diagnosis;
(3) The radiopharmaceutical specifically targets a tumor and stays in the tumor for a long time, which has a good therapeutic effect on the tumor;
(4) The radiopharmaceutical has low uptake by non-target organs, low toxicity and side effects on normal tissues, and good safety.

In one aspect, the present disclosure relates to a compound of formula (I), or an isotopic variant, a hydrate, an ester or solvate, a tautomer or a stereoisomer thereof, or a pharmaceutically acceptable salt thereof:

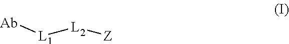

wherein, each variable is as defined herein.

In another aspect, the present disclosure relates to a compound comprising a compound of formula (I), or an isotopic variant, hydrate, ester or solvate, tautomer, stereoisomer, or a pharmaceutically acceptable salt thereof, and M complexed therewith, wherein:

the compound of formula (I) is as defined herein;
M is selected from at least one of radionuclides and non-radioactive elements.

In another aspect, the present disclosure relates to a compound of formula (V), or an isotopic variant, hydrate, ester or solvate, tautomer, stereoisomer, or a pharmaceutically acceptable salt thereof:

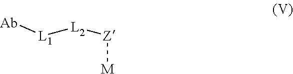

wherein Ab, L₁ and L₂ are as defined herein;
Z′ is a coordination group formed by complexing a chelating group Z derived from a chelating agent with M, and Z is as defined herein;
M is selected from at least one of radionuclides and non-radioactive elements.

In another aspect, the present disclosure relates to a pharmaceutical composition comprising a compound of the present disclosure, or an isotopic variant, a hydrate, an ester or solvate, a tautomer or a stereoisomer thereof, or a pharmaceutically acceptable salt thereof;
optionally, the pharmaceutical composition further comprises a pharmaceutically acceptable carrier or excipient.

In another aspect, the present disclosure relates to the use of a compound of the present disclosure, or an isotopic variant, a hydrate, an ester or solvate, a tautomer or a stereoisomer thereof, or a pharmaceutically acceptable salt thereof, or a pharmaceutical composition of the present disclosure in the manufacture of a medicament for inhibiting the expression of carbonic anhydrase IX.

In another aspect, the present disclosure relates to a compound of the present disclosure, or an isotopic variant, a hydrate, an ester or solvate, a tautomer or a stereoisomer thereof, or a pharmaceutically acceptable salt thereof, or a pharmaceutical composition of the present disclosure for use in inhibiting the expression of carbonic anhydrase IX.

In another aspect, the present disclosure relates to a method for inhibiting the expression of carbonic anhydrase IX, wherein the method comprises administering to a subject a compound of the present disclosure, or an isotopic variant, a hydrate, an ester or solvate, a tautomer or a stereoisomer thereof, or a pharmaceutically acceptable salt thereof, or a pharmaceutical composition of the present disclosure.

In another aspect, the present disclosure relates to the use of a compound of the present disclosure, or an isotopic variant, a hydrate, an ester or solvate, a tautomer or a stereoisomer thereof, or a pharmaceutically acceptable salt thereof, or a pharmaceutical composition of the present disclosure in the manufacture of an agent and/or a medicament for diagnosing and/or treating one or more tumors, cancers or cells expressing carbonic anhydrase IX.

In another aspect, the present disclosure relates to a compound of the present disclosure, or an isotopic variant, a hydrate, an ester or solvate, a tautomer or a stereoisomer thereof, or a pharmaceutically acceptable salt thereof, or a pharmaceutical composition of the present disclosure, for use in diagnosing and/or treating one or more tumors, cancers or cells expressing carbonic anhydrase IX.

In another aspect, the present disclosure relates to a method for diagnosing and/or treating one or more tumors, cancers or cells expressing carbonic anhydrase IX, wherein the method comprises administering to a subject a compound of the present disclosure, or an isotopic variant, a hydrate, an ester or solvate, a tautomer or a stereoisomer thereof, or a pharmaceutically acceptable salt thereof, or a pharmaceutical composition of the present disclosure.

In another aspect, the diagnostic modality is selected from optical imaging and/or nuclear imaging; alternatively, the nuclear imaging is selected from PET imaging and/or SPECT imaging.

In another aspect, the treatment is selected from radiation therapy and/or assisted surgery with fluorescent surgical navigation.

In another aspect, the disease associated with carbonic anhydrase IX expression is selected from tumors.

In another aspect, the disease associated with carbonic anhydrase IX expression is selected from renal cancer, lung cancer, colorectal cancer, gastric cancer, pancreatic cancer, melanoma, breast cancer, cervical cancer, bladder cancer, ovarian cancer, brain cancer, head and neck cancer, astrocytoma and oral cancer.

In another aspect, the present disclosure relates to a method for imaging a tissue expressing carbonic anhydrase IX, comprising administering to the tissue a compound of the present disclosure, or an isotopic variant, a hydrate, an ester or solvate, a tautomer or a stereoisomer thereof, or a pharmaceutically acceptable salt thereof, or a pharmaceutical composition of the present disclosure, and imaging the tissue after administration.

In another aspect, the imaging is emission computed tomography, which is performed by positron emission tomography or single photon emission computed tomography.

### Definition

### Chemical Definition

Definitions of specific functional groups and chemical terms are described in more detail below.

When a numerical range is listed, each value and subrange within the stated range is intended to be included. For example, “C₁₋₆ alkyl” includes C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₁₀ alkyl” refers to a radical of a straight or branched, saturated hydrocarbon group having 1 to 10 carbon atoms. In some embodiments, C₁₋₈ alkyl, C₁₋₆ alkyl, C₁₋₄ alkyl, C₁₋₃ alkyl and C₁₋₂ alkyl are alternative. Examples of C₁₋₆ alkyl include: methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl groups in which one or more (e.g., 1, 2, 3, or 4) carbon atoms are replaced with heteroatoms (e.g., oxygen, sulfur, nitrogen, boron, silicon, phosphorus). The alkyl group can be optionally substituted by one or more substituents, for example, by 1 to 5 substituents, 1 to 3 substituents, or 1 substituent. Conventional abbreviations of alkyl include: Me(-CH₃), Et(-CH₂CH₃), iPr(-CH(CH₃)₂), nPr(-CH₂CH₂CH₃), n-Bu(-CH₂CH₂CH₂CH₃) or i-Bu(-CH₂CH(CH₃)₂). In some embodiments, straight chain alkyl groups are alternative.

“C₂₋₁₀ alkenyl” refers to a radical of a straight or branched hydrocarbon group having 2 to 10 carbon atoms and at least one carbon-carbon double bond. In some embodiments, C₂₋₈ alkenyl, C₂₋₆ alkenyl, C₂₋₄ alkenyl and C₂₋₃ alkenyl are alternative. Examples of C₂₋₆ alkenyl include: vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), and the like. The term “C₂₋₆ alkenyl” also includes heteroalkenyl groups in which one or more (e.g., 1, 2, 3 or 4) carbon atoms are replaced by heteroatoms (e.g., oxygen, sulfur, nitrogen, boron, silicon, phosphorus). An alkenyl group can be optionally substituted with one or more substituents, for example, 1 to 5 substituents, 1 to 3 substituents, or 1 substituent. In some embodiments, straight chain alkenyl groups are alternative.

“C₂₋₁₀ alkynyl” refers to a radical of a straight or branched hydrocarbon group having 2 to 6 carbon atoms, at least one carbon-carbon triple bond, and optionally one or more carbon-carbon double bonds. In some embodiments, C₂₋₈ alkynyl, C₂₋₆ alkynyl, C₂₋₄ alkynyl, and C₂₋₃ alkynyl are alternative. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), and the like. The term “C₂₋₆ alkynyl” also includes heteroalkynyl groups in which one or more (e.g., 1, 2, 3, or 4) carbon atoms are replaced by heteroatoms (e.g., oxygen, sulfur, nitrogen, boron, silicon, phosphorus). The alkynyl groups can be optionally substituted with one or more substituents, for example, 1 to 5 substituents, 1 to 3 substituents, or 1 substituent. In some embodiments, straight chain alkynyl groups are alternative.

“C₁₋₁₀ alkylene” refers to a divalent group formed by removing another hydrogen of a C₁₋₁₀ alkyl, and can be substituted or unsubstituted. In some embodiments, C₁₋₈ alkylene, C₁₋₆ alkylene, C₁₋₄ alkylene, C₁₋₃ alkylene, C₁₋₂ alkylene and methylene are alternative. The unsubstituted alkylene includes, but is not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, for example, alkylene groups substituted with one or more alkyl(methyl) groups, include, but are not limited to, substituted methylene groups (—CH(CH₃)—, —C(CH₃)₂—), substituted ethylene groups (—CH(CH₃)CH₂—, —CH₂CH(CH₃)—, —C(CH₃)₂CH₂—, —CH₂C(CH₃)₂—), substituted propylene groups (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. In some embodiments, straight chain alkylene groups are alternative.

“C₂₋₁₀ alkenylene” refers to a divalent group formed by removing another hydrogen of a C₂₋₁₀ alkenyl group, and can be substituted or unsubstituted. In some embodiments, C₂₋₈ alkenylene, C₂₋₆ alkenylene, C₂₋₄ alkenylene, and C₂₋₃ alkenylene are yet alternative. Exemplary unsubstituted alkenylene groups include, but are not limited to, vinylene (—CH═CH—), propenylene (e.g., —CH═CHCH₂—, —CH₂—CH═CH—), butenylene (e.g., —CH═CHCH₂CH₂—, —CH₂—CH═CH—CH₂—, —CH₂—CH₂—CH═CH—), and the like. Exemplary substituted alkenylene groups, for example, alkenylene groups substituted with one or more alkyl(methyl) groups, include, but are not limited to, substituted ethenylene groups (—C(CH₃)═CH—, —CH═C(CH₃)—), substituted propenylene groups (—C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like. In some embodiments, straight-chain alkenylene groups are alternative.

“C₂₋₁₀ alkynylene” refers to a divalent group formed by removing another hydrogen of a C₂₋₁₀ alkynyl group, and can be substituted or unsubstituted. In some embodiments, C₂₋₈ alkynylene, C₂₋₆ alkynylene, C₂₋₄ alkynylene, and C₂₋₃ alkynylene are yet alternative. Exemplary unsubstituted alkynylene groups include, but are not limited to, ethynylene (—C≡C—), propynylene (e.g., —C≡CCH₂—, —CH₂—C≡C—), butynylene (e.g., —C≡CCH₂CH₂—, —CH₂—C≡C—CH₂—, —CH₂—CH₂—C═C—), and the like. Exemplary substituted alkynylene groups, for example, alkynylene groups substituted with one or more alkyl(methyl) groups, include, but are not limited to, substituted propynylene groups (—C═CCH(CH₃)—, —C≡CC(CH₃)₂—, —CH(CH₃)—C≡C—, —C(CH₃)₂—C≡C—), etc. In some embodiments, straight chain alkynylene groups are alternative.

“C₀₋₆ alkylene” refers to a chemical bond and the above-mentioned “C₁₋₆ alkylene”, and “C₀₋₄ alkylene” refers to a chemical bond and the above-mentioned “C₁₋₄ alkylene”. The same applies to other similar cases.

“Halo” or “halogen” refers to fluorine (F), chlorine (C₁), bromine (Br) and iodine (I).

Therefore, “C₁₋₁₀ haloalkyl” refers to the above-mentioned “C₁₋₁₀ alkyl”, which is substituted with one or more halogen groups. In some embodiments, C₁₋₈ haloalkyl is yet alternative. In some embodiments, C₁₋₆ haloalkyl is yet alternative. In some embodiments, C₁₋₄ haloalkyl is yet alternative, still alternatively C₁₋₃ haloalkyl, still alternatively C₁₋₂ haloalkyl, still alternatively halomethyl. Exemplary haloalkyls include, but are not limited to: —CF₃, —CH₂F, —CHF₂, —CHFCH₂F, —CH₂CHF₂, —CH₂CF₃, —CF₂CF₃, —CCl₃, —CH₂Cl, —CHCl₂, 2,2,2-trifluoro-1,1-dimethyl-ethyl, and the like. The haloalkyl group can be substituted at any available point of attachment, for example, with 1 to 5 substituents, 1 to 3 substituents, or 1 substituent.

“C₃₋₁₀ cycloalkyl” refers to a radical of a non-aromatic cyclic hydrocarbon group having 3 to 10 ring carbon atoms and zero heteroatoms, optionally containing 1, 2 or 3 double bonds or triple bonds. In some embodiments, C₅₋₁₀ cycloalkyl, C₃₋₇ cycloalkyl and C₃₋₆ cycloalkyl are yet alternative, and still alternatively C₅₋₇ cycloalkyl and C₅₋₇ cycloalkyl. The cycloalkyl also includes a ring system in which the cycloalkyl ring described above is fused with one or more aryl or heteroaryl groups, wherein the point of attachment is on the cycloalkyl ring, and in such a case, the number of carbon atoms continues to represent the number of carbon atoms in the cycloalkyl system. The cycloalkyl also includes the above cycloalkyl rings, wherein the substituents on any non-adjacent carbon atoms are connected to form a bridged ring, together forming a polycycloalkane sharing two or more carbon atoms. The cycloalkyl also includes the above cycloalkyl rings, wherein the substituents on the same carbon atom are connected to form a ring, together forming a polycycloalkane sharing one carbon atom. Exemplary cycloalkyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl groups can be optionally substituted with one or more substituents, for example, with 1 to 5 substituents, 1 to 3 substituents or 1 substituent.

“C₃₋₁₀ cycloalkylene” refers to a divalent group formed by removing another hydrogen of a C₃₋₁₀ cycloalkyl group, and can be substituted or unsubstituted. In some embodiments, C₃₋₇ cycloalkylene, C₅₋₇ cycloalkylene, C₃₋₆ cycloalkylene and C₃₋₄ cycloalkylene are yet alternative, such as cyclopropylene, such as cyclobutylene, such as cyclopentylene, such as cyclohexylene, and cyclopropylene is yet alternative.

“3-10 membered heterocyclyl” refers to a saturated or unsaturated radical of a 3- to 10-membered non-aromatic ring system having ring carbon atoms and 1 to 5 ring heteroatoms, wherein each of the heteroatom is independently selected from nitrogen, oxygen, sulfur, boron, phosphorus and silicon, and 1, 2 or 3 double or triple bonds are optionally included. In the heterocyclyl containing one or more nitrogen atoms, the point of attachment can be a carbon or nitrogen atom as long as the valence permits. In some embodiments, a 5- to 10-membered heterocyclyl is alternative, which is a radical of 5- to 10-membered non-aromatic ring system having ring carbon atoms and 1 to 5 ring heteroatoms; in some embodiments, a 3- to 7-membered heterocyclyl is alternative, which is a radical of a 3- to 7-membered non-aromatic ring system having ring carbon atoms and 1 to 4 ring heteroatoms; a 5- to 7-membered heterocyclyl is alternative, which is a radical of a 5- to 7-membered non-aromatic ring system having ring carbon atoms and 1 to 3 ring heteroatoms; a 3- to 6-membered heterocyclyl is alternative, which is a radical of a 3- to 6-membered non-aromatic ring system having ring carbon atoms and 1 to 3 ring heteroatoms; a 4- to 6-membered heterocyclyl is alternative, which is a radical of a 4- to 6-membered non-aromatic ring system having ring carbon atoms and 1 to 3 ring heteroatoms; and a 5- to 6-membered heterocyclyl is yet alternative, which is a radical of a 5- to 6-membered non-aromatic ring system having ring carbon atoms and 1 to 3 ring heteroatoms. The heterocyclyl also includes a ring system in which the above-mentioned heterocyclyl ring is fused with one or more cycloalkyl groups, wherein the point of attachment is on the heterocyclyl ring, or the above-mentioned heterocyclyl ring is fused with one or more aryl or heteroaryl groups, wherein the point of attachment is on the heterocyclyl ring; and in such a case, the number of ring members continues to represent the number of ring members in the heterocyclyl ring system. The heterocyclyl also includes the above-mentioned heterocyclyl ring, wherein the substituents on any non-adjacent carbon or nitrogen atoms are connected to form a bridge ring, together forming a polycyclic heteroalkane sharing two or more carbon or nitrogen atoms. The heterocyclyl also includes the above-mentioned heterocyclyl ring, wherein the substituents on the same carbon atom are connected to form a ring, together forming a polycyclic heteroalkane sharing one carbon atom. Exemplary 3-membered heterocyclyl groups containing one heteroatom include, but are not limited to: aziridinyl, oxiranyl, and thiorenyl. Exemplary 4-membered heterocyclyl groups containing one heteroatom include, but are not limited to: azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl, and pyrrolyl-2,5-dione. Exemplary 5-membered heterocyclyl groups containing two heteroatoms include, but are not limited to, pyrazolidinyl, dioxolanyl, oxasulfuranyl, disulfuranyl, and oxazolidin-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridinyl, and thianyl. Exemplary 6-membered heterocyclyl groups containing two heteroatoms include, but are not limited to, piperazinyl, morpholinyl, dithianyl, and dioxanyl. Exemplary 6-membered heterocyclyl groups containing three heteroatoms include, but are not limited to, hexahydrotriazinyl (triazinanyl). Exemplary 7-membered heterocyclyl groups containing one heteroatom include, but are not limited to, azepanyl, oxepanyl and thiepanyl. Exemplary 5-membered heterocyclyl groups fused to a C₆ aryl ring (also referred to as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused to a C₆ aryl ring (also referred as 6,6-bicyclic heterocyclyl herein) include, but are not limited to, tetrahydroquinolinyl, tetrahydroisoquinolinyl, and the like. The heterocyclyl also includes a bridged ring or a spiral ring formed by the above heterocyclyl and a cycloalkyl, heterocyclyl, aryl or heteroaryl group sharing one or two atoms, and the shared atom can be a carbon or nitrogen atom as long as the valence permits. The heterocyclyl also includes the above heterocyclyl and heterocyclyl groups which can be optionally substituted with one or more substituents, for example, with 1 to 5 substituents, 1 to 3 substituents or 1 substituent.

“3-10 membered heterocyclylene” refers to a divalent group formed by removing another hydrogen of a 3-10 membered heterocyclyl, and can be substituted or unsubstituted. In some embodiments, 3-7 membered heterocyclylenyl, 5-7 membered heterocyclylenyl, 3-6 membered heterocyclylenyl and 3-4 membered heterocyclylenyl are yet alternative, such as cyclopropylenyl, such as tetrahydrofuranylenyl and pyranylenyl.

“C₆₋₁₄ aryl” refers to a radical of monocyclic or polycyclic (e.g., bicyclic) 4n+2 aromatic ring system having 6 to 14 ring carbon atoms and zero heteroatoms (e.g., having 6, 10, or 14 shared π electrons in a cyclic array). In some embodiments, C₆₋₁₀ aryl is alternative. In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). In some embodiments, the aryl has fourteen ring carbon atoms (“C₁₄ aryl”; for example, anthracenyl and phenanthryl, e.g., 1-anthracenyl, 2-anthracenyl, 1-phenanthryl and 2-phenanthryl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl group can be optionally substituted with one or more substituents, for example, with 1 to 5 substituents, 1 to 3 substituents or 1 substituent.

“5- to 14-membered heteroaryl” refers to a radical of 5- to 14-membered monocyclic or bicyclic 4n+2 aromatic ring system (e.g., having 6, 10 or 14 shared π electrons in a cyclic array) having ring carbon atoms and 1 to 4 ring heteroatoms, wherein each heteroatom is independently selected from nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number the carbon atoms continues to represent the number of carbon atoms in the heteroaryl ring system. In some embodiments, 5- to 10-membered heteroaryl groups are alternative, which are radicals of 5- to 10-membered monocyclic or bicyclic 4n+2 aromatic ring systems having ring carbon atoms and 1 to 4 ring heteroatoms. In other embodiments, 5- to 6-membered heteroaryl groups are yet alternative, which are radicals of 5- to 6-membered monocyclic or bicyclic 4n+2 aromatic ring systems having ring carbon atoms and 1 to 4 ring heteroatoms. In other embodiments, 5 membered heteroaryls are yet alternative. Exemplary 5-membered heteroaryl groups containing one heteroatom include, but are not limited to, pyrrolyl, furyl and thienyl. Exemplary 5 membered heteroaryls containing two heteroatoms include, but are not limited to, imidazolyl, imidazolonyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazoly), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl and pyridonyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl, respectively. Exemplary 7-membered heteroaryl groups containing one heteroatom include, but are not limited to, azepinyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

“C₆₋₁₄ arylene” refers to a divalent group formed by removing another hydrogen of a C₆₋₁₄ aryl group, and can be substituted or unsubstituted. In some embodiments, a C₆₋₁₀ arylene group is alternative. In some embodiments, a phenylene group

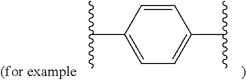 [SMILES: *c1ccc(*)cc1]

is alternative.

“5-14 membered heteroarylene” refers to a divalent group formed by removing another hydrogen of a 5-14 membered heteroarylene, and can be substituted or unsubstituted. In some embodiments, a 5-10 membered heteroarylene is alternative. In some embodiments, a 5-6 membered heteroarylene is alternative, for example, a pyrrolylene

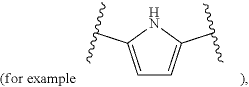 [SMILES: *c1ccc(*)[nH]1]

a furanylene

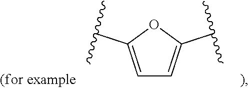 [SMILES: *c1ccc(*)o1]

or a thienylene

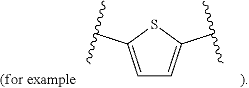 [SMILES: *c1ccc(*)s1]

“Optionally substituted . . . ” means that a group can be substituted by a specific substituent or can be unsubstituted.

The divalent groups formed by removing another hydrogen from the alkyl, alkenyl, alkynyl, cycloalkyl, heterocyclyl, aryl and heteroaryl groups defined above are collectively referred to as “-ylene” or “-ylidene”. Ring-forming groups such as cycloalkyl, heterocyclyl, aryl and heteroaryl are collectively referred to as “cyclyl groups”.

Alkyl, alkenyl, alkynyl, cycloalkyl, heterocyclyl, aryl and heteroaryl as defined herein are optionally substituted groups.

Exemplary substituents on carbon atoms include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(=NR^{bb})OR^{aa}, —C(═NR^{bb})N(R^{bb})₂, —OC(═NR^{bb})N(R^{bb})₂, —NR^{bb}C(=NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(=O)(OR^{cc})₂, —NR^{bb}P(=O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl and heteroaryl, wherein each of the alkyl, alkenyl, alkynyl, cycloalkyl, heterocyclyl, aryl and heteroaryl is independently substituted with 0, 1, 2, 3, 4 or 5 R^{dd} groups;
or two geminal hydrogen on a carbon atom are replaced with ═O, ═S, ═NN(R^{bb})₂, =NNR^{bb}C(═O)R^{aa}, =NNR^{bb}C(═O)OR^{aa}, =NNR^{bb}S(=O)₂R^{aa}, =NR^{bb} or =NORC groups;
each of the R^{aa} is independently selected from alkyl, haloalkyl, alkenyl, alkynyl, cycloalkyl, heterocyclyl, aryl and heteroaryl, or two of the R^{aa} groups are combined to form a heterocyclyl or heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{bb} is independently selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{cc} is independently selected from hydrogen, alkyl, haloalkyl, alkenyl, alkynyl, cycloalkyl, heterocyclyl, aryl and heteroaryl, or two RCC groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;
each of the R^{dd} is independently selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, and heteroaryl, wherein, each of the alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents can be combined to form ═O or =S;
each of the R^{ee} is independently selected from alkyl, haloalkyl, alkenyl, alkynyl, cycloalkyl, aryl, heterocyclyl, and heteroaryl, wherein, each of the alkyl, alkenyl, alkynyl, cycloalkyl, heterocyclyl, aryl and heteroaryl is independently substituted with 0, 1, 2, 3, 4 or 5 R^{gg} groups;
each of the R^{ff} is independently selected from hydrogen, alkyl, haloalkyl, alkenyl, alkynyl, cycloalkyl, heterocyclyl, aryl and heteroaryl, or two R^{ff} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{gg} groups;
each of the R^{gg} is independently halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ cycloalkyl, C₆-C₁₀ aryl, C₃-C₇ heterocyclyl, or C₅-C₁₀ heteroaryl; or two geminal R^{gg} substituents may combine to form ═O or =S; wherein, X⁻ is a counter-ion.

Exemplary substituents on nitrogen atoms include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(=NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two RCC groups attached to a nitrogen atom combine to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

### Other Definitions

The term “treating” as used herein relates to reversing, alleviating or inhibiting the progression or prevention of the disorders or conditions to which the term applies, or of one or more symptoms of such disorders or conditions. The noun “treatment” as used herein relates to the action of treating, which is a verb, and the latter is as just defined.

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

The pharmaceutically acceptable base addition salts are formed with metals or amines, such as alkali metal and alkaline earth metal hydroxides or organic amines. Examples of the metals used as cations include sodium, potassium, magnesium, calcium, etc. Examples of suitable amines are N,N′-dibenzylethylenediamine, chloroprocaine, choline, diethanolamine, ethylenediamine, N-methylglucamine and procaine.

The base addition salt of the acidic compound can be prepared by contacting the free acid form with a sufficient amount of the required base to form a salt in a conventional manner. The free acid can be regenerated by contacting the salt form with an acid in a conventional manner and then isolating the free acid. The free acid forms are somewhat different from their respective salt forms in their physical properties, such as solubility in polar solvents. But for the purposes of the present disclosure, the salts are still equivalent to their respective free acids.

The salts can be prepared from the inorganic acids, which include sulfates, pyrosulfates, bisulfates, sulfites, bisulfites, nitrates, phosphates, monohydrogen phosphates, dihydrogen phosphates, metaphosphates, pyrophosphates, chlorides, bromides and iodides. Examples of the acids include hydrochloric acid, nitric acid, sulfuric acid, hydrobromic acid, hydroiodic acid, phosphoric acid, etc. The representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthalate, methanesulfonate, glucoheptanate, lactobionate, lauryl sulfonate, isethionate, etc. The salts can also be prepared from the organic acids, which include aliphatic monocarboxylic and dicarboxylic acids, phenyl-substituted alkanoic acids, hydroxyalkanoic acids, alkanedioic acid, aromatic acids, aliphatic and aromatic sulfonic acids, etc. The representative salts include acetate, propionate, octanoate, isobutyrate, oxalate, malonate, succinate, suberate, sebacate, fumarate, maleate, mandelate, benzoate, chlorobenzoate, methyl benzoate, dinitrobenzoate, naphthoate, besylate, tosylate, phenylacetate, citrate, lactate, maleate, tartrate, methanesulfonate, etc. The pharmaceutically acceptable salts can include cations based on alkali metals and alkaline earth metals, such as sodium, lithium, potassium, calcium, magnesium, etc., as well as non-toxic ammonium, quaternary ammonium, and amine cations including, but not limited to, ammonium, tetramethylammonium, tetraethylammonium, methylamine, dimethylamine, trimethylamine, triethylamine, ethylamine, etc. Salts of amino acids are also included, such as arginine salts, gluconates, galacturonates, etc. (for example, see Berge S. M. et al., “Pharmaceutical Salts,” J. Pharm. Sci., 1977; 66: 1-19 for reference).

“Subjects” to which administration is contemplated include, but are not limited to, humans (e.g., males or females of any age group, e.g., paediatric subjects (e.g., infants, children, adolescents) or adult subjects (e.g., young adults, middle-aged adults or older adults)) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “human”, “patient” and “subject” can be used interchangeably herein.

“Disease,” “disorder,” and “condition” can be used interchangeably herein.

Unless indicated, otherwise the term “treatment” as used herein includes the effect on a subject who is suffering from a particular disease, disorder, or condition, which reduces the severity of the disease, disorder, or condition, or delays or slows the progression of the disease, disorder or condition (“therapeutic treatment”). The term also includes the effect that occurs before the subject begins to suffer from a specific disease, disorder or condition (“prophylactic treatment”).

Generally, the “effective amount” of a compound refers to an amount sufficient to elicit a target biological response. As understood by those skilled in the art, the effective amount of the compound of the disclosure can vary depending on the following factors, such as the desired biological endpoint, the pharmacokinetics of the compound, the diseases being treated, the mode of administration, and the age, health status and symptoms of the subjects. The effective amount includes therapeutically effective amount and prophylactically effective amount.

Unless indicated, otherwise the “therapeutically effective amount” of the compound as used herein is an amount sufficient to provide therapeutic benefits in the course of treating a disease, disorder or condition, or to delay or minimize one or more symptoms associated with the disease, disorder or condition. The therapeutically effective amount of a compound refers to the amount of the therapeutic agent that, when used alone or in combination with other therapies, provides a therapeutic benefit in the treatment of a disease, disorder or condition. The term “therapeutically effective amount” can include an amount that improves the overall treatment, reduces or avoids the symptoms or causes of the disease or condition, or enhances the therapeutic effect of other therapeutic agents.

Unless indicated, otherwise the “prophylactically effective amount” of the compound as used herein is an amount sufficient to prevent a disease, disorder or condition, or an amount sufficient to prevent one or more symptoms associated with a disease, disorder or condition, or an amount sufficient to prevent the recurrence of a disease, disorder or condition. The prophylactically effective amount of a compound refers to the amount of a therapeutic agent that, when used alone or in combination with other agents, provides a prophylactic benefit in the prevention of a disease, disorder or condition. The term “prophylactically effective amount” can include an amount that improves the overall prevention, or an amount that enhances the prophylactic effect of other preventive agents.

“Combination” and related terms refer to the simultaneous or sequential administration of the compounds of the present disclosure and other therapeutic agents. For example, the compounds of the present disclosure can be administered simultaneously or sequentially in separate unit dosage with other therapeutic agents, or simultaneously in a single unit dosage with other therapeutic agents.

### BRIEF DESCRIPTION OF THE DRAWINGS

FIG. 1 shows the detection diagram of compound 1: (a) LCMS detection diagram, (b) HPLC detection diagram;

FIG. 2 shows the detection diagram of compound 2: (a) LCMS detection diagram, (b) HPLC detection diagram;

FIG. 3 shows the radioactive thin layer chromatography scanning results of compound 1 after radioactive labeling: (a) ⁶⁸Ga-compound 1, (b) ¹⁷⁷Lu-compound 1;

FIG. 4 shows the radioactive thin layer chromatography scanning results of compound 2 after radioactive labeling: (a) ⁶⁸Ga-compound 2, (b) ¹⁷⁷Lu-compound 2;

FIG. 5 shows the PET/CT imaging results of ⁶⁸Ga-compound 1 in OS-RC-2 model mice at different times after administration;

FIG. 6 shows the uptake results of ⁶⁸Ga-compound 1 in different tissues of OS-RC-2 model mice at different times after administration;

FIG. 7 shows the PET/CT imaging results of ⁶⁸Ga-compound 1 in OS-RC-2 model mice at 1 hour after administration;

FIG. 8 shows the in vitro tissue distribution of ⁶⁸Ga-compound 1 in the OS-RC-2 model at 1 hour and 4 hours after administration;

FIG. 9 shows the PET/CT imaging results of ¹⁷⁷Lu-compound 1 in OS-RC-2 model mice at different times after administration;

FIG. 10 shows the in vitro tissue distribution of ¹⁷⁷Lu-compound 1 in the OS-RC-2 model at 4 hours and 48 hours after administration;

FIG. 11 shows the changes in blood concentration of ⁶⁸Ga-compound 1 in ICR mice after administration;

FIG. 12 shows the changes in tumor volume and body weight of OS-RC-2 model mice administered with ¹⁷⁷Lu-compound 1;

FIG. 13 shows the PET/CT imaging results of ⁶⁸Ga-compound 2 in OS-RC-2 model mice at different times after administration;

FIG. 14 shows the uptake results of ⁶⁸Ga-compound 2 in different tissues of OS-RC-2 model mice at different times after administration;

FIG. 15 shows the in vitro tissue distribution of ¹⁷⁷Lu-compound 2 in the OS-RC-2 model at 4 hours and 48 hours after administration;

FIG. 16 shows the changes in (a) tumor volume and (b) body weight of OS-RC-2 model mice administered with ¹⁷⁷Lu-compound 2;

FIG. 17 shows the detection diagram of compound 3: (a) LCMS detection diagram, (b) HPLC detection diagram;

FIG. 18 shows the detection graphs of compound 4: (a) LCMS detection diagram, (b) HPLC detection diagram;

FIG. 19 shows the radioactive thin layer chromatography scanning result of ¹⁶¹Tb-compound 2;

FIG. 20 shows the radioactive thin layer chromatography scanning result of ⁶⁸Ga-compound 3;

FIG. 21 shows the radioactive thin layer chromatography scanning result of ¹⁸F-compound 4;

FIG. 22 shows the in vitro tissue distribution of ¹⁶¹Tb-compound 2 in the OS-RC-2 model at different times after administration;

FIG. 23 shows the changes of (a) tumor volume and (b) body weight of OS-RC-2 model mice administered with ¹⁶¹Tb-compound 2;

FIG. 24 shows the PET/CT imaging results of ⁶⁸Ga-compound 3 in OS-RC-2 model mice at different times after administration;

FIG. 25 shows the uptake results of ⁶⁸Ga-compound 3 in different tissues of OS-RC-2 model mice at different times after administration;

FIG. 26 shows the PET/CT imaging results of ¹⁸F-compound 4 in OS-RC-2 model mice at different times after administration;

FIG. 27 shows the uptake results of ¹⁸F-compound 4 in different tissues of OS-RC-2 model mice at different times after administration;

FIG. 28 shows a maximum intensity projection (MIP) image of a patient in the clinical trial.

FIG. 29 shows the detection diagram of compound 5: (a) LCMS detection diagram, (b) HPLC detection diagram;

FIG. 30 shows the detection diagram of compound 6: (a) LCMS detection diagram, (b)HPLC detection diagram;

FIG. 31 shows the radioactive thin layer chromatography scanning result of ⁶⁸Ga-compound 5;

FIG. 32 shows the radioactive thin layer chromatography scanning result of ⁶⁸Ga-compound 6;

FIG. 33 shows the PET/CT imaging results of ⁶⁸Ga-compound 5 in OS-RC-2 model mice at different times after administration.

### DETAILED DESCRIPTION

As used herein, “compounds of the present disclosure” refers to compounds of the following formula (I), formula (II-1), formula (II-2), formula (II-3), formula (II-4), formula (III-1), formula (III-2), formula (III-3), formula (IV-1), formula (IV-2), formula (IV-3), formula (V), etc., isotopic variants, tautomers, stereoisomers, prodrugs, polymorphs, hydrates or solvates thereof, or pharmaceutically acceptable salts thereof.

In the present disclosure, compounds are named using standard nomenclature. For compounds having an asymmetric center, it should be understood, unless otherwise stated, that all optical isomers and mixtures thereof are included. Furthermore, unless otherwise specified, all isomer compounds and carbon-carbon double bonds included in the present disclosure may occur in the form of Z and E. Compounds which exist in different tautomeric forms, one of which is not limited to any particular tautomer, but is intended to cover all tautomeric forms.

In one embodiment, the present disclosure provides a compound of formula (I), or an isotopic variant, a hydrate, an ester or solvate, a tautomer or a stereoisomer thereof, or a pharmaceutically acceptable salt thereof:

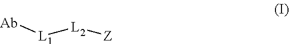

wherein,
Ab is a ligand targeting carbonic anhydrase IX, for example

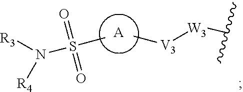

Ring A is selected from C₆₋₁₀ arylene and C₅₋₁₀ heteroarylene; wherein the C₆₋₁₀ arylene and C₅₋₁₀ heteroarylene are optionally substituted by 1, 2, 3 or 4 R_{A};
R_{A} is independently selected from H, D, halogen, CN, —OR, —SRₐ, —NR_{b}R_{c}, C₁₋₆ alkyl, C₁₋₆ haloalkyl, C₃₋₁₀ cycloalkyl, 3-10 membered heterocyclyl, C₆₋₁₀ aryl, and 5-10 membered heteroaryl;
R₃ and R₄ are independently selected from H, halogens, C₁₋₆ alkyl, C₁₋₆ haloalkyl, C₃₋₇ cycloalkyl, and 3-7 membered heterocyclyl;
or R₄ and R_{A} are connected to form C₁₋₄ alkylene, C₂₋₄ alkenylene and C₂₋₄ alkynylene, wherein one or more methylene units are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)— or —S(O)₂—; and the ring formed by conneting R₄ with R_{A} is a ring structure fused with ring A;
V₃ is selected from a chemical bond, a C₁₋₁₀ alkylene group, a C₂₋₁₀ alkenylene group, a C₂₋₁₀ alkynylene group, and

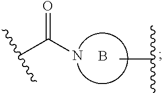

Ring B is a 3-7 membered heterocyclyl;
W₃ is selected from a chemical bond, —NR—, —O—, —S— and —C(O)—;
R* is independently selected from H, D, halogen, C₁₋₆ alkyl, and C₁₋₆ haloalkyl;
R is independently selected from H, C₁₋₆ alkyl, and C₁₋₆ haloalkyl;
Rₐ, R_{b}, and R_{c} are independently selected from H, C₁₋₆ alkyl, and C₁₋₆ haloalkyl; or R_{b}, R_{c} and the atom to which they are attached are taken together to form a 3-10 membered heterocyclyl group;
L₁ is a linker;
L₂ is an amino acid chain formed by connecting 2-8 amino acid residues, wherein each amino acid residue can be further substituted by one or more amino acid residues, and the amino acid residues are optionally substituted by 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl group, phosphate group and phosphite group;
Z is a chelating group derived from a chelating agent.

In another embodiment, the present disclosure provides a compound of formula (I), or an isotopic variant, a hydrate, an ester or solvate, a tautomer or a stereoisomer thereof, or a pharmaceutically acceptable salt thereof:

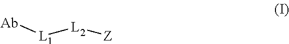

wherein,
Ab is a ligand targeting carbonic anhydrase IX, for example R₄

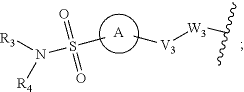

Ring A is selected from C₆₋₁₀ arylene and C₅₋₁₀ heteroarylene; wherein the C₆₋₁₀ arylene and C₅₋₁₀ heteroarylene are optionally substituted by 1, 2, 3 or 4 R_{A};
R_{A} is independently selected from H, D, halogen, CN, —OR, —SR, —NR_{b}R_{c}, C₁₋₆ alkyl, C₁₋₆ haloalkyl, C₃₋₁₀ cycloalkyl, 3-10 membered heterocyclyl, C₆₋₁₀ aryl and 5-10 membered heteroaryl;
R₃ and R₄ are independently selected from H, halogen, C₁₋₆ alkyl, C₁₋₆ haloalkyl, C₃₋₇ cycloalkyl and 3-7 membered heterocyclyl;
or R₄ and R_{A} are connected to form a C₁₋₄ alkylene, a C₂₋₄ alkenylene and a C₂₋₄ alkynylene, wherein one or more methylene units are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)— or —S(O)₂—; and the ring formed by conneting R₄ with R_{A} is a ring structure fused with ring A;
V₃ is selected from a chemical bond, a C₁₋₁₀ alkylene group, a C₂₋₁₀ alkenylene group, and a C₂₋₁₀ alkynylene group;
W₃ is selected from a chemical bond, —NR—, —O—, —S— and —C(O)—;
R* is independently selected from H, D, halogen, C₁₋₆ alkyl and C₁₋₆ haloalkyl;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-10 membered heterocyclyl group;
L₁ is a linker;
L₂ is an amino acid chain formed by connecting 2-8 amino acid residues, wherein each amino acid residue can be further substituted by one or more amino acid residues, and the amino acid residues are optionally substituted by 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl group, phosphate group and phosphite group;
Z is a chelating group derived from a chelating agent.

In another embodiment, the present disclosure provides the above compound, or an isotopic variant, a hydrate, an ester or solvate, a tautomer or a stereoisomer thereof, or a pharmaceutically acceptable salt thereof, having the following structure:

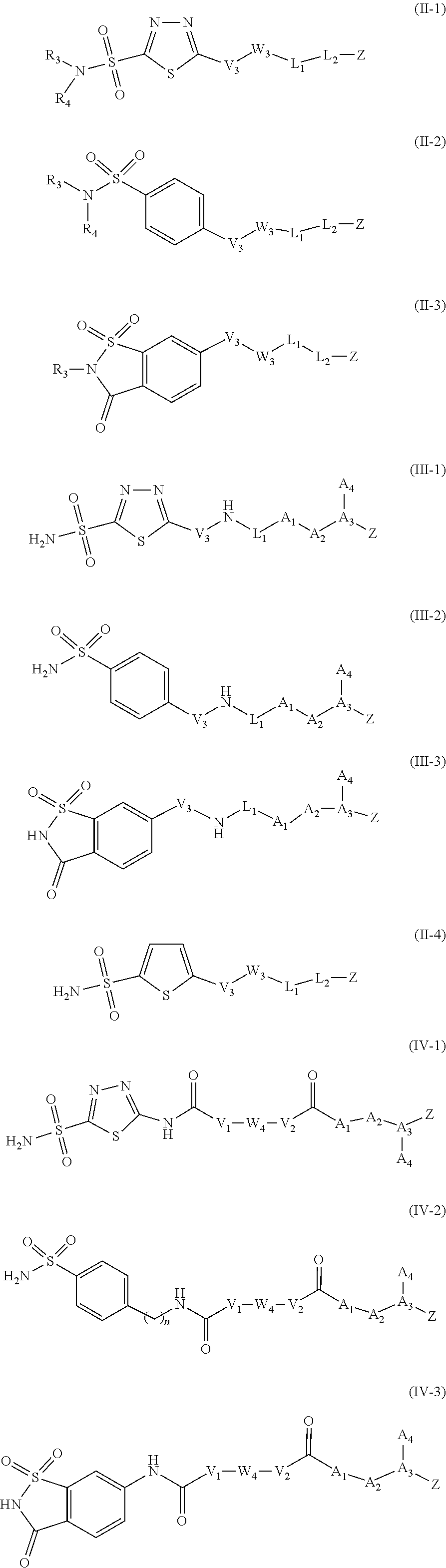

wherein,
A₁, A₂, and A₃ are amino acid residues;
A₄ is H or an amino acid residue;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted with 1, 2, 3, 4, 5 or 6 R₁ₛ;
R₁ₛ is independently selected from H, D, —OR, —SRₐ, —NR_{b}R_{c}, halogen, C₁₋₆ alkyl, C₁₋₆ haloalkyl, C₃₋₇ cycloalkyl and 3-7 membered heterocyclyl;
V₂ is a chemical bond or —(CR′R″)₁₋₆—;
R′ and R″ are independently selected from H, D, halogen, C₁₋₆ alkyl, C₁₋₆ haloalkyl and —C₁₋₆ alkylene-Ar;
Ar is independently selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
n is 0, 1, 2, 3, 4, 5 or 6;
the remaining variables are as defined herein.

In another embodiment, the present disclosure provides a compound comprising a compound of formula (I), or an isotopic variant, a hydrate, an ester or solvate, a tautomer, a stereoisomer, or a pharmaceutically acceptable salt thereof, and M complexed therewith,
wherein,
the compounds of formula (I) are as defined herein;
M is selected from at least one of radionuclides and non-radioactive elements.

In another embodiment, the present disclosure provides a compound of formula (V), or an isotopic variant, a hydrate, an ester or solvate, a tautomer or a stereoisomer thereof, or a pharmaceutically acceptable salt thereof:

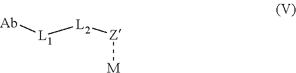

wherein,
Ab, L₁ and L₂ are as defined herein;
Z′ is a coordination group formed by complexing a chelating group Z derived from a chelating agent with M, wherein Z is as defined herein;
M is selected from at least one of radionuclides and non-radioactive elements.

In the compounds of the present disclosure, the variables can be defined as follows.

### Ab

In one embodiment, Ab is a ligand targeting carbonic anhydrase IX, for example

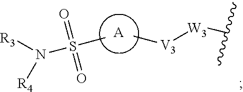

in another embodiment, Ab is

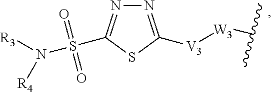

alternatively

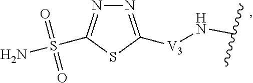

alternatively

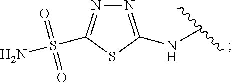 [SMILES: *Nc1nnc(S(N)(=O)=O)s1]

in another embodiment, Ab is

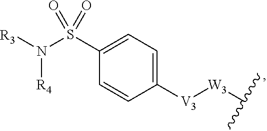

alternatively

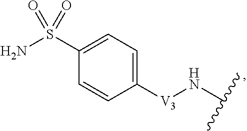

alternatively

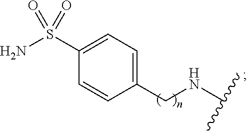

in another embodiment, Ab is

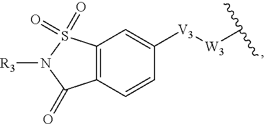

alternatively

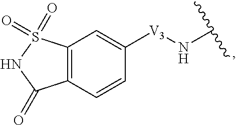

alternatively

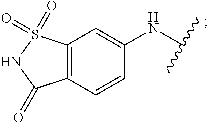 [SMILES: *Nc1ccc2c(c1)S(=O)(=O)NC2=O]

in another embodiment, Ab is

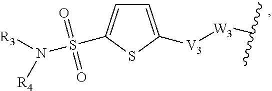

alternatively

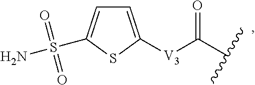

alternatively

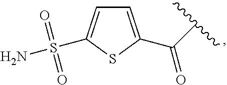 [SMILES: *C(=O)c1ccc(S(N)(=O)=O)s1]

alternatively

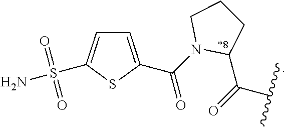 [SMILES: *C(=O)C1CCCN1C(=O)c1ccc(S(N)(=O)=O)s1]

In a more specific embodiment, Ab is selected from:

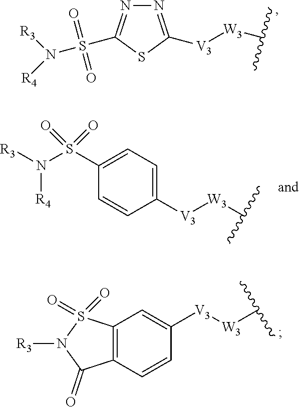

in another more specific embodiment, Ab is selected from:

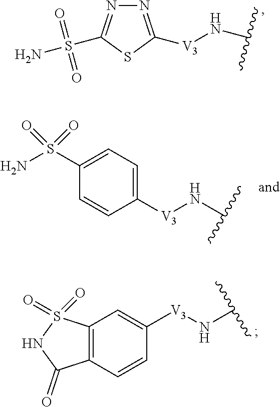

in another more specific embodiment, Ab is selected from:

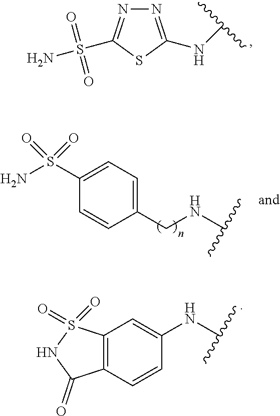

In a more specific embodiment, Ab is selected from:

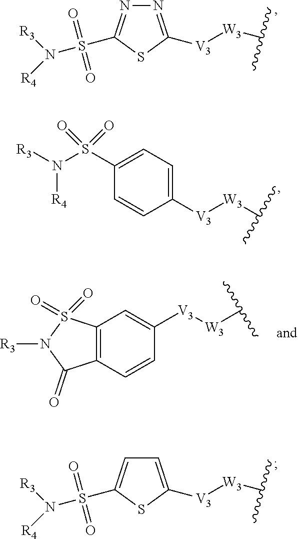

in another more specific embodiment, Ab is selected from:

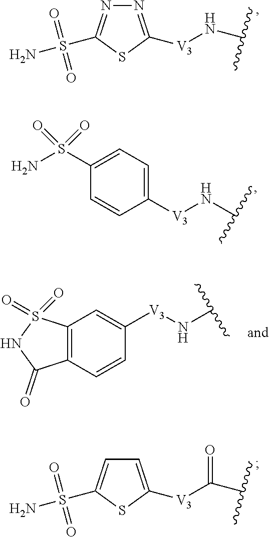

in another more specific embodiment, Ab is selected from:

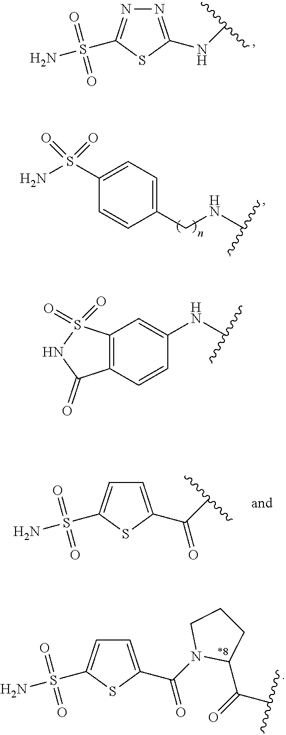

### Ring A

In one embodiment, ring A is a C₆₋₁₀ arylene group, alternatively a phenylene group; in another embodiment, ring A is a C₅₋₁₀ heteroarylene group, alternatively a C₅₋₆ heteroarylene group, such as a 1,3,4-thiadiazolylene group; in another embodiment, ring A is optionally substituted with 1, 2, 3 or 4 R_{A}; in another embodiment, ring A is unsubstituted.

In a more specific embodiment, ring A is selected from C₆₋₁₀ arylene and C₅₋₁₀ heteroarylene; in another more specific embodiment, ring A is selected from phenylene and C₅₋₆ heteroarylene; in another more specific embodiment, ring A is selected from phenylene and 1,3,4-thiadiazolylene.

### R_{A}

In one embodiment, R_{A} is H; in another embodiment, R_{A} is D; in another embodiment, R_{A} is halogen; in another embodiment, R_{A} is CN; in another embodiment, R_{A} is —ORₐ; in another embodiment, R_{A} is —SRₐ; in another embodiment, R_{A} is —NR_{b}R_{c}; in another embodiment, R_{A} is C₁₋₆ alkyl; in another embodiment, R_{A} is C₁₋₆ haloalkyl; in another embodiment, R_{A} is C₃₋₁₀ cycloalkyl, alternatively C₃₋₇ cycloalkyl; in another embodiment, R_{A} is 3-10 membered heterocyclyl, alternatively 3-7 membered heterocyclyl; in another embodiment, R_{A} is C₆₋₁₀ aryl, alternatively phenyl; in another embodiment, R_{A} is 5-10 membered heteroaryl, alternatively 5-6 membered heteroaryl.

In a more specific embodiment, R_{A} is independently selected from H, D, halogen, CN, —ORₐ, —SRₐ, —NR_{b}R_{c}, C₁₋₆ alkyl, C₁₋₆ haloalkyl, C₃₋₁₀ cycloalkyl, 3-10 membered heterocyclyl, C₆₋₁₀ aryl and 5-10 membered heteroaryl; in another more specific embodiment, R_{A} is independently selected from H, D, halogen, CN, —OR, —SRₐ, —NR_{b}R_{c}, C₁₋₆ alkyl, C₁₋₆ haloalkyl, C₃₋₇ cycloalkyl and 3-7 membered heterocyclyl; in another more specific embodiment, R_{A} is independently selected from H, D, halogen, C₁₋₆ alkyl and C₁₋₆ haloalkyl.

R₃ and R₄

In one embodiment, R₃ is H; in another embodiment, R₃ is halogen; in another embodiment, R₃ is C₁₋₆ alkyl; in another embodiment, R₃ is C₁₋₆ haloalkyl; in another embodiment, R₃ is C₃₋₇ cycloalkyl; in another embodiment, R₃ is 3-7 membered heterocyclyl.

In one embodiment, R₄ is H; in another embodiment, R₄ is halogen; in another embodiment, R₄ is C₁₋₆ alkyl; in another embodiment, R₄ is C₁₋₆ haloalkyl; in another embodiment, R₄ is C₃₋₇ cycloalkyl; in another embodiment, R₄ is 3-7 membered heterocyclyl.

In a more specific embodiment, R₃ is independently selected from H, halogen, C₁₋₆ alkyl, C₁₋₆ haloalkyl, C₃₋₇ cycloalkyl and 3-7 membered heterocyclyl; in another more specific embodiment, R₃ is independently selected from H, halogen, C₁₋₆ alkyl and C₁₋₆ haloalkyl.

In a more specific embodiment, R₃ is independently selected from H, halogen, C₁₋₆ alkyl, C₁₋₆ haloalkyl, C₃₋₇ cycloalkyl and 3-7 membered heterocyclyl; in another more specific embodiment, R₄ is independently selected from H, halogen, C₁₋₆ alkyl and C₁₋₆ haloalkyl.

In one embodiment, R₄ and R_{A} are connected to form a C₁₋₄ alkylene group, alternatively a C₁₋₂ alkylene group; in another embodiment, R₄ and R_{A} are connected to form a C₂₋₄ alkenylene group; in another embodiment, R₄ and R_{A} are connected to form a C₂₋₄ alkynylene group; in another embodiment, one or more (alternatively 1, 2 or 3, alternatively 1) methylene units in the group formed by connecting R₄ with R_{A} are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)— or —S(O)₂—; in another embodiment, one or more (alternatively 1, 2 or 3, alternatively 1) methylene units in the group formed by connecting R₄ with R_{A} are optionally and independently replaced by —CR*₂—, —NR*—, —O—, —C(O)—, —OC(O)—, —C(O)O—, —S—, —S(O)— or —S(O)₂—; in another embodiment, one or more (alternatively 1, 2 or 3, alternatively 1) methylene units in the group formed by connecting R₄ with R_{A} are optionally and independently replaced by —CR*₂—, —C(O)—, —OC(O)— or —C(O)O— (alternatively —C(O)—); in another embodiment, the ring formed by connecting R₄ with R_{A} is a ring structure fused with ring A.

In a more specific embodiment, R₄ and R_{A} are connected to form C₁₋₄ alkylene, C₂₋₄ alkenylene and C₂₋₄ alkynylene, wherein one or more methylene units are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)₂—, or —S(O)₂—; in another more specific embodiment, R₄ and R_{A} are connected to form C₁₋₄ alkylene, alternatively C₁₋₂ alkylene, wherein 1, 2 or 3 (alternatively 1) methylene units are optionally and independently replaced by —CR*₂—, —NR*—, —O—, —C(O)—, —OC(O)—, —C(O)O—, —S—, —S(O)— or —S(O)₂—, alternatively replaced by —CR*₂—, —C(O)—, —OC(O)— or —C(O)O—, alternatively replaced by —C(O)—; in another more specific embodiment, the ring formed by connecting R₄ with R_{A} is a ring structure fused with ring A.

### V₃ and Ring B

In one embodiment, V₃ is a chemical bond; in another embodiment, V₃ is C₁₋₁₀ alkylene, alternatively C₁₋₆ alkylene, alternatively C₁₋₄ alkylene, alternatively C₁₋₂ alkylene, such as —CH₂CH₂—, such as methylene; in another embodiment, V₃ is C₂₋₁₀ alkenylene, alternatively C₂₋₆ alkenylene; in another embodiment, V₃ is C₂₋₁₀ alkynylene, alternatively C₂₋₆ alkynylene; in another embodiment, V₃ is

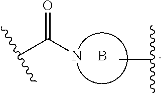

In one embodiment, ring B is a 3-7 membered heterocyclylene group; in another embodiment, ring B is a 4-6 membered heterocyclylene group; in another embodiment, ring B is a 5 membered heterocyclylene group; in another embodiment, ring B is

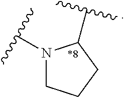 [SMILES: *C1CCCN1*]

In a more specific embodiment, V₃ is selected from a chemical bond, a C₁₋₁₀ alkylene group, a C₂₋₁₀ alkenylene group and a C₂₋₁₀ alkynylene group; in another more specific embodiment, V₃ is selected from a chemical bond, a C₁₋₆ alkylene group, a C₂₋₆ alkenylene group and a C₂₋₆ alkynylene group; in another more specific embodiment, V₃ is selected from a chemical bond and a C₁₋₆ alkylene group (alternatively a C₁₋₄ alkylene group, alternatively a C₁₋₂ alkylene group).

In a more specific embodiment, V₃ is selected from a chemical bond, a C₁₋₆ alkylene group, a C₂₋₆ alkenylene group, a C₂₋₆ alkynylene group and

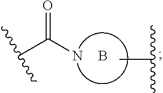

in another more specific embodiment, V₃ is selected from a chemical bond, a C₁₋₆ alkylene group (alternatively a C₁₋₄ alkylene group, alternatively a C₁₋₂ alkylene group), and

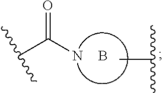

in another more specific embodiment, V₃ is selected from a chemical bond and

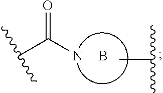

### W₃

In one embodiment, W₃ is a chemical bond; in another embodiment, W₃ is —NR—, alternatively —NH—; in another embodiment, W₃ is —O—; in another embodiment, W₃ is —S—; in another embodiment, W₃ is —C(O)—.

In a more specific embodiment, W₃ is selected from a chemical bond, —NR—, —O—, —S— and —C(O)—; in another more specific embodiment, W₃ is selected from —NR— and —O—.

In a more specific embodiment, W₃ is selected from —NR—, —O— and —C(O)—; in another more specific embodiment, W₃ is selected from —NR— and —O—.

### R*

In one embodiment, R* is H; in another embodiment, R* is D; in another embodiment, R* is halogen; in another embodiment, R* is C₁₋₆ alkyl; in another embodiment, R* is C₁₋₆ haloalkyl.

In a more specific embodiment, R* is independently selected from H, D, halogen, C₁₋₆ alkyl and C₁₋₆ haloalkyl; in another more specific embodiment, R* is independently selected from H, D, halogen, C₁₋₆ alkyl and C₁₋₆ haloalkyl.

### R

In one embodiment, R is H; in another embodiment, R is C₁₋₆ alkyl; in another embodiment, R is C₁₋₆ haloalkyl.

In a more specific embodiment, R is independently selected from H, C₁₋₆ alkyl and C₁₋₆ haloalkyl.

n

In one embodiment, n is 0; in another embodiment, n is 1; in another embodiment, n is 2; in another embodiment, n is 3; in another embodiment, n is 4; in another embodiment, n is 5; in another embodiment, n is 6.

In a more specific embodiment, n is 0, 1, 2, 3, 4, 5 or 6; in another more specific embodiment, n is 0, 1, 2, 3 or 4; in another more specific embodiment, n is 0, 1 or 2; in another more specific embodiment, n is 0 or 2.

Rₐ, R_{b} and R_{c}

In one embodiment, Rₐ is H; in another embodiment, Rₐ is C₁₋₆ alkyl; in another embodiment, Rₐ is C₁₋₆ haloalkyl.

In one embodiment, R_{b} is H; in another embodiment, R_{b} is C₁₋₆ alkyl; in another embodiment, R_{b} is C₁₋₆ haloalkyl.

In one embodiment, R_{c} is H; in another embodiment, R_{c} is C₁₋₆ alkyl; in another embodiment, R_{c} is C₁₋₆ haloalkyl.

In a more specific embodiment, Rₐ, R_{b} and R_{c} are independently selected from H, C₁₋₆ alkyl and C₁₋₆ haloalkyl.

In one embodiment, R_{b}, R_{c} and the atom to which they are attached together form a 3-10 membered heterocyclyl group, alternatively a 3-7 membered heterocyclyl group.

### L₁

In one embodiment, L₁ is a linker; in another embodiment, L₁ is —W₁—V₁—W₄—V₂—W₂—; in another embodiment, L₁ is —C(O)—V₁—W₄—V₂—C(O)—; in another embodiment, L₁ is

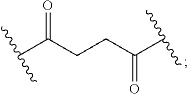 [SMILES: *C(=O)CCC(*)=O]

in another embodiment, L₁ is

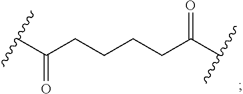 [SMILES: *C(=O)CCCCC(*)=O]

in another embodiment, L₁ is

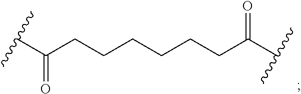 [SMILES: *C(=O)CCCCCCC(*)=O]

in another embodiment, L₁ is

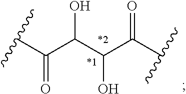 [SMILES: *C(=O)C(O)C(O)C(*)=O]

in another embodiment, L₁ is

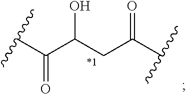 [SMILES: *C(=O)CC(O)C(*)=O]

in another embodiment, L₁ is

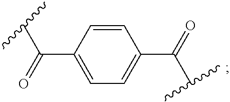 [SMILES: *C(=O)c1ccc(C(*)=O)cc1]

in another embodiment, L₁ is

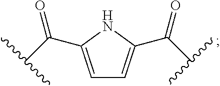 [SMILES: *C(=O)c1ccc(C(*)=O)[nH]1]

in another embodiment, L₁ is

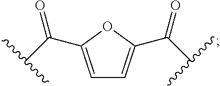 [SMILES: *C(=O)c1ccc(C(*)=O)o1]

in another embodiment, L₁ is

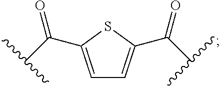 [SMILES: *C(=O)c1ccc(C(*)=O)s1]

in another embodiment, L₁ is

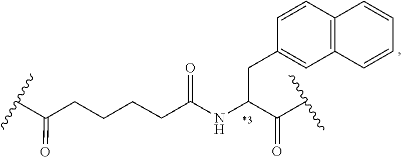 [SMILES: *C(=O)CCCCC(=O)NC(Cc1ccc2ccccc2c1)C(*)=O]

alternatively

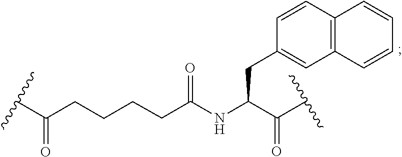 [SMILES: *C(=O)CCCCC(=O)N[C@@H](Cc1ccc2ccccc2c1)C(*)=O]

in another embodiment, L₁ is

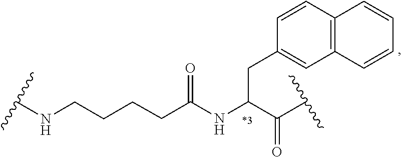 [SMILES: *NCCCCC(=O)NC(Cc1ccc2ccccc2c1)C(*)=O]

alternatively

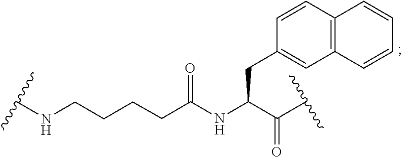 [SMILES: *NCCCCC(=O)N[C@@H](Cc1ccc2ccccc2c1)C(*)=O]

in another embodiment, L₁ is

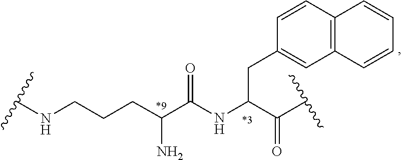 [SMILES: *NCCCC(N)C(=O)NC(Cc1ccc2ccccc2c1)C(*)=O]

alternatively

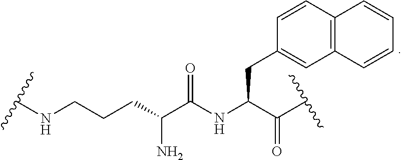 [SMILES: *NCCC[C@@H](N)C(=O)N[C@@H](Cc1ccc2ccccc2c1)C(*)=O]

In a more specific embodiment, L₁ is selected from:

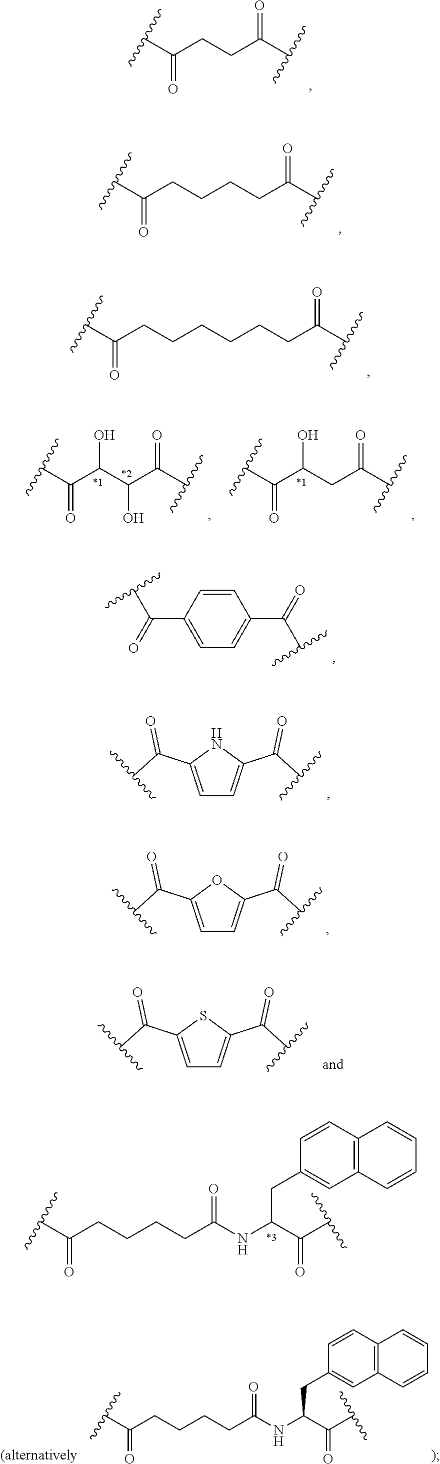

in another more specific embodiment, L₁ is

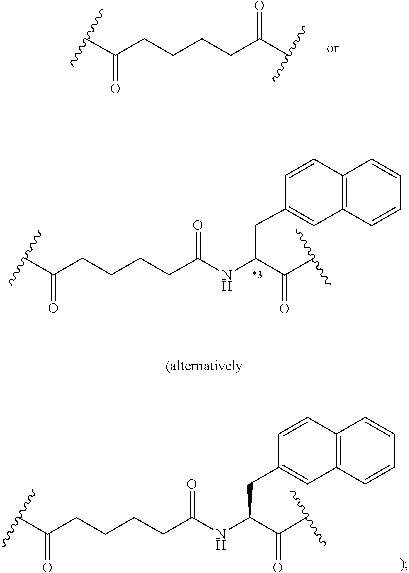 [SMILES: *C(=O)CCCCC(*)=O ; *C(=O)CCCCC(=O)NC(Cc1ccc2ccccc2c1)C(*)=O ; *C(=O)CCCCC(=O)N[C@@H](Cc1ccc2ccccc2c1)C(*)=O]

in another more specific embodiment, L₁ is

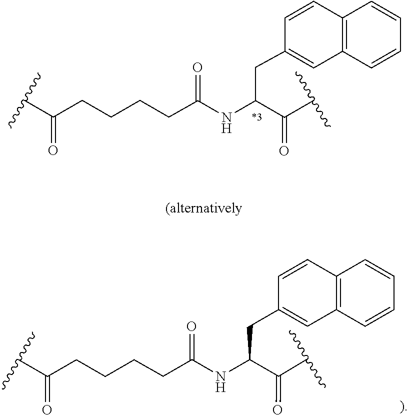 [SMILES: *C(=O)CCCCC(=O)NC(Cc1ccc2ccccc2c1)C(*)=O ; *C(=O)CCCCC(=O)N[C@@H](Cc1ccc2ccccc2c1)C(*)=O]

In a more specific embodiment, L₁ is selected from:

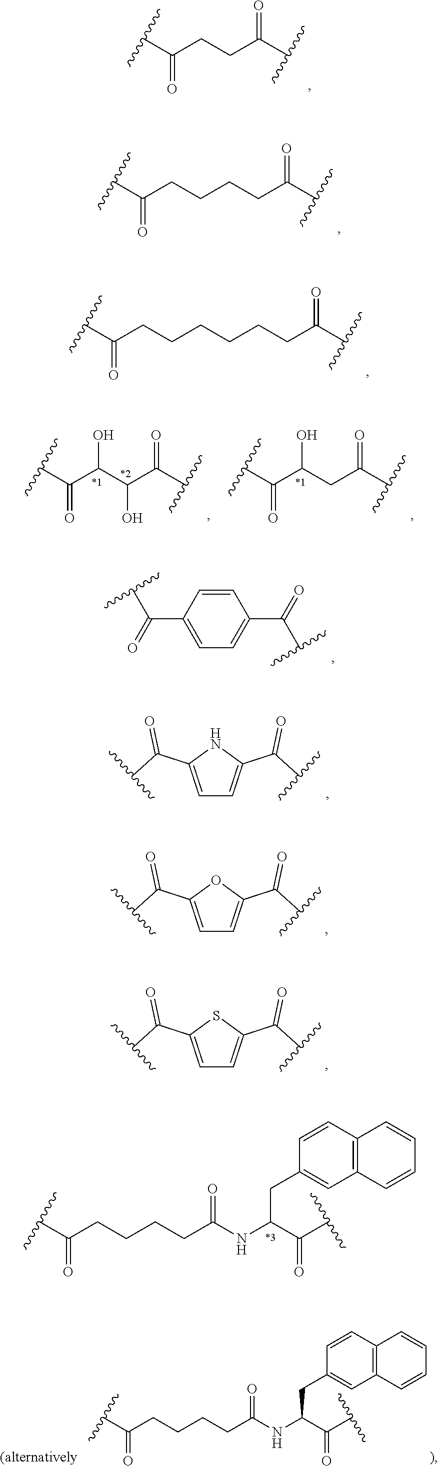

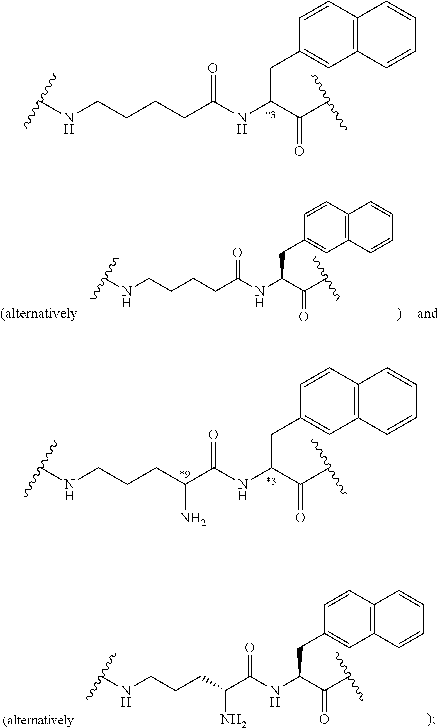 [SMILES: *NCCCCC(=O)NC(Cc1ccc2ccccc2c1)C(*)=O ; *NCCCCC(=O)N[C@@H](Cc1ccc2ccccc2c1)C(*)=O ; *NCCCC(N)C(=O)NC(Cc1ccc2ccccc2c1)C(*)=O ; *NCCC[C@@H](N)C(=O)N[C@@H](Cc1ccc2ccccc2c1)C(*)=O]

in a more specific embodiment, L₁ is

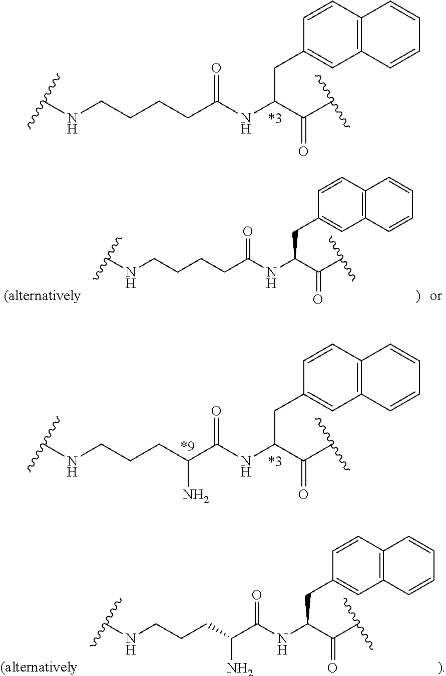

### W₁

In one embodiment, W₁ is a chemical bond; in another embodiment, W₁ is —NR—; in another embodiment, W₁ is —C(O)—; in another embodiment, W₁ is —C(O)O—; in another embodiment, W₁ is —C(O)NR—.

In a more specific embodiment, W₁ is selected from a chemical bond, —NR—, —C(O)—, —C(O)O— and —C(O)NR—; in another more specific embodiment, W₁ is selected from —C(O)—, —C(O)O— and —C(O)NR—.

In a more specific embodiment, W₁ is selected from —NR—, —C(O)—, —C(O)O— and —C(O)NR—; in another more specific embodiment, W₁ is selected from —NR— and —C(O)—.

### W₂

In one embodiment, W₂ is —C(O)—; in another embodiment, W₂ is —OC(O)—; in another embodiment, W₂ is —NRC(O)—; in another embodiment, W₂ is —NR—; in another embodiment, W₂ is —C(O)NR—; in another embodiment, W₂ is —O—.

In a more specific embodiment, W₂ is selected from —C(O)—, —OC(O)—, —NRC(O)—, —NR—, —C(O)NR—, and —O—; in another more specific embodiment, W₂ is selected from —C(O)—, —OC(O)—, and —NRC(O)—.

### V₁ and m

In one embodiment, V₁ is C₁₋₁₀ alkylene, alternatively C₁₋₈ alkylene, alternatively C₁₋₆ alkylene, alternatively C₁₋₄ alkylene, such as —(CH₂)ₘ—; in another embodiment, V₁ is C₂₋₁₀ alkenylene, alternatively C₂₋₈ alkenylene; in another embodiment, V₁ is C₂₋₁₀ alkynylene, alternatively C₂₋₈ alkynylene; in another embodiment, V₁ is C₆₋₁₀ arylene, alternatively phenylene; in another embodiment, V₁ is C₅₋₁₀ heteroarylene, alternatively C₅₋₆ heteroarylene; in another embodiment, V₁ is optionally substituted by 1, 2, 3, 4, 5 or 6 R₁ₛ; in another embodiment, V₁ is unsubstituted; in another embodiment, one or more (alternatively 1, 2 or 3) methylene units in the alkylene, alkenylene and alkynylene groups are optionally and independently replaced by —NR—, —NRC(O)—, —C(O)NR—, —NRS(O)₂—, —S(O)₂NR—, —O—, —C(O)—, —OC(O)—, —C(O)O—, —S—, —S(O)— or —S(O)₂—; in another embodiment, one or more (alternatively 1, 2 or 3) methylene units in the alkylene, alkenylene and alkynylene groups are optionally and independently replaced by —NR—, —NRC(O)—, —C(O)NR—, —C(O)—, —OC(O)— or —C(O)O—.

In a more specific embodiment, V₁ is selected from C₁₋₁₀ alkylene, C₂₋₁₀ alkenylene, C₂₋₁₀ alkynylene, C₆₋₁₀ arylene and C₅₋₁₀ heteroarylene; in another more specific embodiment, V₁ is selected from C₁₋₈ alkylene, C₂₋₈ alkenylene, C₂₋₈ alkynylene, phenylene and C₅₋₆ heteroarylene; in another more specific embodiment, V₁ is selected from C₁₋₆ alkylene, C₂₋₆ alkenylene and C₂₋₆ alkynylene; in another more specific embodiment, V₁ is selected from C₁₋₆ alkylene (e.g., —(CH₂)ₘ—), phenylene and C₅₋₆ heteroarylene; in another more specific embodiment, V₁ is selected from C₁₋₆ alkylene (e.g., —(CH₂)ₘ—), phenylene, pyrrolylene, furanylene or thienylene; in another more specific embodiment, one or more methylene units in the alkylene, alkenylene and alkynylene groups are optionally and independently replaced by —NR—, —NRC(O)—, —C(O)NR—, —NRS(O)₂—, —S(O)₂NR—, —O—, —C(O)—, —OC(O)—, —C(O)O—, —S—, —S(O)— or —S(O)₂—; in another more specific embodiment, 1, 2 or 3 methylene units in the alkylene, alkenylene and alkynylene groups are optionally and independently replaced by —NR—, —NRC(O)—, —C(O)NR—, —C(O)—, —OC(O)— or —C(O)O—.

In one embodiment, m is 1; in another embodiment, m is 2; in another embodiment, m is 3; in another embodiment, m is 4; in another embodiment, m is 5; in another embodiment, m is 6; in another embodiment, m is 7; in another embodiment, m is 8.

In a more specific embodiment, m is independently selected from 1, 2, 3, 4, 5, 6, 7 or 8; in another more specific embodiment, m is independently selected from 1, 2, 3, 4, 5 or 6; in another more specific embodiment, m is independently selected from 1, 2, 3 or 4.

### R₁ₛ

In one embodiment, R₁ₛ is H; in another embodiment, R₁ₛ is D; in another embodiment, R₁ₛ is —ORₐ; in another embodiment, R₁ₛ is —SRₐ; in another embodiment, R₁ₛ is —NR_{b}R_{c}; in another embodiment, R₁ₛ is halogen; in another embodiment, R₁ₛ is C₁₋₆ alkyl; in another embodiment, R₁ₛ is C₁₋₆ haloalkyl; in another embodiment, R₁ₛ is C₃₋₇ cycloalkyl; in another embodiment, R₁ₛ is a 3-7 membered heterocyclyl; in another embodiment, R₁ₛ is in (S) configuration; in another embodiment, R₁ₛ is in (R) configuration; in another embodiment, R₁ₛ is in racemic form.

In a more specific embodiment, R₁ₛ is independently selected from H, D, —OR, —SR, —NR_{b}R_{c}, halogen, C₁₋₆ alkyl, C₁₋₆ haloalkyl, C₃₋₇ cycloalkyl or 3-7 membered heterocyclyl; in another more specific embodiment, R₁ₛ is independently selected from H, D, —ORₐ, —NR_{b}R_{c}, halogen, C₁₋₆ alkyl and C₁₋₆ haloalkyl; in another more specific embodiment, R₁ₛ is independently selected from H, D, —ORₐ and —NR_{b}R_{c}; in another more specific embodiment, R₁ₛ is independently selected from H, D, —OR, C₁₋₆ alkyl and C₁₋₆ haloalkyl; in another more specific embodiment, R₁ₛ is independently selected from H and —ORₐ; in another more specific embodiment, R₁ₛ is independently selected from H and D; in another more specific embodiment, R₁ₛ is independently in (S) or (R) configuration, or is in racemic form.

In a more specific embodiment, R₁ₛ is independently selected from H, —OR, —NR_{b}R_{c}, C₁₋₆ alkyl, and C₁₋₆ haloalkyl; in another more specific embodiment, R₁ₛ is independently selected from H, —ORₐ and —NR_{b}R_{c}; in another more specific embodiment, R₁ₛ is independently selected from H and —NR_{b}R_{c}.

### V₂

In one embodiment, V₂ is a chemical bond; in another embodiment, V₂ is —(CR′R″)₁₋₆—, for example —CR′R″—, for example —(CR′R″)₂—, for example —(CR′R″)₃—, for example —(CR′R″)₄—, for example —(CR′R″)₅—, for example —(CR′R″)₆—, alternatively —(CR′R″)₁₋₄—, alternatively —(CR′R″)₁₋₂—, alternatively —CHR′—.

In a more specific embodiment, V₂ is a chemical bond or —(CR′R″)₁₋₆—; in another more specific embodiment, V₂ is a chemical bond or —(CR′R″)₁₋₄—; in another more specific embodiment, V₂ is a chemical bond or —(CR′R″)₁₋₂—; in another more specific embodiment, V₂ is a chemical bond or —CHR′—.

### R′, R″ and Ar

In one embodiment, R′ is H; in another embodiment, R′ is D; in another embodiment, R′ is halogen; in another embodiment, R′ is C₁₋₆ alkyl; in another embodiment, R′ is C₁₋₆ haloalkyl; in another embodiment, R′ is —C₁₋₆ alkylene-Ar, alternatively —C₁₋₄ alkylene-Ar, alternatively —CH₂—Ar; in another embodiment, R′ is in (S) configuration; in another embodiment, R′ is in (R) configuration; in another embodiment, R′ is in racemic form.

In one embodiment, R″ is H; in another embodiment, R″ is D; in another embodiment, R″ is halogen; in another embodiment, R″ is C₁₋₆ alkyl; in another embodiment, R″ is C₁₋₆ haloalkyl; in another embodiment, R″ is —C₁₋₆ alkylene-Ar, alternatively —C₁₋₄ alkylene-Ar, alternatively —CH₂—Ar; in another embodiment, R″ is in (S) configuration; in another embodiment, R″ is in (R) configuration; in another embodiment, R″ is in racemic form.

In a more specific embodiment, R′ is independently selected from H, D, halogen, C₁₋₆ alkyl, C₁₋₆ haloalkyl and —C₁₋₆ alkylene-Ar; in another more specific embodiment, R′ is independently selected from H, D or —C₁₋₄ alkylene-Ar; in another more specific embodiment, R′ is independently selected from H or —C₁₋₄ alkylene-Ar; in another more specific embodiment, R′ is independently H or —CH₂—Ar; in another more specific embodiment, R′ is independently in (S) or (R) configuration, or is in racemic form.

In a more specific embodiment, R″ is independently selected from H, D, halogen, C₁₋₆ alkyl, C₁₋₆ haloalkyl and —C₁₋₆ alkylene-Ar; in another more specific embodiment, R″ is independently selected from H, D, halogen, C₁₋₆ alkyl and C₁₋₆ haloalkyl; in another more specific embodiment, R″ is independently selected from H, D, C₁₋₆ alkyl and C₁₋₆ haloalkyl; in another more specific embodiment, R″ is independently H or D.

In one embodiment, Ar is a C₆₋₁₄ aryl group, such as phenyl, naphthyl, anthracenyl or phenanthryl, alternatively a C₆₋₁₀ aryl group, such as phenyl or naphthyl, alternatively naphthyl, alternatively 2-naphthyl; in another embodiment, Ar is a 5-14 membered heteroaryl group, alternatively a 5-10 membered heteroaryl group.

In a more specific embodiment, Ar is independently selected from C₆₋₁₄ aryl and 5-14 membered heteroaryl; in another more specific embodiment, Ar is C₆₋₁₀ aryl, such as phenyl or naphthyl, alternatively naphthyl, alternatively 2-naphthyl; in another more specific embodiment, Ar is independently selected from C₆₋₁₀ aryl and 5-10 membered heteroaryl.

### W₄

In one embodiment, W₄ is a chemical bond; in another embodiment, W₄ is —C(O)—; in another embodiment, W₄ is —OC(O)—; in another embodiment, W₄ is —NRC(O)—; in another embodiment, W₄ is —NR—; in another embodiment, W₄ is —C(O)O—; in another embodiment, W₄ is —C(O)NR—, alternatively —C(O)NH—; in another embodiment, W₄ is —O—.

In a more specific embodiment, W₄ is selected from a chemical bond, —C(O)—, —OC(O)—, —NRC(O)—, —NR—, —C(O)O—, —C(O)NR— and —O—; in another more specific embodiment, W₄ is selected from a chemical bond, —OC(O)—, —NRC(O)—, —C(O)O— and —C(O)NR—; in another more specific embodiment, W₄ is selected from a chemical bond, —C(O)O— and —C(O)NR—; in another more specific embodiment, W₄ is a chemical bond or —C(O)NR—.

### L₂

In one embodiment, L₂ is an amino acid chain formed by connecting 2-8 (alternatively 2-5) amino acid residues; in another embodiment, each amino acid residue in L₂ is optionally further substituted by 1 or more (alternatively 1, 2 or 3, alternatively 1) amino acid residues; in another embodiment, each amino acid residue in L₂ is not substituted; in another embodiment, L₂ is

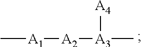

in another embodiment, the amino acid residues in L₂ are optionally substituted by 1, 2, 3, 4, 5 or 6 (alternatively 1, 2, 3 or 4) R_{L}; in another embodiment, the amino acid residues in L₂ are not substituted.

In another embodiment, L₂ is

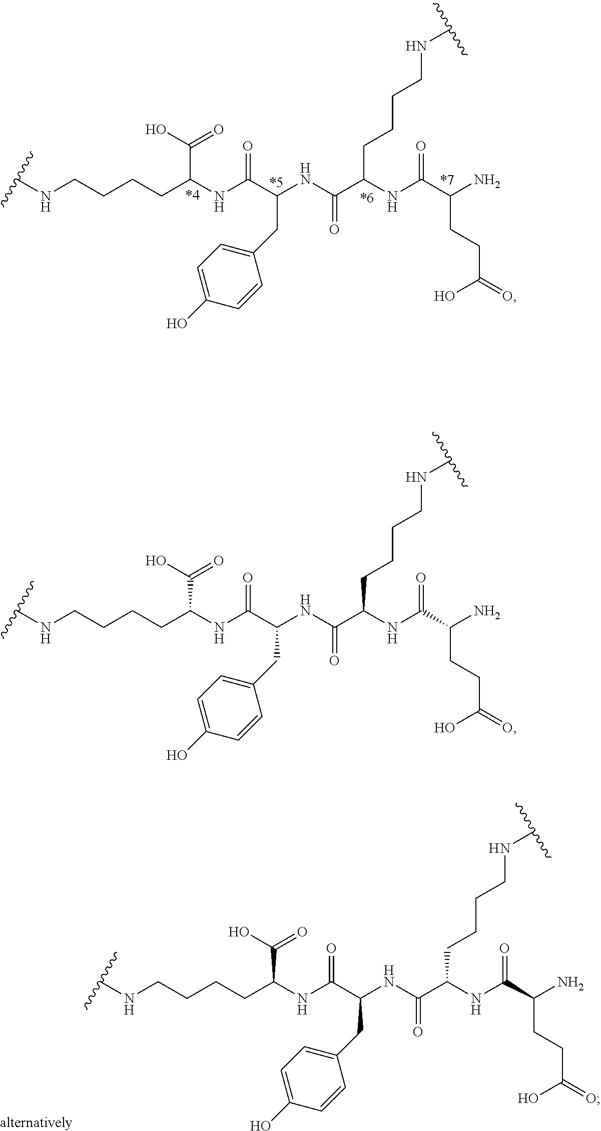 [SMILES: *NCCCCC(NC(=O)C(Cc1ccc(O)cc1)NC(=O)C(CCCCN*)NC(=O)C(N)CCC(=O)O)C(=O)O ; *NCCCC[C@@H](NC(=O)[C@@H](Cc1ccc(O)cc1)NC(=O)[C@@H](CCCCN*)NC(=O)[C@H](N)CCC(=O)O)C(=O)O ; *NCCCC[C@H](NC(=O)[C@H](Cc1ccc(O)cc1)NC(=O)[C@H](CCCCN*)NC(=O)[C@@H](N)CCC(=O)O)C(=O)O]

alternatively in another embodiment, L₂ is

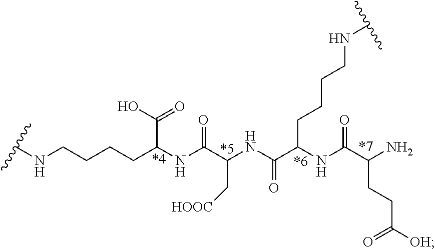 [SMILES: *NCCCCC(NC(=O)C(CC(=O)O)NC(=O)C(CCCCN*)NC(=O)C(N)CCC(=O)O)C(=O)O]

in another embodiment, L₂ is

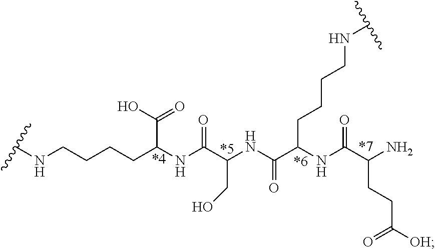 [SMILES: *NCCCCC(NC(=O)C(CO)NC(=O)C(CCCCN*)NC(=O)C(N)CCC(=O)O)C(=O)O]

in another embodiment, L₂ is

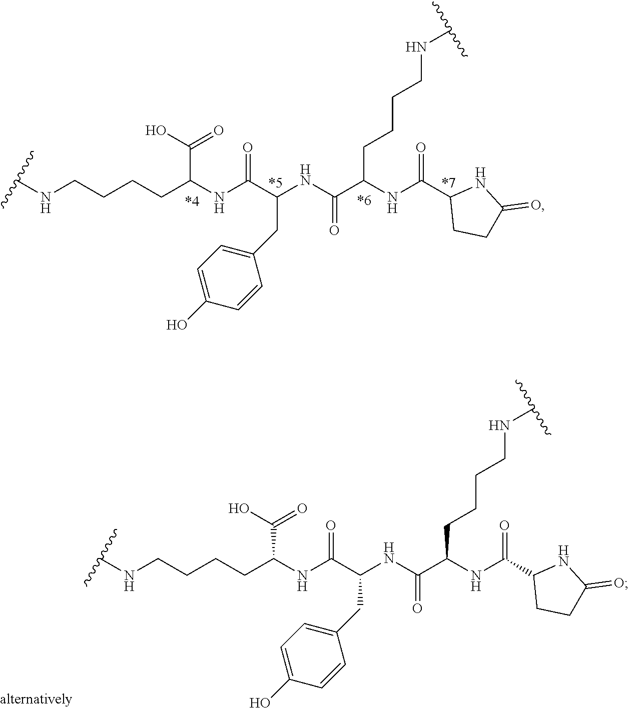

in another embodiment, L₂ is

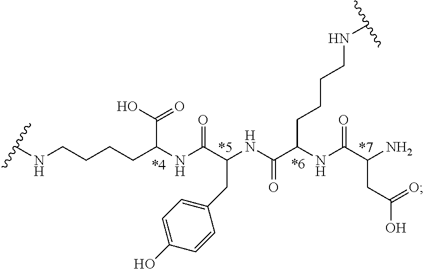 [SMILES: *NCCCCC(NC(=O)C(Cc1ccc(O)cc1)NC(=O)C(CCCCN*)NC(=O)C(N)CC(=O)O)C(=O)O]

in another embodiment, L₂ is

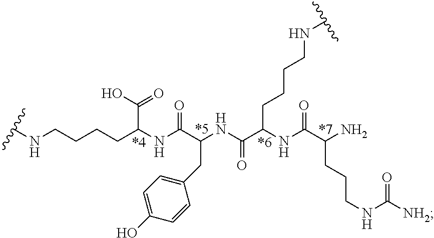 [SMILES: *NCCCCC(NC(=O)C(Cc1ccc(O)cc1)NC(=O)C(CCCCN*)NC(=O)C(N)CCCNC(N)=O)C(=O)O]

in another embodiment, L₂ is

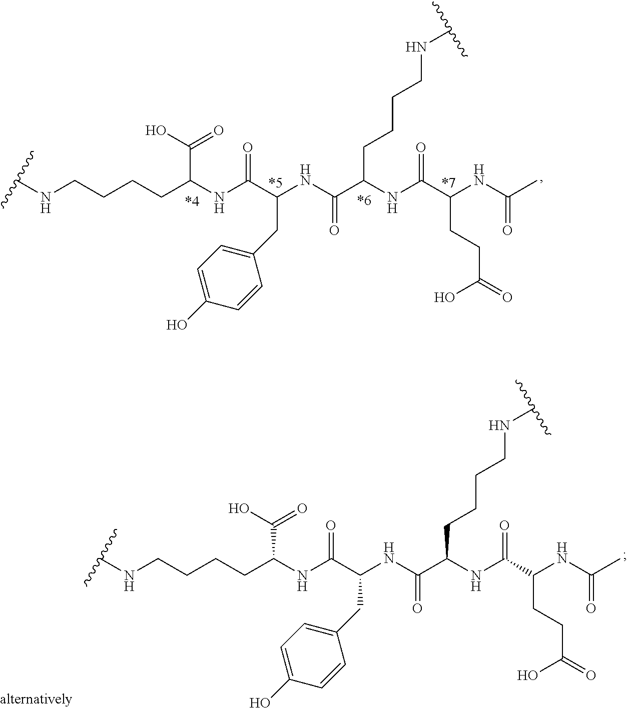

in another embodiment, L₂ is

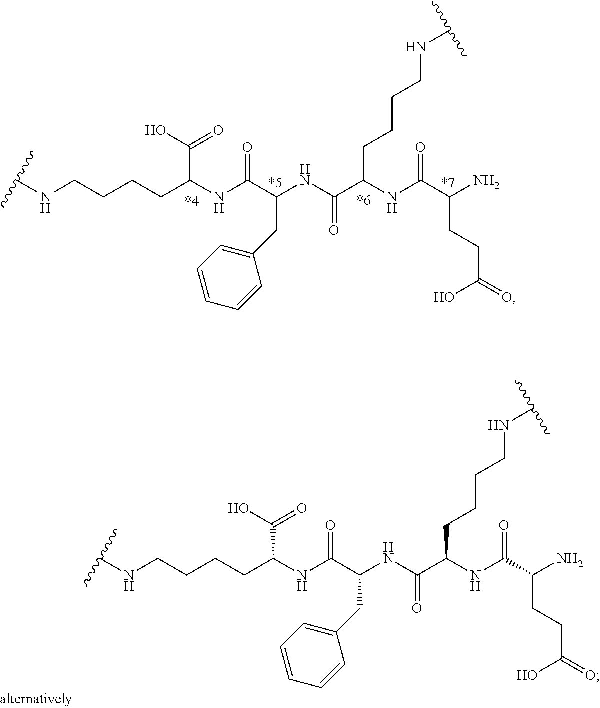 [SMILES: *NCCCCC(NC(=O)C(Cc1ccccc1)NC(=O)C(CCCCN*)NC(=O)C(N)CCC(=O)O)C(=O)O ; *NCCCC[C@@H](NC(=O)[C@@H](Cc1ccccc1)NC(=O)[C@@H](CCCCN*)NC(=O)[C@H](N)CCC(=O)O)C(=O)O]

in another embodiment, L₂ is

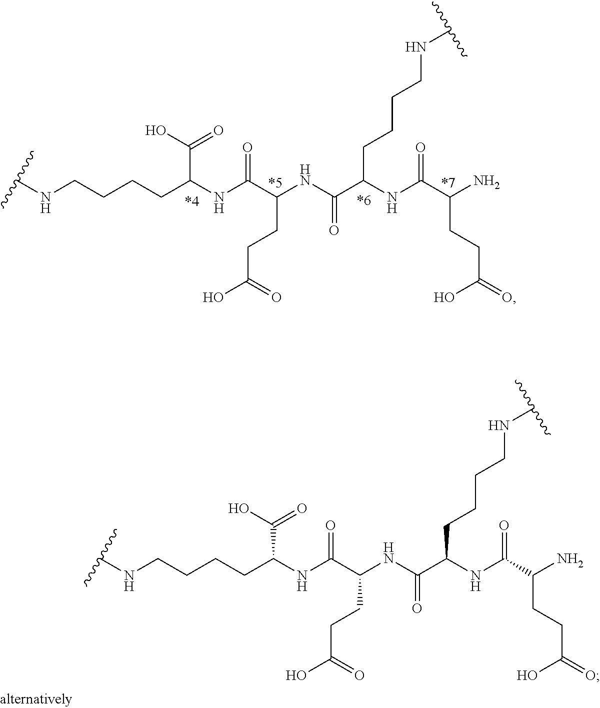

in another embodiment, L₂ is

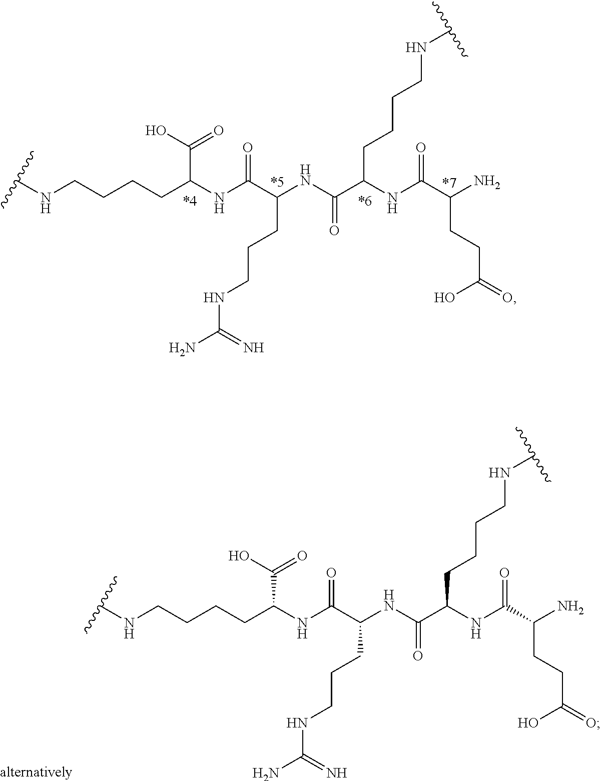 [SMILES: *NCCCCC(NC(=O)C(CCCNC(=N)N)NC(=O)C(CCCCN*)NC(=O)C(N)CCC(=O)O)C(=O)O ; *NCCCC[C@@H](NC(=O)[C@@H](CCCNC(=N)N)NC(=O)[C@@H](CCCCN*)NC(=O)[C@H](N)CCC(=O)O)C(=O)O]

in another embodiment, L₂ is

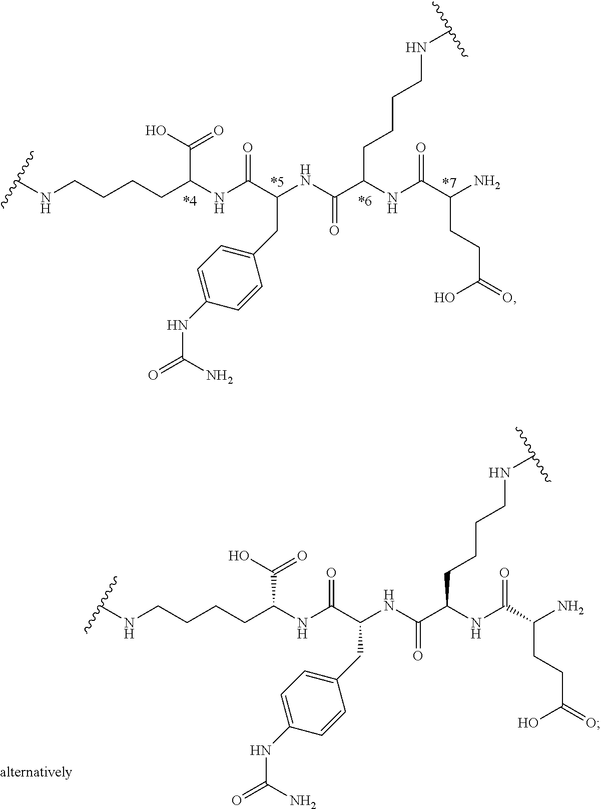 [SMILES: *NCCCCC(NC(=O)C(Cc1ccc(NC(N)=O)cc1)NC(=O)C(CCCCN*)NC(=O)C(N)CCC(=O)O)C(=O)O ; *NCCCC[C@@H](NC(=O)[C@@H](Cc1ccc(NC(N)=O)cc1)NC(=O)[C@@H](CCCCN*)NC(=O)[C@H](N)CCC(=O)O)C(=O)O]

in another embodiment, L₂ is

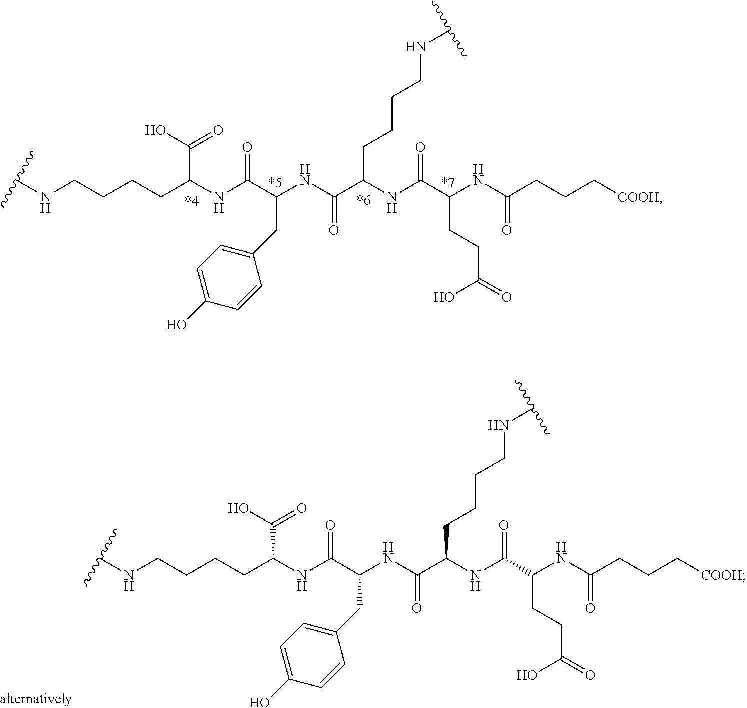

in another embodiment, L₂ is

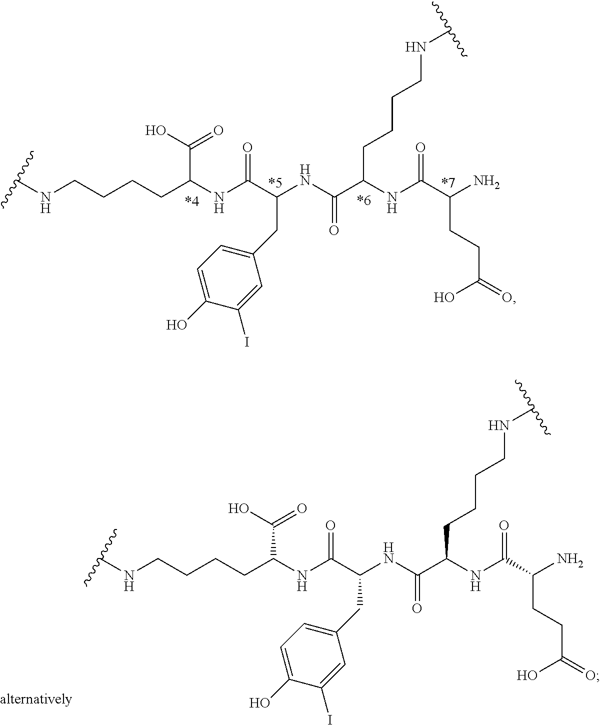

In a more specific embodiment, L₂ is an amino acid chain formed by connecting 2-8 amino acid residues, wherein each amino acid residue is optionally further substituted by 1 or more amino acid residues, and the amino acid residue is optionally substituted by 1, 2, 3, 4, 5 or 6 R_{L}; in another more specific embodiment, L₂ is an amino acid chain formed by connecting 2-5 amino acid residues, wherein each amino acid residue can be optionally further substituted by 1 or more (alternatively 1, 2 or 3, alternatively 1) amino acid residues, and the amino acid residue is optionally substituted by 1, 2, 3, 4, 5 or 6 R_{L}; in another more specific embodiment, L₂ is

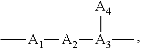

which is optionally substituted by 1, 2, 3, 4, 5 or 6 R_{L}; in another more specific embodiment, L₂ is selected from:

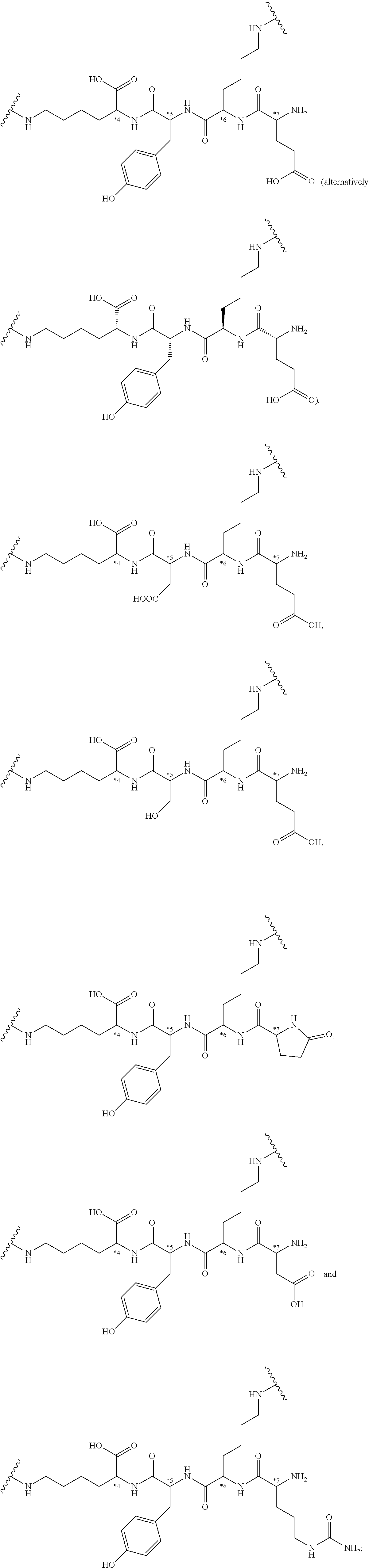

in another more specific embodiment, L₂ is selected from:

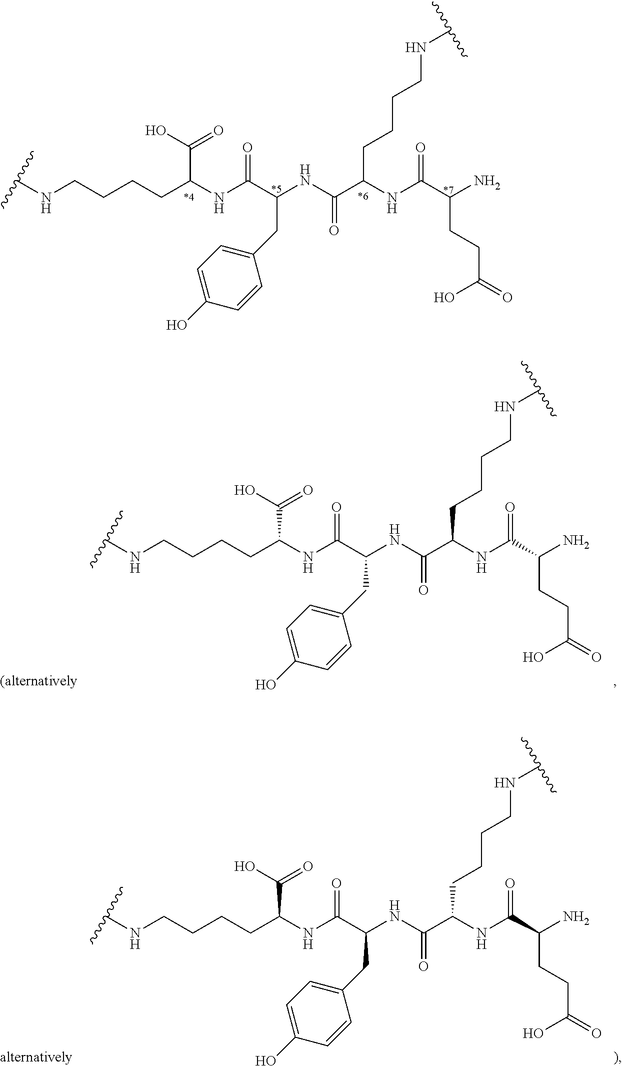 [SMILES: *NCCCCC(NC(=O)C(Cc1ccc(O)cc1)NC(=O)C(CCCCN*)NC(=O)C(N)CCC(=O)O)C(=O)O ; *NCCCC[C@@H](NC(=O)[C@@H](Cc1ccc(O)cc1)NC(=O)[C@@H](CCCCN*)NC(=O)[C@H](N)CCC(=O)O)C(=O)O ; *NCCCC[C@H](NC(=O)[C@H](Cc1ccc(O)cc1)NC(=O)[C@H](CCCCN*)NC(=O)[C@@H](N)CCC(=O)O)C(=O)O]

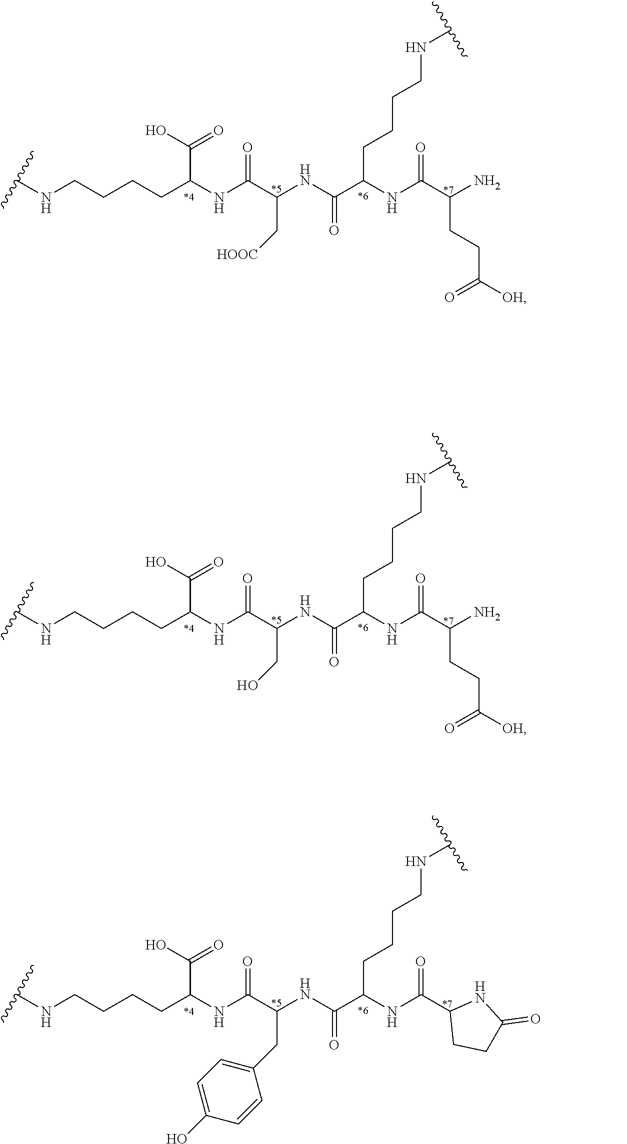 [SMILES: *NCCCCC(NC(=O)C(CC(=O)O)NC(=O)C(CCCCN*)NC(=O)C(N)CCC(=O)O)C(=O)O ; *NCCCCC(NC(=O)C(CO)NC(=O)C(CCCCN*)NC(=O)C(N)CCC(=O)O)C(=O)O ; *NCCCCC(NC(=O)C(Cc1ccc(O)cc1)NC(=O)C(CCCCN*)NC(=O)C1CCC(=O)N1)C(=O)O]

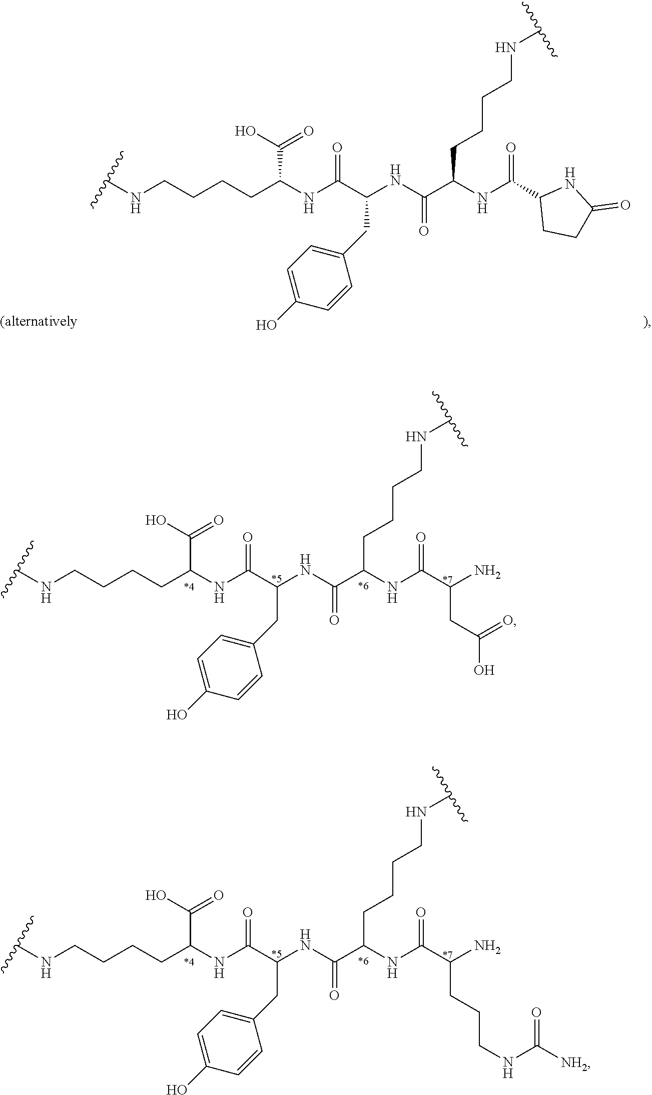

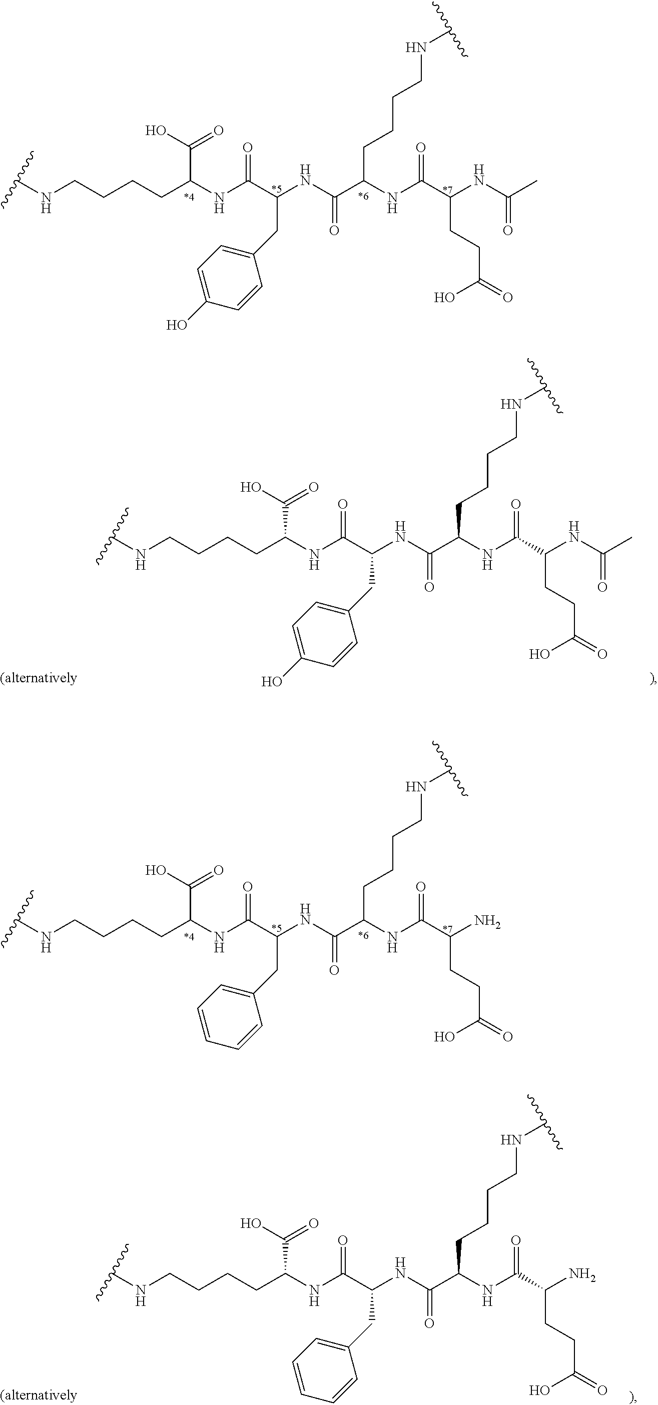

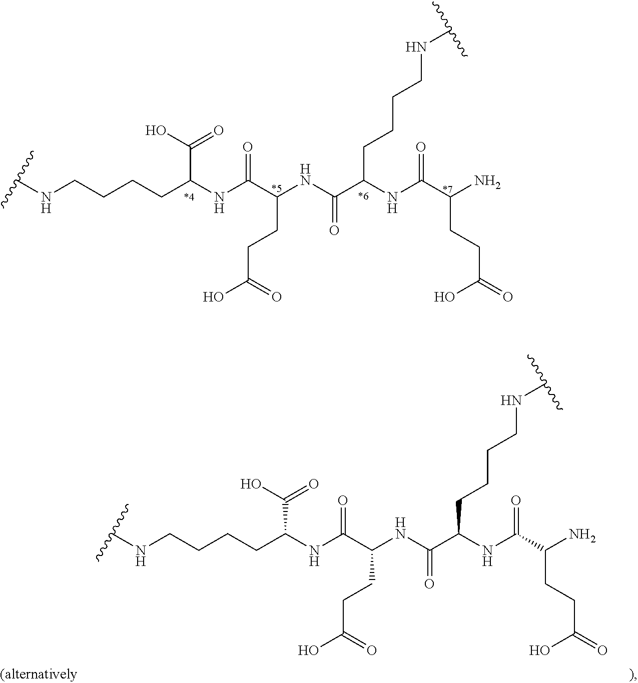

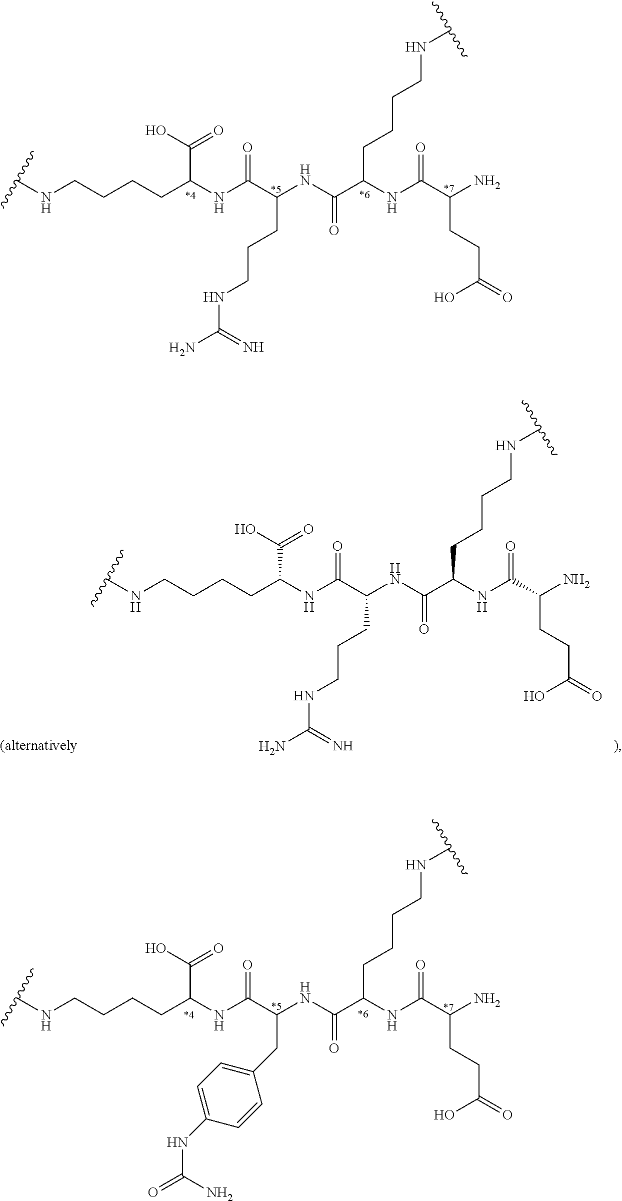 [SMILES: *NCCCCC(NC(=O)C(CCCNC(=N)N)NC(=O)C(CCCCN*)NC(=O)C(N)CCC(=O)O)C(=O)O ; *NCCCC[C@@H](NC(=O)[C@@H](CCCNC(=N)N)NC(=O)[C@@H](CCCCN*)NC(=O)[C@H](N)CCC(=O)O)C(=O)O ; *NCCCCC(NC(=O)C(Cc1ccc(NC(N)=O)cc1)NC(=O)C(CCCCN*)NC(=O)C(N)CCC(=O)O)C(=O)O]

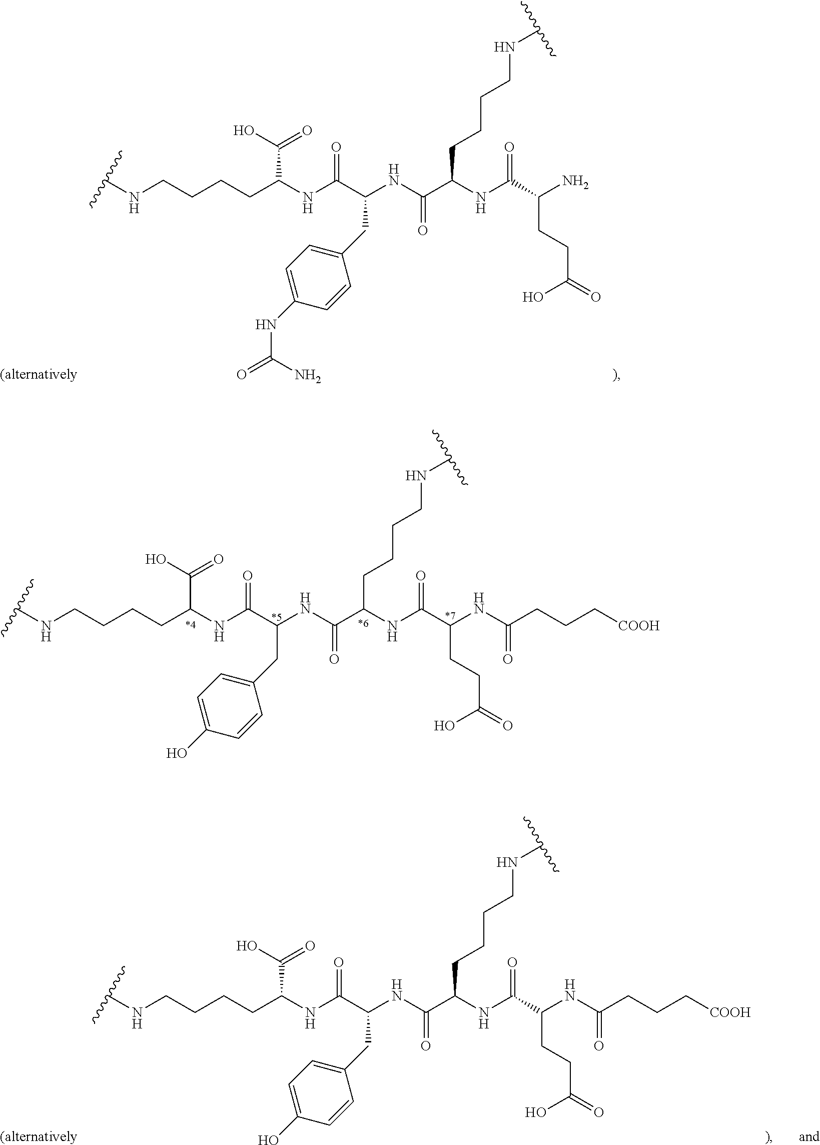

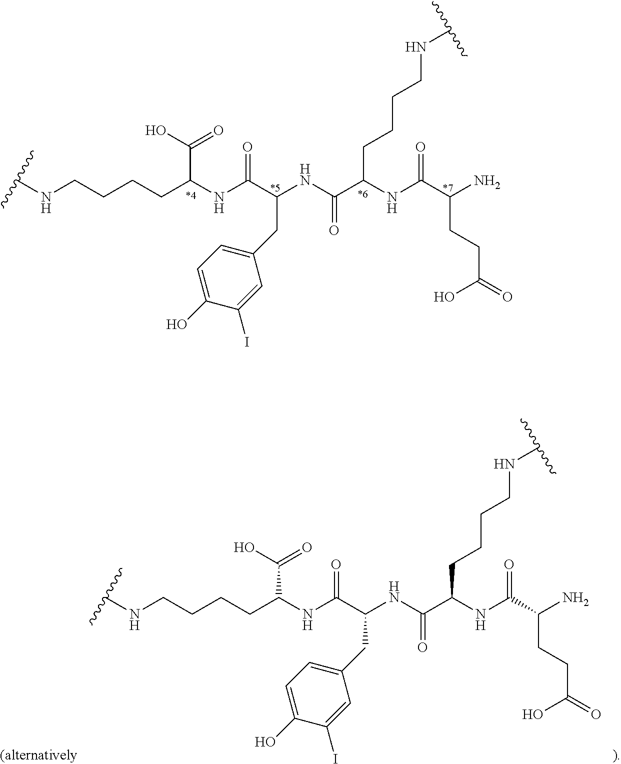

### R_{L}

In one embodiment, R_{L} is a halogen, such as I; in another embodiment, R_{L} is NO₂; in another embodiment, R_{L} is —ORₐ; in another embodiment, R_{L} is —NR_{b}R_{c}; in another embodiment, R_{L} IS C₁₋₆ alkyl; in another embodiment, R_{L} IS C₁₋₆ haloalkyl; in another embodiment, R_{L} is urea group (—NHC(O)NH₂); in another embodiment, R_{L} IS C₁₋₆ acyl, such as acetyl; in another embodiment, R_{L} is —C(O)—C₁₋₆ alkylene-COOH, such as —C(O)—(CH₂)₄—COOH; in another embodiment, R_{L} is sulfonic acid group; in another embodiment, R_{L} is methylsulfonyl; in another embodiment, R_{L} is phosphate group; in another embodiment, R_{L} is phosphite group.

In a more specific embodiment, R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl group, phosphate group and phosphite group; in another more specific embodiment, R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; in another more specific embodiment, R_{L} is independently selected from halogen, C₁₋₆ alkyl, C₁₋₆ haloalkyl, urea group, C₁₋₆ acyl and —C(O)—C₁₋₆ alkylene-COOH; in another more specific embodiment, R_{L} is independently selected from halogen, —OR, —NR_{b}R_{c}, C₁₋₆ alkyl, C₁₋₆ haloalkyl, and urea group. In another more specific embodiment, R_{L} is independently selected from halogen, C₁₋₆ alkyl, C₁₋₆ haloalkyl and urea group.

A₁, A₂, A₃ and A₄

In one embodiment, A₁ is an amino acid residue; in another embodiment, A₁ is a glycine residue; in another embodiment, A₁ is an alanine residue; in another embodiment, A₁ is a phenylalanine residue; in another embodiment, A₁ is a lysine residue; in another embodiment, A₁ is a tyrosine residue; in another embodiment, A₁ is an aspartic acid residue; in another embodiment, A₁ is a serine residue; in another embodiment, A₁ is a glutamic acid residue; in another embodiment, A₁ is a pyroglutamic acid residue; in another embodiment, A₁ is a citrulline residue; in another embodiment, A₁ is an arginine residue; in another embodiment, A₁ is COOH

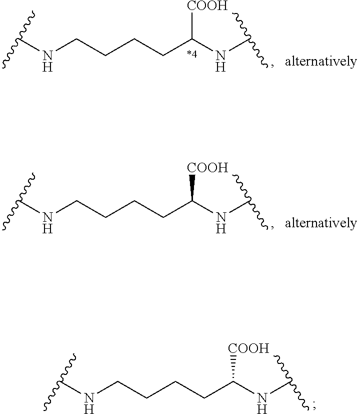 [SMILES: *NCCCCC(N*)C(=O)O ; *NCCCC[C@H](N*)C(=O)O ; *NCCCC[C@@H](N*)C(=O)O]

in another embodiment, A₁ is optionally substituted with 1, 2, 3, 4, 5 or 6 R_{L}; in another embodiment, A₁ is not substituted.

In one embodiment, A₂ is an amino acid residue; in another embodiment, A₂ is a glycine residue; in another embodiment, A₂ is an alanine residue; in another embodiment, A₂ is a phenylalanine residue; in another embodiment, A₂ is a lysine residue; in another embodiment, A₂ is a tyrosine residue; in another embodiment, A₂ is an aspartic acid residue; in another embodiment, A₂ is a serine residue; in another embodiment, A₂ is a glutamic acid residue; in another embodiment, A₂ is a pyroglutamic acid residue; in another embodiment, A₂ is a citrulline residue; in another embodiment, A₂ is an arginine residue; in another embodiment, A₂ is

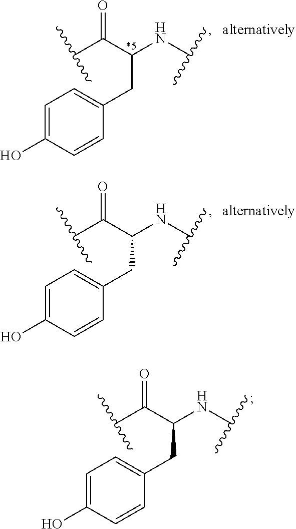

in another embodiment, A₂ is

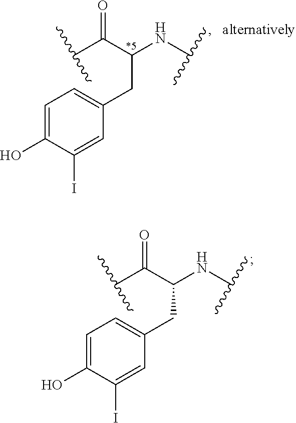 [SMILES: *NC(Cc1ccc(O)c(I)c1)C(*)=O ; *N[C@H](Cc1ccc(O)c(I)c1)C(*)=O]

in another embodiment, A₂ is

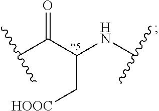 [SMILES: *NC(CC(=O)O)C(*)=O]

in another embodiment, A₂ is

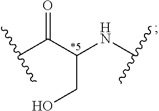 [SMILES: *NC(CO)C(*)=O]

in another embodiment, A₂ is

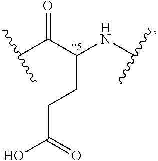 [SMILES: *NC(CCC(=O)O)C(*)=O]

alternatively

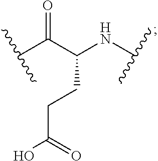 [SMILES: *N[C@H](CCC(=O)O)C(*)=O]

in another embodiment, A₂ is

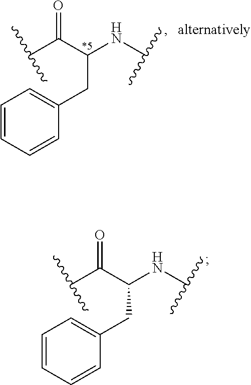 [SMILES: *NC(Cc1ccccc1)C(*)=O ; *N[C@H](Cc1ccccc1)C(*)=O]

in another embodiment, A₂ is

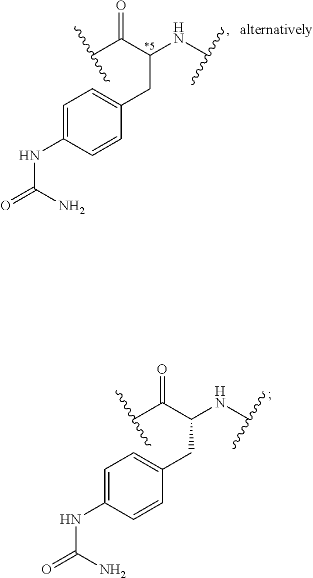 [SMILES: *NC(Cc1ccc(NC(N)=O)cc1)C(*)=O ; *N[C@H](Cc1ccc(NC(N)=O)cc1)C(*)=O]

in another embodiment, A₂ is

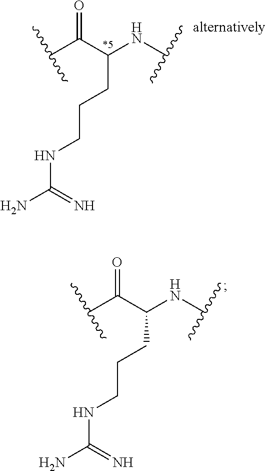 [SMILES: *NC(CCCNC(=N)N)C(*)=O ; *N[C@H](CCCNC(=N)N)C(*)=O]

in another embodiment, A₂ is optionally substituted with 1, 2, 3, 4, 5 or 6 R_{L}; in another embodiment, A₂ is not substituted.

In one embodiment, A₃ is an amino acid residue; in another embodiment, A₃ is a glycine residue; in another embodiment, A₃ is an alanine residue; in another embodiment, A₃ is a phenylalanine residue; in another embodiment, A₃ is a lysine residue; in another embodiment, A₃ is a tyrosine residue; in another embodiment, A₃ is an aspartic acid residue; in another embodiment, A₃ is a serine residue; in another embodiment, A₃ is a glutamic acid residue; in another embodiment, A₃ is a pyroglutamic acid residue; in another embodiment, A₃ is a citrulline residue; in another embodiment, A₃ is an arginine residue; in another embodiment, A₃ is

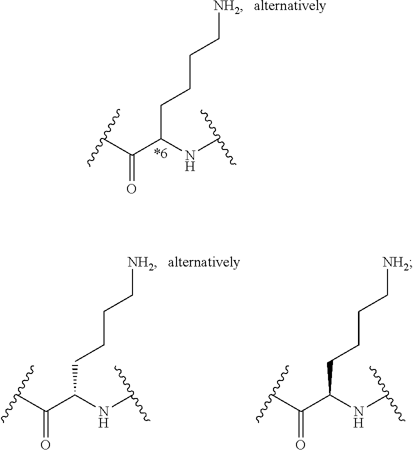

in another embodiment, A₃ is optionally substituted with 1, 2, 3, 4, 5 or 6 R_{L}; in another embodiment, A₃ is unsubstituted.

In one embodiment, A₄ is H; in another embodiment, A₄ is an amino acid residue; in another embodiment, A₄ is a glycine residue; in another embodiment, A₄ is an alanine residue; in another embodiment, A₄ is a phenylalanine residue; in another embodiment, A₄ is a lysine residue; in another embodiment, A₄ is a tyrosine residue; in another embodiment, A₄ is an aspartic acid residue; in another embodiment, A₄ is a serine residue; in another embodiment, A₄ is a glutamic acid residue; in another embodiment, A₄ is a pyroglutamic acid residue; in another embodiment, A₄ is a citrulline residue; in another embodiment, A₄ is an arginine residue; in another embodiment, A₄ is

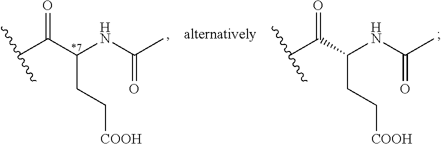 [SMILES: *C(=O)C(CCC(=O)O)NC(C)=O ; *C(=O)[C@@H](CCC(=O)O)NC(C)=O]

in another embodiment, A₄ is

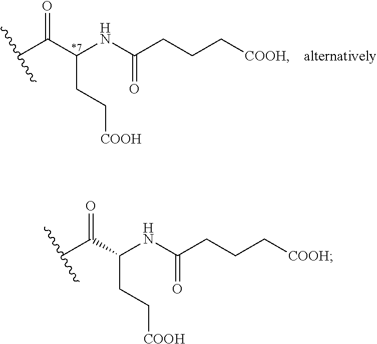 [SMILES: *C(=O)C(CCC(=O)O)NC(=O)CCCC(=O)O ; "alternatively" ; *C(=O)[C@@H](CCC(=O)O)NC(=O)CCCC(=O)O]

in another embodiment, A₄ is

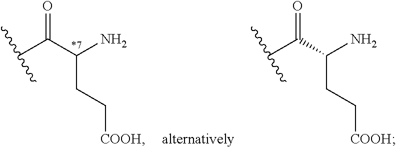 [SMILES: *C(=O)C(N)CCC(=O)O ; *C(=O)[C@H](N)CCC(=O)O]

in another embodiment, A₄ is

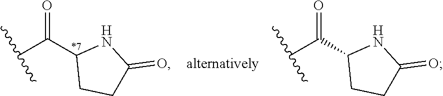 [SMILES: *C(=O)C1CCC(=O)N1 ; *C(=O)[C@H]1CCC(=O)N1]

in another embodiment, A₄ is

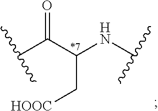 [SMILES: *NC(CC(=O)O)C(*)=O]

in another embodiment, A₄ is

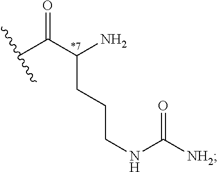 [SMILES: *C(=O)C(N)CCCNC(N)=O]

in another embodiment, A₄ is optionally substituted with 1, 2, 3, 4, 5 or 6 R_{L}, for example, A₄ is an N-acetylglutamic acid residue, for example, A₄ is N-(4-carboxybutyryl)glutamate; in another embodiment, A₄ is unsubstituted.

In a more specific embodiment, A₁ is selected from a glycine residue, an alanine residue, a phenylalanine residue, a lysine residue, a tyrosine residue, an aspartic acid residue, a serine residue, a glutamic acid residue, a pyroglutamic acid residue, a citrulline residue or an arginine residue; in another more specific embodiment, A₁ is selected from a lysine residue, a tyrosine residue, an aspartic acid residue, a serine residue, a glutamic acid residue, a pyroglutamic acid residue, a citrulline residue, a phenylalanine residue or an arginine residue; in another more specific embodiment, A₁ is a lysine residue, alternatively

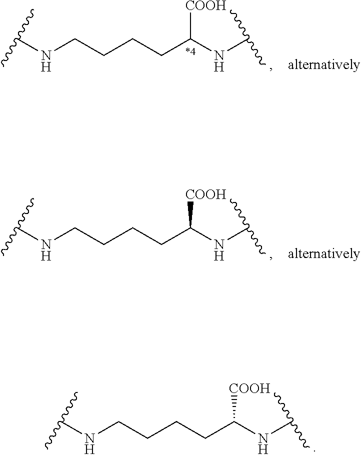 [SMILES: *NCCCCC(N*)C(=O)O ; *NCCCC[C@H](N*)C(=O)O ; *NCCCC[C@@H](N*)C(=O)O]

In a more specific embodiment, A₂ is selected from a glycine residue, an alanine residue, a phenylalanine residue, a lysine residue, a tyrosine residue, an aspartic acid residue, a serine residue, a glutamic acid residue, a pyroglutamic acid residue, a citrulline residue or an arginine residue; in another more specific embodiment, A₂ is selected from a lysine residue, a tyrosine residue, an aspartic acid residue, a serine residue, a glutamic acid residue, a pyroglutamic acid residue, a citrulline residue, a phenylalanine residue or an arginine residue; in another more specific embodiment, A₂ is selected from a tyrosine residue, an aspartic acid residue, a serine residue, a glutamic acid residue, a phenylalanine residue or an arginine residue; in another more specific embodiment, A₂ is selected from

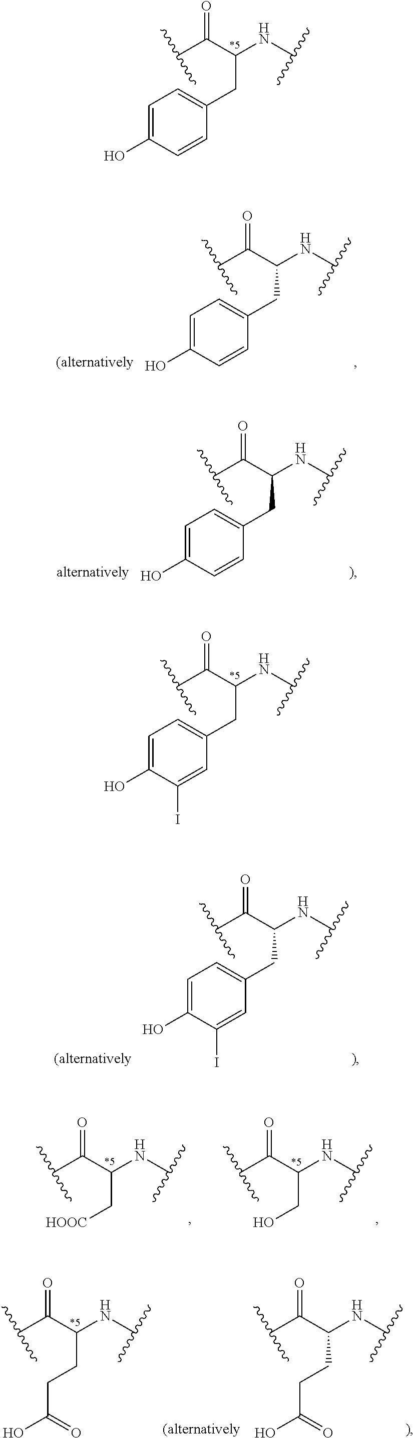

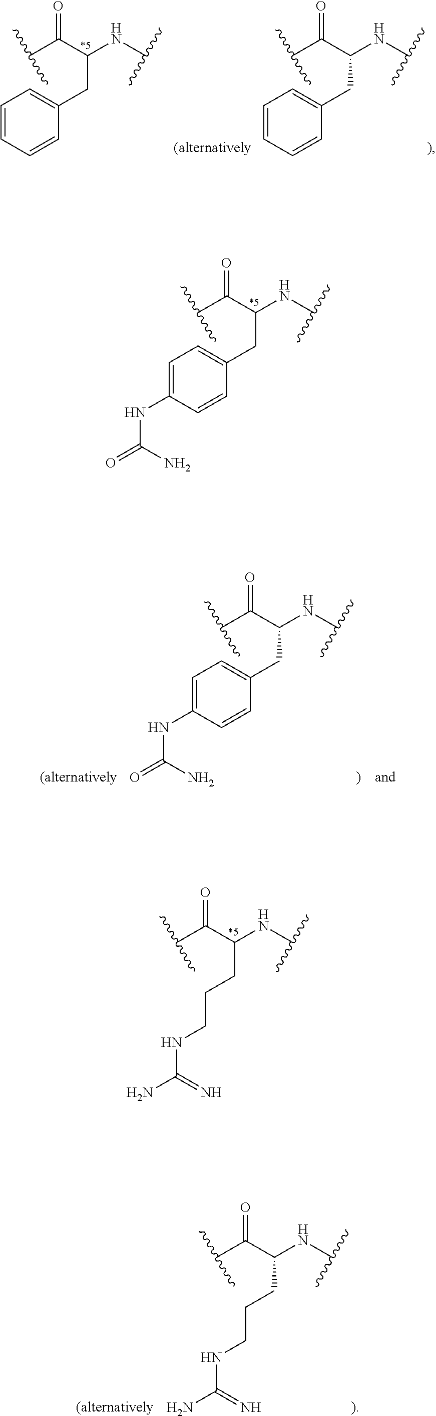 [SMILES: *NC(Cc1ccccc1)C(*)=O ; *N[C@H](Cc1ccccc1)C(*)=O ; *NC(Cc1ccc(NC(N)=O)cc1)C(*)=O ; *N[C@H](Cc1ccc(NC(N)=O)cc1)C(*)=O ; *NC(CCCNC(=N)N)C(*)=O ; *N[C@H](CCCNC(=N)N)C(*)=O]

In a more specific embodiment, A₃ is selected from a glycine residue, an alanine residue, a phenylalanine residue, a lysine residue, a tyrosine residue, an aspartic acid residue, a serine residue, a glutamic acid residue, a pyroglutamic acid residue, a citrulline residue or an arginine residue; in another more specific embodiment, A₃ is selected from a lysine residue, a tyrosine residue, an aspartic acid residue, a serine residue, a glutamic acid residue, a pyroglutamic acid residue, a citrulline residue, a phenylalanine residue or an arginine residue; in another more specific embodiment, A₃ is a lysine residue, alternatively

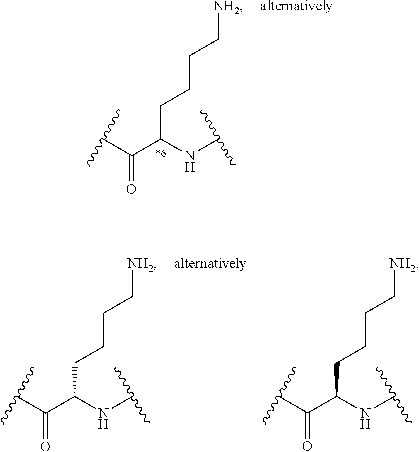

In a more specific embodiment, A₄ is H or an amino acid residue; in another more specific embodiment, A₄ is an amino acid residue; in another more specific embodiment, A₄ is selected from a glycine residue, an alanine residue, a phenylalanine residue, a lysine residue, a tyrosine residue, an aspartic acid residue, a serine residue, a glutamic acid residue, a pyroglutamic acid residue, a citrulline residue or an arginine residue; in another more specific embodiment, A₄ is selected from a lysine residue, a tyrosine residue, an aspartic acid residue, a serine residue, a glutamic acid residue, a pyroglutamic acid residue, a citrulline residue, a phenylalanine residue or an arginine residue; in another more specific embodiment, A₄ is selected from a N-acetylglutamic acid residue, N-(4-carboxybutyryl)glutamate, a glutamic acid residue, a pyroglutamic acid residue, a citrulline residue and an aspartic acid residue; in another more specific embodiment, A₄ is selected from

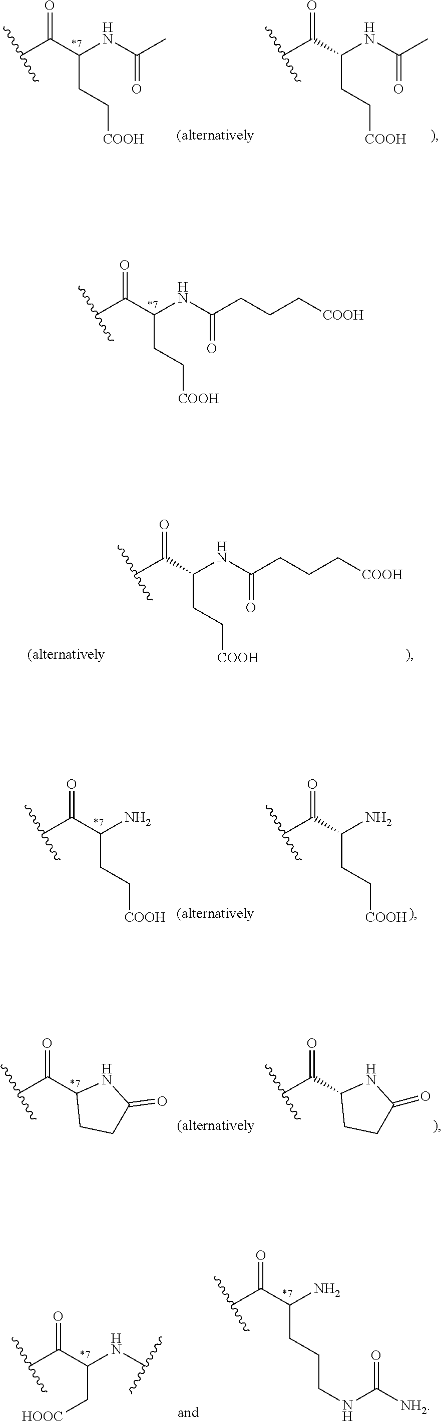

### Z

In one embodiment, Z is a chelating group derived from a chelating agent; in another embodiment, Z is 1,4,7,10-tetraazacyclododecane-N,N′,N″,N′″-tetraacetic acid (DOTA); in another embodiment, Z is N,N″-bis[2-hydroxy-5-(carboxyethyl)benzyl]ethylenediamine-N,N″-diacetic acid (HBED-CC); in another embodiment, Z is 1,4,7-triazacyclononane-1,4,7-triacetic acid (NOTA); in another embodiment, Z is 2-(4,7-bis(carboxymethyl)-1,4,7-triazononan-1-yl)glutaric acid (NODAGA); in another embodiment, Z is 2-(4,7,10-tris(carboxymethyl)-1,4,7,10-tetraazacyclododecane-1-yl)glutaric acid (DOTAGA); in another embodiment, Z is 1,4,7-triazacyclononanephosphinic acid (TRAP); in another embodiment, Z is 1,4,7-triazacyclononane-1-[methyl(2-carboxyethyl)phosphinic acid]-4,7-bis[methyl(2-hydroxymethyl)phosphinic acid](NOPO); in another embodiment, Z is 3,6,9,15-tetraazabicyclo[9.3.1.]pentadeca-1(15),11,13-triene-3,6,9-triacetic acid (PCTA); in another embodiment, Z is N′-{5-[acetyl(hydroxy)amino]pentyl}-N-[5-({4-[(5-aminopentyl)(hydroxy)amino]-4-oxobutanoyl}amino)pentyl]-N-hydroxysuccinamide (DFO); in another embodiment, Z is diethylenetriaminepentaacetic acid (DTPA); in another embodiment, Z is trans-cyclohexyl-diethylenetriaminepentaacetic acid (CHX-DTPA); in another embodiment, Z is 1-oxa-4,7,10-triazacyclododecane-4,7,10-triacetic acid (oxy-Do3A); in another embodiment, Z is p-isocyanatobenzyl-DTPA (SCN-Bz-DTPA); in another embodiment, Z is 1-(p-isocyanatobenzyl)-3-methyl-DTPA (1B3M); in another embodiment, Z is 2-(p-isocyanatobenzyl)-4-methyl-DTPA (1M3B); in another embodiment, Z is 1-(2)-methyl-4-isocyanatobenzyl-DTPA (MX-DTPA); in another embodiment, Z is (R)-2-amino-3-(4-isothiocyanatophenyl)propyl]-trans-(S,S)-cyclohexane-1,2-diaminepentaacetic acid (p-SCN-Bn-CHX-A″-DTPA); in another embodiment, Z is 6-hydrazinopyridine-3-carboxylic acid (HYNIC); in another embodiment, Z is 2-(4-isothiocyanatophenyl)-1,4,7-triazacyclononane-1,4,7-triacetic acid (p-SCN-Bn-NOTA); in another embodiment, Z is 2-[(4-isothiocyanatophenyl)methyl]-1,4,7,10-tetraazacyclododecane-1,4,7,10-tetraacetic acid (p-SCN-Bn-DOTA).

In one embodiment, Z is

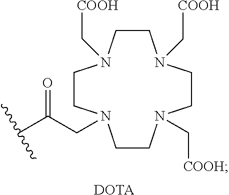 [SMILES: *C(=O)CN1CCN(CC(=O)O)CCN(CC(=O)O)CCN(CC(=O)O)CC1 ; "DOTA"]

in another embodiment, Z is

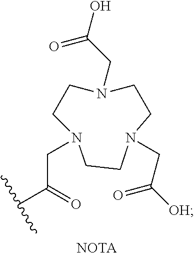

in another embodiment, Z is

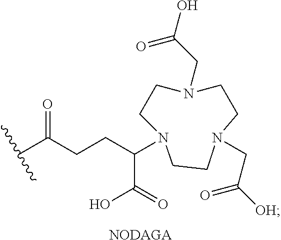

in another embodiment, Z is

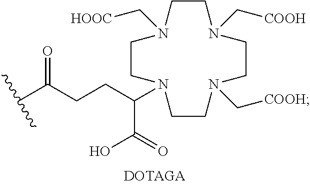 [SMILES: *C(=O)CCC(C(=O)O)N1CCN(CC(=O)O)CCN(CC(=O)O)CCN(CC(=O)O)CC1]

in another embodiment, Z is

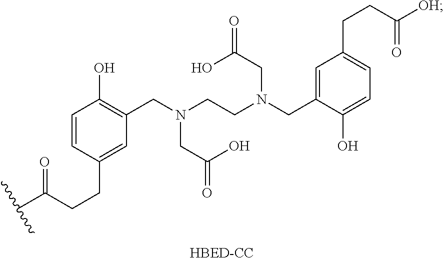 [SMILES: *C(=O)CCc1ccc(O)c(CN(CCN(CC(=O)O)Cc2cc(CCC(=O)O)ccc2O)CC(=O)O)c1]

in another embodiment, Z is

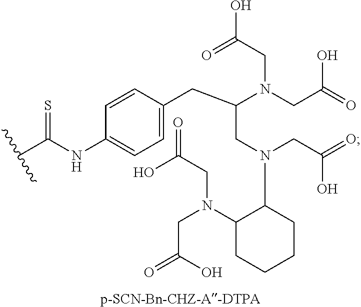 [SMILES: *C(=S)Nc1ccc(CC(CN(CC(=O)O)C2CCCCC2N(CC(=O)O)CC(=O)O)N(CC(=O)O)CC(=O)O)cc1]

in another embodiment, Z is

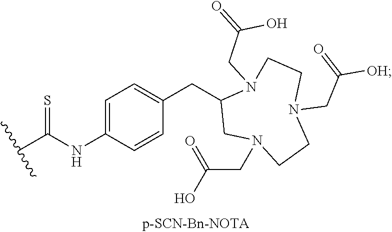 [SMILES: *C(=S)Nc1ccc(CC2CN(CC(=O)O)CCN(CC(=O)O)CCN2CC(=O)O)cc1]

in another embodiment, Z is

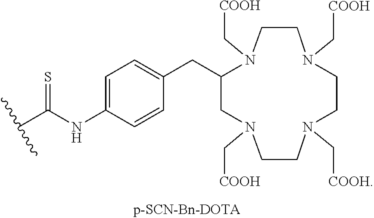 [SMILES: *C(=S)Nc1ccc(CC2CN(CC(=O)O)CCN(CC(=O)O)CCN(CC(=O)O)CCN2CC(=O)O)cc1]

In a more specific embodiment, Z is a chelating group derived from a chelating agent selected from the following: 1,4,7,10-tetraazacyclododecane-N,N′,N″,N′″-tetraacetic acid (DOTA),
N,N″-bis[2-hydroxy-5-(carboxyethyl)benzyl]ethylenediamine-N,N″-diacetic acid (HBED-CC),
1,4,7-triazacyclononane-1,4,7-triacetic acid (NOTA),
2-(4,7-bis(carboxymethyl)-1,4,7-triazononan-1-yl)glutaric acid (NODAGA),
2-(4,7,10-tris(carboxymethyl)-1,4,7,10-tetraazacyclododecane-1-yl)glutaric acid (DOTAGA),
1,4,7-triazacyclononanephosphinic acid (TRAP),
1,4,7-triazacyclononane-1-[methyl(2-carboxyethyl)phosphinic acid]-4,7-bis[methyl(2-hydroxymethyl)phosphinic acid](NOPO),
3,6,9,15-tetraazabicyclo[9.3.1.]pentadeca-1(15),11,13-triene-3,6,9-triacetic acid (PCTA),
N′-{5-[acetyl(hydroxy)amino]pentyl}-N-[5-({4-[(5-aminopentyl)(hydroxy)amino]-4-oxobutanoyl}amino)pentyl]-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (oxy-Do3A),
p-isocyanatobenzyl-DTPA (SCN-Bz-DTPA),
1-(p-isocyanatobenzyl)-3-methyl-DTPA (1B3M),
2-(p-isocyanatobenzyl)-4-methyl-DTPA (1M3B),
1-(2)-methyl-4-isocyanatobenzyl-DTPA (MX-DTPA),
(R)-2-amino-3-(4-isothiocyanatophenyl)propyl]-trans-(S,S)-cyclohexane-1,2-diaminepentaacetic acid (p-SCN-Bn-CHX-A″-DTPA),
6-hydrazinopyridine-3-carboxylic acid (HYNIC),
2-(4-isothiocyanatophenyl)-1,4,7-triazacyclononane-1,4,7-triacetic acid (p-SCN-Bn-NOTA) or
2-[(4-isothiocyanatophenyl)methyl]-1,4,7,10-tetraazacyclododecane-1,4,7,10-tetraacetic acid (p-SCN-Bn-DOTA).

In another more specific embodiment, Z is selected from

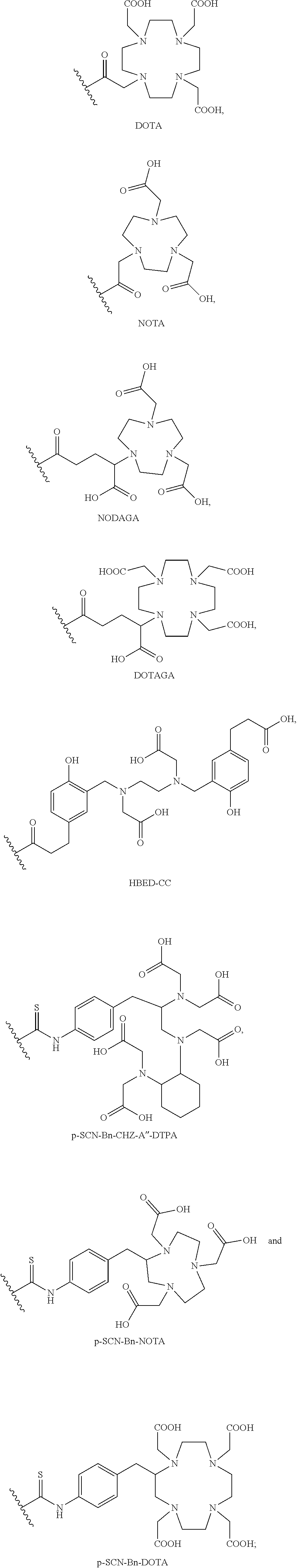

in another more specific embodiment, Z is selected from

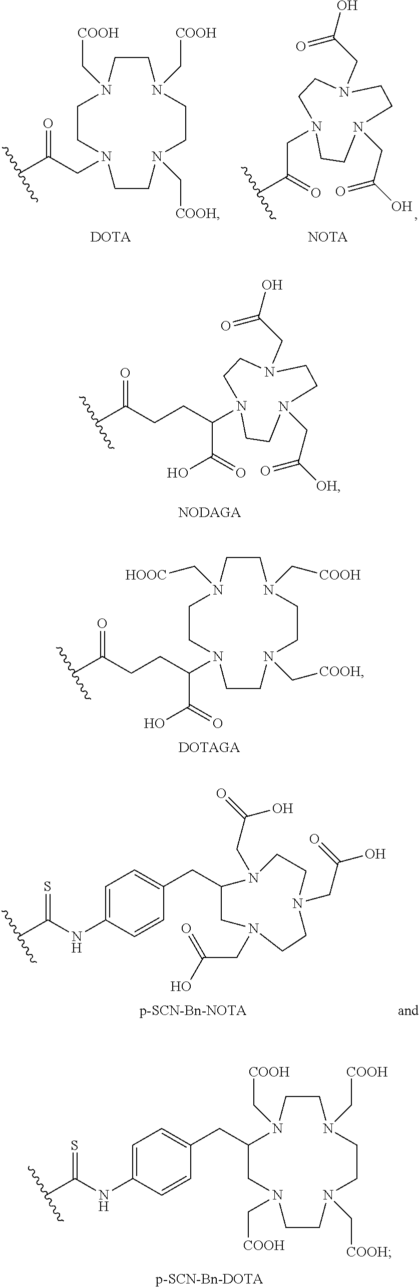

in another more specific embodiment, Z is

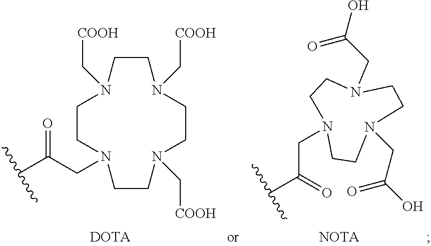

in another more specific embodiment, Z is

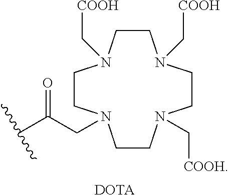 [SMILES: *C(=O)CN1CCN(CC(=O)O)CCN(CC(=O)O)CCN(CC(=O)O)CC1 ; "DOTA"]

### Chiral Centers *1, *2, *3, *4, *5, *6 and *7

In one embodiment, *1 is in (S) configuration; in another embodiment, *1 is in (R) configuration; in another embodiment, *1 is in racemic form.

In one embodiment, *2 is in (S) configuration; in another embodiment, *2 is in (R) configuration; in another embodiment, *2 is in racemic form.

In one embodiment, *3 is in (S) configuration; in another embodiment, *3 is in (R) configuration; in another embodiment, *3 is in racemic form.

In one embodiment, *4 is in (S) configuration; in another embodiment, *4 is in (R) configuration; in another embodiment, *4 is in racemic form.

In one embodiment, *5 is in (S) configuration; in another embodiment, *5 is in (R) configuration; in another embodiment, *5 is in racemic form.

In one embodiment, *6 is in (S) configuration; in another embodiment, *6 is in (R) configuration; in another embodiment, *6 is in racemic form.

In one embodiment, *7 is in (S) configuration; in another embodiment, *7 is in (R) configuration; in another embodiment, *7 is in racemic form.

In one embodiment, *8 is in (S) configuration; in another embodiment, *8 is in (R) configuration; in another embodiment, *8 is in racemic form.

In one embodiment, *9 is in (S) configuration; in another embodiment, *9 is in (R) configuration; in another embodiment, *9 is in racemic form.

*1, *2, *3, *4, *5, *6, *7, *8 and *9 are independently selected from in (S) or (R) configuration, or are in racemic form.

### M

In one embodiment, M is a radionuclide; in another embodiment, M is a non-radioactive element; in another embodiment, M is a diagnostic nuclide; in another embodiment, M is a therapeutic nuclide; in another embodiment, M is ⁶⁸Ga; in another embodiment, M is ¹⁸F; in another embodiment, M is ⁹⁹mTc; in another embodiment, M is ⁸⁹Zr; in another embodiment, M is ¹²⁴I; in another embodiment, M is ⁷⁶Br; in another embodiment, M is ⁴³Sc; in another embodiment, M is ¹¹³In; in another embodiment, M is ⁴⁵Ti; in another embodiment, M is ⁵²Mn; in another embodiment, M is ⁵⁹Fe; in another embodiment, M is ⁶⁴Cu; in another embodiment, M is ⁹⁴mTc; in another embodiment, M is ⁶⁷Ga; in another embodiment, M is ^{71/72/74}As; in another embodiment, M is ^{82m}R_{b}; in another embodiment, M is ⁸⁶Y; in another embodiment, M is ¹⁷⁷Lu; in another embodiment, M is ⁹⁰Y; in another embodiment, M is ¹³¹I; in another embodiment, M is ¹⁵³Sm; in another embodiment, M is ⁶⁷Cu; in another embodiment, M is ⁸⁹Sr; in another embodiment, M is ¹⁶⁶Ho; in another embodiment, M is ¹⁷⁷Yb; in another embodiment, M is ⁴⁷Sc; in another embodiment, M is ^{186/188}Re; in another embodiment, M is ^{212/213}Bi; in another embodiment, M is ¹⁴⁹Pm; in another embodiment, M is ²¹²Ph; in another embodiment, M is ²¹¹At; in another embodiment, M is ²²³Ra; in another embodiment, M is ¹⁶¹Tb; in another embodiment, M is ²²⁵Ac; in another embodiment, M is ²²⁷Th.

In a more specific embodiment, M is selected from at least one of a radionuclide or a non-radioactive element; in another more specific embodiment, the radionuclide is selected from at least one of a diagnostic nuclide or a therapeutic nuclide; in another more specific embodiment, the diagnostic nuclide is selected from ⁶⁸Ga, ¹⁸F, ⁹⁹mTc, ⁸⁹Zr, ¹²⁴I, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or ⁸⁶Y; in another more specific embodiment, the therapeutic nuclide is selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb, ⁴⁷Sc, ^{186/188}Re, 2^{12/213}Bi, ¹⁴⁹Pm, ²¹²Pb, ²¹¹At, ²²³Ra, ¹⁶¹Tb, ²²⁵Ac or ²²⁷Th; in another more specific embodiment, the radionuclide is selected from ⁶⁸Ga, ¹⁸F, ⁸⁹Zr, ⁹⁹mTc or ¹⁷⁷Lu; in another more specific embodiment, the radionuclide ¹⁸F is formed by complexing ¹⁸FA1 with the compound of formula (I); in another more specific embodiment, M is selected from ⁶⁸Ga, ¹⁸F or ¹⁷⁷Lu; in another more specific embodiment, M is selected from ⁶⁸Ga or ¹⁷⁷Lu.

### Z′

In one embodiment, Z′ is a coordination group formed by complexing a chelating group Z derived from a chelating agent with M.

Any technical solution or any combination thereof in any of the above specific embodiments can be combined with any technical solution or any combination thereof in other specific embodiments. For example, any technical solution of Ab or any combination thereof can be combined with any technical solution of ring A, R_{A}, R₃, R₄, V₃, W₃, Ring B, R*, R, n, Rₐ, R_{b}, R_{c}, L₁, W₁, W₂, V₁, m, R₁ₛ, V₂, R′, R″, Ar, W₄, L₂, A₁, A₂, A₃, A₄, Z, M, Z′, *1, *2, *3, *4, *5, *6, *7, *8 and *9 or any combination thereof. The present disclosure is intended to include all combinations of these technical solutions, which are not listed one by one due to space limitations.

In a more specific embodiment, the present disclosure provides a compound of formula (I), or an isotopic variant, a hydrate, an ester or solvate, a tautomer or a stereoisomer thereof, or a pharmaceutically acceptable salt thereof:

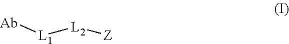

wherein,
Ab is a ligand targeting carbonic anhydrase IX, for example R₄

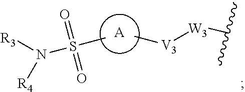

Ring A is selected from C₆₋₁₀ arylene and C₅₋₁₀ heteroarylene; wherein the C₆₋₁₀ arylene and C₅₋₁₀ heteroarylene are optionally substituted by 1, 2, 3 or 4 R_{A};
R_{A} is independently selected from H, D, halogen, CN, —OR, —SR, —NR_{b}R_{c}, C₁₋₆ alkyl, C₁₋₆ haloalkyl, C₃₋₁₀ cycloalkyl, 3-10 membered heterocyclyl, C₆₋₁₀ aryl and 5-10 membered heteroaryl;
R₃ and R₄ are independently selected from H, halogen, C₁₋₆ alkyl, C₁₋₆ haloalkyl, C₃₋₇ cycloalkyl and 3-7 membered heterocyclyl;
or R₄ and R_{A} are connected to form a C₁₋₄ alkylene, a C₂₋₄ alkenylene and a C₂₋₄ alkynylene, wherein one or more methylene units are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)— or —S(O)₂—; and the ring formed by conneting R₄ with R_{A} is a ring structure fused with ring A;
V₃ is selected from a chemical bond, a C₁₋₁₀ alkylene group, a C₂₋₁₀ alkenylene group, a C₂₋₁₀ alkynylene group and

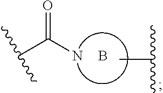

Ring B is a 3-7 membered heterocyclylene group;
W₃ is selected from a chemical bond, —NR—, —O—, —S— and —C(O)—;
R* is independently selected from H, D, halogen, C₁₋₆ alkyl and C₁₋₆ haloalkyl;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-10 membered heterocyclyl group;
L₁ is a linker;
L₂ is an amino acid chain formed by connecting 2-8 amino acid residues, wherein each amino acid residue can be optionally further substituted by one or more amino acid residues, and the amino acid residues are optionally substituted by 1, 2, 3, 4, 5 or 6 R_{L};
R_{L} is independently selected from halogen, NO₂, —ORₐ, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent.

In a more specific embodiment, the present disclosure provides a compound of formula (I), or an isotopic variant, a hydrate, an ester or solvate, a tautomer or a stereoisomer thereof, or a pharmaceutically acceptable salt thereof:

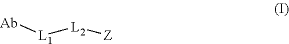

wherein,
Ab is a ligand targeting carbonic anhydrase IX, for example

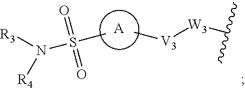

Ring A is selected from C₆₋₁₀ arylene and C₅₋₁₀ heteroarylene; wherein the C₆₋₁₀ arylene and C₅₋₁₀ heteroarylene are optionally substituted by 1, 2, 3 or 4 R_{A};
R_{A} is independently selected from H, D, halogen, CN, —ORₐ, —SRₐ, —NR_{b}R_{c}, C₁₋₆ alkyl, C₁₋₆ haloalkyl, C₃₋₁₀ cycloalkyl, 3-10 membered heterocyclyl, C₆₋₁₀ amyl and 5-10 membered heteroaryl;
R₃ and R₄ are independently selected from H, halogen, C₁₋₆ alkyl, C₁₋₆ haloalkyl, C₃₋₇ cycloalkyl and 3-7 membered heterocyclyl;
or R₄ and R_{A} are connected to form a C₁₋₄ alkylene, a C₂₋₄ alkenylene and a C₂₋₄ alkynylene, wherein one or more methylene units are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)— or —S(O)₂—; and the ring formed by conneting R₄ with R_{A} is a ring structure fused with ring A;
V₃ is selected from a chemical bond, a C₅₋₁₀ alkylene group, a C₂₋₁₀ alkenylene group, and a C₂₋₁₀ alkynylene group;
W₃ is selected from a chemical bond, —NR—, —O—, —S— and —C(O)—;
R* is independently selected from H, D, halogen, C₁₋₆ alkyl and C₁₋₆ haloalkyl;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-10 membered heterocyclyl group;
L₁ is a linker;
L₂ is an amino acid chain formed by connecting 2-8 amino acid residues, wherein each amino acid residue can be further substituted by one or more amino acid residues, and the amino acid residues are optionally substituted by 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent.

In a more specific embodiment, the present disclosure provides the above compound, which has the following structure:

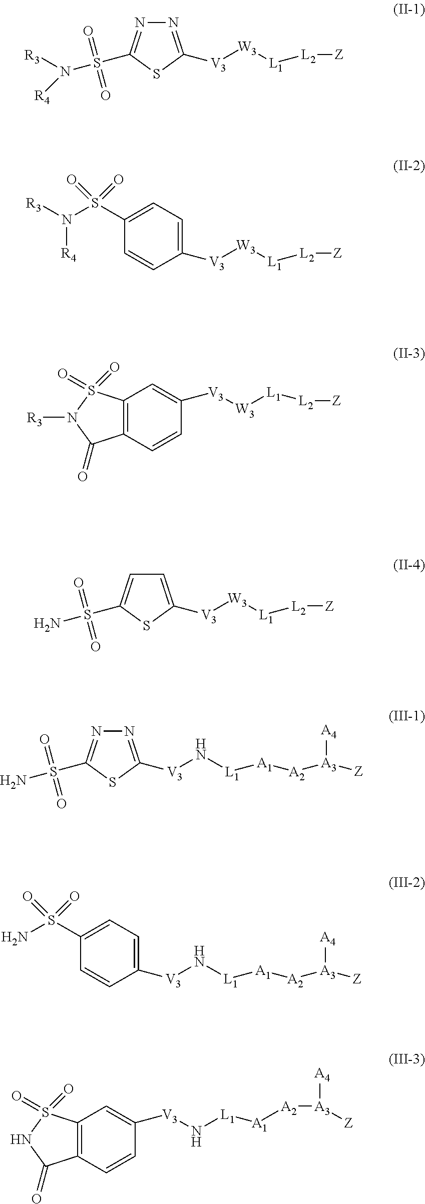

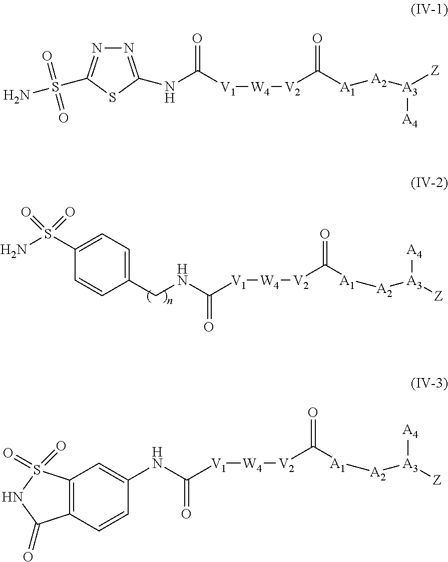

wherein,
A₁, A₂, and A₃ are amino acid residues;
A₄ is H or an amino acid residue;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted with 1, 2, 3, 4, 5 or 6 R₁ₛ;
R₁ₛ is independently selected from H, D, —ORₐ, —SRₐ, —NR_{b}R_{c}, halogen, C₁₋₆ alkyl, C₁₋₆ haloalkyl, C₃₋₇ cycloalkyl and 3-7 membered heterocyclyl;
V₂ is a chemical bond or —(CR′R″)₁₋₆—;
R′ and R″ are independently selected from H, D, halogen, C₁₋₆ alkyl, C₁₋₆ haloalkyl and —C₁₋₆ alkylene-Ar;
Ar is selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
n is 0, 1, 2, 3, 4, 5 or 6;
the remaining variables a as defined herein.

In a more specific embodiment, the present disclosure provides the above-mentioned compound, wherein,
L₁ is —W₁—V₁—W₄—V₂—W₂—;
W₁ is selected from a chemical bond, —NR—, —C(O)—, —C(O)O—, and —C(O)NR—;
W₂ is selected from —C(O)—, —OC(O)—, —NRC(O)—, —NR—, —C(O)NR—, and —O—;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted by 1, 2, 3, 4, 5 or 6 R₁ₛ; wherein one or more methylene units in the alkylene, alkenylene and alkynylene are optionally and independently replaced by —NR—, —NRC(O)—, —C(O)NR—, —NRS(O)₂—, —S(O)₂NR—, —O—, —C(O)—, —OC(O)—, —C(O)O—, —S—, —S(O)— or —S(O)₂—;
R is independently selected from H, C₁₋₆ alkyl or C₁₋₆ haloalkyl;
R₁ₛ is independently selected from H, D, —OR, —SRₐ, —NR_{b}R_{c}, halogen, C₁₋₆ alkyl, C₁₋₆ haloalkyl, C₃₋₇ cycloalkyl or 3-7 membered heterocyclyl; alternatively, R₁ₛ is independently in (S) or (R) configuration, or is in racemic form;
V₂ is a chemical bond or —(CR′R″)₁₋₆—;
R′ and R″ are independently selected from H, D, halogen, C₁₋₆ alkyl, C₁₋₆ haloalkyl and —C₁₋₆ alkylene-Ar; alternatively, R′ is independently in (S) or (R) configuration, or is in racemic form;
Ar is independently selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group.

Alternatively,
L₁ is —W₁—V₁—W₄—V₂—W₂—;
W₁ is selected from —C(O)—, —C(O)O— and —C(O)NR—; alternatively —C(O)—;
W₂ is selected from —C(O)—, —OC(O)— and —NRC(O)—; alternatively —C(O)—;
V₁ is selected from C₁₋₈ alkylene, C₂₋₈ alkenylene, C₂₋₈ alkynylene, phenylene and C₅₋₆ heteroarylene, alternatively C₁₋₈ alkylene (e.g. —(CH₂)ₘ—); which is optionally substituted by 1, 2, 3, 4, 5 or 6 R₁ₛ; wherein 1, 2 or 3 methylene units in the alkylene, alkenylene and alkynylene are optionally and independently replaced by —NR—, —NRC(O)—, —C(O)NR—, —C(O)—, —OC(O)— or —C(O)O—;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —OR, —NR_{b}R_{c}, halogen, C₁₋₆ alkyl and C₁₋₆ haloalkyl; alternatively H, D, —ORₐ and —NR_{b}R_{c};
V₂ is a chemical bond or —(CR′R″)₁₋₄—;
R′ is independently selected from H, D or —C₁₋₄ alkylene-Ar; alternatively, R′ is independently in (S) or (R) configuration, or in racemic form; alternatively in (S) configuration; alternatively in (R) configuration;
R″ is independently selected from H, D, halogen, C₁₋₆ alkyl and C₁₋₆ haloalkyl;
Ar is independently selected from C₆₋₁₄ aryl and 5-14 membered heteroaryl, alternatively C₆₋₁₄ aryl;
W₄ is selected from a chemical bond, —OC(O)—, —NRC(O)—, —C(O)O— and —C(O)NR—;

Alternatively,
L₁ is —C(O)—V₁—W₄—V₂—C(O)—;
wherein V₁ is selected from C₁₋₆ alkylene (e.g. —(CH₂)ₘ—), phenylene and C₅₋₆ heteroarylene; alternatively selected from C₁₋₆ alkylene (e.g. —(CH₂)ₘ—), phenylene, pyrrolylene, furylene or thienylene; alternatively C₁₋₆ alkylene (e.g. —(CH₂)ₘ—); alternatively C₁₋₄ alkylene (e.g. —(CH₂)ₘ—); which is optionally substituted by 1, 2, 3, 4, 5 or 6 R₁ₛ; wherein 1, 2 or 3 methylene units in the alkylene are optionally and independently replaced by —NR—, —NRC(O)—, —C(O)NR—, —C(O)—, —OC(O)— or —C(O)O—;
m is independently selected from 1, 2, 3, 4, 5 or 6, alternatively 1, 2, 3 or 4;
R is independently selected from H, C₁₋₆ alkyl and C₁₋₆ haloalkyl;
R₁ₛ is independently selected from H, D, —ORₐ, C₁₋₆ alkyl and C₁₋₆ haloalkyl; alternatively selected from H and —ORₐ;
V₂ is a chemical bond or —(CR′R″)₁₋₂— (alternatively —CHR′—);
R′ is independently selected from H or —C₁₋₄ alkylene-Ar, alternatively H or —CH₂—Ar; alternatively, R′ is independently in (S) or (R) configuration, or in racemic form; alternatively in (S) configuration; alternatively in (R) configuration;
R″ is independently selected from H, D, C₁₋₆ alkyl and C₁₋₆ haloalkyl; alternatively H or D;
Ar is independently C₆₋₁₄ aryl, such as phenyl, naphthyl, anthracenyl or phenanthryl; alternatively C₆₋₁₀ aryl, such as phenyl or naphthyl, alternatively naphthyl, alternatively 2-naphthyl;
W₄ is selected from a chemical bond, —OC(O)—, —NRC(O)—, —C(O)O— and —C(O)NR—; alternatively a chemical bond, —C(O)O— and —C(O)NR—; alternatively a chemical bond or —C(O)NR—;
for example,
L₁ is selected from:

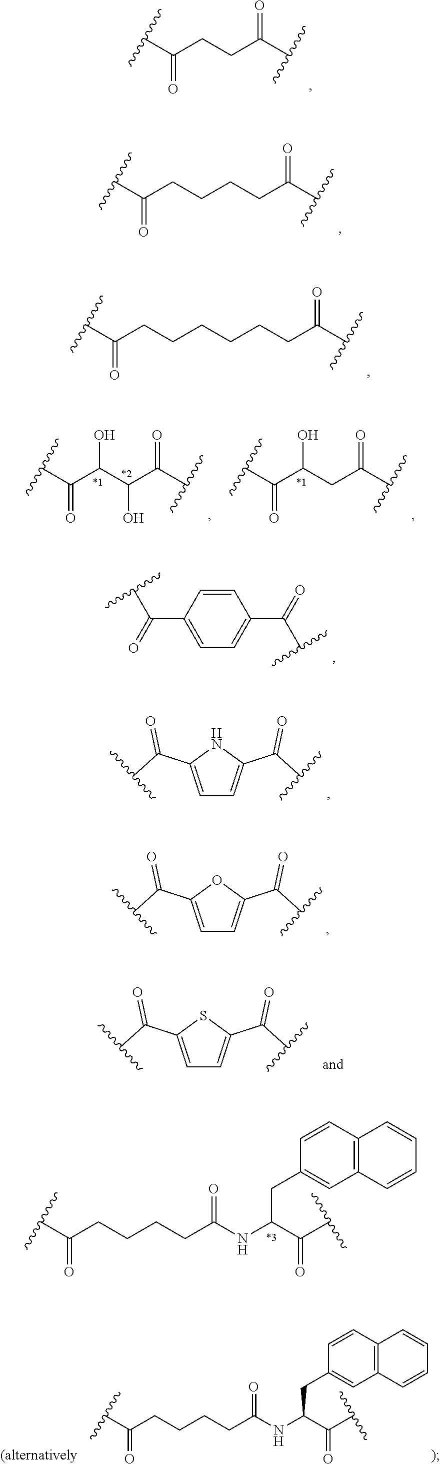

wherein, *1, *2 and *3 are chiral centers, independently selected from in (S) or (R) configuration, or in racemic form; alternatively in (S) configuration; alternatively in (R) configuration.

In a more specific embodiment, the present disclosure provides the above-mentioned compound, wherein,
L₂ is an amino acid chain formed by connecting 2-5 amino acid residues, wherein each amino acid residue can be optionally further substituted with one or more (alternatively 1, 2 or 3, alternatively 1) amino acid residues, and the amino acid residues can be optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
alternatively,
L₂ is

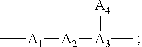

A₁, A₂, A₃ and A₄ are amino acid residues, and the amino acid residues are optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
alternatively,
L₂ is

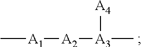

A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
still alternatively,
L₂ is

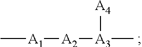

A₁ is a lysine residue; alternatively

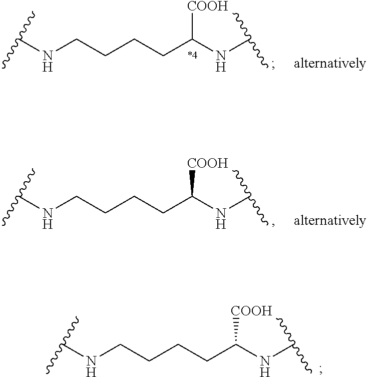 [SMILES: *NCCCCC(N*)C(=O)O ; *NCCCC[C@H](N*)C(=O)O ; *NCCCC[C@@H](N*)C(=O)O]

A₂ is selected from a tyrosine residue, an aspartic acid residue, a serine residue, a glutamic acid residue, a phenylalanine residue or an arginine residue, which is optionally substituted by 1, 2, 3 or 4 R_{L};
R_{L} is independently selected from halogen, —OR, —NR_{b}R_{c}, C₁₋₆ alkyl, C₁₋₆ haloalkyl and urea group; alternatively halogen, C₁₋₆ alkyl, C₁₋₆ haloalkyl and urea group;
alternatively A₂ is selected from

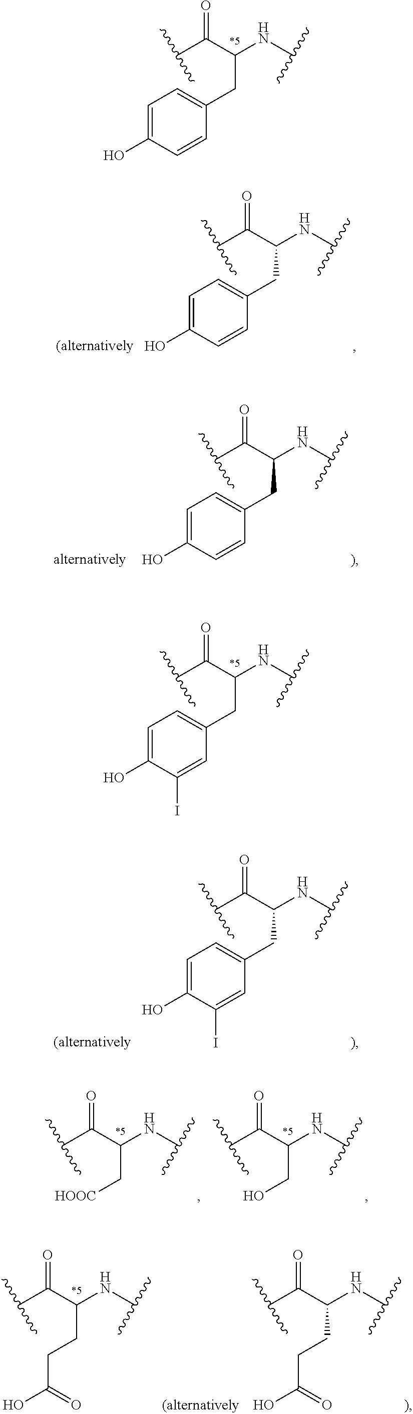

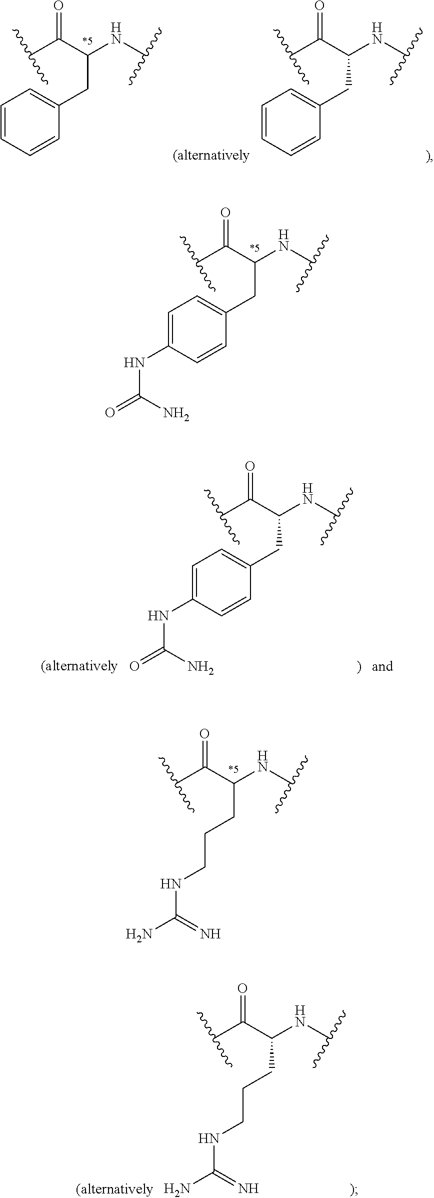 [SMILES: *NC(Cc1ccccc1)C(*)=O ; *N[C@H](Cc1ccccc1)C(*)=O ; *NC(Cc1ccc(NC(N)=O)cc1)C(*)=O ; *N[C@H](Cc1ccc(NC(N)=O)cc1)C(*)=O ; *NC(CCCNC(=N)N)C(*)=O ; *N[C@H](CCCNC(=N)N)C(*)=O]

A₃ is a lysine residue; alternatively

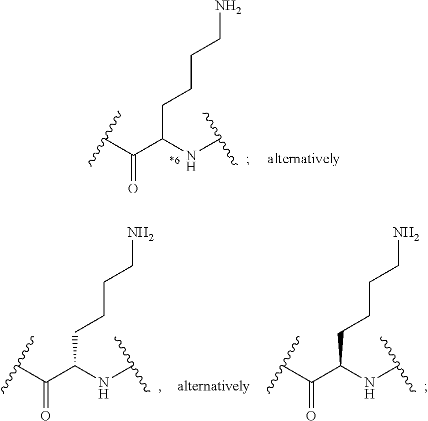

A₄ is selected from the group consisting of N-acetylglutamate residue, N-(4-carboxybutyryl)glutamate, glutamic acid residue, pyroglutamic acid residue, citrulline residue and aspartic acid residue; alternatively

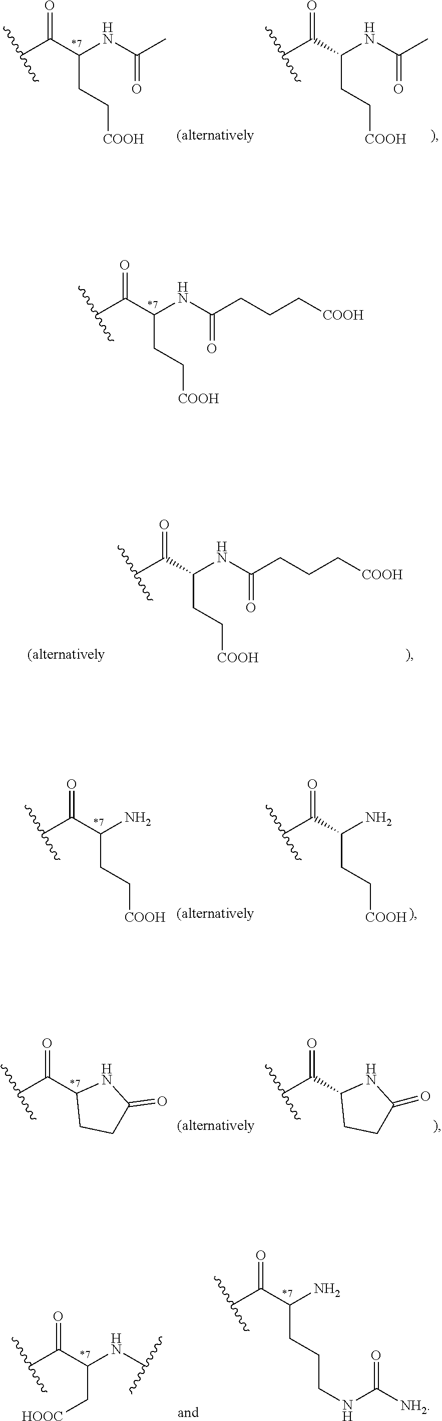

wherein, *4, *5, *6 and *7 are chiral centers, independently selected from in (S) or (R) configuration, or in racemic form; alternatively in (S) configuration; alternatively in (R) configuration;
for example,
L₂ is selected from:

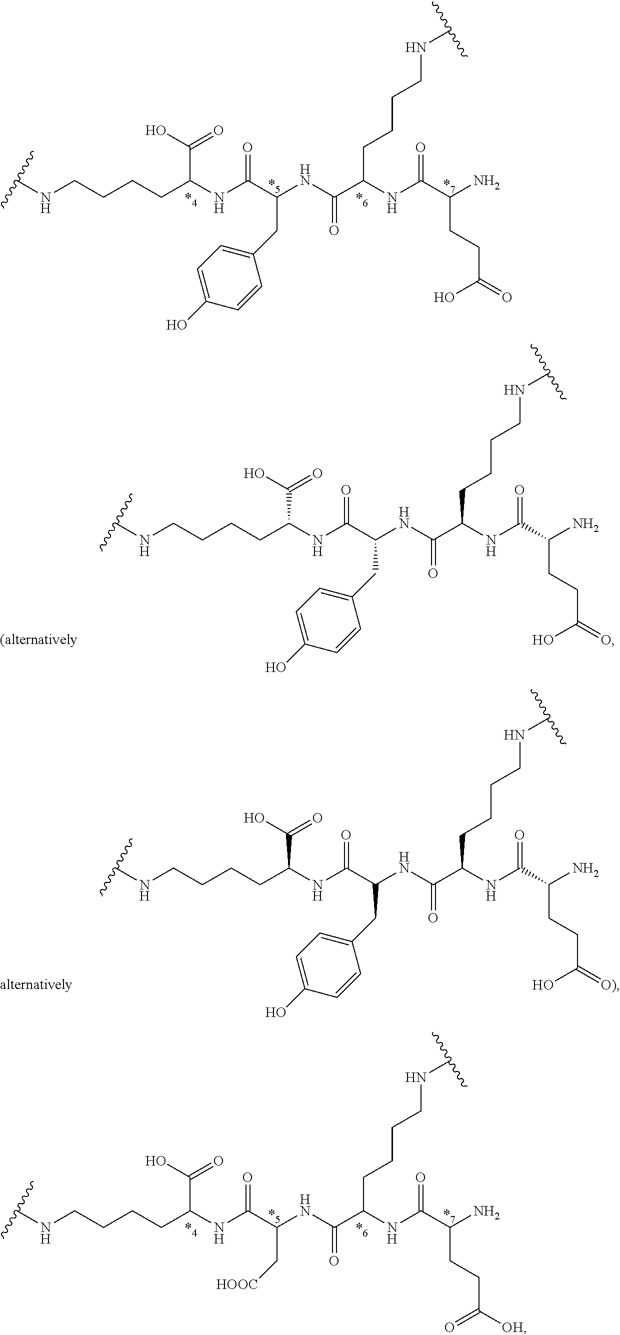

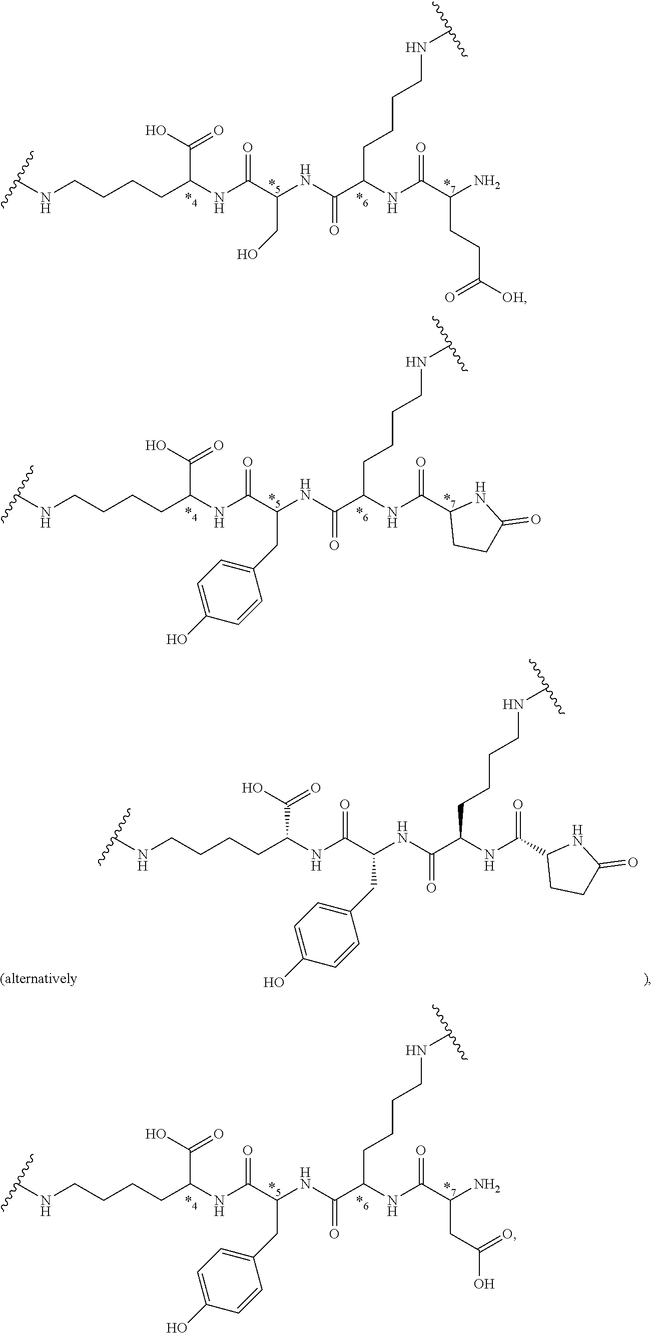

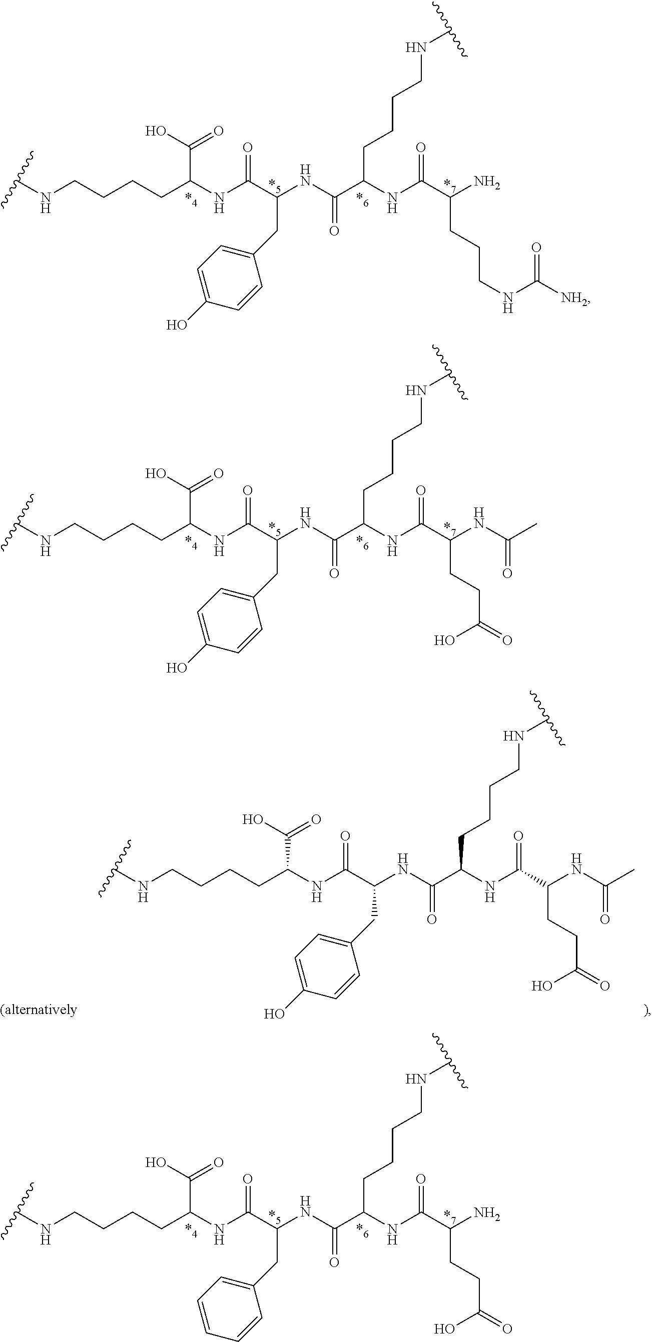

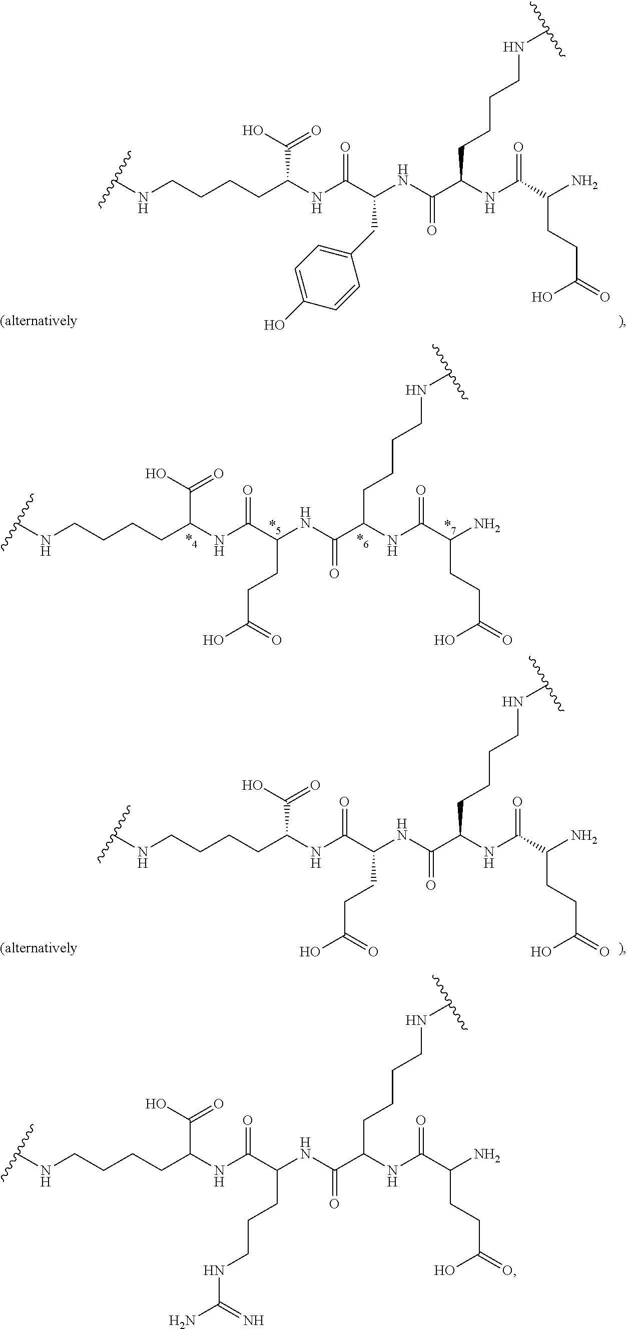

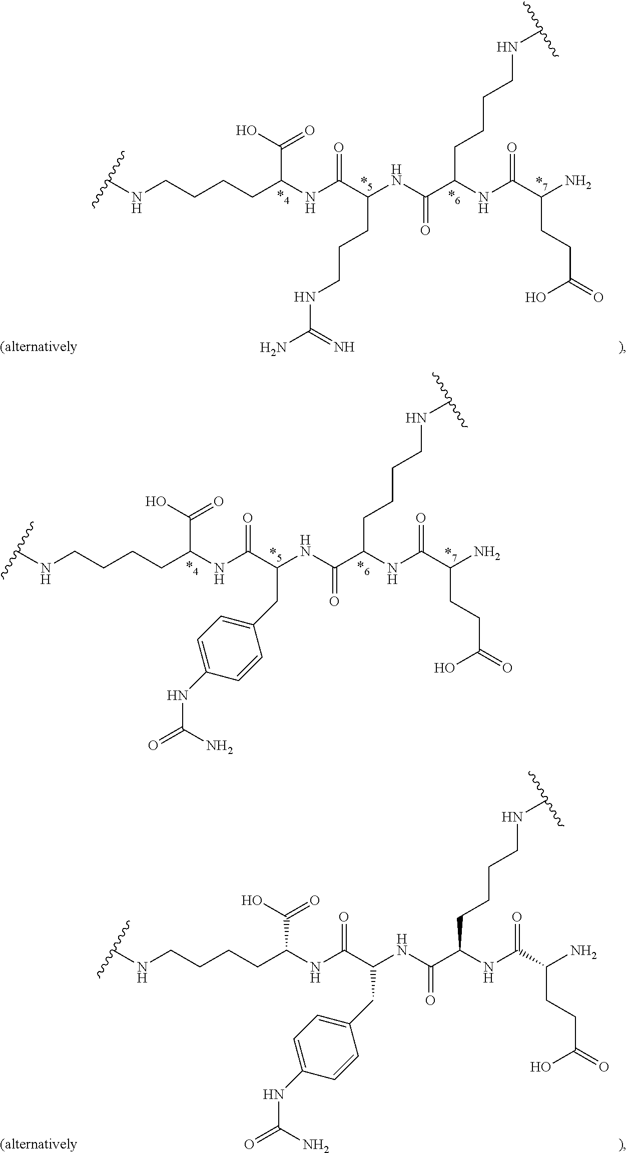

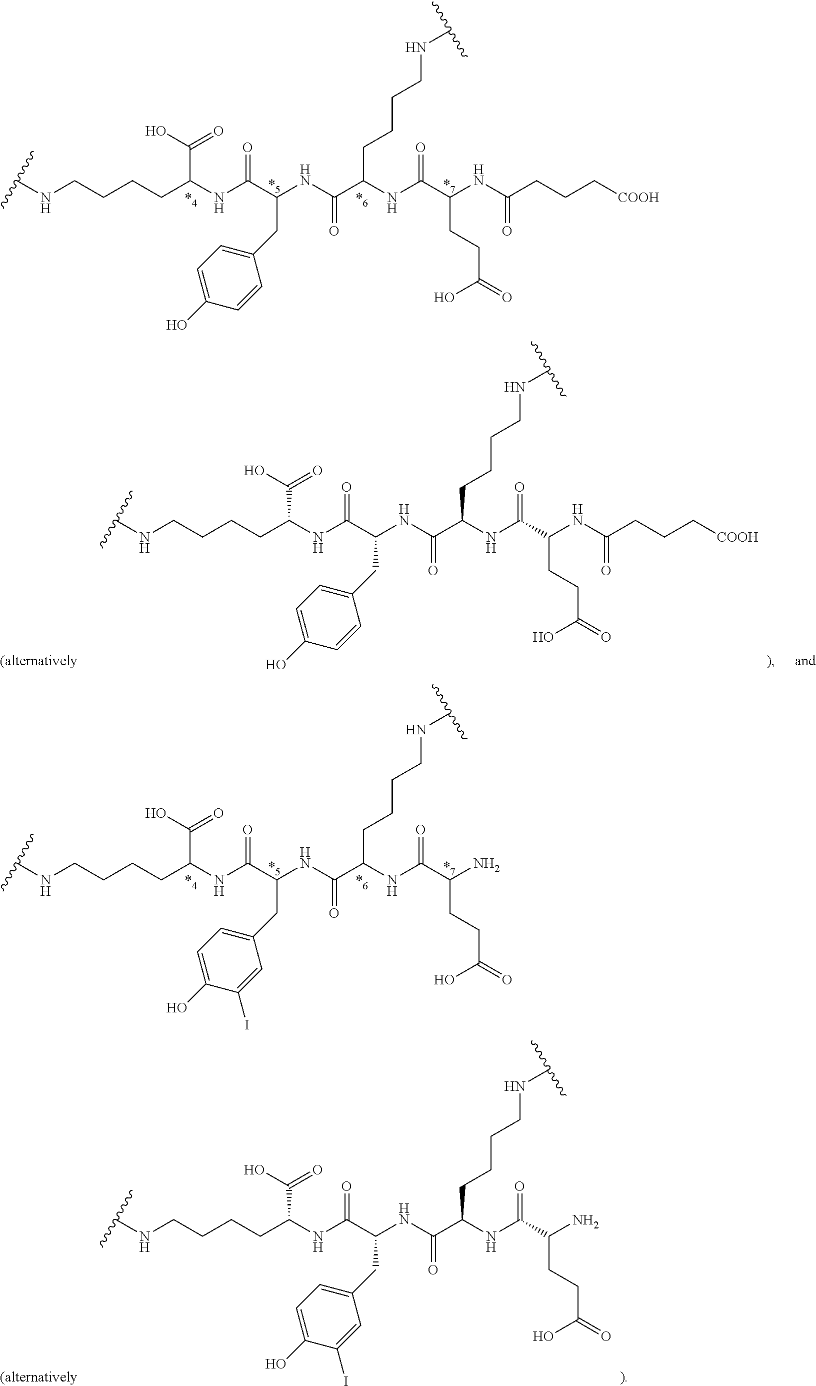

In a more specific embodiment, the present disclosure provides the above-mentioned compound, wherein,
Z is a chelating group derived from a chelating agent selected from the following:
1,4,7,10-tetraazacyclododecane-N,N′,N″,N′″-tetraacetic acid (DOTA),
N,N″-bis[2-hydroxy-5-(carboxyethyl)benzyl]ethylenediamine-N,N″-diacetic acid (HBED-CC),
1,4,7-triazacyclononane-1,4,7-triacetic acid (NOTA),
2-(4,7-bis(carboxymethyl)-1,4,7-triazononan-1-yl)glutaric acid (NODAGA),
2-(4,7,10-tris(carboxymethyl)-1,4,7,10-tetraazacyclododecane-1-yl)glutaric acid (DOTAGA),
1,4,7-triazacyclononanephosphinic acid (TRAP),
1,4,7-triazacyclononane-1-[methyl(2-carboxyethyl)phosphinic acid]-4,7-bis[methyl(2-hydroxymethyl)phosphinic acid](NOPO),
3,6,9,15-tetraazabicyclo[9.3.1.]pentadeca-1(15),11,13-triene-3,6,9-triacetic acid (PCTA),
N′-{5-[acetyl(hydroxy)amino]pentyl}-N-[5-({4-[(5-aminopentyl)(hydroxy)amino]-4-oxobutanoyl}amino)pentyl]-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (oxy-Do3A),
p-isocyanatobenzyl-DTPA (SCN-Bz-DTPA),
1-(p-isocyanatobenzyl)-3-methyl-DTPA (1B3M),
2-(p-isocyanatobenzyl)-4-methyl-DTPA (1M3B),
1-(2)-methyl-4-isocyanatobenzyl-DTPA (MX-DTPA),
(R)-2-amino-3-(4-isothiocyanatophenyl)propyl]-trans-(S,S)-cyclohexane-1,2-diaminepentaacetic acid (p-SCN-Bn-CHX-A″-DTPA),
6-hydrazinopyridine-3-carboxylic acid (HYNIC),
2-(4-isothiocyanatophenyl)-1,4,7-triazacyclononane-1,4,7-triacetic acid (p-SCN-Bn-NOTA) or
2-[(4-isothiocyanatophenyl)methyl]-1,4,7,10-tetraazacyclododecane-1,4,7,10-tetraacetic acid (p-SCN-Bn-DOTA);
alternatively,
Z is selected from

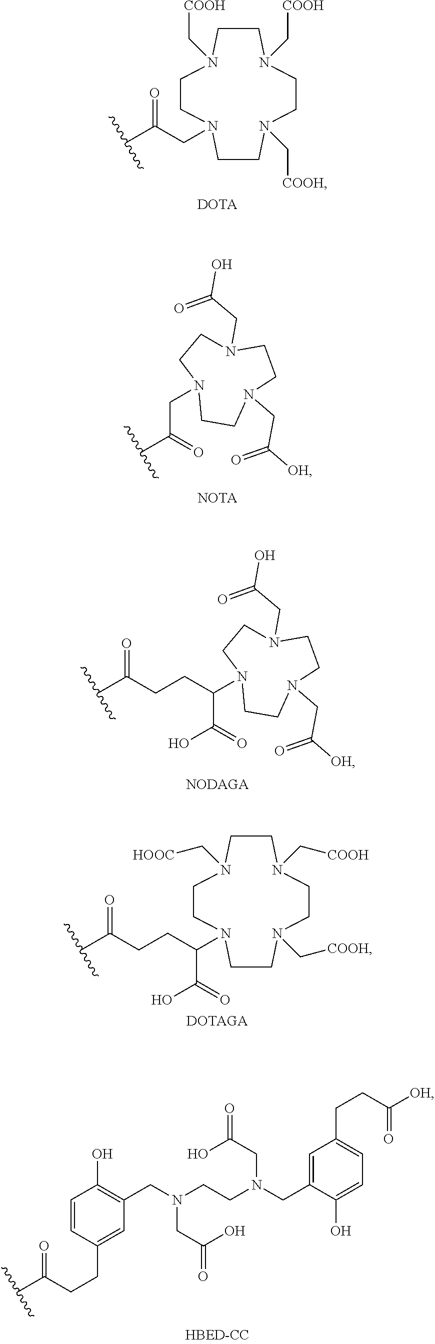

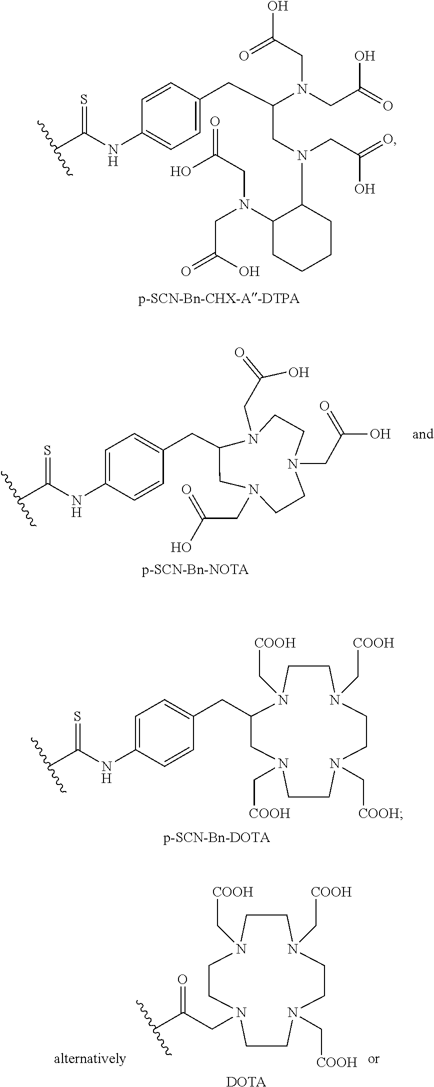

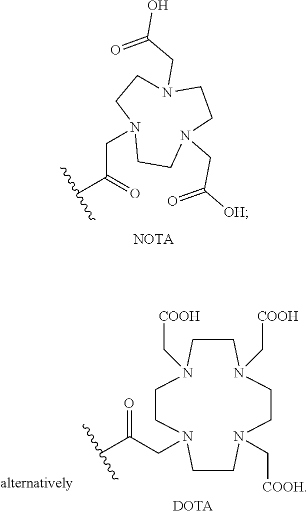

In a more specific embodiment, the present disclosure provides the above-mentioned compound, wherein,
Ab is

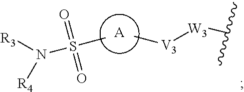

Ring A is selected from phenylene and C₅₋₆ heteroarylene; alternatively selected from phenylene and 1,3,4-thiadiazolylene; which is optionally substituted by 1, 2, 3 or 4 R_{A};
R_{A} is independently selected from H, D, halogen, CN, —OR, —SR, —NR_{b}R_{c}, C₁₋₆ alkyl, C₁₋₆ haloalkyl, C₃₋₇ cycloalkyl and 3-7 membered heterocyclyl; alternatively selected from H, D, halogen, C₁₋₆ alkyl and C₁₋₆ haloalkyl;
R₃ and R₄ are independently selected from H, halogen, C₁₋₆ alkyl and C₁₋₆ haloalkyl;
or R₄ and R_{A} are connected to form a C₁₋₄ alkylene group, alternatively a C₁₋₂ alkylene group, wherein 1, 2 or 3 (alternatively 1) methylene units are optionally and independently replaced by —CR*₂—, —NR*—, —O—, —C(O)—, —OC(O)—, —C(O)O—, —S—, —S(O)— or —S(O)₂—, alternatively replaced by —CR*₂—, —C(O)—, —OC(O)— or —C(O)O—, alternatively replaced by —C(O)—; and the ring formed by connecting R₄ with R_{A} is a ring structure fused with ring A;
V₃ is selected from a chemical bond, a C₁₋₆ alkylene group, a C₂₋₆ alkenylene group, and a C₂₋₆ alkynylene group;
W₃ is selected from —NR— and —O—;
R* is independently selected from H, D, halogen, C₁₋₆ alkyl and C₁₋₆ haloalkyl;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
alternatively,
Ab is selected from:

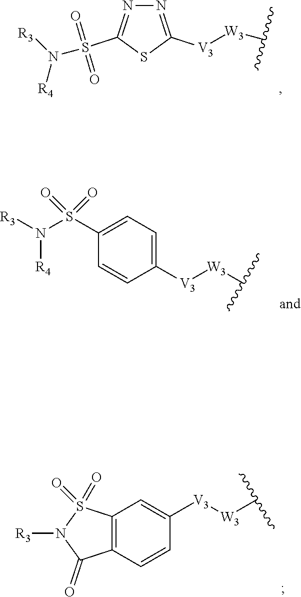

R₃ and R₄ are independently selected from H, halogen, C₁₋₆ alkyl and C₁₋₆ haloalkyl;
V₃ is selected from a chemical bond, a C₁₋₆ alkylene group, a C₂₋₆ alkenylene group, and a C₂₋₆ alkynylene group;
W₃ is selected from —NR— and —O—;
R is independently selected from H, C₁₋₆ alkyl and C₁₋₆ haloalkyl;
alternatively,
Ab is selected from:

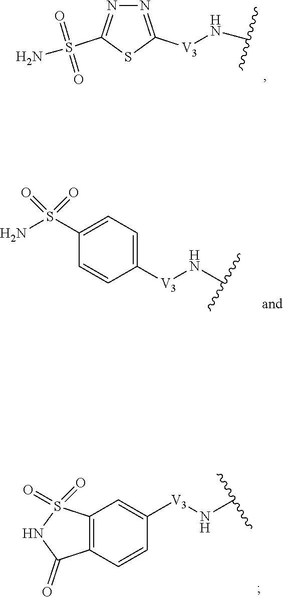

V₃ is selected from a chemical bond and a C₁₋₆ alkylene group (alternatively a C₁₋₄ alkylene group, alternatively a C₁₋₂ alkylene group);
still alternatively,
Ab is selected from:

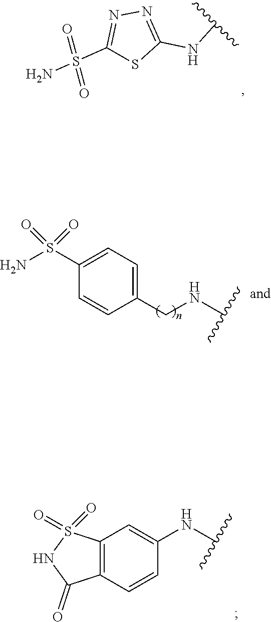

n is 0, 1, 2, 3, 4, 5 or 6, alternatively 0, 1, 2, 3 or 4, alternatively 0 or 2.

In a more specific embodiment, the present disclosure provides the above-mentioned compound, wherein,
Ab is

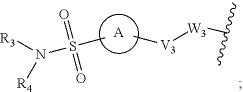

Ring A is selected from phenylene and C₅₋₆ heteroarylene; alternatively selected from phenylene and 1,3,4-thiadiazolylene; which is optionally substituted by 1, 2, 3 or 4 R_{A};
R_{A} is independently selected from H, D, halogen, CN, —OR, —SRₐ, —NR_{b}R_{c}, C₁₋₆ alkyl, C₁₋₆ haloalkyl, C₃₋₇ cycloalkyl and 3-7 membered heterocyclyl; alternatively selected from H, D, halogen, C₁₋₆ alkyl and C₁₋₆ haloalkyl;
R₃ and R₄ are independently selected from H, halogen, C₁₋₆ alkyl and C₁₋₆ haloalkyl;
or R₄ and R_{A} are connected to form a C₁₋₄ alkylene group, alternatively a C₁₋₂ alkylene group, wherein 1, 2 or 3 (alternatively 1) methylene units are optionally and independently replaced by —CR*₂—, —NR*—, —O—, —C(O)—, —OC(O)—, —C(O)O—, —S—, —S(O)— or —S(O)₂—, alternatively replaced by —CR*₂—, —C(O)—, —OC(O)— or —C(O)O—, alternatively replaced by —C(O)—; and the ring formed by connecting R₄ with R_{A} is a ring structure fused with ring A;
V₃ is selected from a chemical bond, a C₁₋₆ alkylene group, a C₂₋₆ alkenylene group, and a C₂₋₆ alkynylene group;
W₃ is selected from —NR— and —O—;
R* is independently selected from H, D, halogen, C₁₋₆ alkyl and C₁₋₆ haloalkyl;
L₁ is —W₁—V₁—W₄—V₂—W₂—;
W₁ is selected from a chemical bond, —NR—, —C(O)—, —C(O)O—, and —C(O)NR—;
W₂ is selected from —C(O)—, —OC(O)—, —NRC(O)—, —NR—, —C(O)NR—, and —O—;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted by 1, 2, 3, 4, 5 or 6 R₁ₛ; wherein one or more methylene units in the alkylene, alkenylene and alkynylene are optionally and independently replaced by —NR—, —NRC(O)—, —C(O)NR—, —NRS(O)₂—, —S(O)₂NR—, —O—, —C(O)—, —OC(O)—, —C(O)O—, —S—, —S(O)— or —S(O)₂—;
R₁ₛ is independently selected from H, D, —OR, —SRₐ, —NR_{b}R_{c}, halogen, C₁₋₆ alkyl, C₁₋₆ haloalkyl, C₃₋₇ cycloalkyl or 3-7 membered heterocyclyl; alternatively, R₁ₛ is independently in (S) or (R) configuration, or is in racemic form;
V₂ is a chemical bond or —(CR′R″)₁₋₆—;
R′ and R″ are independently selected from H, halogen, C₁₋₆ alkyl, C₁₋₆ haloalkyl, —C₁₋₆ alkylene-Ar; alternatively, R′ is independently in (S) or (R) configuration, or is in racemic form;
Ar is independently selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
L₂ is an amino acid chain formed by connecting 2-5 amino acid residues, wherein each amino acid residue can be optionally further substituted with one or more (alternatively 1, 2 or 3, alternatively 1) amino acid residues, and the amino acid residues can be optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent.

In a more specific embodiment, the present disclosure provides the above-mentioned compound, wherein,
Ab is selected from:

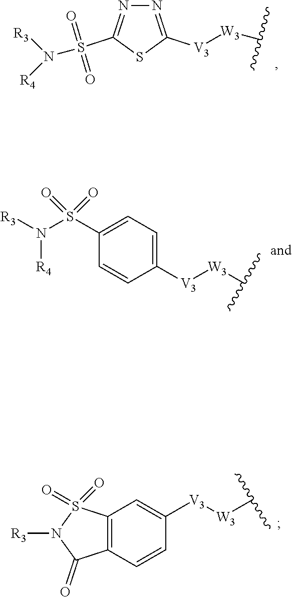

R₃ and R₄ are independently selected from H, halogen, C₁₋₆ alkyl and C₁₋₆ haloalkyl;
V₃ is selected from a chemical bond, a C₁₋₆ alkylene group, a C₂₋₆ alkenylene group, and a C₂₋₆ alkynylene group;
W₃ is selected from —NR— and —O—;
L₁ is —W₁—V₁—W₄—V₂—W₂—;
W₁ is selected from —C(O)—, —C(O)O— and —C(O)NR—; alternatively —C(O)—;
W₂ is selected from —C(O)—, —OC(O)— and —NRC(O)—; alternatively —C(O)—;
V₁ is selected from C₁₋₈ alkylene, C₂₋₈ alkenylene, C₂₋₈ alkynylene, phenylene and C₅₋₆ heteroarylene, alternatively C₁₋₈ alkylene (e.g. —(CH₂)ₘ—); which is optionally substituted by 1, 2, 3, 4, 5 or 6 R₁ₛ; wherein 1, 2 or 3 methylene units in the alkylene, alkenylene and alkynylene are optionally and independently replaced by —NR—, —NRC(O)—, —C(O)NR—, —C(O)—, —OC(O)— or —C(O)O—;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —OR, —NR_{b}R_{c}, halogen, C₁₋₆ alkyl and C₁₋₆ haloalkyl; alternatively H, D, —ORₐ and —NR_{b}R_{c};
V₂ is a chemical bond or —(CR′R″)₁₋₄—;
R′ is independently selected from H, D or —C₁₋₄ alkylene-Ar; alternatively, R′ is independently in (S) or (R) configuration, or in racemic form; alternatively in (S) configuration; alternatively in (R) configuration;
R″ is independently selected from H, D, halogen, C₁₋₆ alkyl and C₁₋₆ haloalkyl;
Ar is independently selected from C₆₋₁₄ aryl and 5-14 membered heteroaryl, alternatively C₆₋₁₄ aryl;
W₄ is selected from a chemical bond, —OC(O)—, —NRC(O)—, —C(O)O— and —C(O)NR—;

L₂ is

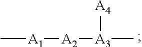

A₁, A₂, A₃ and A₄ are amino acid residues, and the amino acid residues are optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent.

In a more specific embodiment, the present disclosure provides the above-mentioned compound, wherein,
Ab is selected from:

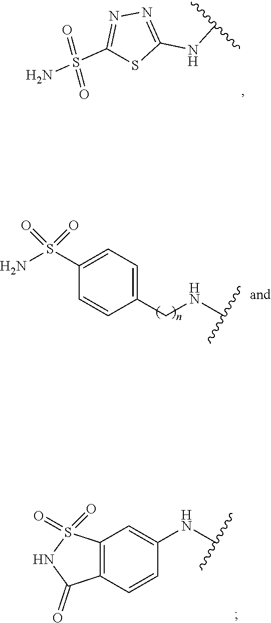

n is 0, 1, 2, 3, 4, 5 or 6, alternatively 0, 1, 2, 3 or 4, alternatively 0 or 2;
L₁ is —C(O)—V₁—W₄—V₂—C(O)—;
wherein V₁ is selected from C₁₋₆ alkylene (e.g. —(CH₂)ₘ—), phenylene and C₅₋₆ heteroarylene; alternatively selected from C₁₋₆ alkylene (e.g. —(CH₂)ₘ—), phenylene, pyrrolylene, furylene or thienylene; alternatively C₁₋₆ alkylene (e.g. —(CH₂)ₘ—); alternatively C₁₋₄ alkylene (e.g. —(CH₂)ₘ—); which is optionally substituted by 1, 2, 3, 4, 5 or 6 R₁ₛ; wherein 1, 2 or 3 methylene units in the alkylene are optionally and independently replaced by —NR—, —NRC(O)—, —C(O)NR—, —C(O)—, —OC(O)— or —C(O)O—;
m is independently selected from 1, 2, 3, 4, 5 or 6, alternatively 1, 2, 3 or 4;
R is independently selected from H, C₁₋₆ alkyl and C₁₋₆ haloalkyl;
R₁ₛ is independently selected from H, D, —OR, C₁₋₆ alkyl and C₁₋₆ haloalkyl; alternatively selected from H and —ORₐ;
V₂ is a chemical bond or —(CR′R″)₁₋₂— (alternatively —CHR′—);
R′ is independently selected from H or —C₁₋₄ alkylene-Ar; alternatively H or —CH₂—Ar; alternatively, R′ is independently in (S) or (R) configuration, or in racemic form; alternatively in (S) configuration; alternatively in (R) configuration;
R″ is independently selected from H, D, C₁₋₆ alkyl and C₁₋₆ haloalkyl; alternatively H or D;
Ar is independently C₆₋₁₄ aryl, such as phenyl, naphthyl, anthracenyl or phenanthryl; alternatively C₆₋₁₀ aryl, such as phenyl or naphthyl, alternatively naphthyl, alternatively 2-naphthyl;
W₄ is selected from a chemical bond, —OC(O)—, —NRC(O)—, —C(O)O— and —C(O)NR—; alternatively a chemical bond, —C(O)O— and —C(O)NR—; alternatively a chemical bond or —C(O)NR—;
L₂ is;

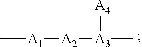

A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
Z is a chelating group derived from a chelating agent selected from the following:
1,4,7,10-tetraazacyclododecane-N,N′,N″,N′″-tetraacetic acid (DOTA),
N,N″-bis[2-hydroxy-5-(carboxyethyl)benzyl]ethylenediamine-N,N″-diacetic acid (HBED-CC),
1,4,7-triazacyclononane-1,4,7-triacetic acid (NOTA),
2-(4,7-bis(carboxymethyl)-1,4,7-triazononan-1-yl)glutaric acid (NODAGA),
2-(4,7,10-tris(carboxymethyl)-1,4,7,10-tetraazacyclododecane-1-yl)glutaric acid (DOTAGA),
1,4,7-triazacyclononanephosphinic acid (TRAP),
1,4,7-triazacyclononane-1-[methyl(2-carboxyethyl)phosphinic acid]-4,7-bis[methyl(2-hydroxymethyl)phosphinic acid](NOPO),
3,6,9,15-tetraazabicyclo[9.3.1.]pentadeca-1(15),11,13-triene-3,6,9-triacetic acid (PCTA),
N′-{5-[acetyl(hydroxy)amino]pentyl}-N-[5-({4-[(5-aminopentyl)(hydroxy)amino]-4-oxobutanoyl}amino)pentyl]-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (oxy-Do3A),
p-isocyanatobenzyl-DTPA (SCN-Bz-DTPA),
1-(p-isocyanatobenzyl)-3-methyl-DTPA (1B3M),
2-(p-isocyanatobenzyl)-4-methyl-DTPA (1M3B),
1-(2)-methyl-4-isocyanatobenzyl-DTPA (MX-DTPA),
(R)-2-amino-3-(4-isothiocyanatophenyl)propyl]-trans-(S,S)-cyclohexane-1,2-diaminepentaacetic acid (p-SCN-Bn-CHX-A″-DTPA),
6-hydrazinopyridine-3-carboxylic acid (HYNIC),
2-(4-isothiocyanatophenyl)-1,4,7-triazacyclononane-1,4,7-triacetic acid (p-SCN-Bn-NOTA) or
2-[(4-isothiocyanatophenyl)methyl]-1,4,7,10-tetraazacyclododecane-1,4,7,10-tetraacetic acid (p-SCN-Bn-DOTA).

In a more specific embodiment, the present disclosure provides the above-mentioned compound, wherein,
Ab is selected from:

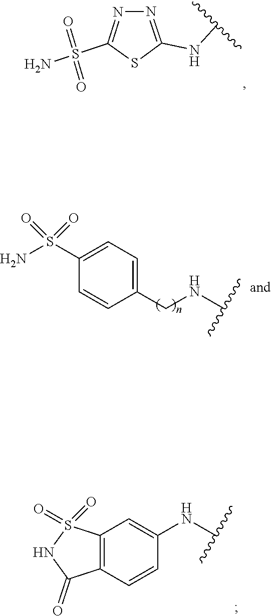

n is 0, 1, 2, 3, 4, 5 or 6, alternatively 0, 1, 2, 3 or 4, alternatively 0 or 2;
L₁ is selected from:

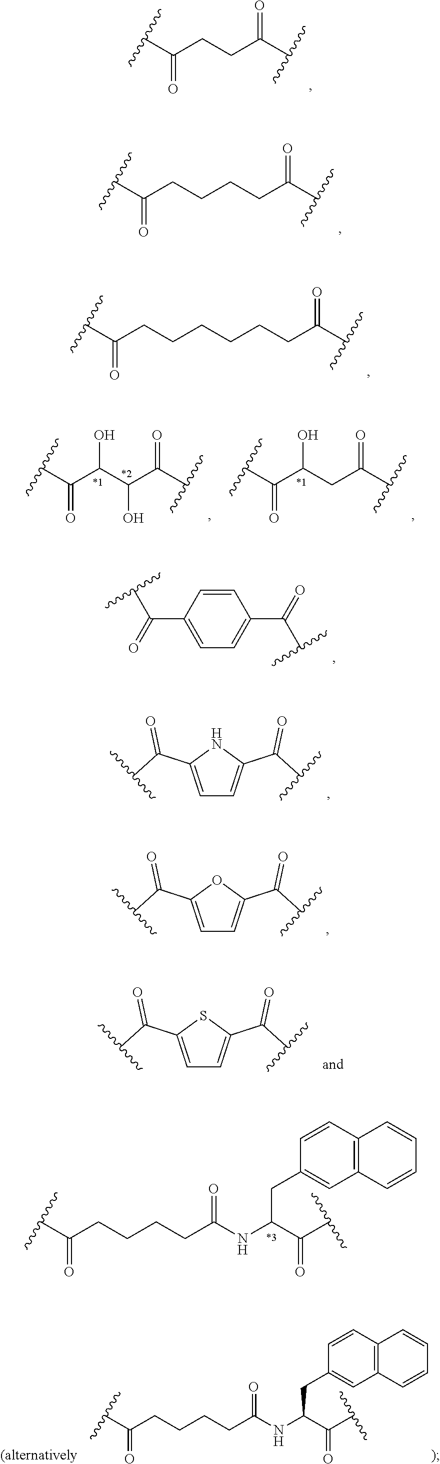

wherein, *1, *2 and *3 are chiral centers, independently selected from in (S) or (R) configuration, or in racemic form; alternatively in (S) configuration; alternatively in (R) configuration;
L₂ is

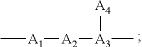

A₁ is a lysine residue;

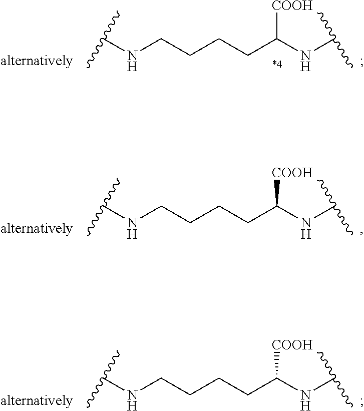 [SMILES: *NCCCCC(N*)C(=O)O ; *NCCCC[C@H](N*)C(=O)O ; *NCCCC[C@@H](N*)C(=O)O]

A₂ is selected from a tyrosine residue, an aspartic acid residue, a serine residue, a glutamic acid residue, a phenylalanine residue or an arginine residue, which is optionally substituted by 1, 2, 3 or 4 R_{L};
R_{L} is independently selected from halogen, —OR, —NR_{b}R_{c}, C₁₋₆ alkyl, C₁₋₆ haloalkyl and urea group; alternatively halogen, C₁₋₆ alkyl, C₁₋₆ haloalkyl and urea group;
alternatively A₂ is selected from

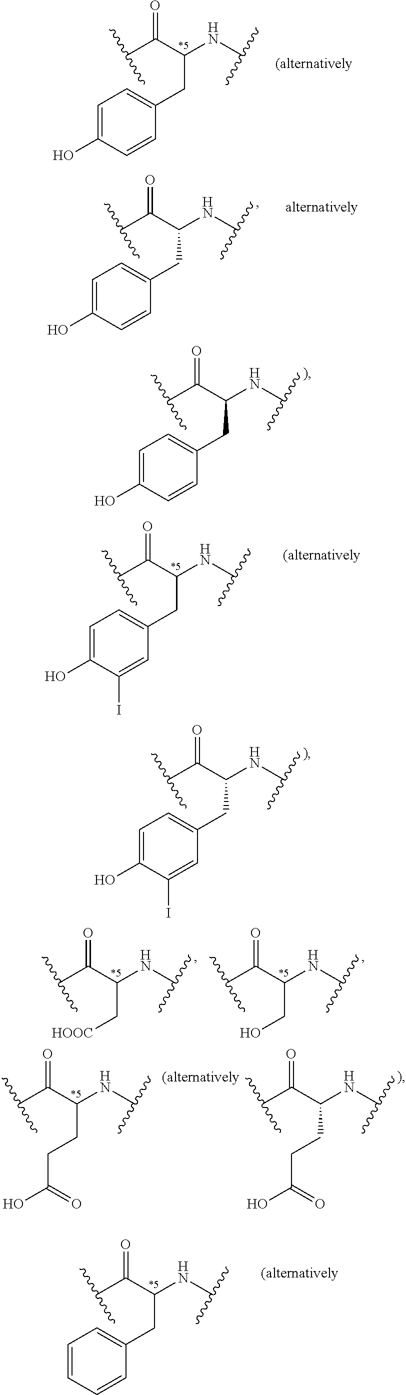

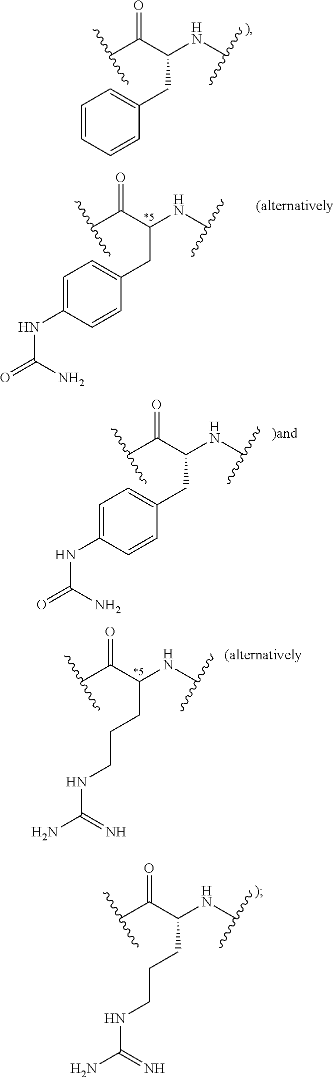

A₃ is a lysine residue; alternatively

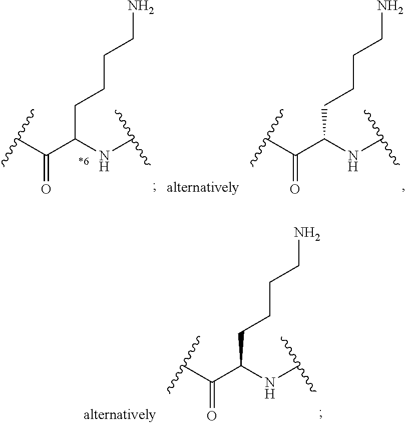

A₄ is selected from N-acetylglutamate residue, N-(4-carboxybutyryl)glutamate, glutamic acid residue, pyroglutamic acid residue, citrulline residue or aspartic acid residue; alternatively

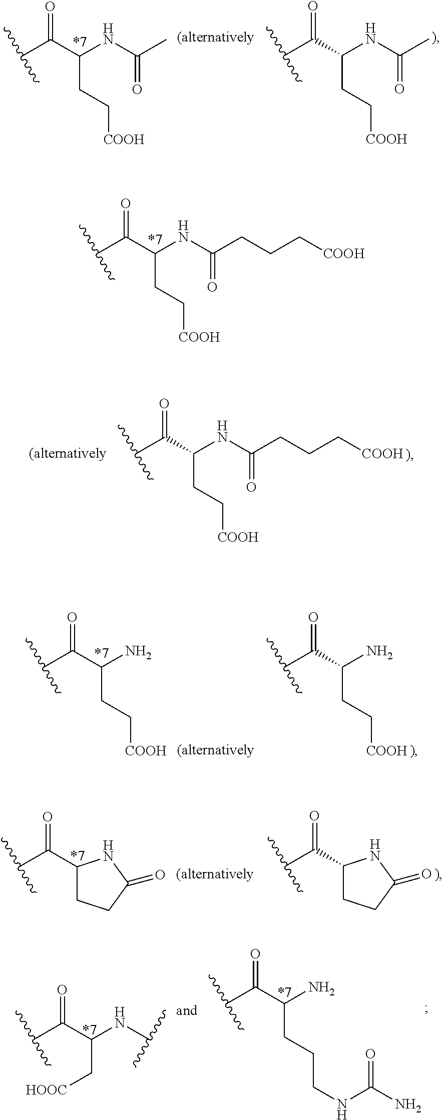

wherein, *4, *5, *6 and *7 are chiral centers, independently selected from in (S) or (R) configuration, or in racemic form; alternatively in (S) configuration; alternatively in (R) configuration;
Z is selected from

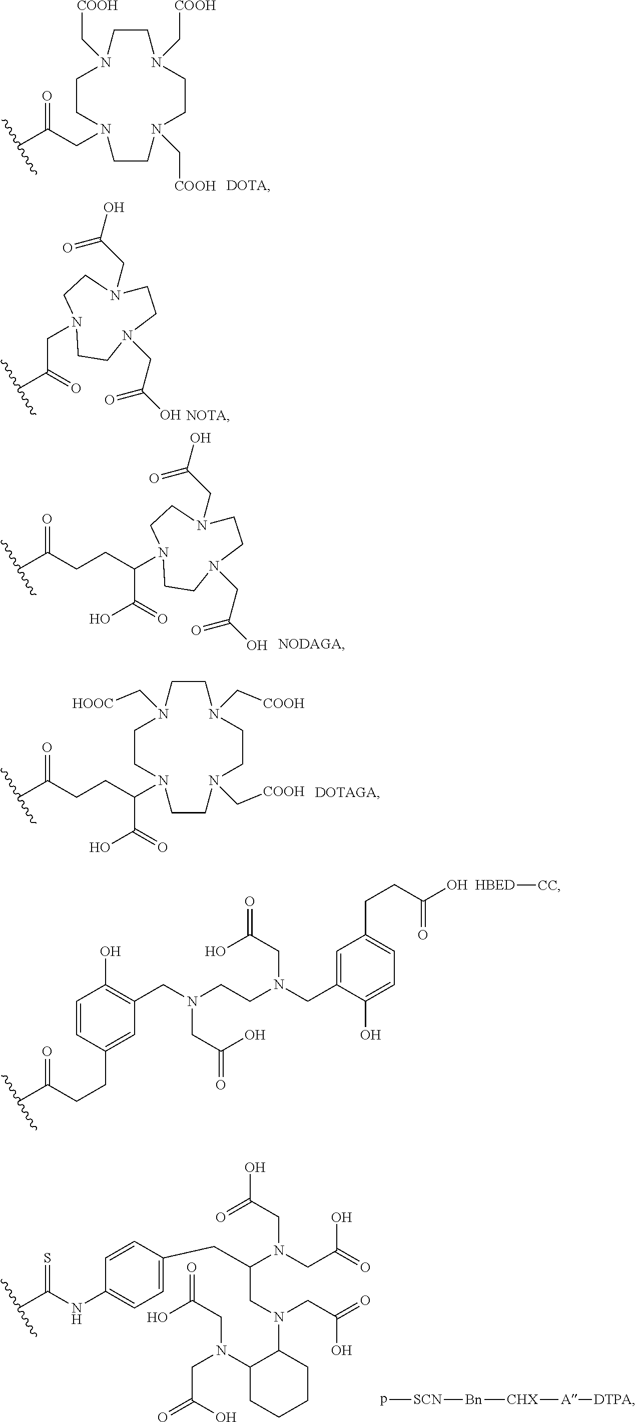

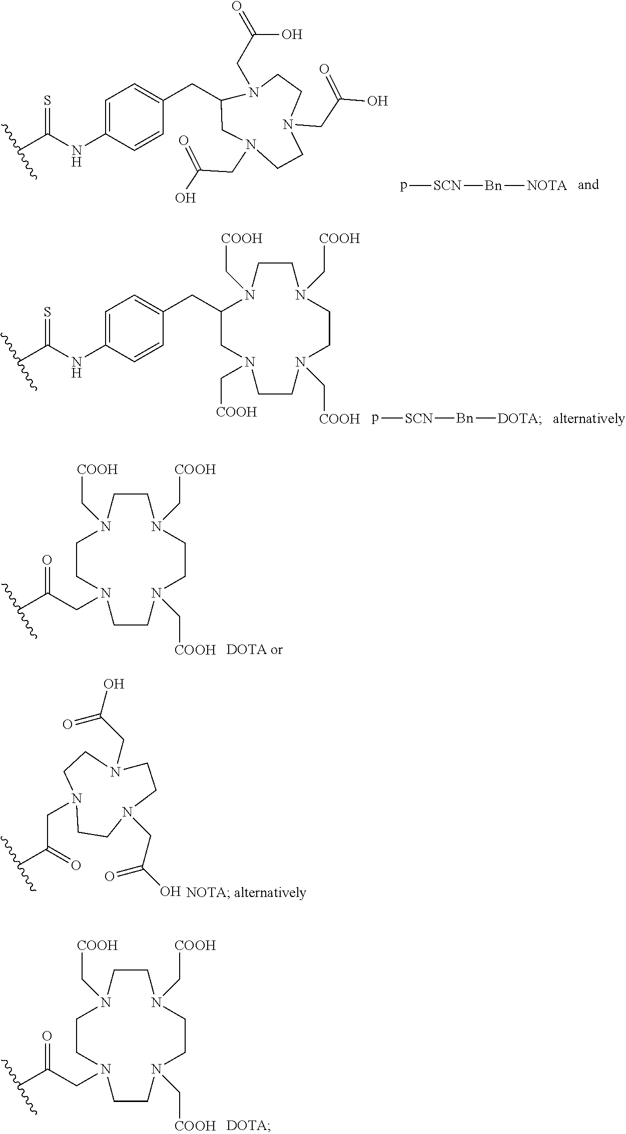

alternatively,
L₂ is selected from:

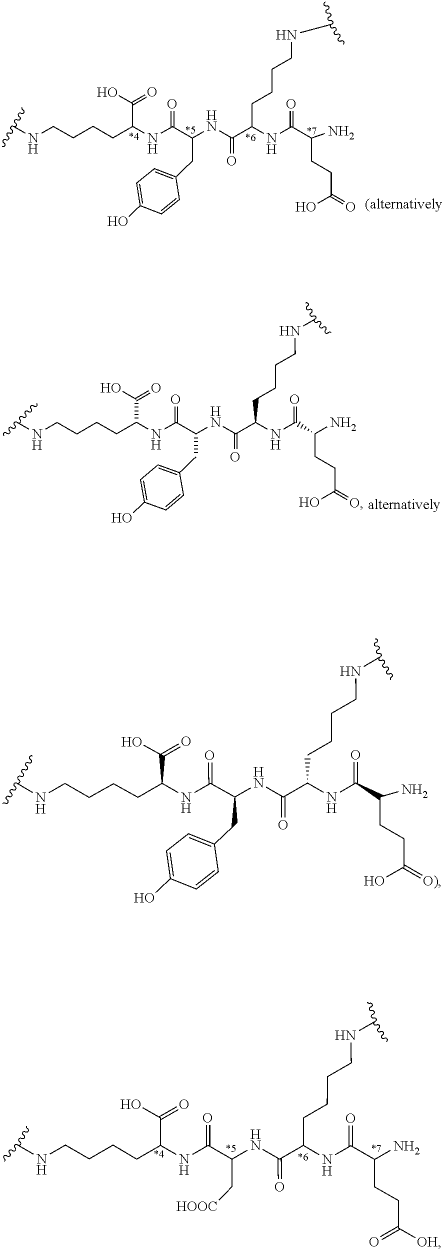

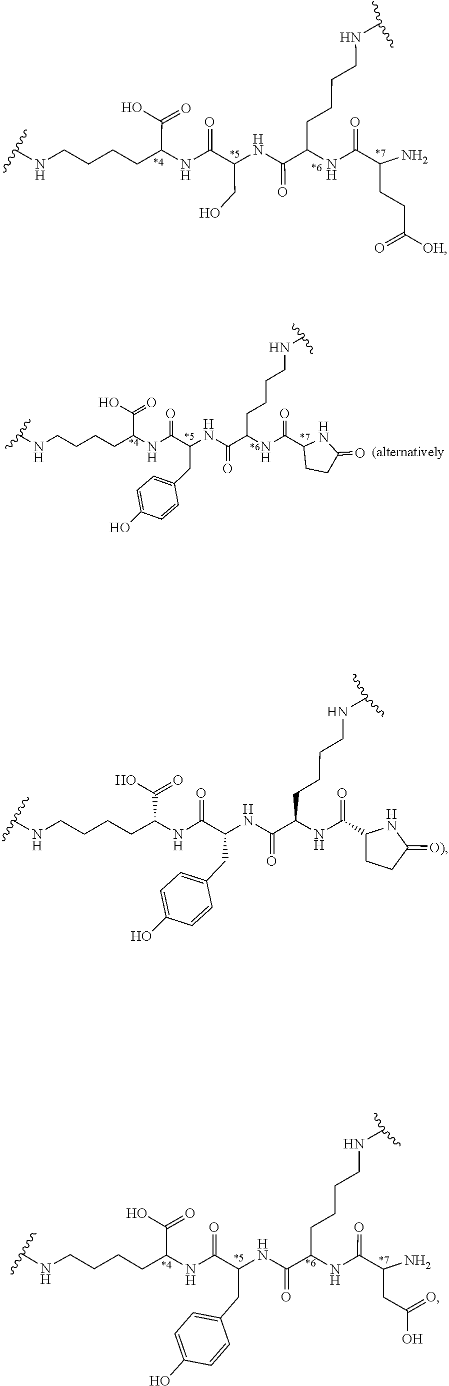

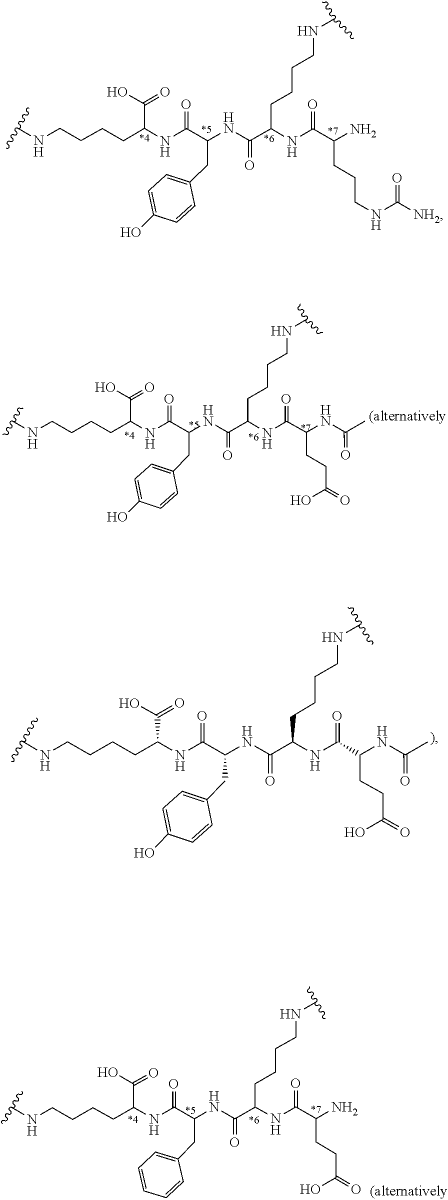

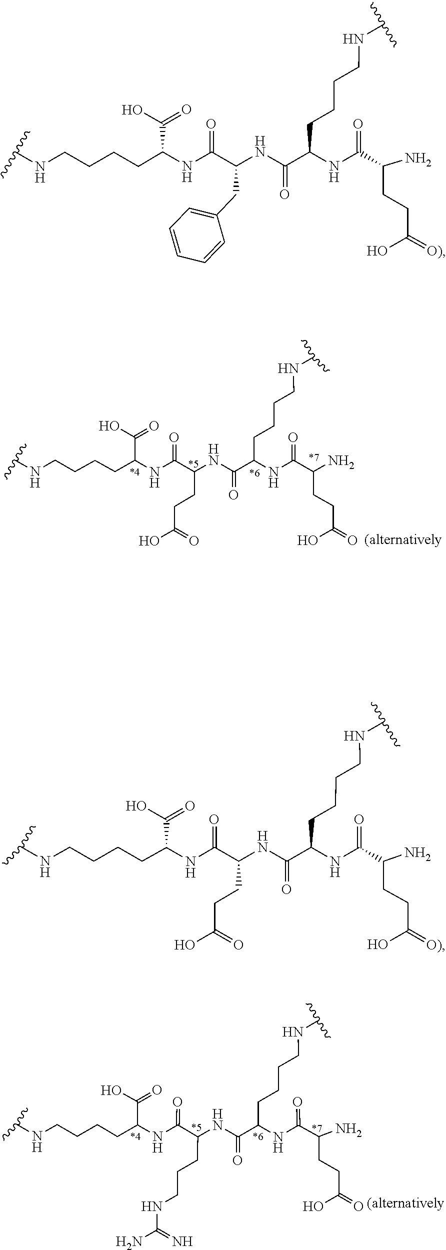

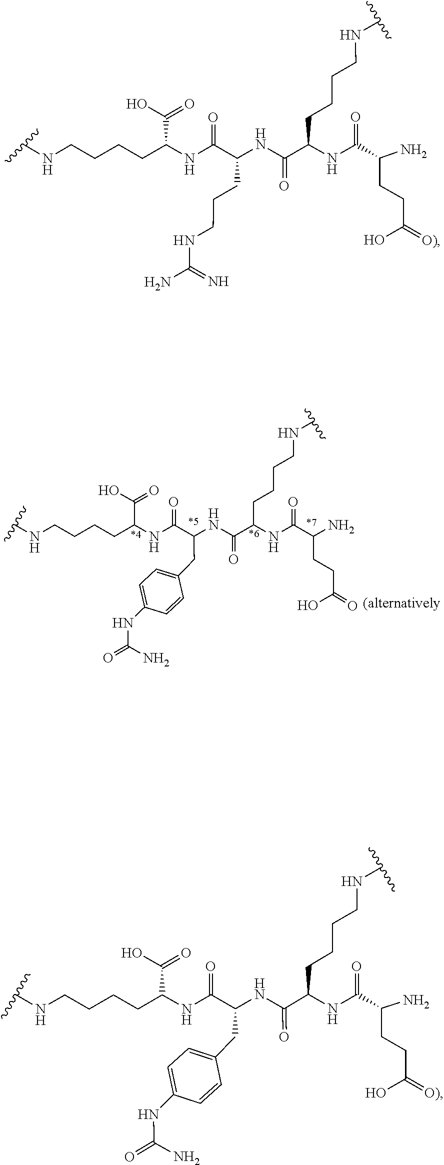

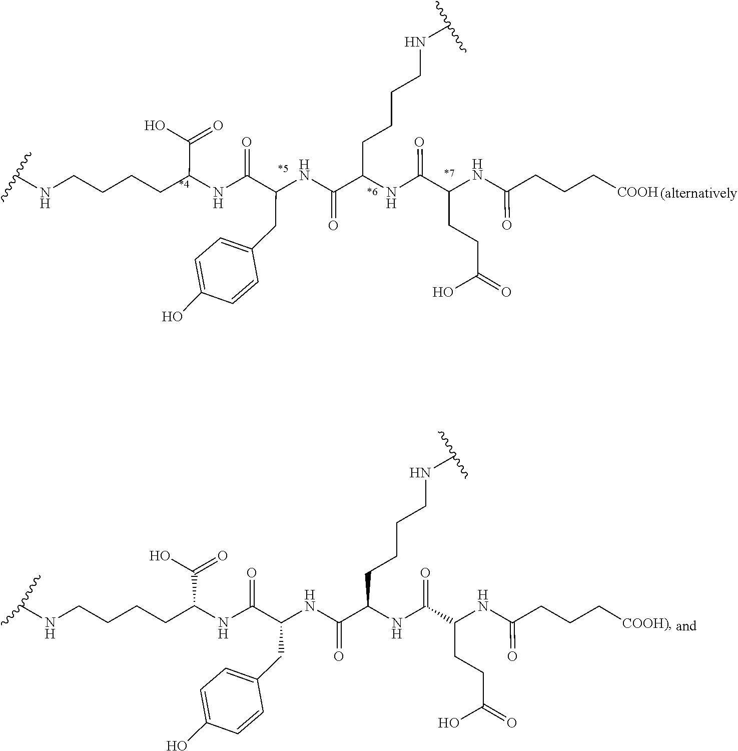

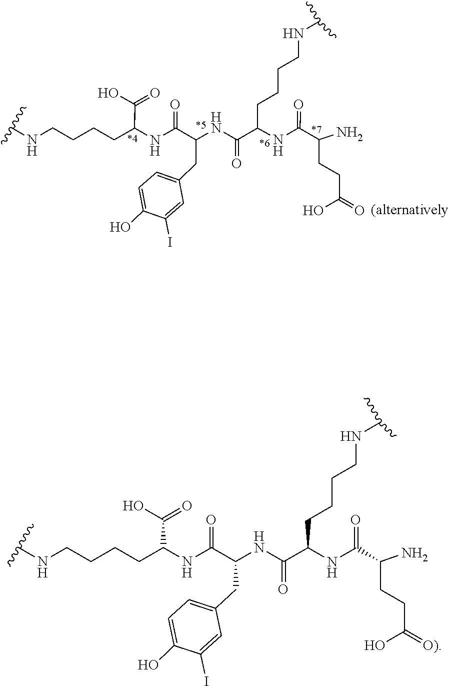

In a more specific embodiment, the present disclosure provides the above-mentioned compound, wherein, V₃ is selected from a chemical bond, a C₁₋₆ alkylene group, a C₂₋₆ alkenylene group, a C₂₋₆ alkynylene group and

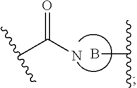

alternatively, a chemical bond, a C₁₋₆ alkylene group (alternatively a C₁₋₄ alkylene group, alternatively a C₁₋₂ alkylene group), and

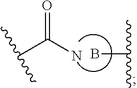

alternatively, a chemical bond and

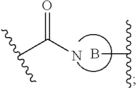

Alternatively,

Ring B is a 3-7 membered heterocyclylene group; alternatively, ring B is a 4-6 membered heterocyclylene group; alternatively, ring B is a 5 membered heterocyclylene group, for example

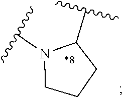

wherein, *8 is a chiral center, which is independently in (S) configuration; alternatively, in (R) configuration;
the remaining variables are as defined in the present disclosure.

In a more specific embodiment, the present disclosure provides the above-mentioned compound, wherein, W₃ is selected from —NR—, —O— and —C(O)—; alternatively, W₃ is selected from —NR— and —C(O)—; the remaining variables are as defined in the present disclosure.

In a more specific embodiment, the present disclosure provides the above-mentioned compound, wherein, Ab is selected from

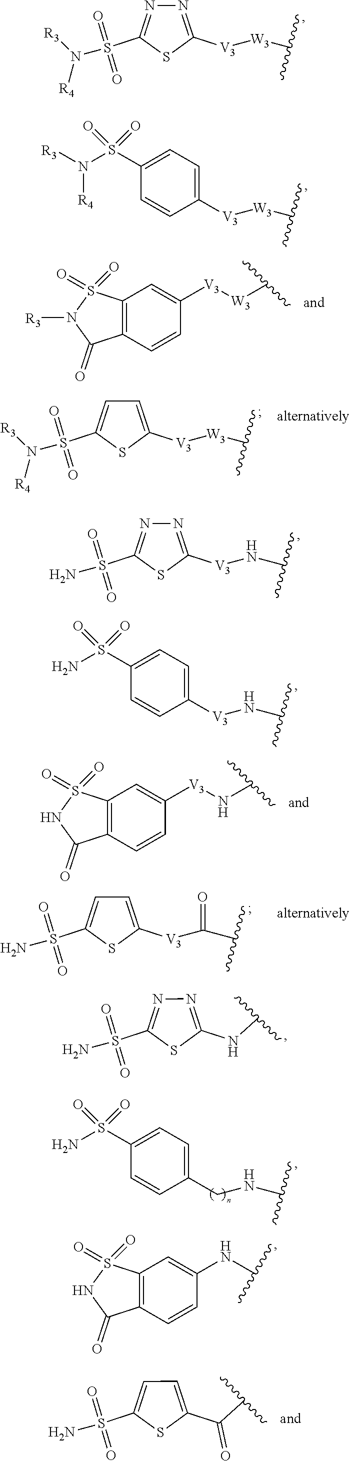

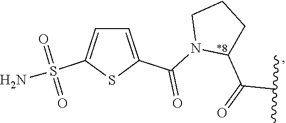 [SMILES: *C(=O)C1CCCN1C(=O)c1ccc(S(N)(=O)=O)s1]

wherein *8 is a chiral center, which is independently in (S) or (R) configuration, or is in racemic form; alternatively, in (S) configuration; alternatively, in (R) configuration;
the remaining variables are as defined in the present disclosure.

In a more specific embodiment, the present disclosure provides the above-mentioned compound, wherein, L₁ is —W₁—V₁—W₄—V₂—W₂—;
W₁ is selected from —NR—, —C(O)—, —C(O)O— and —C(O)NR—; alternatively —NR— or —C(O)—;
alternatively, R₁ₛ is independently selected from H, —OR, —NR_{b}R_{c}, C₁₋₆ alkyl and C₁₋₆ haloalkyl; alternatively H, —ORₐ and —NR_{b}R_{c}; alternatively H and —NR_{b}R_{c};
the remaining variables are described in the present disclosure;
alternatively,
L₁ is selected from:

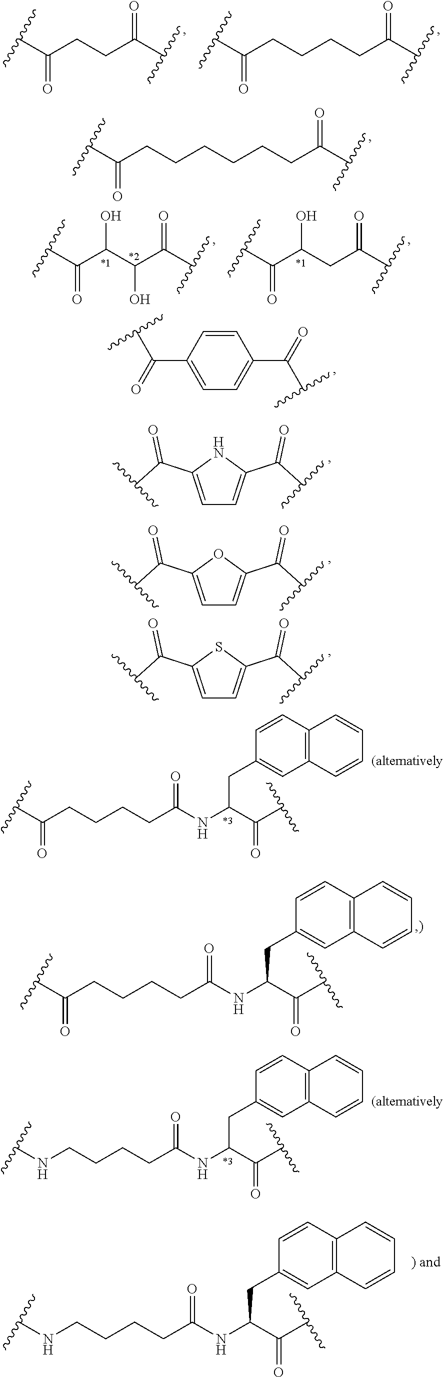

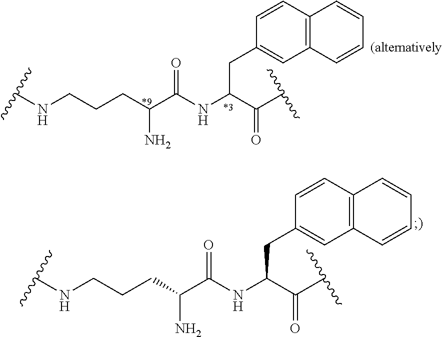 [SMILES: *NCCCC(N)C(=O)NC(Cc1ccc2ccccc2c1)C(*)=O ; *NCCC[C@@H](N)C(=O)N[C@@H](Cc1ccc2ccccc2c1)C(*)=O]

wherein *1, *2, *3 and *9 are chiral centers, which are independently in (S) or (R) configurations, or in racemic form; alternatively in (S) configuration; alternatively in (R) configuration;
the remaining variables are described in the present disclosure.

In a more specific embodiment, the present disclosure provides the above-mentioned compound, which has a structure shown in formula (III-1) or formula (IV-1),

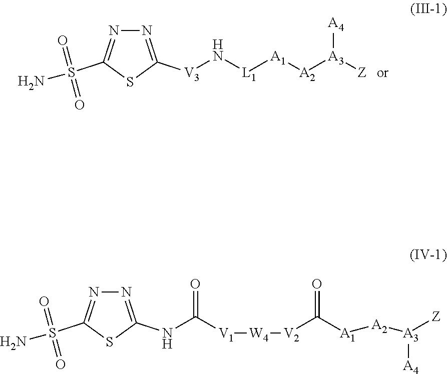

wherein,
V is selected from a chemical bond, a C₁₋₆ alkylene group, a C₂₋₆ alkenylene group, and a C₂₋₆ alkynylene group;
L₁ is —W₁—V₁—W₄—V₂—W₂—;
W₁ is selected from —C(O)—, —C(O)O— and —C(O)NR—; alternatively —C(O)—;
W₂ is selected from —C(O)—, —OC(O)— and —NRC(O)—; alternatively —C(O)—;
V₁ is selected from C₁₋₈ alkylene, C₂₋₆ alkenylene and C₂₋₆ alkynylene, alternatively C₁₋₈ alkylene (e.g. —(CH₂)ₘ—), which is optionally substituted by 1, 2, 3, 4, 5 or 6 R₁ₛ;
m is independently selected from 1, 2, 3, 4, 5, 6, 7 or 8;
R is independently selected from H, C₁₋₆ alkyl and C₁₋₆ haloalkyl;
R₁ₛ are independently selected from H, D, —OR, —NR_{b}R_{c}, halogen, C₁₋₆ alkyl and C₁₋₆ haloalkyl; alternatively H, D, —ORₐ and —NR_{b}R_{c}; alternatively, R₁ₛ are independently in (S) or (R) configuration, or are in racemic form;
V₂ is a chemical bond or —(CR′R″)₁₋₆—; alternatively —(CR′R″)₁₋₆—;
R′ is independently selected from H, D or —C₁₋₄ alkylene-Ar; alternatively, R′ is independently in (S) or (R) configuration, or is in racemic form;
R″ is independently selected from H, D, halogen, C₁₋₆ alkyl and C₁₋₆ haloalkyl;
Ar is independently selected from C₆₋₁₄ aryl and 5-14 membered heteroaryl; alternatively C₆₋₁₄ aryl;
W₄ is selected from a chemical bond, —OC(O)—, —NRC(O)—, —C(O)O— and —C(O)NR—;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
Z is as defined herein.

In a more specific embodiment, the present disclosure provides the above-mentioned compound, wherein,
V₃ is selected from a chemical bond and a C₁₋₆ alkylene group (alternatively a C₁₋₄ alkylene group, alternatively a C₁₋₂ alkylene group); alternatively a chemical bond;
L₁ is —W₁—V₁—W₄—V₂—W₂—;
W₁ is —C(O)—;
W₂ is —C(O)—;
V₁ is selected from C₁₋₆ alkylene, C₂₋₆ alkenylene and C₂₋₆ alkynylene, alternatively C₁₋₆ alkylene (e.g. —(CH₂)ₘ—), alternatively C₁₋₄ alkylene (e.g. —(CH₂)ₘ—), which is optionally substituted by 1, 2, 3, 4, 5 or 6 R₁ₛ;
m is independently selected from 1, 2, 3, 4, 5 or 6, alternatively 1, 2, 3 or 4;
R₁ₛ is independently selected from H, D, —ORₐ, C₁₋₆ alkyl and C₁₋₆ haloalkyl; alternatively selected from H and —ORₐ; alternatively selected from H and D;
V₂ is a chemical bond or —(CR′R″)₁₋₄—; alternatively a chemical bond or —(CR′R″)₁₋₂— (alternatively —CHR′—); alternatively —(CR′R″)₁₋₂— (alternatively —CHR′—);
R′ is independently selected from H or —C₁₋₄ alkylene-Ar, alternatively H or —CH₂—Ar; alternatively, R′ is independently in (S) or (R) configuration, or in racemic form; alternatively in (S) configuration;
R″ is independently selected from H, D, C₁₋₆ alkyl and C₁₋₆ haloalkyl; alternatively H or D;
Ar is independently C₆₋₁₄ aryl, such as phenyl, naphthyl, anthracenyl or phenanthryl; alternatively C₆₋₁₀ aryl, such as phenyl or naphthyl, alternatively naphthyl, alternatively 2-naphthyl;
W₄ is selected from a chemical bond or —C(O)NR—;
R is independently selected form H, C₁₋₆ alkyl and C₁₋₆ haloalkyl;
Rₐ is independently selected from H, C₁₋₆ alkyl and C₁₋₆ haloalkyl;
A₁ is a lysine residue;
A₂ is a tyrosine residue;
A₃ is a lysine residue;
A₄ is a glutamic acid residue or an N-acetylglutamic acid residue, alternatively a glutamic acid residue;
Z is selected from

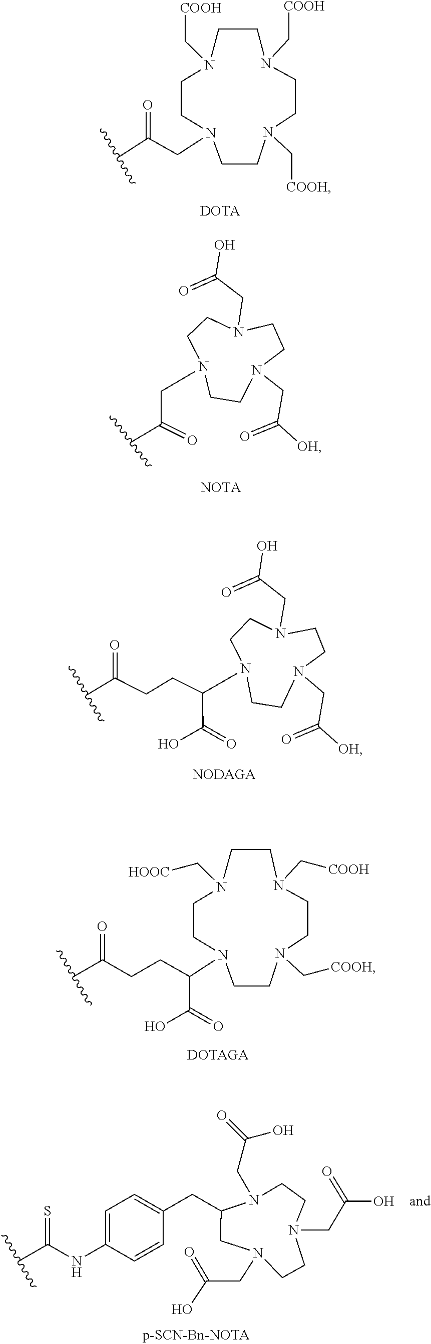

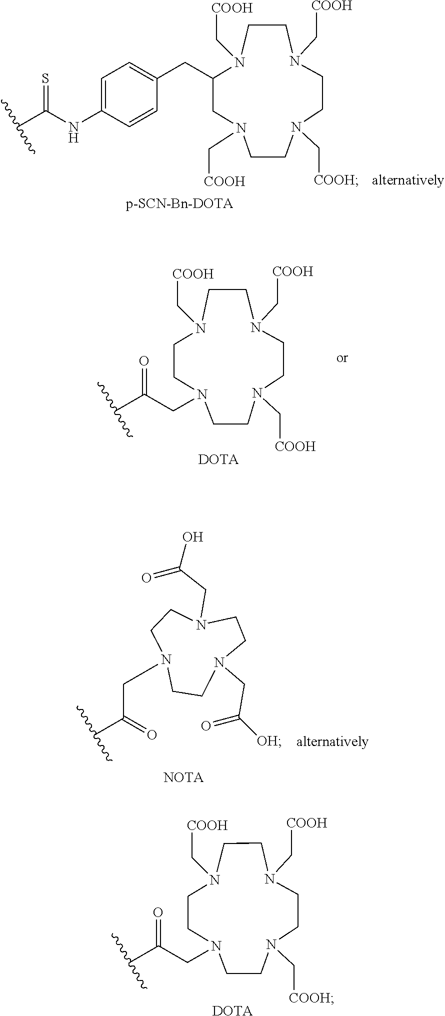

alternatively

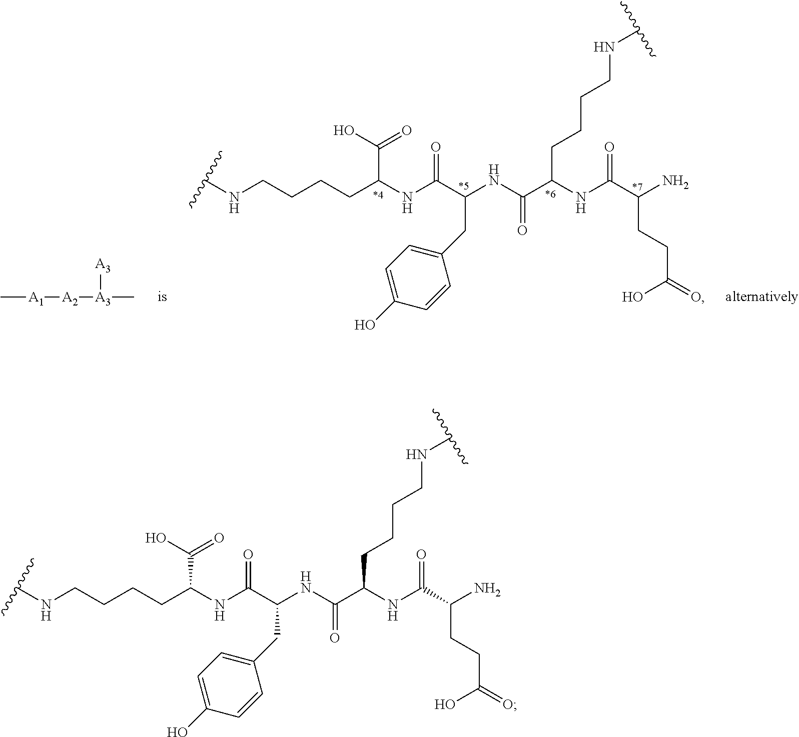

wherein, *4, *5, *6 and *7 are chiral centers, independently selected from in (S) or (R) configuration, or in racemic form; alternatively in (R) configuration;
alternatively, L₁ is

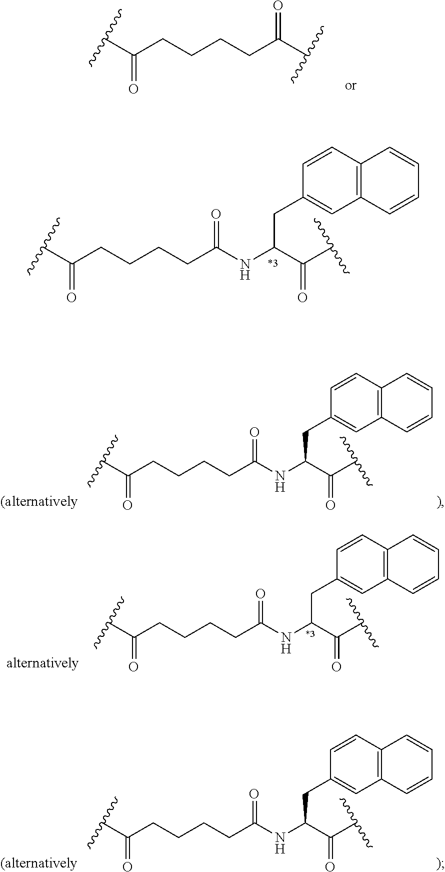

wherein, *3 is a chiral center, independently selected from in (S) or (R) configuration, or in racemic form; alternatively in (S) configuration.

In a more specific embodiment, the present disclosure provides the above-mentioned compound, which has a structure represented by formula (II-4),

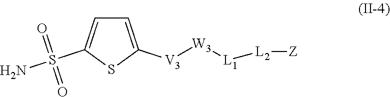

wherein,
V₃ is selected from a chemical bond, a C₁₋₆ alkylene group, a C₂₋₆ alkenylene group and a C₂₋₆ alkynylene group, and

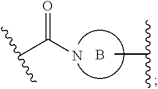

Ring B is a 3-7 membered heterocyclyl group;
W₃ is selected from —NR— and —C(O)—; alternatively —C(O)—;
L₁ is —W₁—V₁—W₄—V₂—W₂—;
W₁ is selected from —NR—, —C(O)—, —C(O)O— and —C(O)NR—; alternatively —NR— or —C(O)—;
W₂ is selected from —C(O)—, —OC(O)— and —NRC(O)—; alternatively —C(O)—;
V₁ is selected from C₁₋₈ alkylene, C₂₋₆ alkenylene and C₂₋₆ alkynylene, alternatively C₁₋₈ alkylene (e.g. —(CH₂)ₘ—), which is optionally substituted by 1, 2, 3, 4, 5 or 6 R₁ₛ;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —OR, —NR_{b}R_{c}, halogen, C₁₋₆ alkyl and C₁₋₆ haloalkyl; alternatively selected from H, D, —ORₐ and —NR_{b}R_{c}; alternatively, R₁ₛ is independently in (S) or (R) configuration, or in racemic form;
V₂ is a chemical bond or —(CR′R″)₁₋₆—; alternatively —(CR′R″)₁₋₆—;
R′ is independently selected from H, D or —C₁₋₄ alkylene-Ar, alternatively, R′ is independently in (S) or (R) configuration, or in racemic form;
R″ is independently selected from H, D, halogen, C₁₋₆ alkyl and C₁₋₆ haloalkyl;
Ar is independently selected from C₆₋₁₄ aryl and 5-14 membered heteroaryl; alternatively C₆₋₁₄ aryl;
W₄ is selected from a chemical bond, —OC(O)—, —NRC(O)—, —C(O)O— and —C(O)NR—;
Rₐ, R_{b} and R_{c} are independently selected from H, C₁₋₆ alkyl and C₁₋₆ haloalkyl; or R_{b}, R_{c} and the atom to which they are attached together form a 3-7 membered heterocyclyl group;
L₂ is

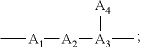

A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
Z is as defined in claim 1 or 5.

In a more specific embodiment, the present disclosure provides the above-mentioned compound, wherein,
V₃ is selected from a chemical bond, a C₁₋₆ alkylene group (alternatively a C₁₋₄ alkylene group, alternatively a C₁₋₂ alkylene group), and

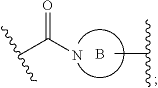

alternatively a chemical bond and

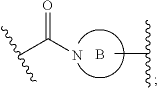

Ring B is a 4-6 membered heterocyclyl group; alternatively a 5-membered heterocyclyl group; for example

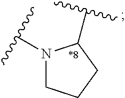 [SMILES: *C1CCCN1*]

W₃ is selected from —NR— and —C(O)—; alternatively —C(O)—;
L₁ is —W₁—V₁—W₄—V₂—W₂—;
W₁ is —NR—; alternatively —NH—;
W₂ is —C(O)—;
V₁ is selected from C₁₋₆ alkylene, C₂₋₆ alkenylene and C₂₋₆ alkynylene, alternatively C₁₋₆ alkylene (e.g. —(CH₂)ₘ—), alternatively C₁₋₄ alkylene (e.g. —(CH₂)ₘ—), which is optionally substituted by 1, 2, 3, 4, 5 or 6 R₁ₛ;
m is independently selected from 1, 2, 3, 4, 5, and 6, alternatively 1, 2, 3, and 4;
R₁ₛ is independently selected from H, D, —ORₐ, —NR_{b}R_{c}, C₁₋₆ alkyl and C₁₋₆ haloalkyl; alternatively selected from H, —ORₐ and —NR_{b}R_{c}; alternatively H and —NR_{b}R_{c};
V₂ is a chemical bond or —(CR′R″)₁₋₄—; alternatively a chemical bond or —(CR′R″)₁₋₂— (alternatively —CHR′—); alternatively —(CR′R″)₁₋₂— (alternatively —CHR′—);
R′ is independently selected from H or —C₁₋₄ alkylene-Ar, alternatively, H or —CH₂—Ar; alternatively, R′ is independently in (S) or (R) configuration, or in racemic form; alternatively in (S) configuration;
R″ is independently selected from H, D, C₁₋₆ alkyl and C₁₋₆ haloalkyl; alternatively H, or D;
Ar is independently C₆₋₁₄ aryl, such as phenyl, naphthyl, anthracyl or phenanthryl; alternatively C₆₋₁₀ aryl, such as phenyl, or naphthyl, alternatively naphthyl, alternatively 2-naphthyl;
W₄ is selected from a chemical bond and —C(O)NR—; alternatively —C(O)NR—;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; alternatively H;
L₂ is

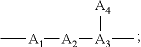

A₁ is a lysine residue;
A₂ is a tyrosine residue;
A₃ is a lysine residue;
A₄ is a glutamic acid residue or an N-acetylglutamic acid residue, alternatively a glutamic acid residue;
Z is selected from

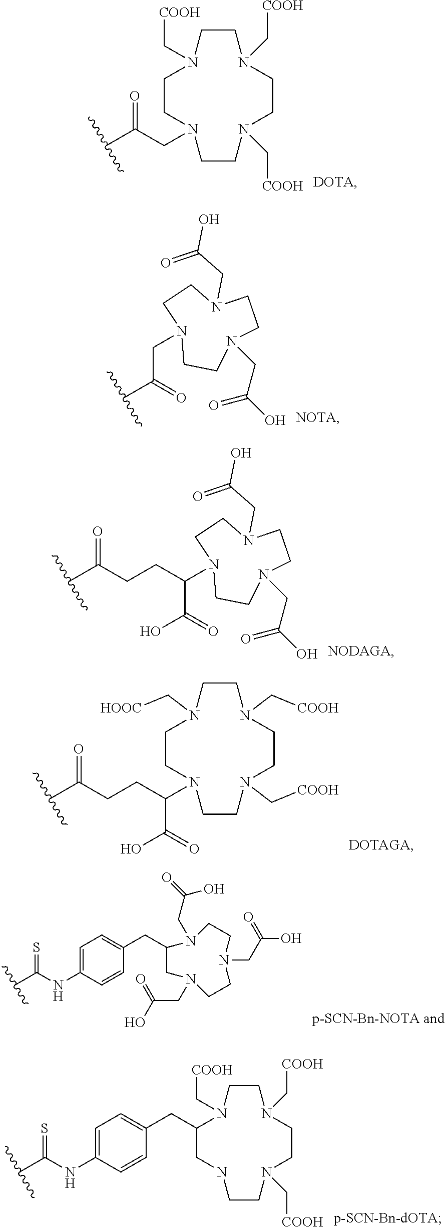

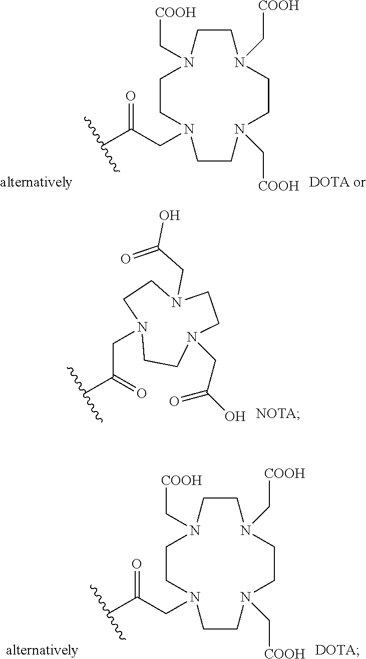 [SMILES: *C(=O)CN1CCN(CC(=O)O)CCN(CC(=O)O)CCN(CC(=O)O)CC1 ; *C(=O)CN1CCN(CC(=O)O)CCN(CC(=O)O)CC1 ; *C(=O)CN1CCN(CC(=O)O)CCN(CC(=O)O)CCN(CC(=O)O)CC1]

alternatively,

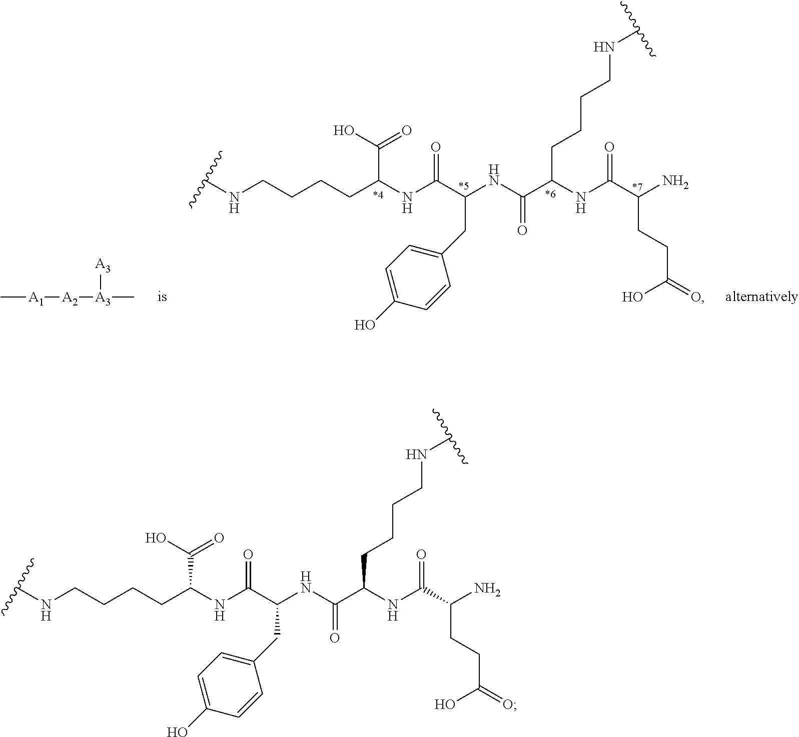

wherein, *4, *5, *6, *7, and *8 are chiral centers, independently selected from in (S) or (R) configuration, or in racemic form; alternatively in (R) configuration;
alternatively, L₁ is

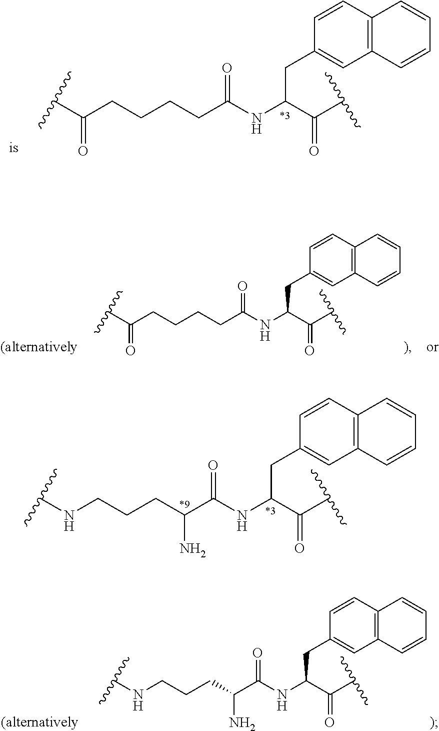

wherein, *3 and *9 are chiral centers, independently selected from in (S) or (R) configuration, or in racemic form; alternatively in (S) configuration.

In a more specific embodiment, the present disclosure provides the above-mentioned compound, wherein the compound is selected from:

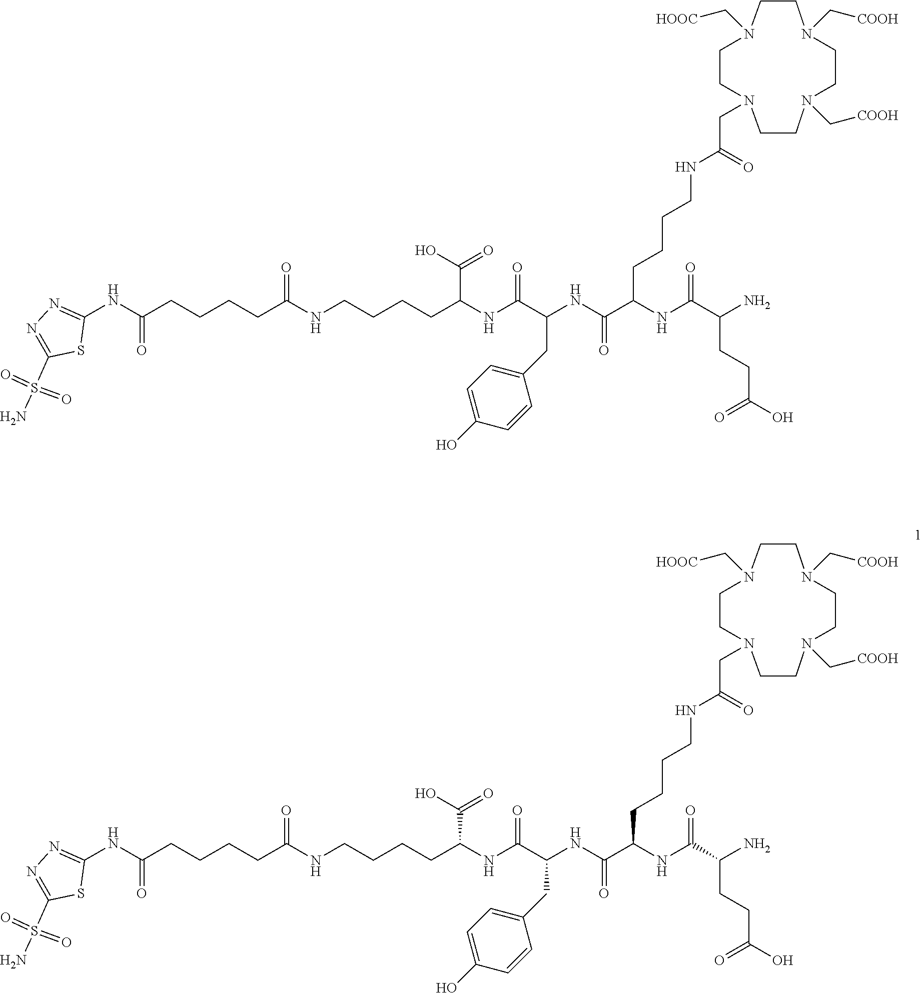 [SMILES: NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O ; N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)CCCCC(=O)Nc1nnc(S(N)(=O)=O)s1)C(=O)O]

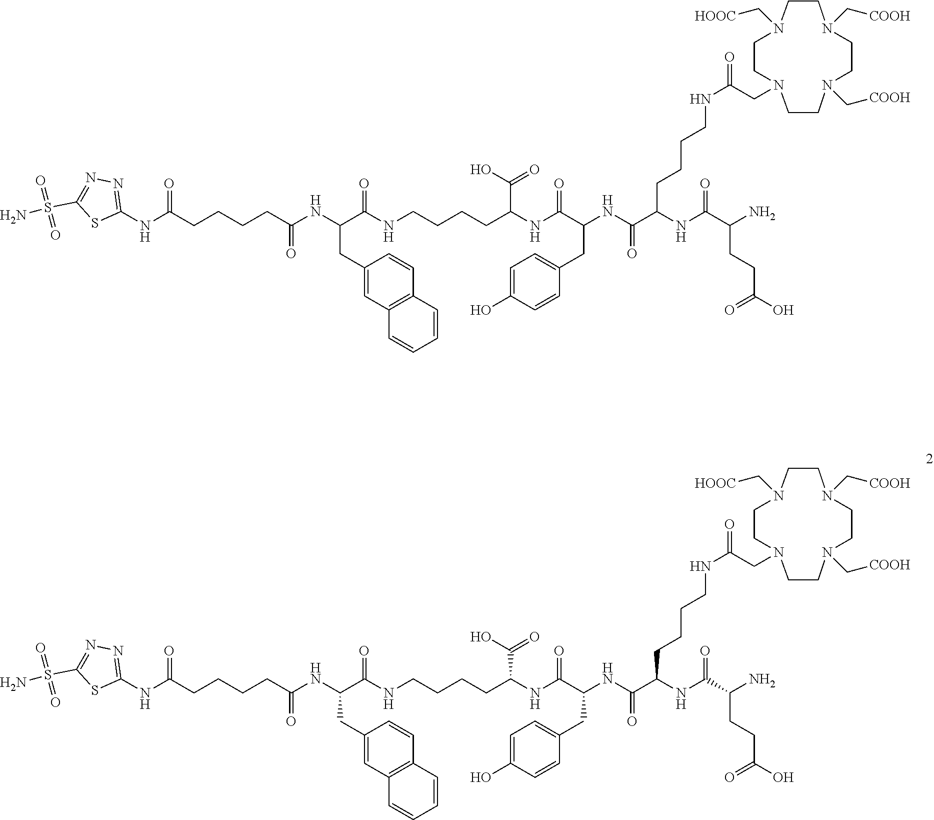 [SMILES: NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)C(Cc1ccc2ccccc2c1)NC(=O)CCCCC(=O)Nc1nnc(S(N)(=O)=O)s1)C(=O)O ; N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)[C@H](Cc1ccc2ccccc2c1)NC(=O)CCCCC(=O)Nc1nnc(S(N)(=O)=O)s1)C(=O)O]

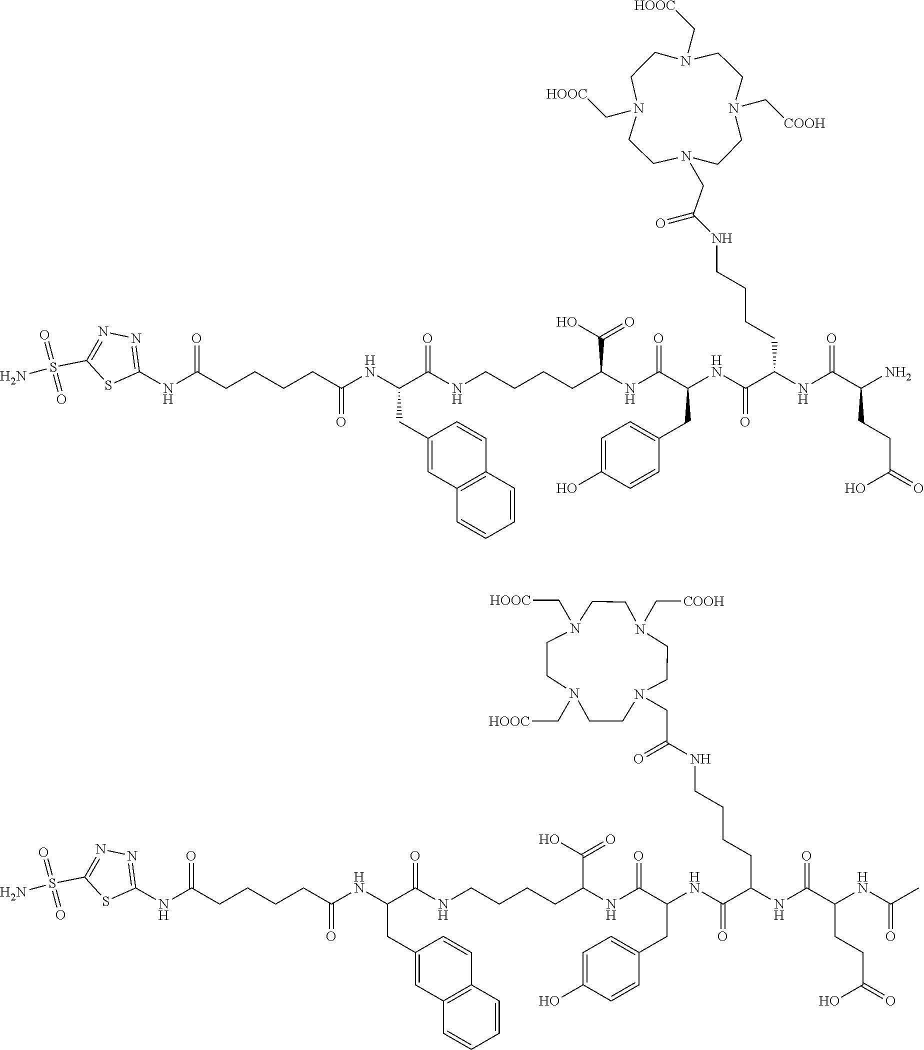 [SMILES: N[C@@H](CCC(=O)O)C(=O)N[C@@H](CCCCNC(=O)CN1CCN(CC(=O)O)CCN(CC(=O)O)CCN(CC(=O)O)CC1)C(=O)N[C@@H](Cc1ccc(O)cc1)C(=O)N[C@@H](CCCCNC(=O)[C@H](Cc1ccc2ccccc2c1)NC(=O)CCCCC(=O)Nc1nnc(S(N)(=O)=O)s1)C(=O)O ; CC(=O)NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)C(Cc1ccc2ccccc2c1)NC(=O)CCCCC(=O)Nc1nnc(S(N)(=O)=O)s1)C(=O)O]

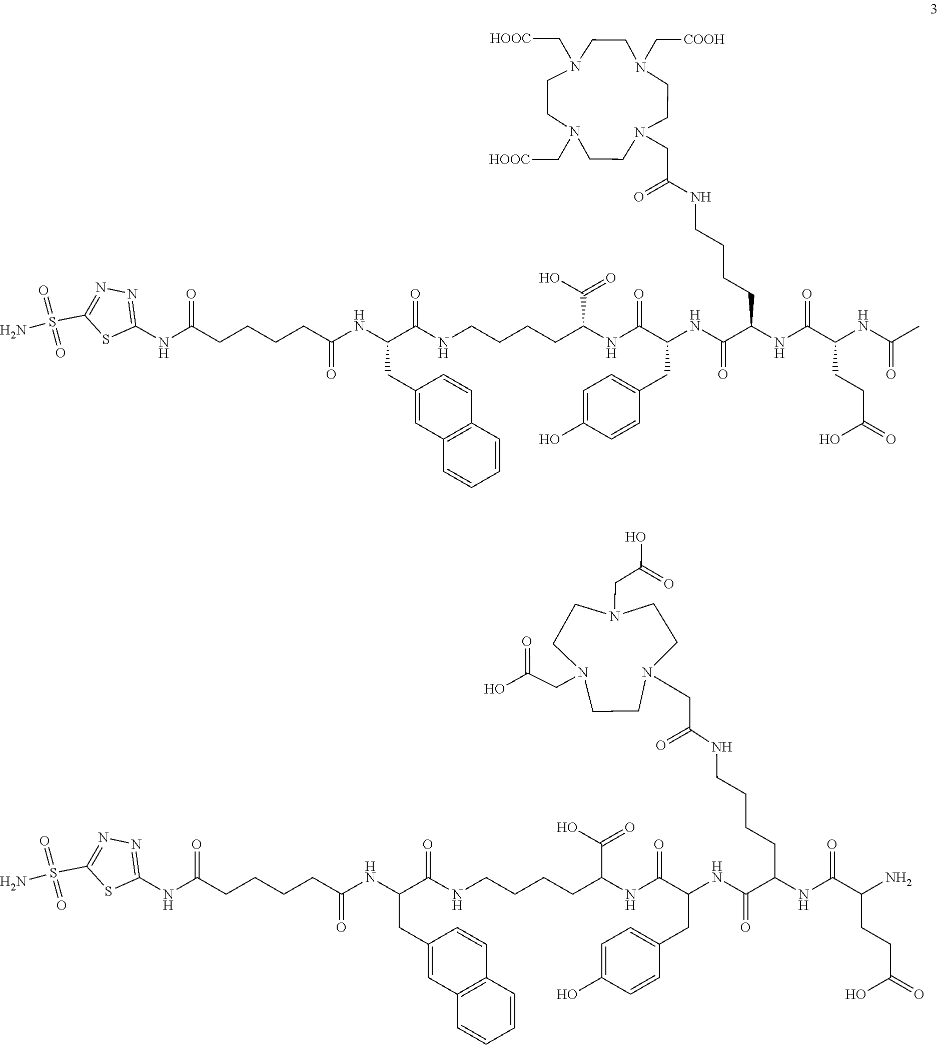 [SMILES: CC(=O)N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)[C@H](Cc1ccc2ccccc2c1)NC(=O)CCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CC1)C(=O)NC(Cc1ccc(O)cc1)C(=O)NC(CCCCNC(=O)C(Cc1ccc2ccccc2c1)NC(=O)CCCCC(=O)Nc1nnc(S(N)(=O)=O)s1)C(=O)O]

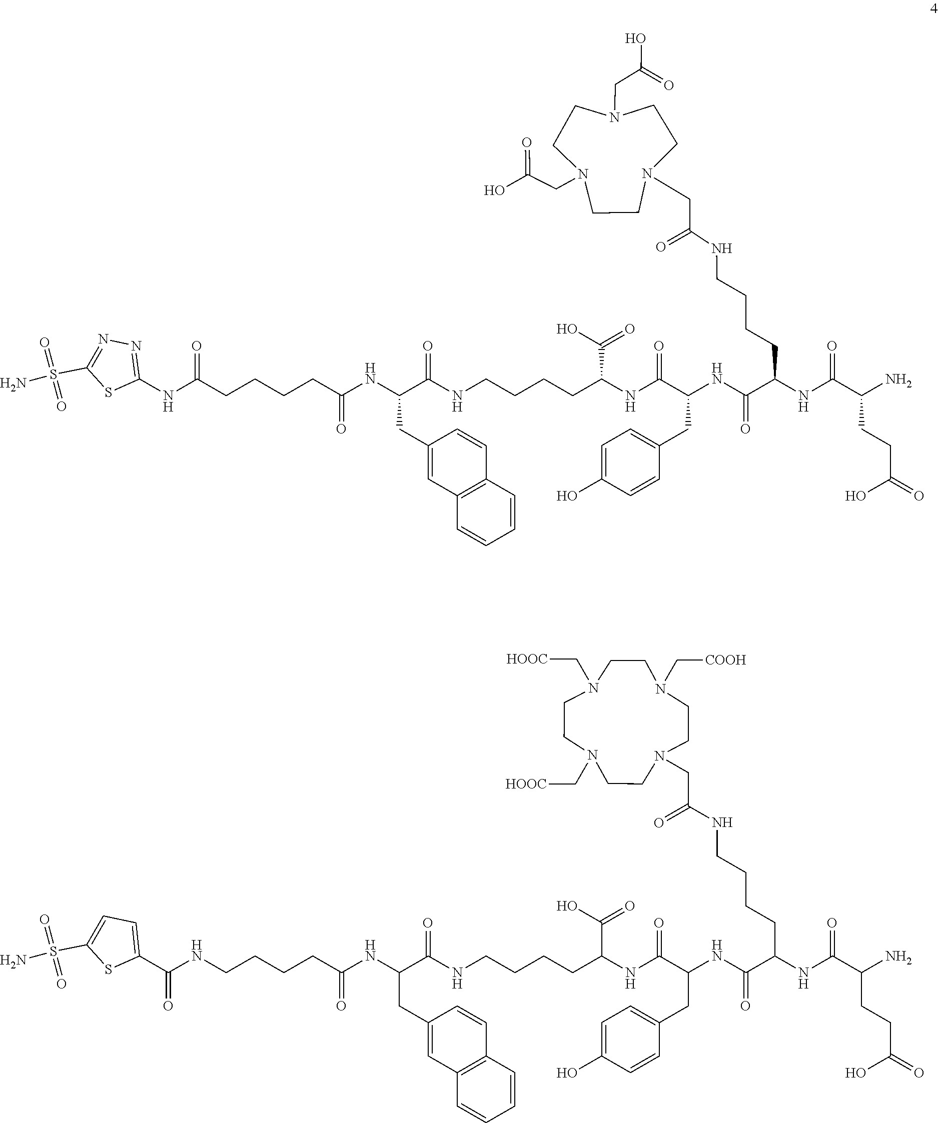 [SMILES: N[C@H](CCC(=O)O)C(=O)N[C@H](CCCCNC(=O)CN1CCN(CC(=O)O)CCN(CC(=O)O)CC1)C(=O)N[C@H](Cc1ccc(O)cc1)C(=O)N[C@H](CCCCNC(=O)[C@H](Cc1ccc2ccccc2c1)NC(=O)CCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)C(Cc1ccc2ccccc2c1)NC(=O)CCCCNC(=O)c1ccc(S(N)(=O)=O)s1)C(=O)O]

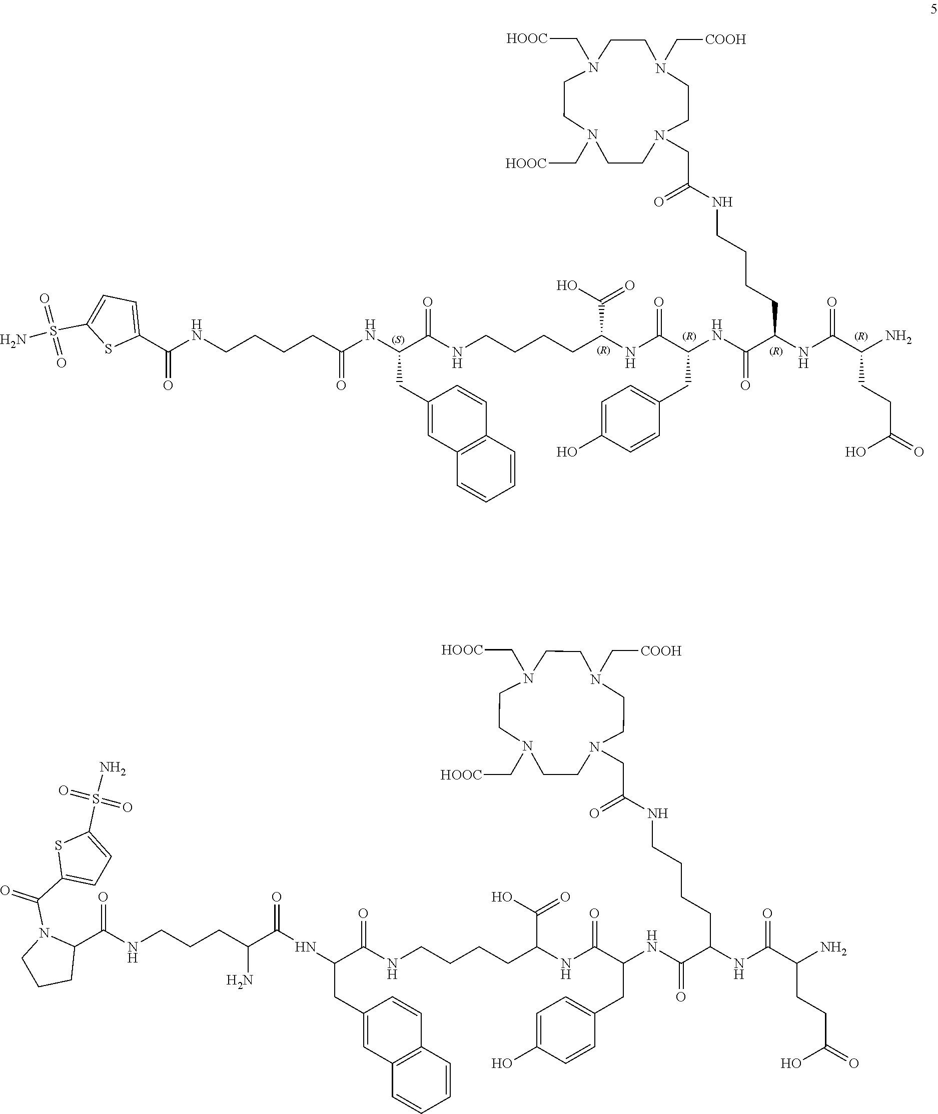

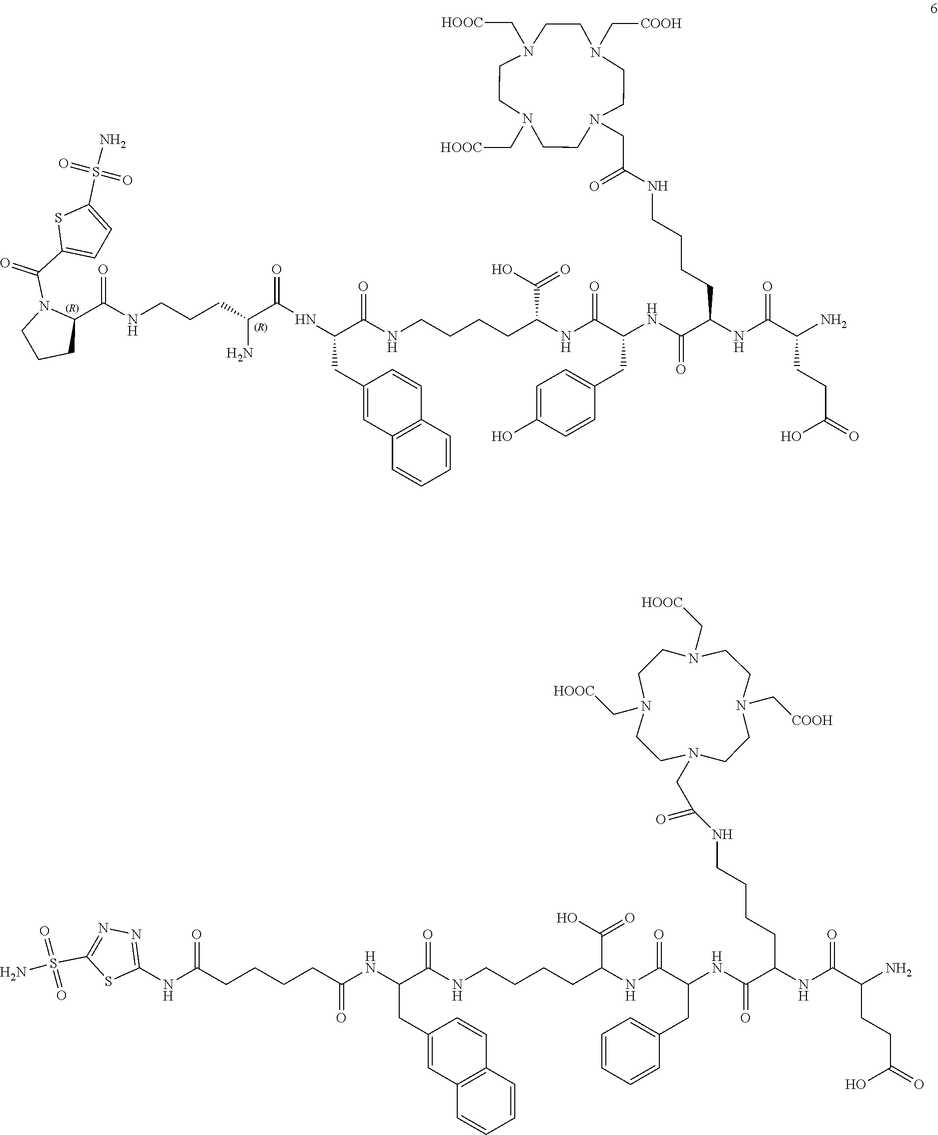

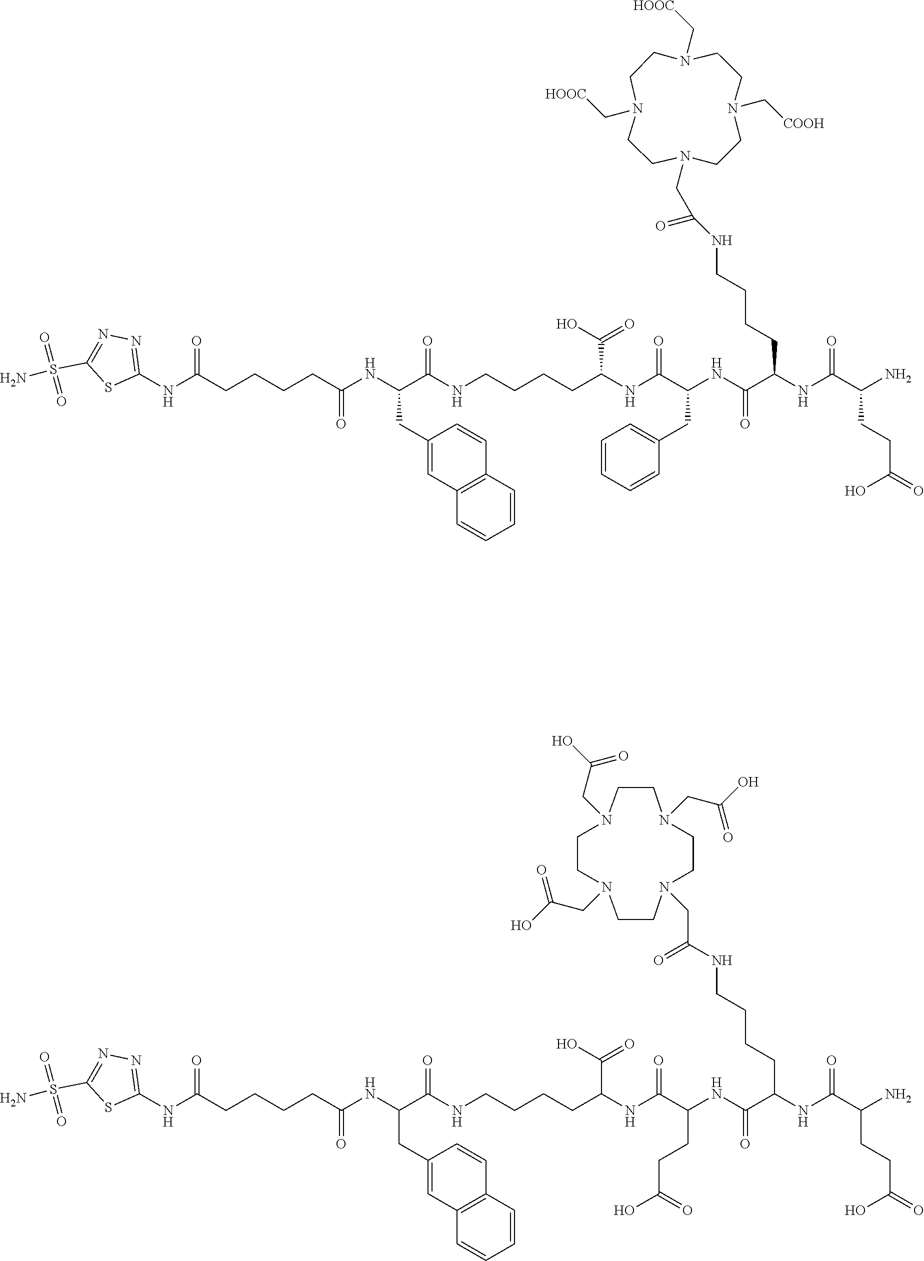 [SMILES: N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccccc1)C(=O)N[C@H](CCCCNC(=O)[C@H](Cc1ccc2ccccc2c1)NC(=O)CCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(CCC(=O)O)C(=O)NC(CCCCNC(=O)C(Cc1ccc2ccccc2c1)NC(=O)CCCCC(=O)Nc1nnc(S(N)(=O)=O)s1)C(=O)O]

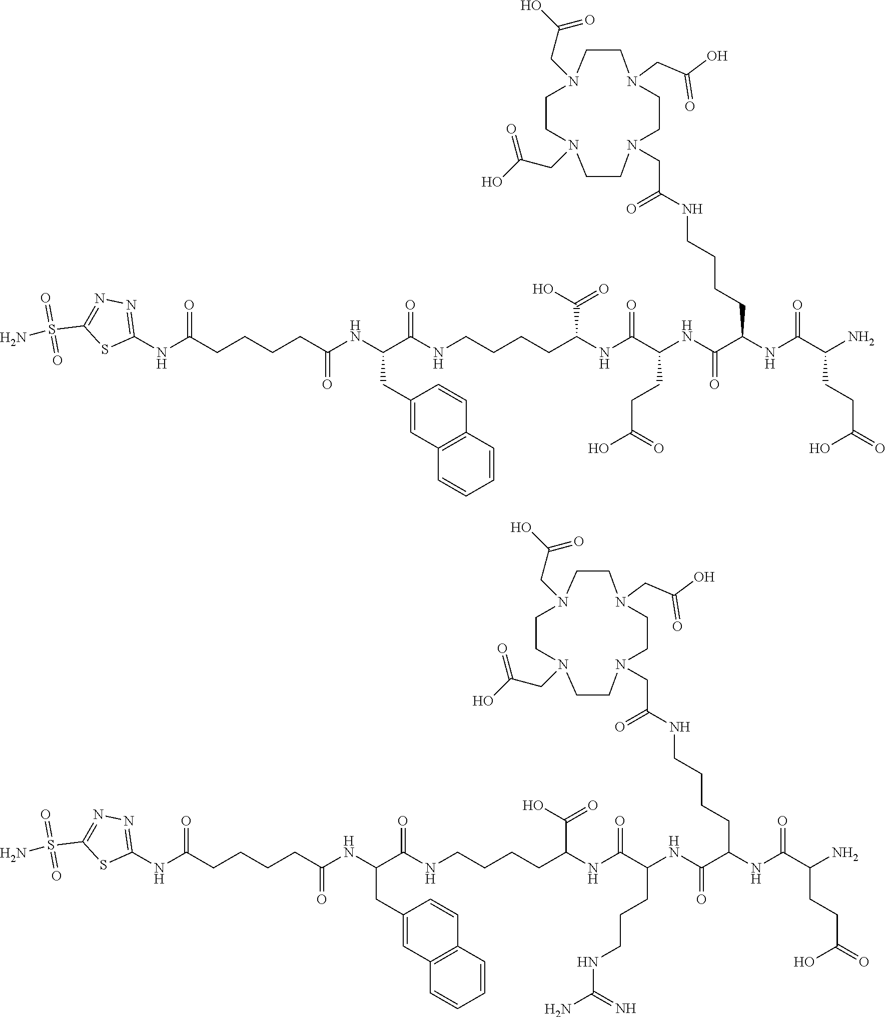 [SMILES: N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](CCC(=O)O)C(=O)N[C@H](CCCCNC(=O)[C@H](Cc1ccc2ccccc2c1)NC(=O)CCCCC(=O)Nc1nnc(S(N)(=O)=O)s1)C(=O)O ; N=C(N)NCCCC(NC(=O)C(CCCCNC(=O)CN1CCN(CC(=O)O)CCN(CC(=O)O)CCN(CC(=O)O)CC1)NC(=O)C(N)CCC(=O)O)C(=O)NC(CCCCNC(=O)C(Cc1ccc2ccccc2c1)NC(=O)CCCCC(=O)Nc1nnc(S(N)(=O)=O)s1)C(=O)O]

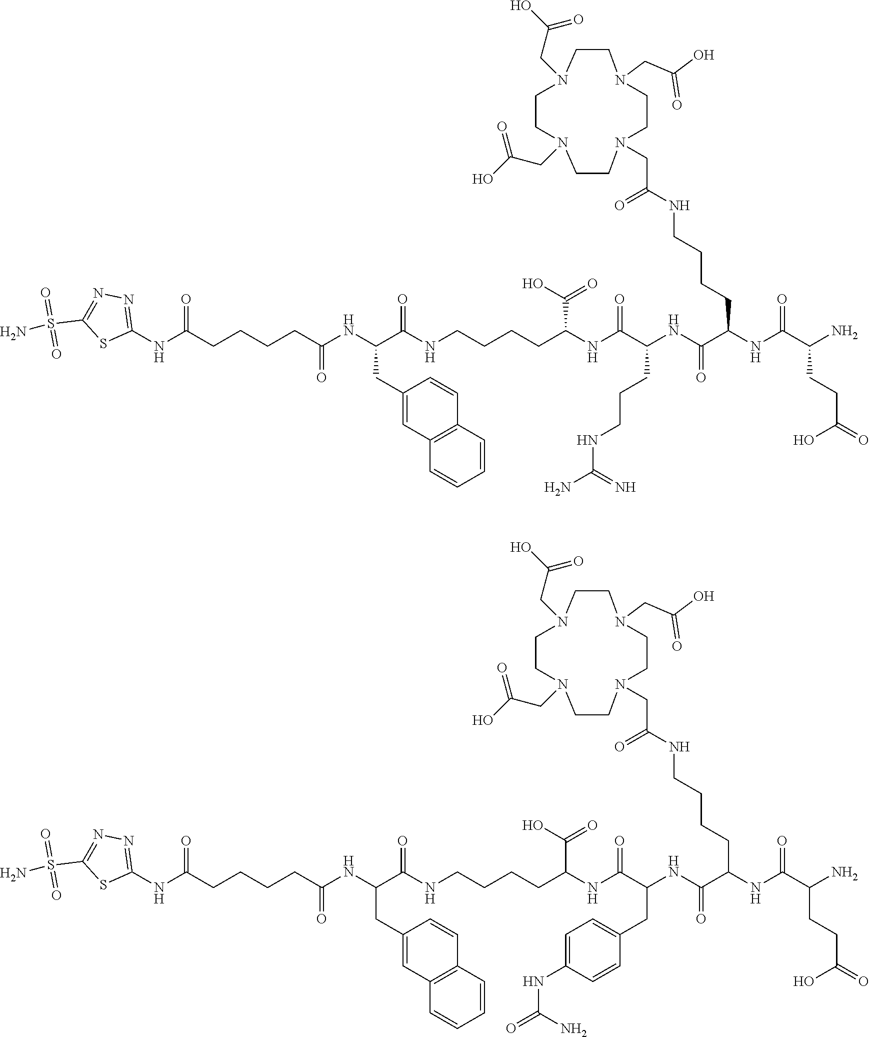 [SMILES: N=C(N)NCCC[C@@H](NC(=O)[C@@H](CCCCNC(=O)CN1CCN(CC(=O)O)CCN(CC(=O)O)CCN(CC(=O)O)CC1)NC(=O)[C@H](N)CCC(=O)O)C(=O)N[C@H](CCCCNC(=O)[C@H](Cc1ccc2ccccc2c1)NC(=O)CCCCC(=O)Nc1nnc(S(N)(=O)=O)s1)C(=O)O ; NC(=O)Nc1ccc(CC(NC(=O)C(CCCCNC(=O)CN2CCN(CC(=O)O)CCN(CC(=O)O)CCN(CC(=O)O)CC2)NC(=O)C(N)CCC(=O)O)C(=O)NC(CCCCNC(=O)C(Cc2ccc3ccccc3c2)NC(=O)CCCCC(=O)Nc2nnc(S(N)(=O)=O)s2)C(=O)O)cc1]

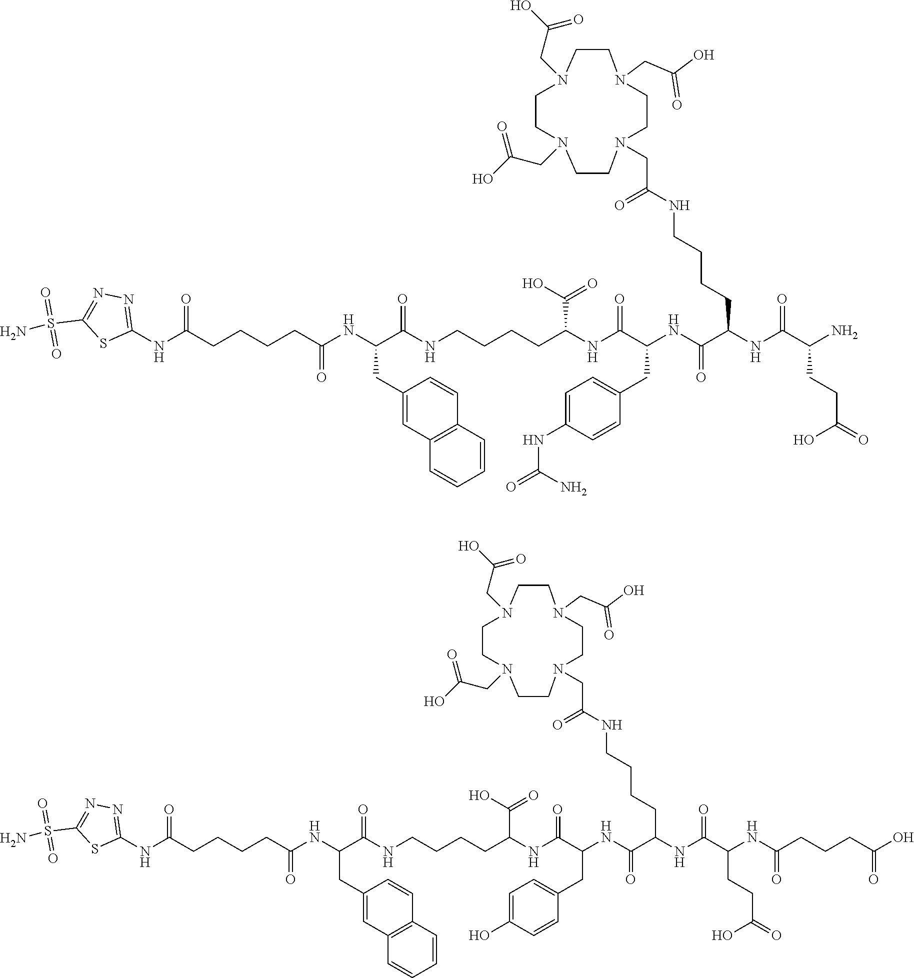 [SMILES: NC(=O)Nc1ccc(C[C@@H](NC(=O)[C@@H](CCCCNC(=O)CN2CCN(CC(=O)O)CCN(CC(=O)O)CCN(CC(=O)O)CC2)NC(=O)[C@H](N)CCC(=O)O)C(=O)N[C@H](CCCCNC(=O)[C@H](Cc2ccc3ccccc3c2)NC(=O)CCCCC(=O)Nc2nnc(S(N)(=O)=O)s2)C(=O)O)cc1 ; NS(=O)(=O)c1nnc(NC(=O)CCCCC(=O)NC(Cc2ccc3ccccc3c2)C(=O)NCCCCC(NC(=O)C(Cc2ccc(O)cc2)NC(=O)C(CCCCNC(=O)CN2CCN(CC(=O)O)CCN(CC(=O)O)CCN(CC(=O)O)CC2)NC(=O)C(CCC(=O)O)NC(=O)CCCC(=O)O)C(=O)O)s1]

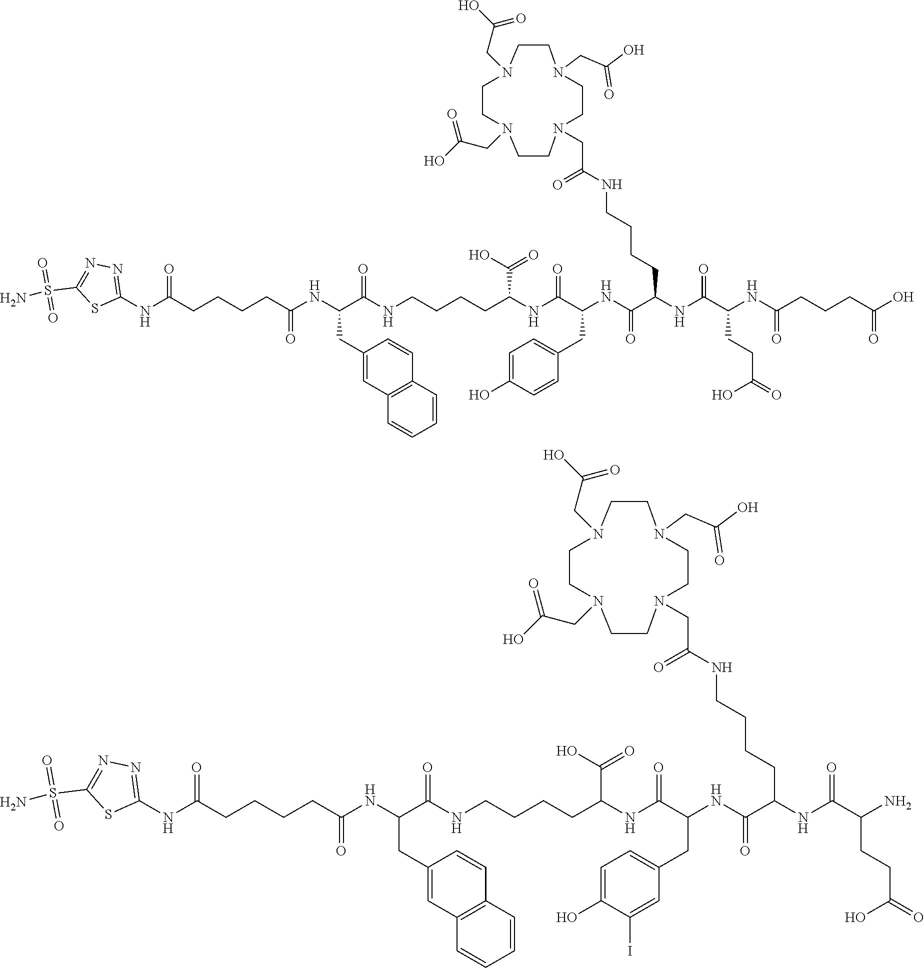 [SMILES: NS(=O)(=O)c1nnc(NC(=O)CCCCC(=O)N[C@@H](Cc2ccc3ccccc3c2)C(=O)NCCCC[C@@H](NC(=O)[C@@H](Cc2ccc(O)cc2)NC(=O)[C@@H](CCCCNC(=O)CN2CCN(CC(=O)O)CCN(CC(=O)O)CCN(CC(=O)O)CC2)NC(=O)[C@@H](CCC(=O)O)NC(=O)CCCC(=O)O)C(=O)O)s1 ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)c(I)c1)C(=O)NC(CCCCNC(=O)C(Cc1ccc2ccccc2c1)NC(=O)CCCCC(=O)Nc1nnc(S(N)(=O)=O)s1)C(=O)O]

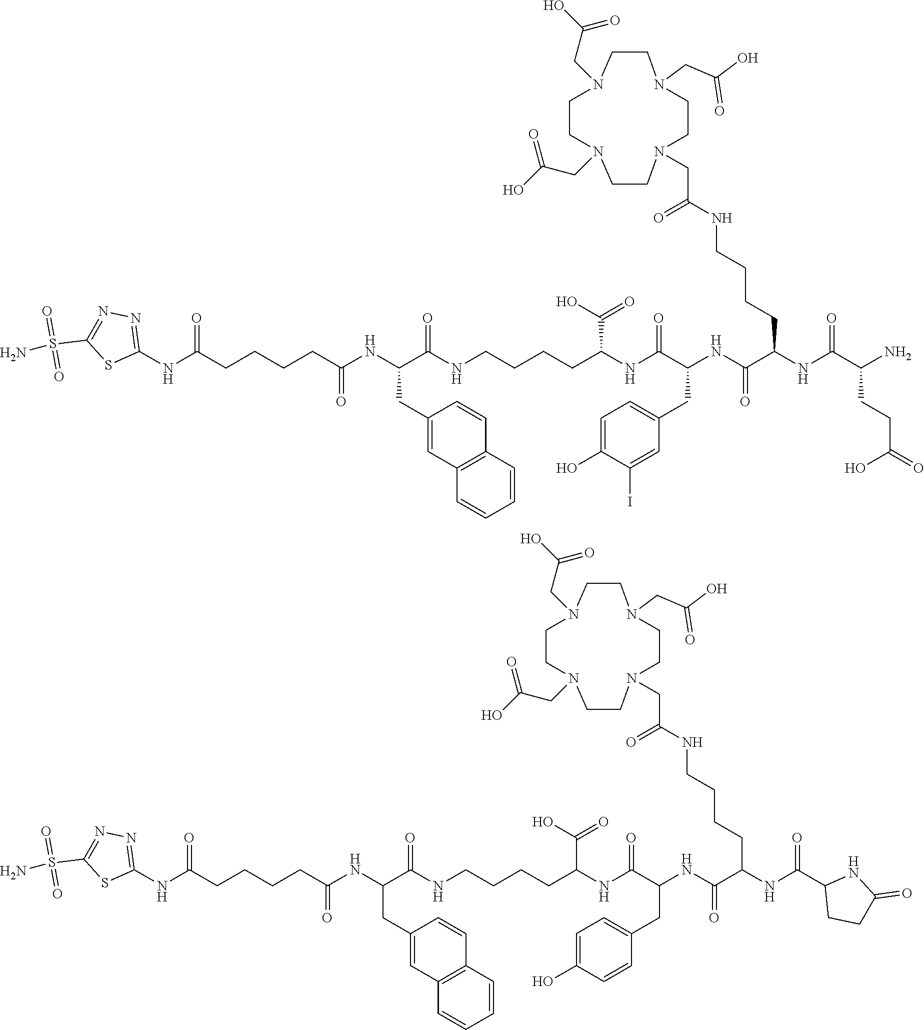

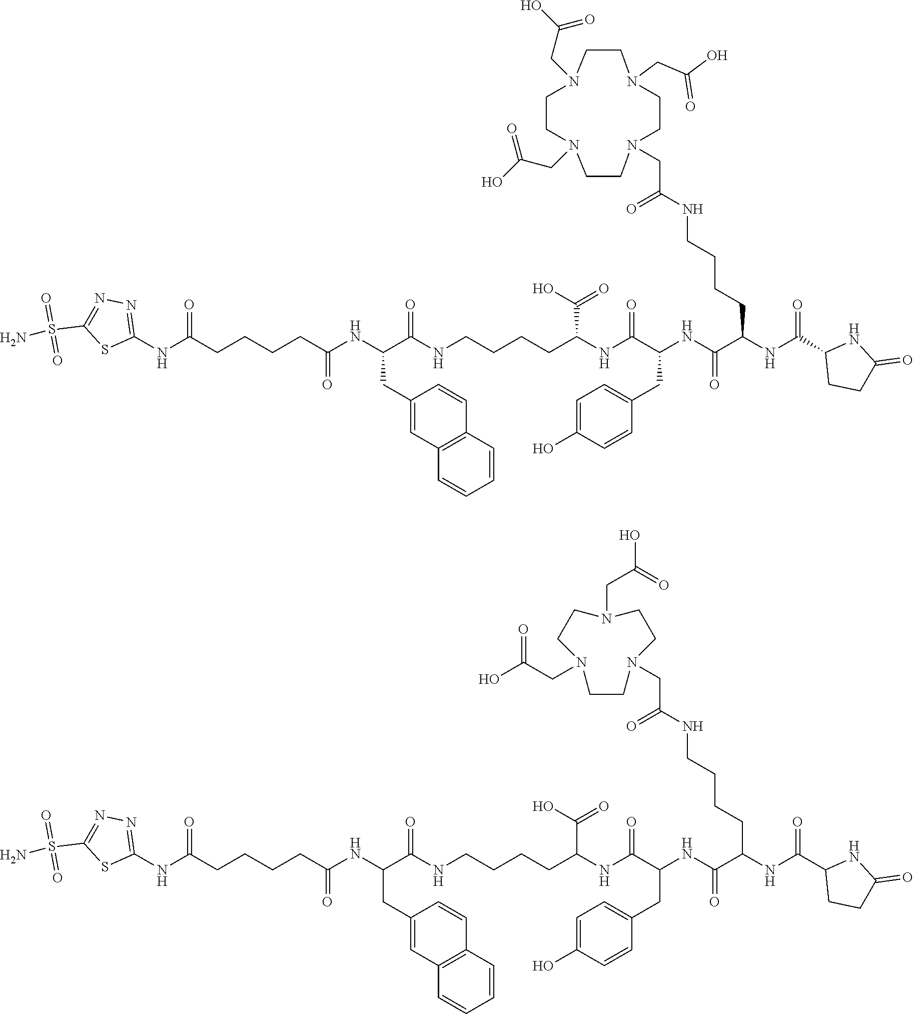 [SMILES: NS(=O)(=O)c1nnc(NC(=O)CCCCC(=O)N[C@@H](Cc2ccc3ccccc3c2)C(=O)NCCCC[C@@H](NC(=O)[C@@H](Cc2ccc(O)cc2)NC(=O)[C@@H](CCCCNC(=O)CN2CCN(CC(=O)O)CCN(CC(=O)O)CCN(CC(=O)O)CC2)NC(=O)[C@H]2CCC(=O)N2)C(=O)O)s1 ; NS(=O)(=O)c1nnc(NC(=O)CCCCC(=O)NC(Cc2ccc3ccccc3c2)C(=O)NCCCCC(NC(=O)C(Cc2ccc(O)cc2)NC(=O)C(CCCCNC(=O)CN2CCN(CC(=O)O)CCN(CC(=O)O)CC2)NC(=O)C2CCC(=O)N2)C(=O)O)s1]

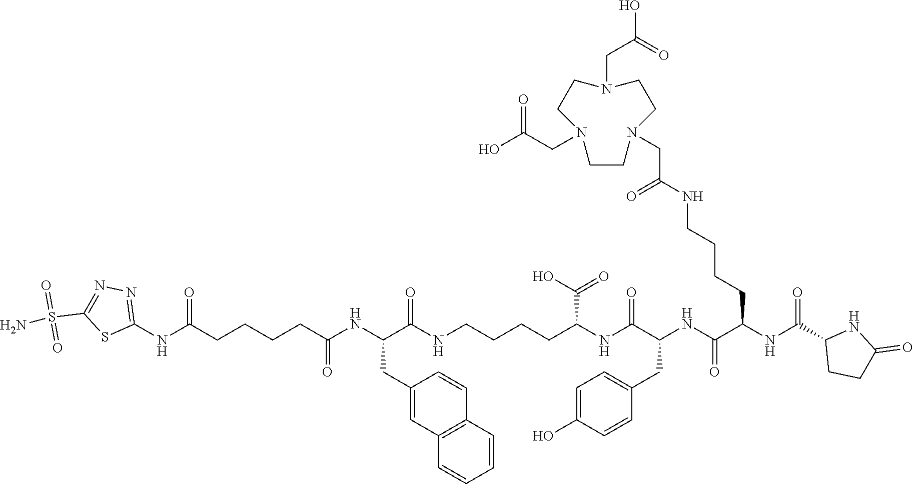 [SMILES: NS(=O)(=O)c1nnc(NC(=O)CCCCC(=O)N[C@@H](Cc2ccc3ccccc3c2)C(=O)NCCCC[C@@H](NC(=O)[C@@H](Cc2ccc(O)cc2)NC(=O)[C@@H](CCCCNC(=O)CN2CCN(CC(=O)O)CCN(CC(=O)O)CC2)NC(=O)[C@H]2CCC(=O)N2)C(=O)O)s1]

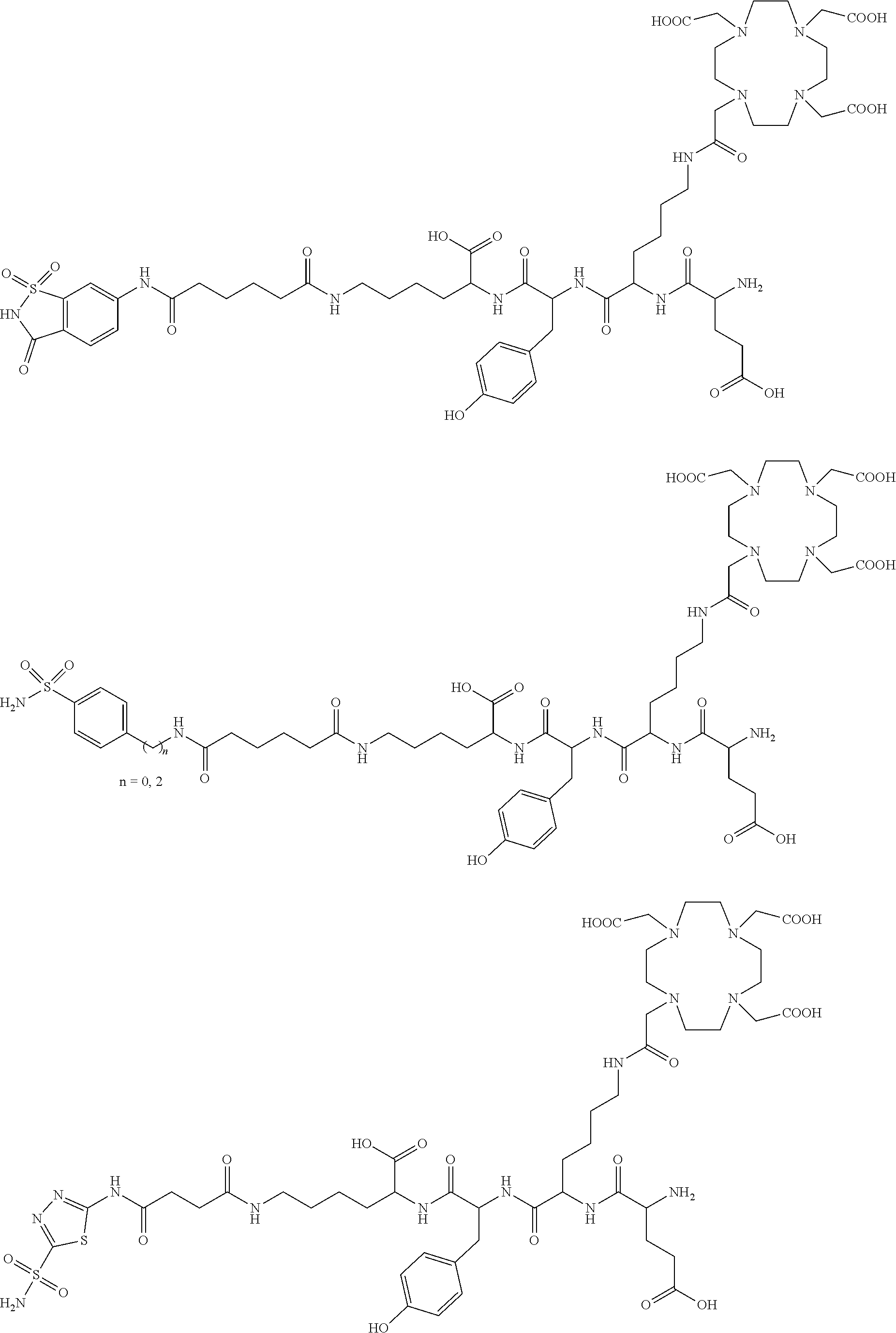

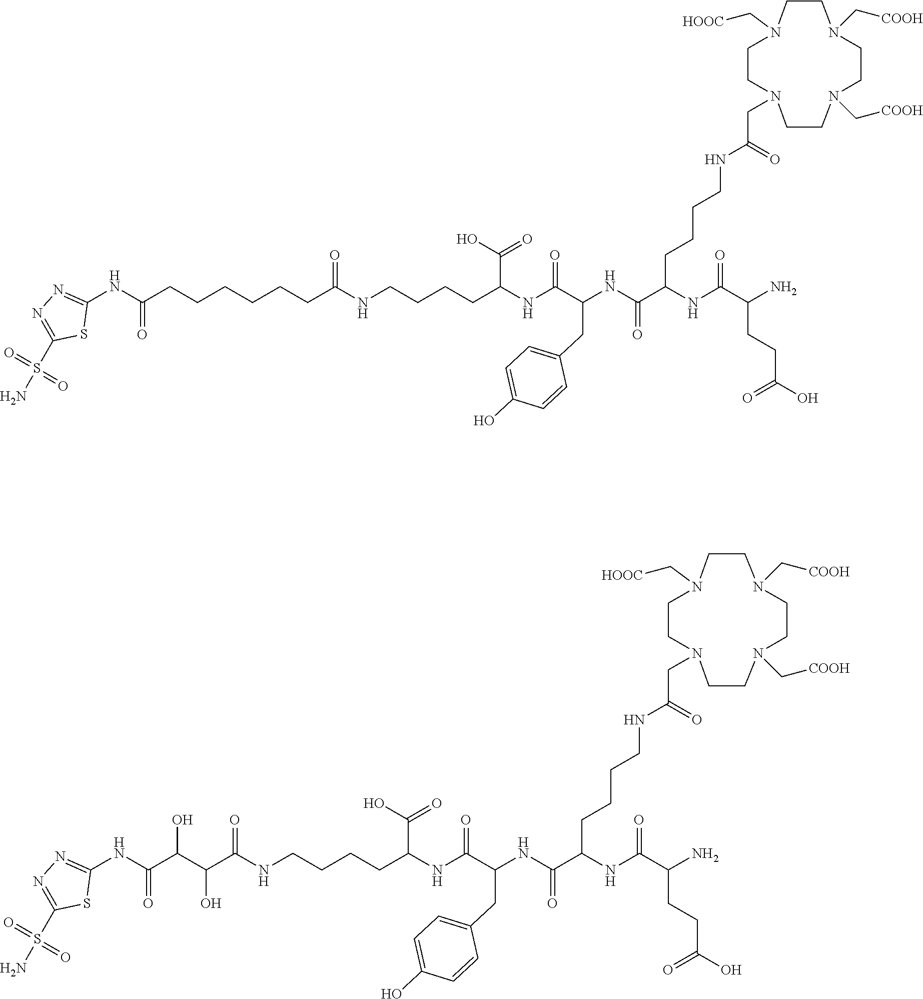 [SMILES: NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCCCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)C(O)C(O)C(=O)Nc1nnc(S(N)(=O)=O)s1)C(=O)O]

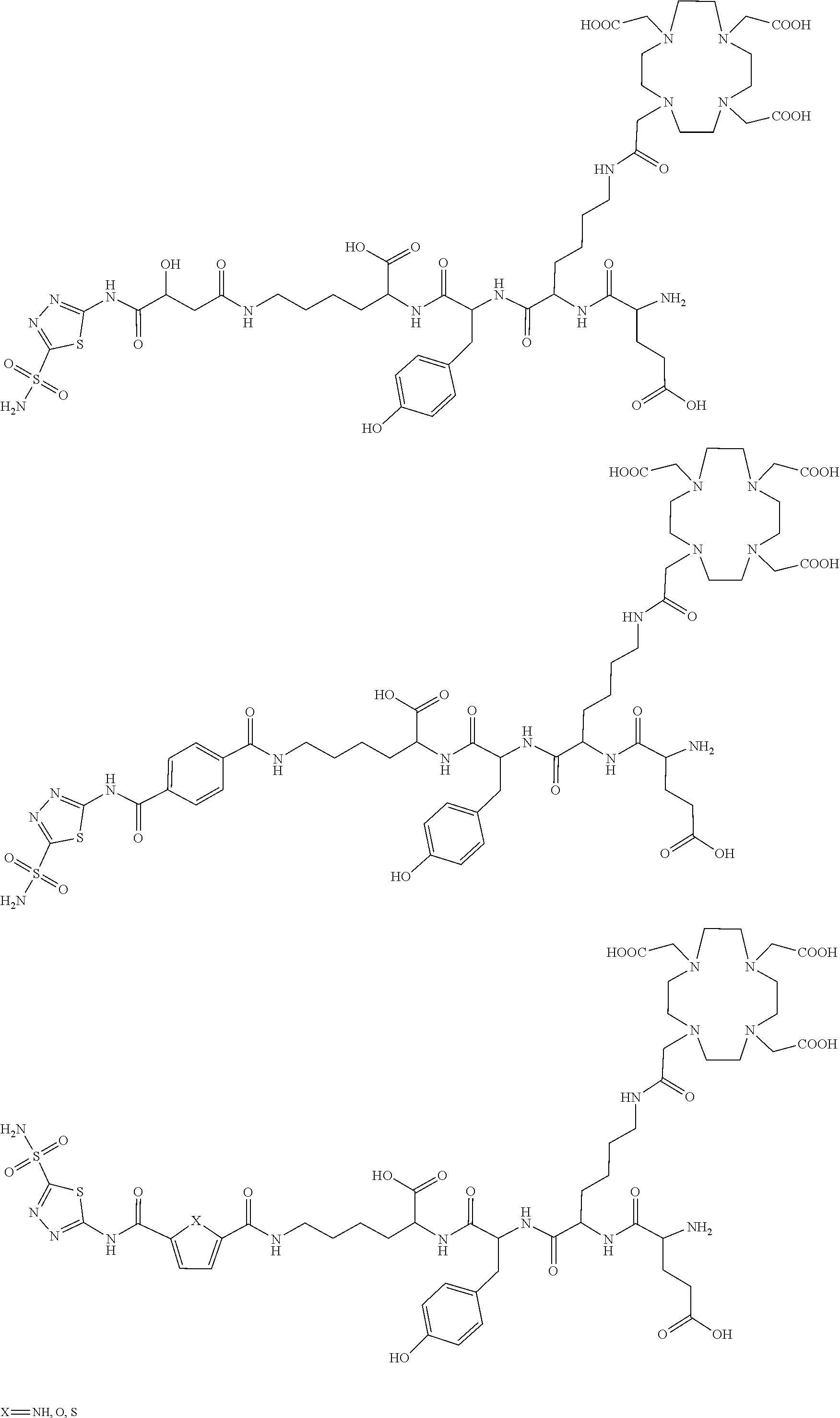

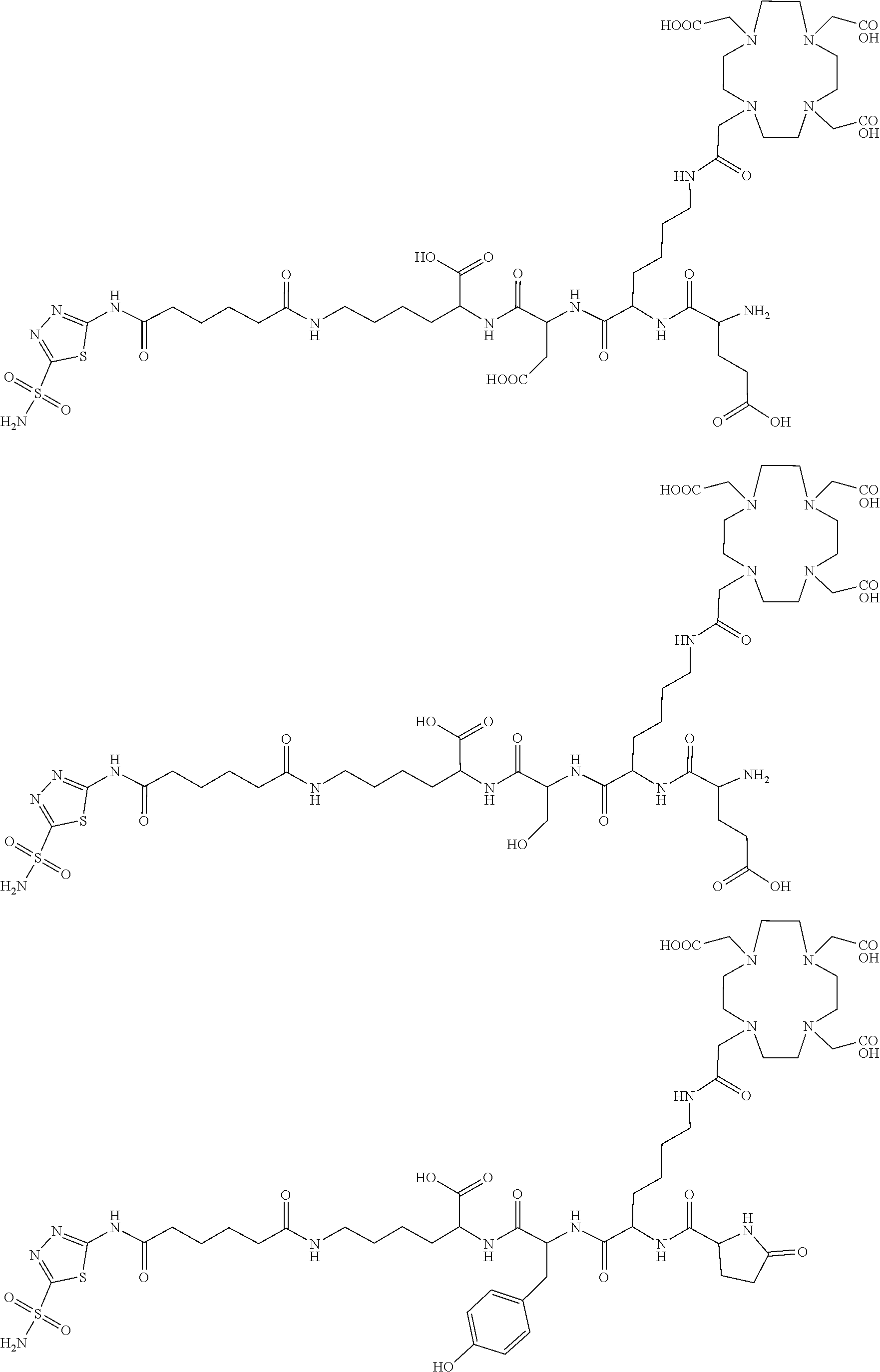

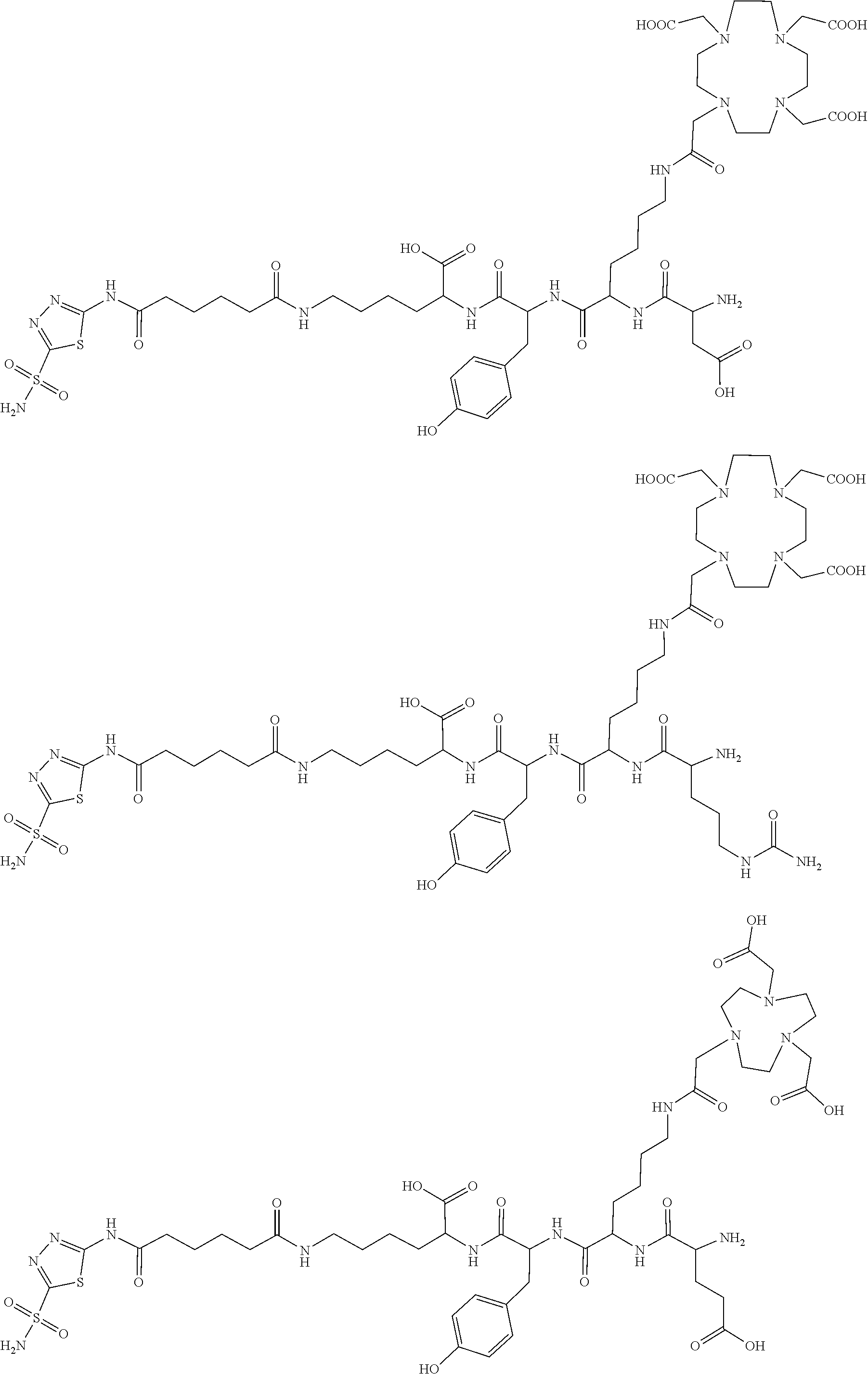 [SMILES: NC(CC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O ; NC(=O)NCCCC(N)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O]

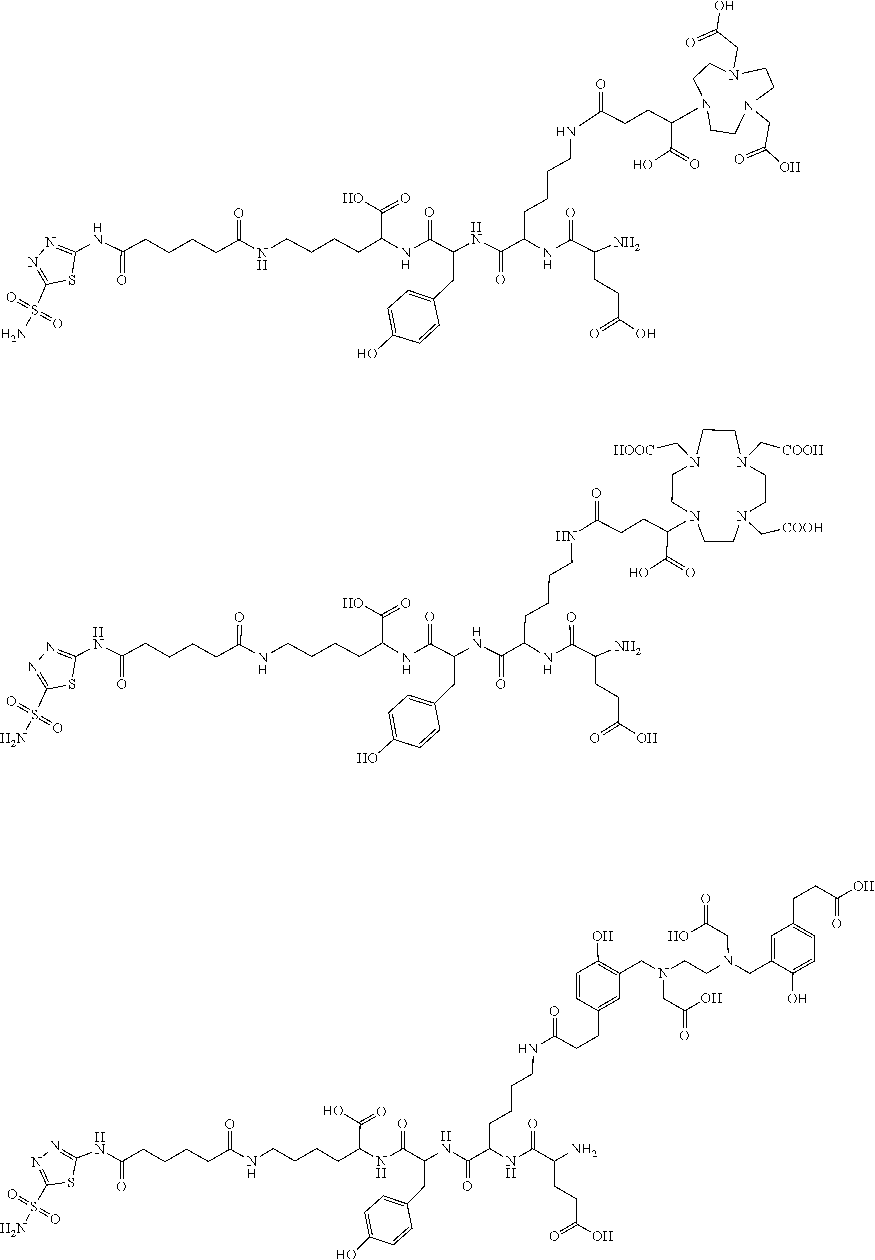 [SMILES: NC(CCC(=O)O)C(=O)NC(CCCCNC(=O)CCC(C(=O)O)N1CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O ; NC(CCC(=O)O)C(=O)NC(CCCCNC(=O)CCC(C(=O)O)N1CCN(CC(=O)O)CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O ; NC(CCC(=O)O)C(=O)NC(CCCCNC(=O)CCc1ccc(O)c(CN(CCN(CC(=O)O)Cc2cc(CCC(=O)O)ccc2O)CC(=O)O)c1)C(=O)NC(Cc1ccc(O)cc1)C(=O)NC(CCCCNC(=O)CCCCC(=O)Nc1nnc(S(N)(=O)=O)s1)C(=O)O]

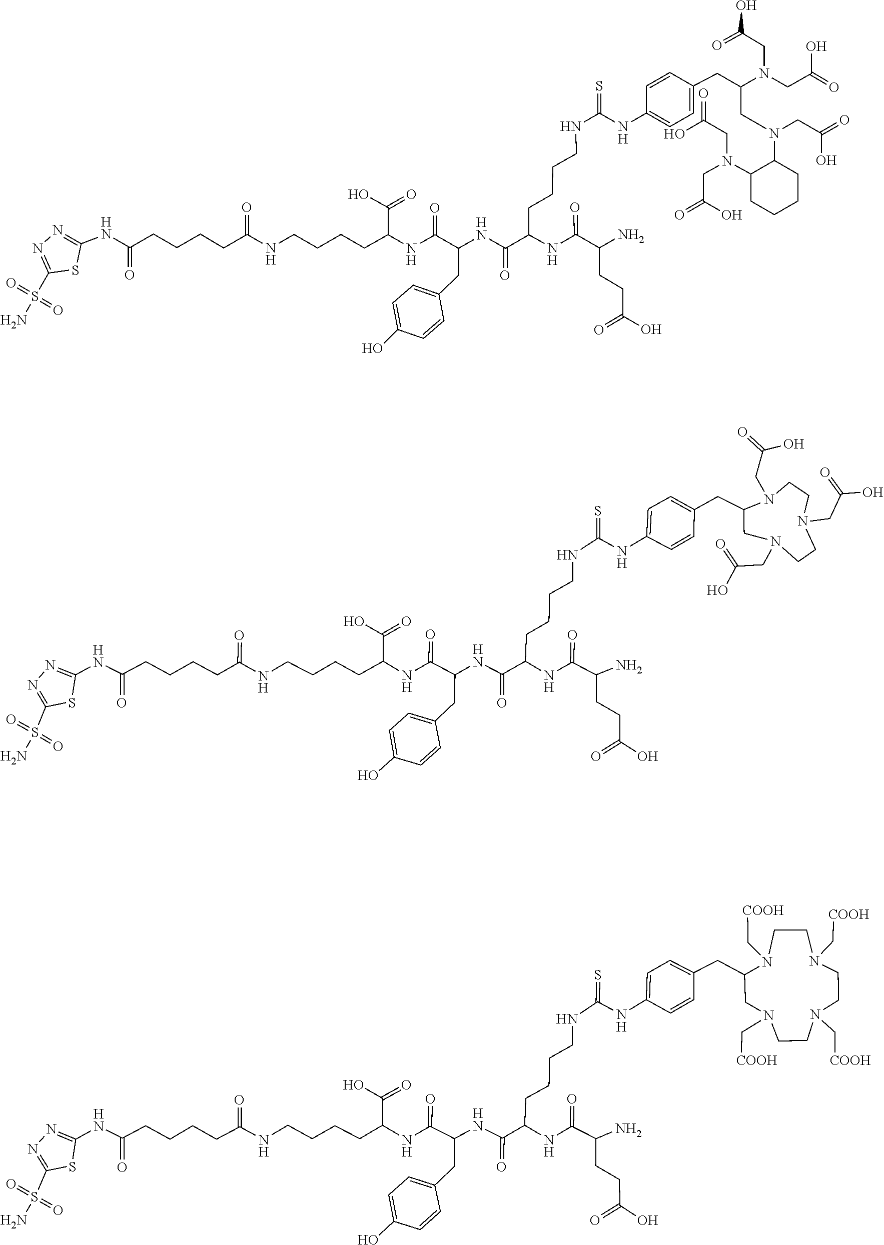

In a more specific embodiment, the present disclosure provides a compound comprising a compound of formula (I), or an isotopic variant, a hydrate, an ester or solvate, a tautomer, a stereoisomer, or a pharmaceutically acceptable salt thereof, and M complexed therewith,
wherein,
the compounds of formula (I) are as defined herein;
M is selected from at least one of radionuclides or non-radioactive elements (alternatively radionuclides);
alternatively, the radionuclide is selected from at least one of a diagnostic nuclide or a therapeutic nuclide;
alternatively, the radionuclide is selected from ⁶⁸Ga, ¹⁸F, ⁹⁹mTc, ⁸⁹Zr, ¹²⁴I, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or ⁸⁶Y; or selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb, ⁴⁷Sc, ^{186/188}Re, ^{212/213}Bi, ¹⁴⁹Pm, ²¹²Pb, ²¹¹At, ²²³Ra, ¹⁶¹Tb, ²²⁵Ac or ²²⁷Th;
alternatively, the diagnostic nuclide is selected from ⁶⁸Ga, ¹⁸F, ⁹⁹mTc, ⁸⁹Zr, ¹²⁴I, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or ⁸⁶Y
alternatively, the therapeutic nuclide is selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb, ⁴⁷Sc, ^{186/188}Re, ^{212/213}Bi, ¹⁴⁹Pm, ²¹²Pb, ²¹¹At, ²²³Ra, ¹⁶¹Tb, ²²⁵Ac or ²²⁷Th;
alternatively, the radionuclide is selected from ⁶⁸Ga, ¹⁸F, ⁸⁹Zr, ⁹⁹mTc, ⁶⁴Cu, ¹⁶¹Tb or ¹⁷⁷Lu;
alternatively, the radionuclide ¹⁸F is formed by complexing ¹⁸FA1 with the compound of formula (I);
alternatively, M is selected from ⁶⁸Ga, ¹⁸F, ¹⁶¹Tb or ¹⁷⁷Lu; alternatively ⁶⁸Ga or ¹⁷⁷Lu.
In a more specific embodiment, the present disclosure provides a compound of formula (V), or an isotopic variant, a hydrate, an ester or solvate, a tautomer or a stereoisomer thereof, or a pharmaceutically acceptable salt thereof:

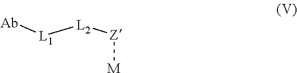

wherein,
Ab, L₁ and L₂ are as defined herein;
Z′ is a coordination group formed by complexing a chelating group Z derived from a chelating agent with M, wherein Z is as defined in any one of the items herein;
M is selected from at least one of radionuclides or non-radioactive elements (alternatively radionuclides);
alternatively, the radionuclide is selected from at least one of a diagnostic nuclide or a therapeutic nuclide;
alternatively, the radionuclide is selected from ⁶⁸Ga, ¹⁸F, ⁹⁹mTc, ⁸⁹Zr, ¹²⁴I, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or 16Y, or is selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb, ⁴⁷Sc, ^{186/188}Re, ^{212/213}Bi, ¹⁴⁹Pm, ²¹²Pb, ²¹¹At, ²²³Ra, ¹⁶¹Tb, ²²⁵Ac or ²²⁷Th;
alternatively, the diagnostic nuclide is selected from ⁶⁸Ga, ¹⁸F, ⁹⁹mTc, ⁸⁹Zr, ¹²⁴I, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or ⁸⁶Y;
alternatively, the therapeutic nuclide is selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb, ⁴⁷Sc, ^{186/188}Re, ^{212/213}Bi, ¹⁴⁹Pm, ²¹²Pb, ²¹¹At, ²²³Ra, ¹⁶¹Tb, ²²⁵Ac or ²²⁷Th;
alternatively, the radionuclide is selected from ⁶⁸Ga, ¹⁸F, ⁸⁹Zr, ⁹⁹mTc, ⁶⁴Cu, ¹⁶¹Tb or ¹⁷⁷Lu;
alternatively, the radionuclide ¹⁸F is formed by ¹⁸FA1 complexation;
alternatively, M is selected from ⁶⁸Ga, ¹⁸F, ¹⁶¹Tb or ¹⁷⁷Lu; alternatively ⁶⁸Ga or ¹⁷⁷Lu.

The compounds of the present disclosure may include one or more asymmetric centers, and thus may exist in a variety of stereoisomeric forms, for example, enantiomers and/or diastereomers. For example, the compounds of the present disclosure may be in the form of an individual enantiomer, diastereomer or geometric isomer (e.g., cis- and trans-isomers), or may be in the form of a mixture of stereoisomers, including racemic mixture and a mixture enriched in one or more stereoisomers. The isomers can be separated from the mixture by the methods known to those skilled in the art, including chiral high pressure liquid chromatography (HPLC) and the formation and crystallization of chiral salts; or alternative isomers can be prepared by asymmetric synthesis.

The compounds of the present disclosure may exist in tautomeric forms. Tautomers are functional group isomers produced by the rapid movement of an atom in a molecule between two positions. The tautomers are special functional group isomers. A pair of tautomers can be converted into each other, but usually a relatively stable isomer is the main form of existence. The most important examples are the enol and keto tautomers.

It will be understood by those skilled in the art that the organic compounds can form complexes with solvents in which they are reacted or from which they are precipitated or crystallized. These complexes are known as “solvates.” When the solvent is water, the complex is known as “hydrate.” The present disclosure encompasses all solvates of the compounds of the present disclosure.

The term “solvate” refers to forms of a compound or a salt thereof, which are associated with a solvent, usually by a solvolysis reaction. This physical association may include hydrogen bonding. Conventional solvents include water, methanol, ethanol, acetic acid, DMSO, THF, diethyl ether, etc. The compounds described herein can be prepared, for example, in crystalline form, and can be solvated. Suitable solvates include pharmaceutically acceptable solvates and further include both stoichiometric solvates and non-stoichiometric solvates. In some cases, the solvates will be capable of isolation, for example, when one or more solvent molecules are incorporated into the crystal lattice of a crystalline solid. “Solvate” includes both solution-phase and isolatable solvates. Representative solvates include hydrates, ethanolates and methanolates.

The term “hydrate” refers to a compound that is associated with water. Generally, the number of water molecules contained in a hydrate of a compound is in a definite ratio to the number of the compound molecules in the hydrate. Therefore, hydrates of a compound can be represented, for example, by a general formula R·x H₂O, wherein R is the compound, and x is a number greater than 0. Given compounds can form more than one type of hydrates, including, for example, monohydrates (x is 1), lower hydrates (x is a number greater than 0 and smaller than 1, for example, hemihydrates (R·0.5 H₂O)) and polyhydrates (x is a number greater than 1, for example, dihydrates (R·2 H₂O) and hexahydrates (R·6 H₂O)).

Compounds of the present disclosure may be in an amorphous or a crystalline form (polymorph). Furthermore, the compounds of the present disclosure may exist in one or more crystalline forms. Therefore, the present disclosure includes all amorphous or crystalline forms of the compounds of the present disclosure within its scope. The term “polymorph” refers to a crystalline form of a compound (or a salt, hydrate or solvate thereof) in a particular crystal packing arrangement. All polymorphs have the same elemental composition. Different crystalline forms generally have different X-ray diffraction patterns, infrared spectra, melting points, density, hardness, crystal shapes, optical and electrical properties, stability, and solubility. Recrystallization solvents, rate of crystallization, storage temperatures, and other factors may cause one crystalline form to dominate. Various polymorphs of a compound can be prepared by crystallization under different conditions.

The present disclosure also comprises compounds that are labeled with isotopes (isotopic variants), which are equivalent to those described in formula (I), but one or more atoms are replaced by atoms having an atom mass or mass number that are different from that of atoms that are common in nature. Examples of isotopes which may be introduced into the compounds of the disclosure include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl, respectively. Compounds of the present disclosure that comprise the above isotopes and/or other isotopes of other atoms, prodrugs thereof and pharmaceutically acceptable salts of said compounds or prodrugs all are within the scope of the present disclosure. Certain isotope-labeled compounds of the present disclosure, such as those incorporating radioactive isotopes (e.g., ³H and ¹⁴C), can be used for the measurement of the distribution of drug and/or substrate in tissue. Tritium, which is ³H and carbon-14, which is ¹⁴C isotope, are yet alternative, because they are easy to prepare and detect. Furthermore, replaced by heavier isotopes, such as deuterium, which is ²H, may provide therapeutic benefits due to the higher metabolic stability, such as prolonging the half-life in vivo or decreasing the dosage requirements, and thus may be alternative in some cases. Isotope-labeled compounds of formula (I) of the present disclosure and prodrugs thereof can be prepared generally by using readily available isotope-labeled reagents to replace non-isotope-labeled reagents in the following schemes and/or the procedures disclosed in the examples and preparation examples.

In addition, prodrugs are also included within the context of the present disclosure. The term “prodrug” as used herein refers to a compound that is converted into an active form that has medical effects in vivo by, for example, hydrolysis in blood. Pharmaceutically acceptable prodrugs are described in T. Higuchi and V. Stella, Prodrugs as Novel Delivery Systems, A.C.S. Symposium Series, Vol. 14, Edward B. Roche, ed., Bioreversible Carriers in Drug Design, American Pharmaceutical Association and Pergamon Press, 1987, and D. Fleisher, S. Ramon and H. Barbra “Improved oral drug delivery: solubility limitations overcome by the use of prodrugs”, Advanced Drug Delivery Reviews (1996) 19(2) 115-130, each of which is incorporated herein by reference.

The prodrugs are any covalently bonded compounds of the present disclosure, which release the parent compound in vivo when the prodrug is administered to a patient. Prodrugs are typically prepared by modifying functional groups in such a way that the modifications can be cleaved either by routine manipulation or decompose in vivo to yield the parent compound. Prodrugs include, for example, compounds of the present disclosure wherein the hydroxyl, amino or sulfhydryl groups are bonded to any group that, when administered to a patient, cleaves to form the hydroxyl, amino or sulfhydryl groups. Thus, representative examples of prodrugs include (but are not limited to) the acetate/acetamide, formate/formamide and benzoate/benzamide derivatives of the hydroxyl, sulfhydryl or amino functional groups of the compounds of formula (I). Furthermore, in the case of carboxylic acid (—COOH), esters such as methyl esters and ethyl esters, etc. can be employed. The ester itself may be active in their own and/or hydrolyzable under in vivo conditions in the human body. Suitable pharmaceutically acceptable in vivo hydrolysable ester groups include those groups that can readily break down in the human body to release the parent acids or salts thereof.

The present disclosure also provides a pharmaceutical formulation comprising a therapeutically effective amount of a compound of formula (I), or therapeutically acceptable salts thereof, and pharmaceutically acceptable carriers, diluents or excipients thereof. All of these forms belong to the present disclosure.

### Pharmaceutical Compositions and Kits

In another aspect, the present disclosure provides a pharmaceutical composition comprising a compound of the present disclosure (also referred to as the “active ingredient”) and a pharmaceutically acceptable excipient. In certain embodiments, the pharmaceutical composition comprises an effective amount of the compound of the present disclosure. In certain embodiments, the pharmaceutical composition comprises a therapeutically effective amount of the compound of the present disclosure. In certain embodiments, the pharmaceutical composition comprises a prophylactically effective amount of the compound of the present disclosure.

A pharmaceutically acceptable excipient for use in the present disclosure refers to a non-toxic carrier, adjuvant or vehicle which does not destroy the pharmacological activity of the compound formulated together. Pharmaceutically acceptable carriers, adjuvants, or vehicles that may be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

Suitable formulations for administering the compounds of the present disclosure will be apparent to those of ordinary skill in the art and include, for example, tablets, pills, capsules, suppositories, lozenges, troches, solutions (particularly solutions for injection (subcutaneous, intravenous, intramuscular) and infusion (injection)), elixirs, syrups, cachets, emulsions, inhalants and dispersible powders. The content of one or more pharmaceutically active compounds should range from 0.1 to 90 wt %, alternatively from 0.5 to 50 wt %, of the composition as a whole, i.e., an amount sufficient to achieve the dosage range specified below. If necessary, the specified dosage can be administered several times a day.

The present disclosure also includes kits (e.g., pharmaceutical packs). Kits provided may include a compound disclosed herein, other therapeutic agents, and a first and a second containers (e.g., vials, ampoules, bottles, syringes, and/or dispersible packages or other suitable containers) containing the compound disclosed herein or other therapeutic agents. In some embodiments, kits provided can also optionally include a third container containing a pharmaceutically acceptable excipient for diluting or suspending the compound disclosed herein and/or other therapeutic agent. In some embodiments, the compound disclosed herein provided in the first container and the other therapeutic agents provided in the second container is combined to form a unit dosage form.

### Administration

The pharmaceutical composition provided by the present disclosure can be administered by a variety of routes including, but not limited to, oral administration, parenteral administration, inhalation administration, topical administration, rectal administration, nasal administration, oral administration, vaginal administration, administration by implant or other means of administration. For example, parenteral administration as used herein includes subcutaneous administration, intradermal administration, intravenous administration, intramuscular administration, intra-articular administration, intraarterial administration, intrasynovial administration, intrasternal administration, intracerebroventricular administration, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the disorder disclosed herein, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or alternatively from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with alternative doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, alternatively from about 0.1 to about 20% by weight, alternatively from about 0.1 to about 10% by weight, and still alternatively from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound of the present disclosure. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, for example, sulfobutyl ether β-cyclodextrin, also known as Captisol. See, e.g., U.S. Pat. No. 5,376,645. In certain embodiments, the formulation comprises hexapropyl-5-cyclodextrin (e.g., 10-50% in water).

### Indications

For tumors that overexpress CAIX, the development of CAIX inhibitors can provide therapeutic benefits for a large number of tumor patients. The compounds of the present disclosure exert therapeutic effects by negatively regulating the activity of CAIX in tumor cells, and after being labeled with diagnostic and therapeutic radionuclides, they can be used for molecular imaging diagnosis and treatment of tumors.

In some embodiments, the compounds or complexes of the present disclosure can be used to diagnose and treat a variety of cancers, including but not limited to tumor types such as renal cancer, lung cancer, colorectal cancer, gastric cancer, pancreatic cancer, melanoma, breast cancer, cervical cancer, bladder cancer, ovarian cancer, brain cancer, head and neck cancer, astrocytoma and oral cancer.

### Examples

The compounds and preparation methods of the present disclosure will be described in further detail below in conjunction with specific examples. It should be understood that the following examples are only intended to illustrate and explain the present disclosure and should not be construed as limiting the scope of protection of the present disclosure. All technical solutions implemented based on the content of the present disclosure are included in the scope that the present disclosure is intended to protect.

Unless otherwise specified, the experimental methods used in the following examples are conventional methods in the art; the reagents, raw materials, instruments, equipment, etc. used in the following examples can all be obtained from commercial channels.

TABLE 1

| Abbreviation | |
|---|---|
| of chemical | |
| substance | |
| name | Full name |
| 2-CTC resin | 2-Chlorotrityl chloride resin |
| DCM | Dichloromethane |
| DMF | N,N-Dimethylformamide |
| DIEA | N,N-Diisopropylethylamine |
| HBTU | Benzotriazole-N,N,N′,N′-tetramethyluronium |
| | hexafluorophosphate |
| DOTA | 1,4,7,10-Tetraazacyclododecane-1,4,7,10-tetraacetic acid |
| HATU | N,N,N′,N′-Tetramethyl-O-(7-azabenzotriazol-1-yl)uronium |
| | hexafluorophosphate |
| HOAt | 1-Hydroxy-7-azabenzotriazole |
| DIC | N,N′-Diisopropylcarbodiimide |
| TFA | Trifluoroacetic acid |
| TIS | Triisopropylsilane |
| 3-Mpr | 3-Mercaptopropionic acid |

### Example 1 Synthesis of Compounds

### 1.1 Synthesis of Compound 1

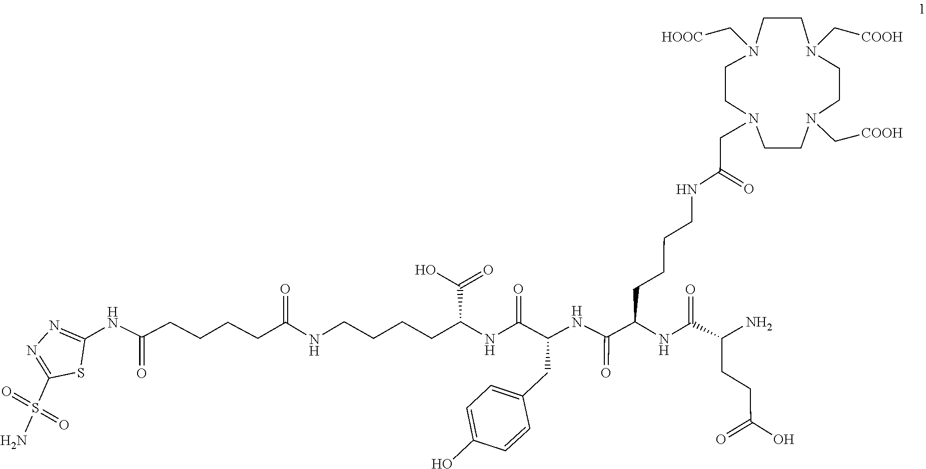 [SMILES: N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)CCCCC(=O)Nc1nnc(S(N)(=O)=O)s1)C(=O)O]

### Synthesis of Peptide

1) 0.20 mmol of 2-CTC resin (Sub=0.50 mmol/g) and Fmoc-D-Lys(Dde)-OH (0.20 mmol) were added into a reaction column. 20.0 mL of DCM was added to the mixture, then DIEA (0.80 mmol) was added dropwise to the mixture. The nitrogen was adjusted to ensure uniform bubbling of the resin. After the mixture was reacted at 20° C. for 2 hours, MeOH (0.40 mL) was added dropwise into the reaction column. The mixture was bubbled with nitrogen for 30 minutes, and washed with DMF. The waste was discarded.

2) Deprotection: 20% piperidine/DMF (V:V) (40.0 mL) was added to the resin. The mixture was bubbled with nitrogen for 15 minutes. The resin was washed with DMF and suctioned to dryness to obtain a resin.

3) Coupling of amino acids: Fmoc-D-Tyr(tBu)-OH (0.60 mmol) and HBTU (0.57 mmol) were added into the resin obtained in the previous step. 30.0 mL of DMF was added to the mixture, and then DIEA (1.20 mmol) was added dropwise into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

4) Deprotection: 20% piperidine/DMF (V:V) (40.0 mL) was added to the resin. The mixture was bubbled with nitrogen for 15 minutes. The resin was washed with DMF and suctioned to dryness to obtain a resin.

5) Coupling of amino acids: Fmoc-D-Lys (Alloc)-OH (0.60 mmol) and HBTU (0.57 mmol) were added into the resin obtained in the previous step. 30.0 mL of DMF was added to the mixture, and then DIEA (1.20 mmol) was added dropwise into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

6) Deprotection: 20% piperidine/DMF (V:V) (40.0 mL) was added to the resin. The mixture was bubbled with nitrogen for 15 minutes. The resin was washed with DMF and suctioned to dryness to obtain a resin.

7) Coupling of amino acids: Fmoc-D-Glu (tBu)-OH (0.60 mmol) and HBTU (0.57 mmol) were added into the resin obtained in the previous step. 30.0 mL of DMF was added to the mixture, and then DIEA (1.20 mmol) was added dropwise into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

8) Deprotection: 20% piperidine/DMF (V:V) (40.0 mL) was added to the resin. The mixture was bubbled with nitrogen for 15 minutes. The resin was washed with DMF and suctioned to dryness to obtain a resin.

9) Boc protection: Preparation of end-capping solution: Boc anhydride:DIEA:DMF (V:V:V)=10:5:85. The end-capping solution (30.0 mL) was added into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

10) De-Allocation: The resin was washed with DMF (40.0 mL) and DCM (40.0 mL) in sequence. An appropriate amount of DCM (20.0 mL) was added into the resin and the mixture was bubbled with nitrogen. PhSiH₃ (2.00 mmol) and Pd(PPh₃)₄ (0.02 mmol) were added into the mixture in sequence. The mixture was reacted for 20 minutes, and the reaction was repeated for 3 times. The resin was washed with DMF and suctioned to dryness to obtain a resin.

11) Coupling of a chelating group: DOTA (0.30 mmol) and HATU (0.28 mmol) were added into the resin obtained in the previous step. 30.0 mL of DMF was added, and then DIEA (0.60 mmol) was added dropwise to the reaction column. The nitrogen was adjusted to ensure uniform bubbling of the resin. The mixture was reacted at 20° C. for 1 hour. The reaction solution was filtered by suction, and washed with DMF. The waste was discarded until no liquid flowed out.

12) De-Dde: A hydrazine hydrate/DMF solution with a hydrazine hydrate mass fraction of 3% was prepared. The hydrazine hydrate solution (40.0 mL) was added to the resin. The reaction was bubbled with nitrogen. The resin was washed with DMF and suctioned to dryness to obtain a resin.

13) Coupling of ligands: Int C (0.24 mmol) and HOAt (0.24 mmol) were added into the resin obtained in the previous step. 30.0 mL of DMF was added to the mixture, and then DIC (0.24 mmol) was added dropwise into the reaction column. The nitrogen was adjusted to ensure uniform bubbling of the resin. The mixture was reacted at 20° C. The reaction solution was filtered by suction, and washed with DMF. The waste was discarded until no liquid flowed out.

14) MeOH (30.0 mL) was used to shrink the resin and the waste was discarded until no liquid flowed out. The resin was poured out and dried for later use.

### Peptide Cleavage:

At room temperature, the dried resin was added to a prepared cutting solution (90% TFA/2.5% H₂O/5% TIS/2.5% 3-Mpr, 20.0 mL) and cut for 2 h. The mixture was filtered, and the filtrate was added to ice isopropyl ether (100 mL) for sedimentation centrifugation. The residue was then washed twice with isopropyl ether (100 mL), and dried under vacuum for 2 hours to obtain the unpurified crude Compound 1 (250 mg).

### Peptide Purification:

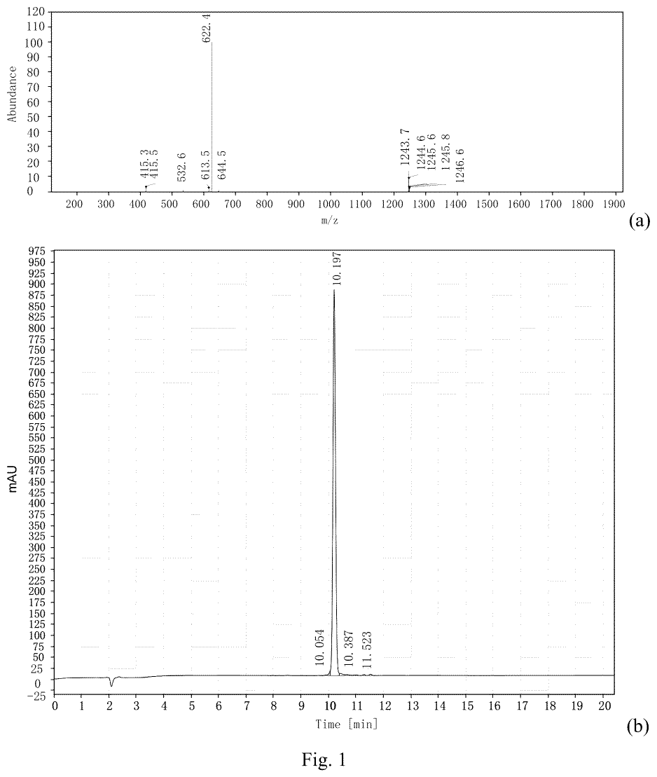

The crude product was purified by preparative HPLC to obtain the final product, Compound 1 (15.0 mg, 97.5% purity). The detection results are shown in FIG. 1.
wherein, the structures of Fmoc-D-Lys (Dde)-OH, Fmoc-D-Tyr(tBu)-OH, Fmoc-D-Lys(Alloc)-OH, Fmoc-D-Glu(tBu)-OH and intermediate Int C are as follows:

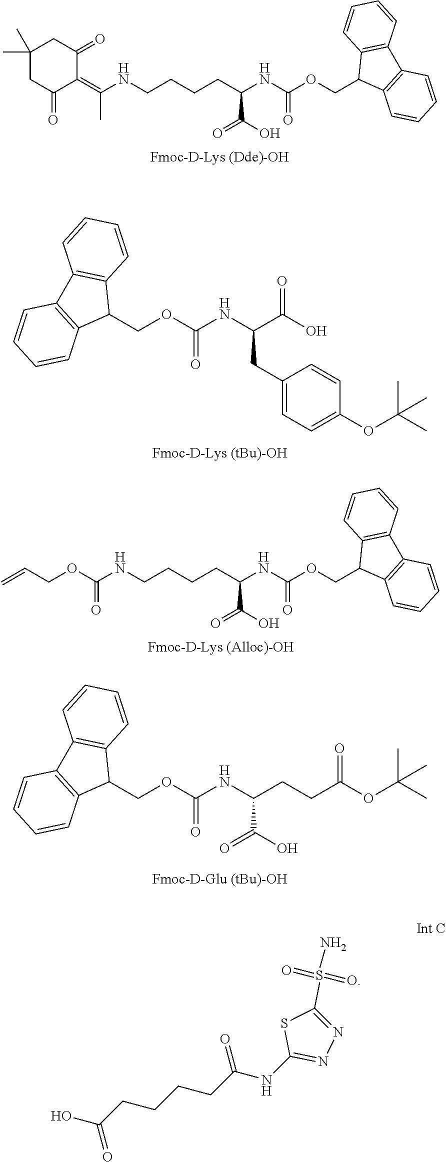

wherein, the synthesis method of intermediate Int C is as follows:

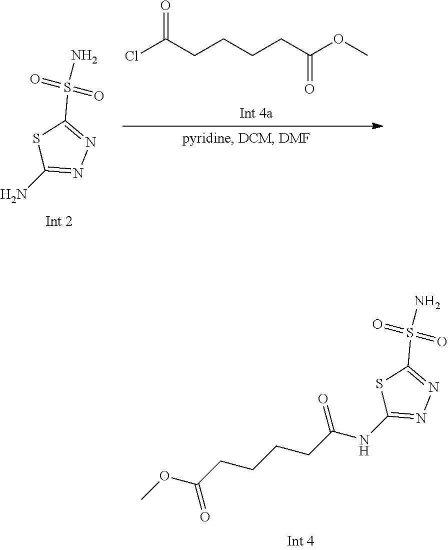 [SMILES: Nc1nnc(S(N)(=O)=O)s1 ; COC(=O)CCCCC(=O)Cl ; COC(=O)CCCCC(=O)Nc1nnc(S(N)(=O)=O)s1]

1) Compound Int 2 (3.00 g, 1.00 eq) and pyridine (2.69 mL, 2.00 eq) were dissolved in DMF (30.0 mL). Compound Int 4a (2.97 g, 1.00 eq) was dissolved in DCM (20.0 mL) at 0° C. and slowly added to the reaction solution. The reaction solution was stirred at 25° C. for 14 hours. The reaction solution was concentrated. Water and saturated sodium bicarbonate aqueous solution were added to the residue, and the mixture was filtered to obtain Compound Int 4 (5.00 g, 96.7% purity).

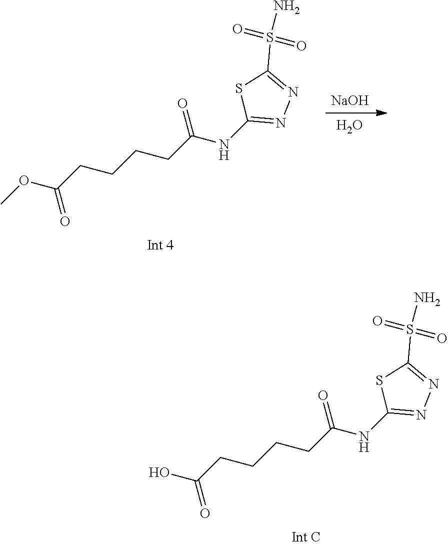

2) Compound Int 4 (5.00 g, 1.00 eq) and NaOH (3.45 g, 5.76 eq) were dissolved in H₂O (70.0 mL), and the reaction solution was stirred at 60° C. for 1 hour. The reaction solution was cooled to room temperature, and the pH was adjusted to 4 with 1N HCl. The solid was precipitated, filtered and purified to obtain Int C (2.00 g, 95.9% purity).

The relevant characterization data of Int C are as follows:

LCMS: MS: [M+H]⁺=308.8.

HPLC: Purity: 95.9%

¹H NMR: 400 MHz DMSO-d6

δ ppm: 12.51 (br s, 2H), 8.30 (br s, 2H), 2.56-2.52 (m, 2H), 2.23 (t, J=7.2 Hz, 2H), 1.70-1.58 (m, 2H), 1.58-1.47 (m, 2H).

### 1.2 Synthesis of Compound 2

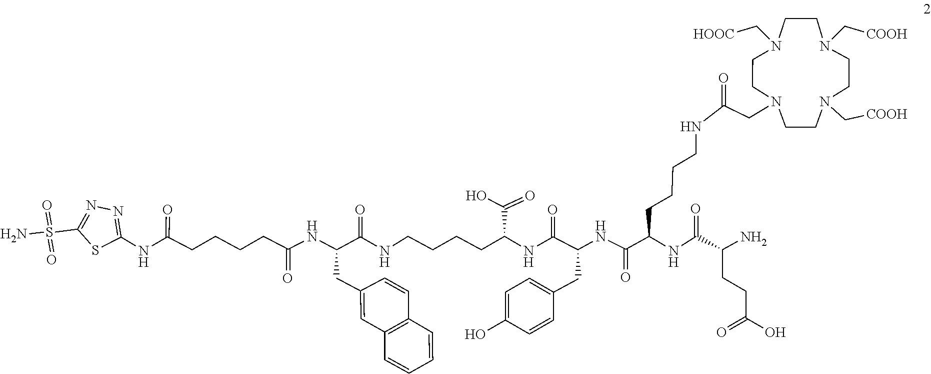 [SMILES: N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)[C@H](Cc1ccc2ccccc2c1)NC(=O)CCCCC(=O)Nc1nnc(S(N)(=O)=O)s1)C(=O)O]

### Synthesis of Peptides:

The peptide was synthesized using standard Fmoc chemical method.

1) 0.30 mmol of 2-CTC resin (Sub=0.30 mmol/g) and Fmoc-D-Lys(Dde)-OH (0.30 mmol, 1.00 eq) were added into a reaction column. 20.0 mL of DCM was added to the mixture, then DIEA (1.20 mmol, 4.00 eq) was added dropwise to the mixture. The nitrogen was adjusted to ensure uniform bubbling of the resin. After the mixture was reacted at 20° C. for 2 hours, MeOH (1.00 mL) was added dropwise into the reaction column. The mixture was bubbled with nitrogen for 30 minutes, and the waste was discarded. The residue was washed with DMF and the waste was discarded until no liquid flowed out.

2) Deprotection: 20% piperidine/DMF (V:V) (30.0 mL) was added to the resin. The mixture was bubbled with nitrogen for 15 minutes. The resin was washed with DMF and suctioned to dryness to obtain a resin.

3) Coupling of amino acids: Fmoc-D-Tyr(tBu)-OH (0.90 mmol, 3.00 eq) and HBTU (0.85 mmol, 2.85 eq) were added into the resin obtained in the previous step. 30.0 mL of DMF was added to the mixture, and then DIEA (1.80 mmol, 6.00 eq) was added dropwise into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

The above steps 2) and 3) were repeated to couple the following amino acids:

TABLE 2

| # | Ingredients/Steps | Reagents |
|---|---|---|
| 1 | Fmoc-D-Lys(Alloc)-OH(3.00 eq) | HBTU (2.85 eq), DIEA (6.00 eq) |
| 2 | Fmoc-D-Glu(tBu)-OH(3.00 eq) | HBTU (2.85 eq), DIEA (6.00 eq) |
| # | Boc anhydride (2.00 eq) | DIEA (4.00 eq) |
| # | De-Alloc | PhSiH₃ (10.0 eq), Pd(PPh₃)₄ (0.10 eq) |
| 3 | DOTA (1.50 eq) | HATU (1.42 eq), DIEA (3.00 eq) |
| # | De-Dde | 3% mass fraction of hydrazine hydrate/DMF |
| 4 | Fmoc-2-Nal-OH (3.00 eq) | HBTU (2.85 eq), DIEA (6.00 eq) |
| 5 | Int C (1.50 eq) | HOAT (1.50 eq), DIC (1.50 eq) |

MeOH (100 mL) was used to shrink the resin and the waste was discarded until no liquid flowed out. The resin was poured out and dried for later use.

Note: De-Alloc conditions: PhSiH₃ (5.00 mmol, 10.0 eq), Pd(PPh₃)₄ (0.05 mmol, 0.10 eq), 10 min*3.

De-Dde conditions: 3% hydrazine hydrate/DMF, 15 min*2.

The structure of Fmoc-2-Nal-OH is as follows:

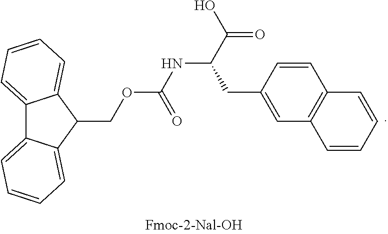 [SMILES: O=C(N[C@@H](Cc1ccc2ccccc2c1)C(=O)O)OCC1c2ccccc2-c2ccccc21]

### Peptide Cleavage:

At room temperature, the dried resin was added to a prepared cutting solution (90% TFA/2.5% H₂O/5% TIS/2.5% 3-Mpr, 30.0 mL) and cut. The mixture was filtered, and the filtrate was added to ice isopropyl ether for sedimentation centrifugation. The residue was then washed with isopropyl ether, and dried under vacuum to obtain the unpurified crude Compound 2 (450 mg).

### Peptide Purification:

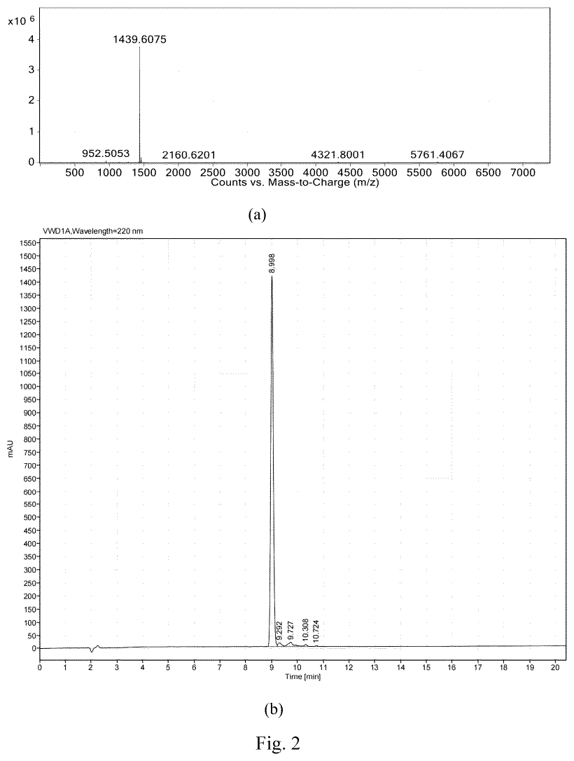

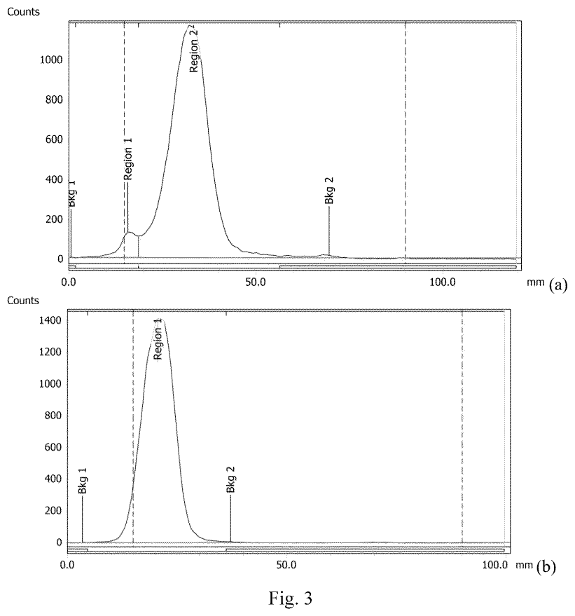

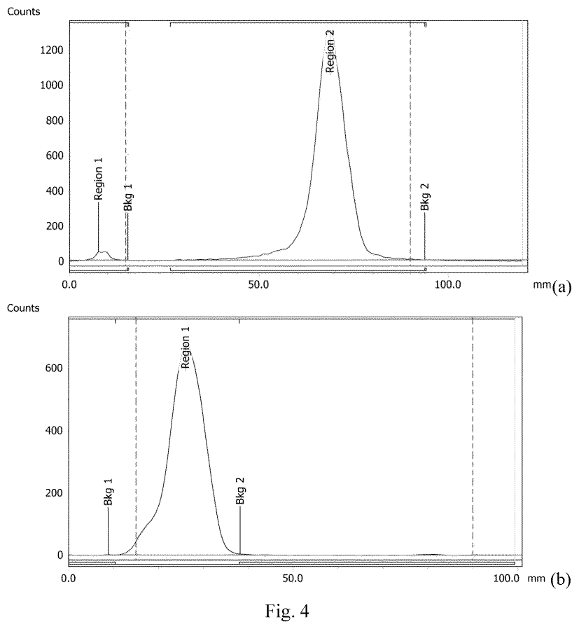

The crude peptide was purified by preparative HPLC (A: 0.075% TFA in water, B: acetonitrile) to obtain the final product, Compound 2 (13.0 mg, purity 95.4%, TFA). The detection results are shown in FIG. 2.

LCMS: MS: [M+H]⁺=1440.6.

HPLC: Purity: 95.4%.

### 1.3 Synthesis of Compound 3

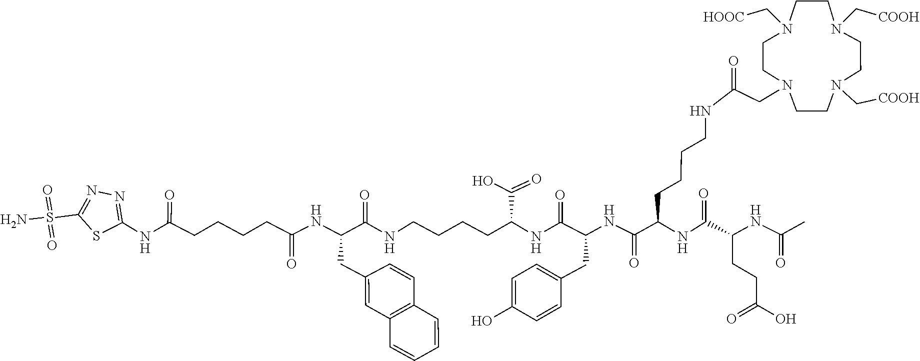 [SMILES: CC(=O)N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)[C@H](Cc1ccc2ccccc2c1)NC(=O)CCCCC(=O)Nc1nnc(S(N)(=O)=O)s1)C(=O)O]

### Synthesis of Peptides:

The peptide was synthesized using standard Fmoc chemical method.

1) 0.30 mmol of 2-CTC resin (Sub=0.30 mmol/g) and Fmoc-D-Lys(Dde)-OH (0.30 mmol) were added into a reaction column. 20.0 mL of DCM was added to the mixture, then DIEA (1.20 mmol) was added dropwise to the mixture. The nitrogen was adjusted to ensure uniform bubbling of the resin. After the mixture was reacted at 20° C. for 2 hours, MeOH (1.00 mL) was added dropwise into the reaction column. The mixture was bubbled with nitrogen for 30 minutes, and washed with DMF. The waste was discarded.

2) Deprotection: 20% piperidine/DMF (V:V) (30.0 mL) was added to the resin. The mixture was bubbled with nitrogen for 15 minutes. The resin was washed with DMF and suctioned to dryness to obtain a resin.

3) Coupling of amino acids: Fmoc-D-Tyr(tBu)-OH (0.90 mmol) and HBTU (0.85 mmol) were added into the resin obtained in the previous step. 30.0 mL of DMF was added to the mixture, and then DIEA (1.80 mmol) was added dropwise into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

4) The above steps 2) and 3) were repeated to couple the following amino acids:

TABLE 3

| # | Ingredients/Steps | Reagents |
|---|---|---|
| 1 | Fmoc-D-Lys (Alloc)-OH(3.00 | HBTU (2.85 eq), DIEA (6.00 eq) |
| | eq) | |
| 2 | Fmoc-D-Glu (tBu)-OH(3.00 eq) | HBTU (2.85 eq), DIEA (6.00 eq) |
| # | Acetic anhydride (2.00 eq) | DIEA (4.00 eq) |
| # | De-Alloc | PhSiH₃ (10.0 eq), |
| | | Pd(PPh₃)₄ (0.10 eq) |
| 3 | DOTA (1.50 eq) | HATU (1.42 eq), DIEA (3.00 eq) |
| # | De-Dde | 3% mass fraction of hydrazine |
| | | hydrate/DMF |
| 4 | Fmoc-2-Nal-OH (3.00 eq) | HBTU (2.85 eq), DIEA (6.00 eq) |
| 5 | Int C (1.50 eq) | HOAT (1.50 eq), DIC (1.50 eq) |

MeOH (100 mL) was used to shrink the resin and the waste was discarded until no liquid flowed out. The resin was poured out and dried for later use.

Note: De-Alloc conditions: PhSiH₃ (5.00 mmol, 10.0 eq), Pd(PPh₃)₄ (0.05 mmol, 0.10 eq), 10 min*3

De-Dde conditions: 3% hydrazine hydrate/DMF, 15 min*2

### Peptide Cleavage:

At room temperature, the dried resin was added to a prepared cutting solution (90% TFA/2.5% H₂O/5% TIS/2.5% 3-Mpr, 30.0 mL) and cut. The mixture was filtered, and the filtrate was added to ice isopropyl ether for sedimentation centrifugation. The residue was then washed with isopropyl ether, and dried under vacuum to obtain the crude peptide Compound 3.

### Peptide Purification:

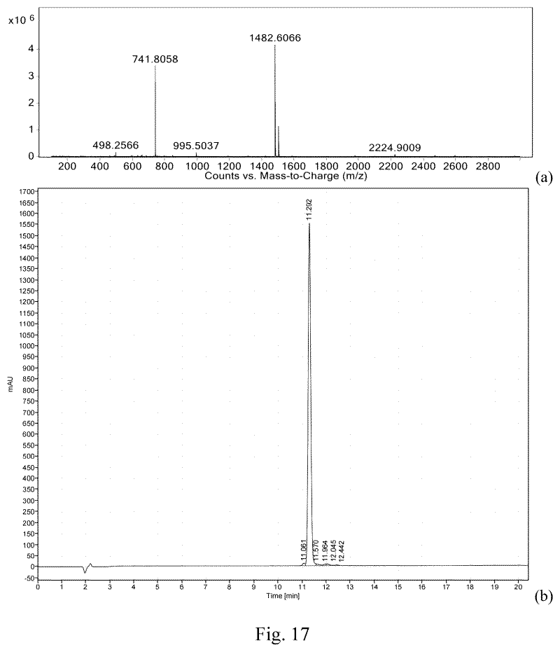

The crude peptide was purified by preparative HPLC to obtain the final product, Compound 3 (13.0 mg, purity 97.2%, TFA). The detection results are shown in FIG. 17.

### 1.4 Synthesis of Compound 4

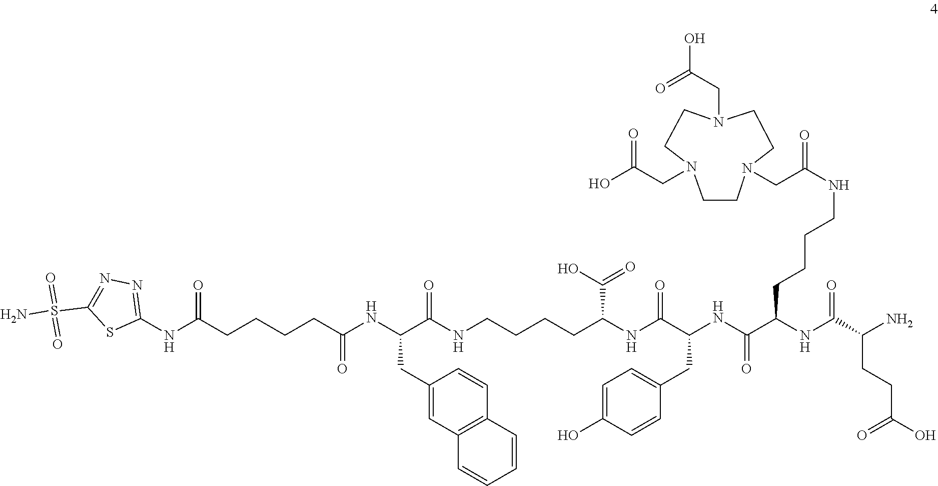 [SMILES: N[C@H](CCC(=O)O)C(=O)N[C@H](CCCCNC(=O)CN1CCN(CC(=O)O)CCN(CC(=O)O)CC1)C(=O)N[C@H](Cc1ccc(O)cc1)C(=O)N[C@H](CCCCNC(=O)[C@H](Cc1ccc2ccccc2c1)NC(=O)CCCCC(=O)Nc1nnc(S(N)(=O)=O)s1)C(=O)O]

### Synthesis of Peptides:

The peptide was synthesized using standard Fmoc chemical method.

1) 0.50 mmol of 2-CTC resin (Sub=0.50 mmol/g) and Fmoc-D-Lys(Dde)-OH (1.00 mmol) were added into a reaction column. 50.0 mL of DCM was added to the mixture, then DIEA (4.00 mmol) was added dropwise to the mixture. The nitrogen was adjusted to ensure uniform bubbling of the resin. After the mixture was reacted at 20° C. for 2 hours, MeOH (1.00 mL) was added dropwise into the reaction column. The mixture was bubbled with nitrogen for 30 minutes, and washed with DMF. The waste was discarded.

2) Deprotection: 20% piperidine/DMF (V:V (50.0 mL)) was added to the resin. The mixture was bubbled with nitrogen for 15 minutes. The resin was washed with DMF and suctioned to dryness to obtain a resin.

3) Coupling of amino acids: Fmoc-D-Tyr (tBu)-OH (3.00 mmol) and HBTU (2.85 mmol) were added into the resin obtained in the previous step. 50.0 mL of DMF was added to the mixture, and then DIEA (6.00 mmol) was added dropwise into the reaction column. The nitrogen was adjusted to ensure uniform bubbling of the resin. The mixture was reacted at 20° C. for 30 minutes. The reaction solution was filtered by suction, and washed with DMF. The waste was discarded until no liquid flowed out.

The above steps 2) and 3) were repeated to couple the following amino acids:

TABLE 4

| # | Ingredients/Steps | Reagents |
|---|---|---|
| 3 | Fmoc-D-Lys (Alloc)-OH(3.00 | HBTU (2.85 eq), DIEA (6.00 eq) |
| | eq) | |
| 4 | Fmoc-D-Glu (tBu)-OH (3.00 eq) | HATU (1.20 eq), DIEA (3.00 eq) |
| # | Boc anhydride (1.00 eq) | DIEA (1.00 eq), DMF (8:00 eq) |
| # | De-Alloc | PhSiH₃ (10.0 eq), |
| | | Pd(PPh₃)₄ (0.10 eq) |
| 5 | NOTA (tBu)2 (1.5 eq) | HATU (1.45 eq), DIEA (3.00 eq) |
| # | De-Dde | 3% mass fraction of hydrazine |
| | | hydrate/DMF |
| 6 | Fmoc-2-Nal-OH (3.00 eq) | HBTU (2.85 eq), DIEA (6.00 eq) |
| 7 | Int C (1.50 eq) | HBTU (1.40 eq), DIEA (3.00 eq) |

MeOH (100 mL) was used to shrink the resin and the waste was discarded until no liquid flowed out. The resin was poured out and dried for later use.

Note: De-Alloc conditions: PhSiH3 (10.0 mmol, 10.0 eq), Pd(PPh3)₄ (0.10 mmol, 0.10 eq), 15 min*3

De-Dde conditions: 3% hydrazine hydrate/DMF, 10 min*2

### Peptide Cleavage:

At room temperature, the dried resin was added to a prepared cleavage solution (92.5% TFA/2.5% H₂O/2.5% TIS/2.5% 3-Mpr, 6.00) and cut. The mixture was filtered, and the filtrate was added to ice isopropyl ether for sedimentation centrifugation. The residue was then washed with isopropyl ether, and dried under vacuum to obtain the unpurified crude peptide Compound 4 (450 mg).

### Peptide Purification:

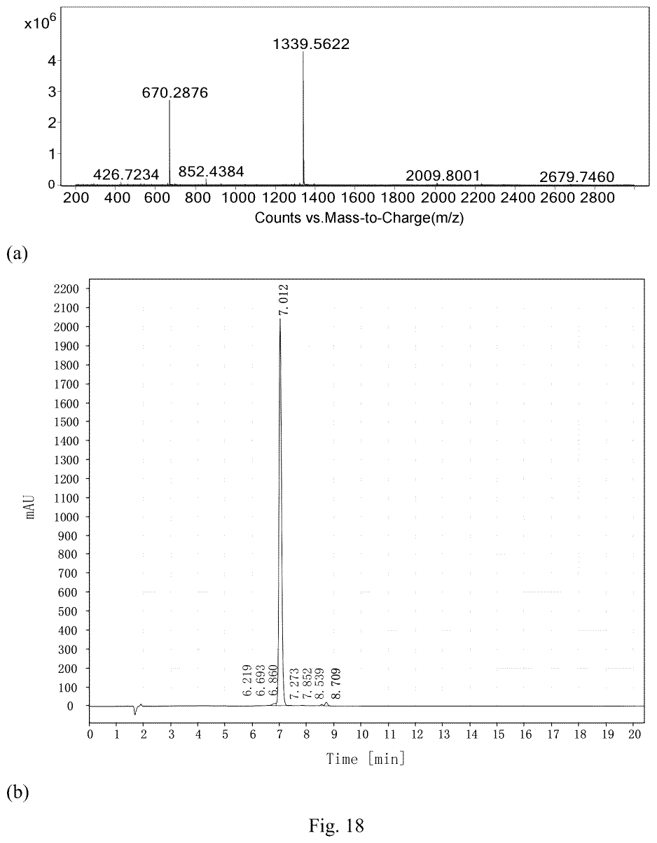

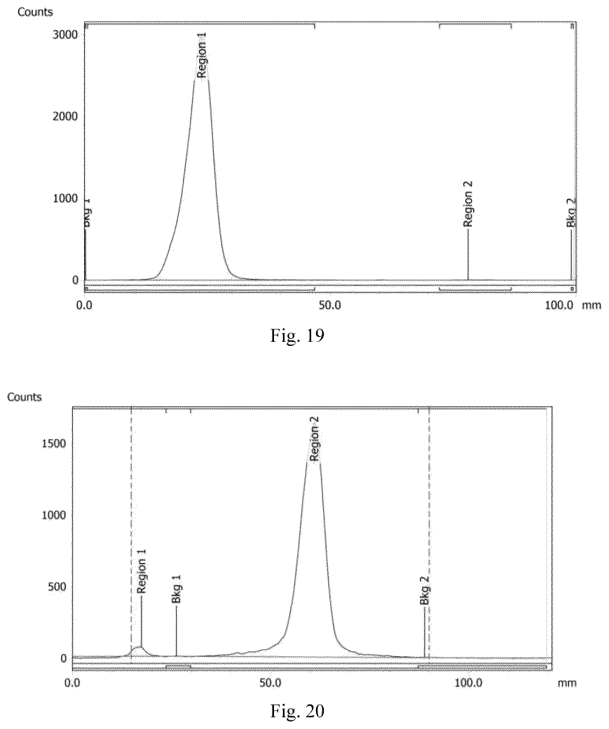

The crude peptide was purified by preparative HPLC to obtain the final product, Compound 4 (22.2 mg, purity 97.8%, TFA). The detection results are shown in FIG. 18.

### 1.5 Synthesis of Compound 5

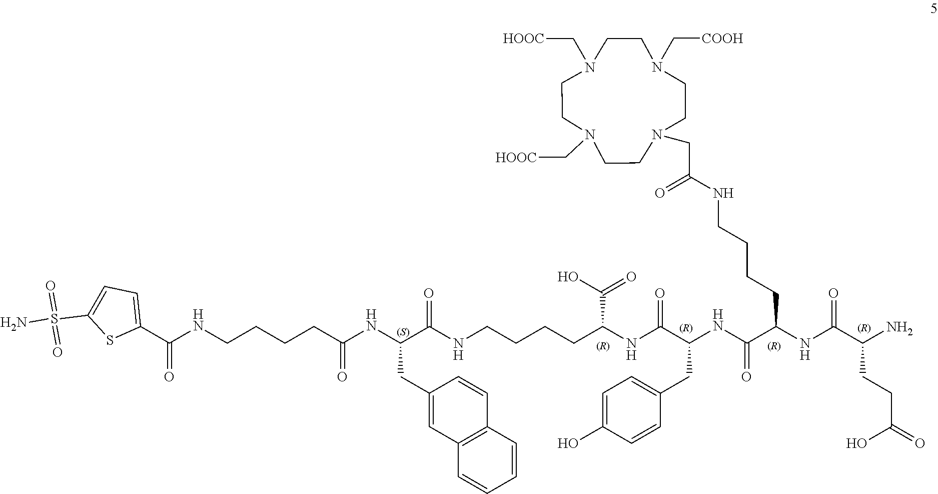

### Synthesis of Peptides:

The peptide was synthesized using standard Fmoc chemical method.

1) In a solid-phase synthesis column, 0.15 mmol of 2-CTC resin (Sub=0.50 mmol/g) and Fmoc-D-Lys(Dde)-OH (1.0 eq) were reacted in DCM at 20° C. for 2 hours. A small amount of MeOH was added into the reaction column and continued the reaction for 30 mins. The mixture was washed to remove the residual liquid and washed with DMF for several times.

2) Deprotection of Fmoc: The reaction was treated with 20% piperidine/DMF (10 mL) for 15 minutes, and washed with DMF (30 mL) for 5 times to obtain a deprotected resin.

3) Coupling reaction: Fmoc-D-Tyr (tBu)-OH, HBTU and DIEA were added to the resin and reacted in DMF at 20° C. for 1 hour. After the completion of the reaction, the waste was discarded by washing.

The above steps 2) and 3) were repeated to couple the following amino acids (3-8):

TABLE 5

| # | Ingredients | Reagents |
|---|---|---|
| 3 | Fmoc-D-Lys (Alloc)-OH(3.00 eq) | HBTU (2.85 eq), and DIEA (6.00 eq) |
| 4 | Boc-D-Glu(OtBu)-OH (3.00 eq) | HOAt (3.00 eq) and DIC (3.00 eq) |
| # | De-Alloc | PhSiH₃ (10.0 eq) and Pd(PPh₃)₄ (0.10 eq) |
| 5 | DOTA (3.00 eq) | HATU (2.85 eq) and DIEA (6.00 eq) |
| # | De-Dde | 3% hydrazine hydrate/DMF (10.0 mL) |
| 6 | Fmoc-2-Nal-OH (3.00 eq) | HATU (2.85 eq) and DIEA (6.00 eq) |
| 7 | Fmoc-5-Ava-OH (3.00 eq) | HOAt (3.00 eq) and DIC (3.00 eq) |
| 8 | 5-sulfamoylthiophene-2-carboxylic | HATU (1.40 eq), and DIEA (3.00 eq) |
| | acid (1.50 eq) | |

MeOH was used to shrink the resin and the waste was discarded until no liquid flowed out. The resin was poured out and dried for later use.

### Note:

De-Alloc: DCM (30.0 mL) was added into the reaction column. PhSiH3 (10.0 eq) and Pd(PPh3)4 (0.10 eq) were added to the reaction sequently. The nitrogen was adjusted to ensure uniform bubbling of the resin. After the mixture was reacted at 20° C. for 15 mins, the reaction solution was filtered and washed with DMF for 5 times.

De-Dde: 3% hydrazine hydrate/DMF (10.0 mL) was added into the reaction column. The nitrogen was adjusted to ensure uniform bubbling of the resin. After the mixture was reacted at 20° C. for 15 mins, the reaction solution was filtered, washed with DMF, and the waste was discarded.

### Peptide Cleavage:

At room temperature, a prepared cleavage solution (90.0% TFA/5.0% TIS/2.5% H2O/2.5%3-Mpr) was added to the dried resin and the mixture was stirred at room temperature for 2 hours. The mixture was added to ice methyl tert-butyl ether for sedimentation centrifugation. The residue was then washed with methyl tert-butyl ether, and dried.

### Peptide Purification:

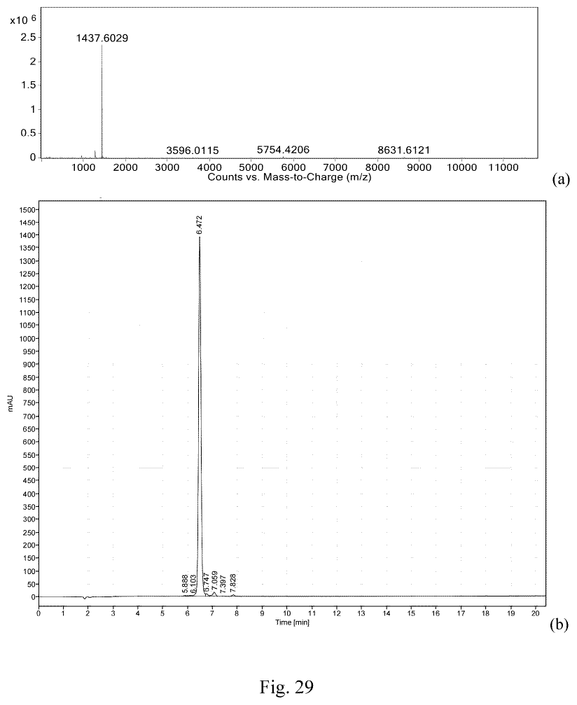

The concentrated solution was purified by preparative HPLC (A: 0.075% TFA in water, B: acetonitrile) to obtain Compound 5 (18.0 mg, purity 97.32%, TFA). The detection results are shown in FIG. 29.

### 1.6 Synthesis of Compound 6

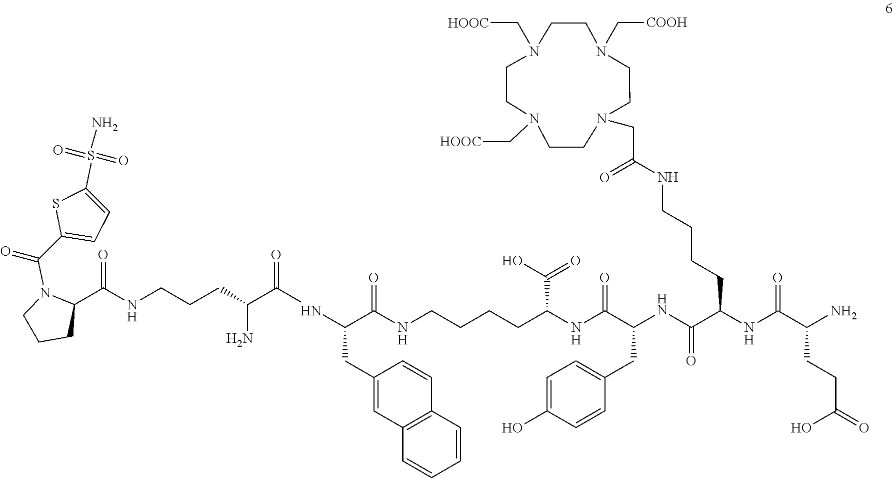 [SMILES: N[C@H](CCCNC(=O)[C@H]1CCCN1C(=O)c1ccc(S(N)(=O)=O)s1)C(=O)N[C@@H](Cc1ccc2ccccc2c1)C(=O)NCCCC[C@@H](NC(=O)[C@@H](Cc1ccc(O)cc1)NC(=O)[C@@H](CCCCNC(=O)CN1CCN(CC(=O)O)CCN(CC(=O)O)CCN(CC(=O)O)CC1)NC(=O)[C@H](N)CCC(=O)O)C(=O)O]

### Synthesis of Peptides:

The peptide was synthesized using standard Fmoc chemical method.

1) In a solid-phase synthesis column, 0.15 mmol of 2-CTC resin (Sub=0.50 mmol/g) and Fmoc-D-Lys(Dde)-OH (1.0 eq) were reacted in DCM at 20° C. for 2 hours. A small amount of MeOH was added into the reaction column and continued the reaction for 30 mins. The mixture was washed to remove the residual liquid and washed with DMF for several times.

2) Deprotection of Fmoc: The reaction was treated with 20% piperidine/DMF (10 mL) for 15 minutes, and washed with DMF (30 mL) for 5 times to obtain the deprotected resin.

3) Coupling reaction: Fmoc-D-Tyr (tBu)-OH, HBTU and DIEA were added to the resin and reacted in DMF at 20° C. for 1 hour. After the completion of the reaction, the waste was discarded by washing.

The above steps 2) and 3) were repeated to couple the following amino acids (3-9):

TABLE 6

| # | Ingredients | Reagents |
|---|---|---|
| 3 | Fmoc-D-Lys(Alloc)-OH (3.00 | HBTU (2.85 eq) and DIEA (6.00 eq) |
| | eq) | |
| 4 | Boc-D-Glu(OtBu)-OH (3.00 | HOAt (3.00 eq) and DIC (3.00 eq) |
| | eq) | |
| # | De-Alloc | PhSiH3 (10.0 eq) and |
| | | Pd(PPh3)4 (0.10 eq) |
| 5 | DOTA (3.00 eq) | HATU (2.85 eq) and DIEA (6.00 eq) |
| # | De-Dde | 3% hydrazine hydrate/DMF |
| | | (10.0 mL) |
| 6 | Fmoc-2-Nal-OH (3.00 eq) | HATU (2.85 eq) and DIEA (6.00 eq) |
| 7 | BOC-D-Orn(Fmoc)-OH (3.00 | HOAt (3.00 eq) and DIC (3.00 eq) |
| | eq) | |
| 8 | Fmoc-D-Pro-OH (3.00 eq) | HATU (2.85 eq) and DIEA (6.00 eq) |
| 9 | 5-sulfamoylthiophene-2- | HATU (1.40 eq), and DIEA (3.00 eq) |
| | carboxylic acid (1.50 eq) | |

MeOH was used to shrink the resin and the waste was discarded until no liquid flowed out. The resin was poured out and dried for later use.

### Note:

De-Alloc: DCM (30.0 mL) was added into the reaction column. PhSiH3 (10.0 eq) and Pd(PPh3)4 (0.10 eq) were added to the reaction sequently. The nitrogen was adjusted to ensure uniform bubbling of the resin. After the mixture was reacted at 20° C. for 15 mins, the reaction solution was filtered and washed with DMF for 5 times.

De-Dde: 3% hydrazine hydrate/DMF (10.0 mL) was added into the reaction column. The nitrogen was adjusted to ensure uniform bubbling of the resin. After the mixture was reacted at 20° C. for 15 mins, the reaction solution was filtered, washed with DMF, and the waste was discarded.

### Peptide Cleavage:

At room temperature, a prepared cleavage solution (90.0% TFA/5.0% TIS/2.5% H₂O/2.5% 3-Mpr) was added to the dried resin and the mixture was stirred at room temperature for 2 hours. The mixture was added to ice methyl tert-butyl ether for sedimentation centrifugation. The residue was then washed with methyl tert-butyl ether, and dried.

### Peptide Purification:

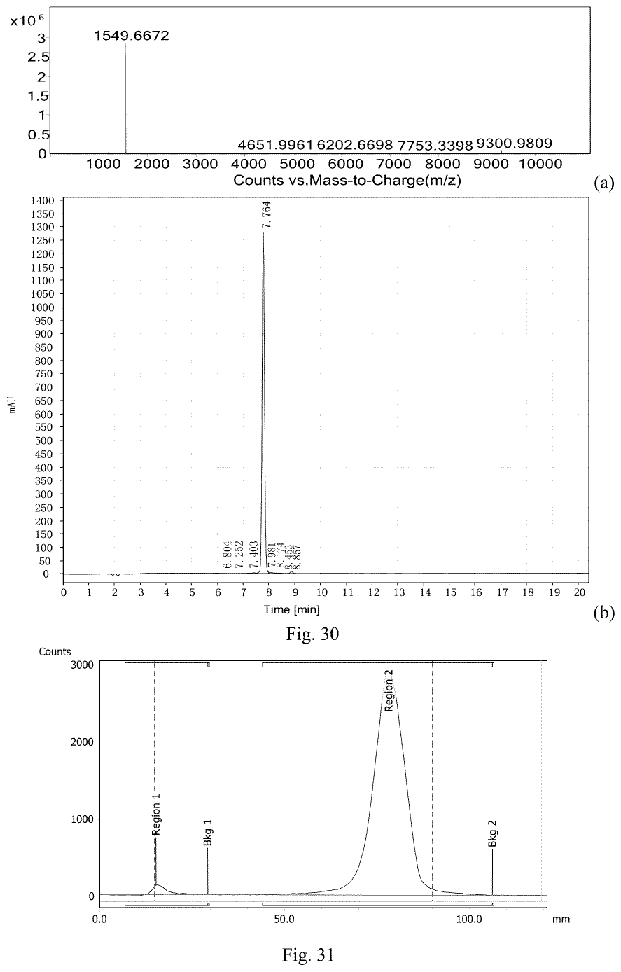

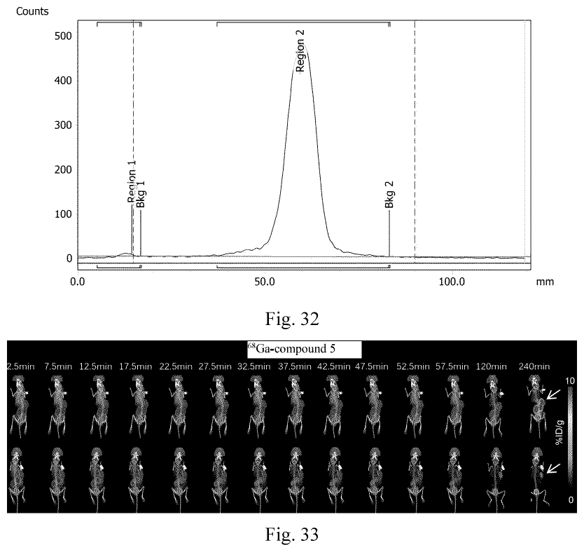

The concentrated solution was purified by preparative HPLC (A: 0.075% TFA in water, B: acetonitrile) to obtain Compound 6 (12.3 mg, purity 98.3%, TFA). The detection results are shown in FIG. 30.

### Example 2 Radiolabeling

2.1 Labeling Process of ⁶⁸Ga-Compound 1, ⁶⁸Ga-Compound 2, ⁶⁸Ga-Compound 3, ⁶⁸Ga-Compound 5, ⁶⁸Ga-Compound 6

1 mL of sodium acetate/acetic acid buffer solution (pH=7.2) was added to a vial, then 60 μL of the precursor aqueous solution of compound 1, Compound 2, Compound 3, Compound 5, or Compound 6 (containing 60 μg) was added and mixed thoroughly. Then, 1 mL of 0.1M hydrochloric acid solution of ⁶⁸GaCl₃ was added to the mixture and the mixture was reacted at 80-100° C. for 10 min. The reaction was quenched. The reaction solution was purified by C18 column: first, the reaction solution was washed with sterile water and the washings were discarded; then the reaction solution was washed with 1 ml of 70% ethanol, and the collected eluent was diluted with 6 mL of physiological saline and filtered aseptically to obtain the product solution, which was sampled for detection. The radiochemical purity of the product was determined by Radio-iTLC method, and the radiochemical purity was >95%.

### 2.2 Labeling Process of ¹⁷⁷Lu-Compound 1

0.45 mL of sodium acetate/acetic acid buffer solution (pH=7.2) was added to a reaction bottle, then 60 μL of the precursor aqueous solution of compound 1 (containing 60 g precursor) was added and mixed thoroughly. 0.45 mL of 0.05M hydrochloric acid solution of ¹⁷⁷LuCl₃ was added to the mixture and the mixture was reacted at 80° C. for 15 min. The reaction was quenched, purified and sampled for detection. The radiochemical purity of the product was determined by Radio-iTLC method, and the radiochemical purity was >95%.

### 2.3 Labeling Process of ¹⁷⁷Lu-Compound 2

1 mL of sodium acetate/acetic acid buffer solution (pH=7.2) was added to a vial, then 60 μL of precursor aqueous solution of compound 2 (containing 60 μg of compound 2) was added and mixed thoroughly. 1 mL of 0.05 M hydrochloric acid solution of ¹⁷⁷LuCl₃ was added to the mixture and the reaction solution was reacted at 80° C. for 15 min. The reaction was quenched, purified and sampled for detection. The radiochemical purity of the product was determined by Radio-iTLC method, and the radiochemical purity was >95%.

### 2.4 Labeling Process of ¹⁶¹Tb-Compound 2

1 mL of sodium acetate/acetic acid buffer solution (pH=7.2) was added to a vial, then 60 μL of the precursor aqueous solution of compound 2 (containing 60 μg of compound 2) was added and mixed thoroughly. Then, 1 mL of 0.05 M hydrochloric acid solution of [¹⁶Tb]-terbium chloride was added to the mixture and the reaction solution was reacted at 80° C. for 15 min. The reaction was quenched, purified and sampled for detection. The radiochemical purity of the product was determined by Radio-iTLC method, and the radiochemical purity was >95%.

### 2.5 Labeling Process of ¹⁸F-Compound 4

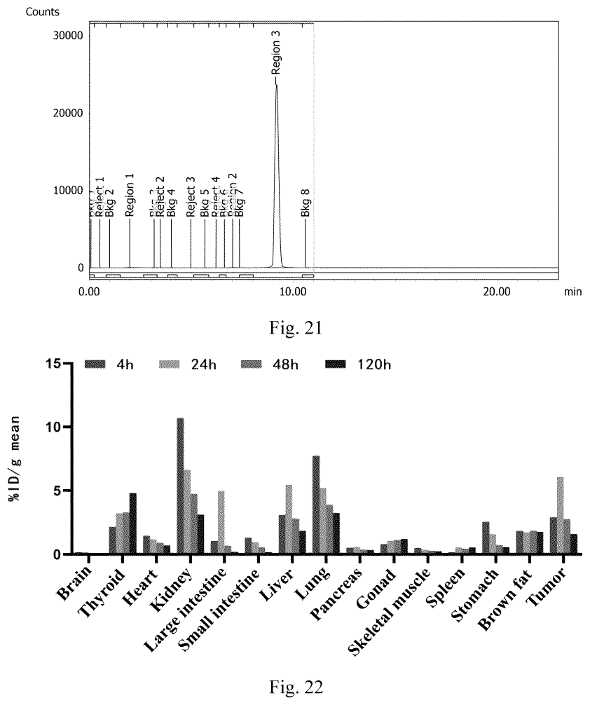

2.5 mL of acetonitrile solution, 27 μL of AlCl₃, 45 μL of acetic acid and 40 μL of precursor compound 1 aqueous solution (containing 200 g of precursor compound 1) were added to a vial and mixed thoroughly. A certain amount of the generated fluorine [18F] aqueous solution was collected and the radioactivity was measured through the activity meter and recorded (the fluorine [18F] aqueous solution can be generated by a cyclotron). The fluorine [18F] aqueous solution was added to a pretreated QMA column, and then eluted with 0.5 mL of saline. The eluent directly flowed into a reaction bottle, which was added with a rubber stopper and covered. The mixture was reacted at 80° C. for 15 min. The reaction was cooled to room temperature. The reaction solution was diluted with sterile injection water, and purified with a C18 column. The effluent was discarded. The C18 column was rinsed with 1.5 mL of 70% ethanol solution slowly. The effluent was collected into the product bottle, diluted with 9 mL of saline, and filtered through a sterile filter membrane into a sterile vacuum bottle. Sampling was performed and the radiochemical purity of the product was determined by HPLC method to be >95%. The result is shown in FIG. 21.

### Preclinical Experiments:

### Mouse Model:

The OS-RC-2 model experimental mice were purchased from Shanghai Bioduro Biologics Co., Ltd. The model was a subcutaneous heterotopic OS-RC-2 tumor transplantation model based on BALB/c nude mice. This model was a mouse model constructed from human renal cancer cells.

ICR mice were purchased from Hengjia Biotechnology (Suzhou) Co., Ltd.

MicroPET/CT equipment: SNPC-303 Super Nova, PINGSENG Healthcare (KunShan) Inc.

Scanning step: the pre-anesthetized animals were placed in a MicroPET/CT imaging cabin for scanning at a specified time after drug administration. The acquired image data was reconstructed by the equipment software, and then processed and analyzed by PMOD software to generate tissue distribution maps and the radioactive uptake values of each tissue and organ were quantitatively determined (expressed as % ID/g).

### Experimental Example 1 SPR Affinity Test

The affinity of compound 1 to CAIX was determined by the Biacore 8K protein interaction system. CAIX (purchased from ACROBiosystems Inc) was coupled to the surface of a CM5 chip. Compound 1 molecule to be tested was diluted with buffer solution to a series of sample solutions with different concentrations. The sample was injected and the affinity of the test compound 1 to CAIX was measured. The affinity of the test compound 1 to CAIX was expressed by the equilibrium dissociation constant K_{D} (K_{d}/Kₐ) value, where K_{d} is the dissociation constant and Kₐ is the binding constant. The smaller the K_{D} value represents the higher the affinity of the compound to the protein.

The results are as follows: the equilibrium dissociation constants K_{D} values of compound 1 and compound 2 binding to CAIX are (9.69±0.32) E-9 M and (8.16±0.51) E-11 M, respectively. It can be seen that compound 1 and compound 2 molecules have a strong affinity to the CAIX protein.

### Experimental Example 2 Tissue Distribution and Targeting Experiment of ⁶⁸Ga-Compound 1 in OS-RC-2 Model Mice

### Experimental Steps:

Four experimental mice in OS-RC-2 model were selected. Each animal was given 80 μCi of ⁶⁸Ga-Compound 1, and the pre-anesthetized animals were placed in a MicroPET/CT imaging cabin (SNPC-303 Super Nova, PINGSENG Healthcare (KunShan) Inc.). Dynamic 30-min MicroPET/CT scanning was performed after drug administration, and static MicroPET/CT scanning was performed 1 and 2 hours after drug administration. The acquired image data was reconstructed by the equipment software, and then processed and analyzed by PMOD software to generate tissue distribution maps and the radioactive uptake values of each tissue and organ were quantitatively determined (expressed as % ID/g).

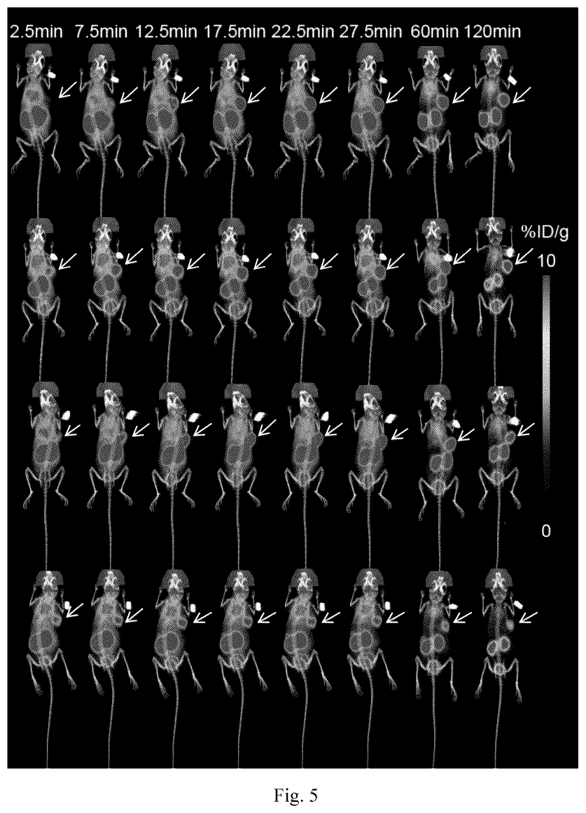

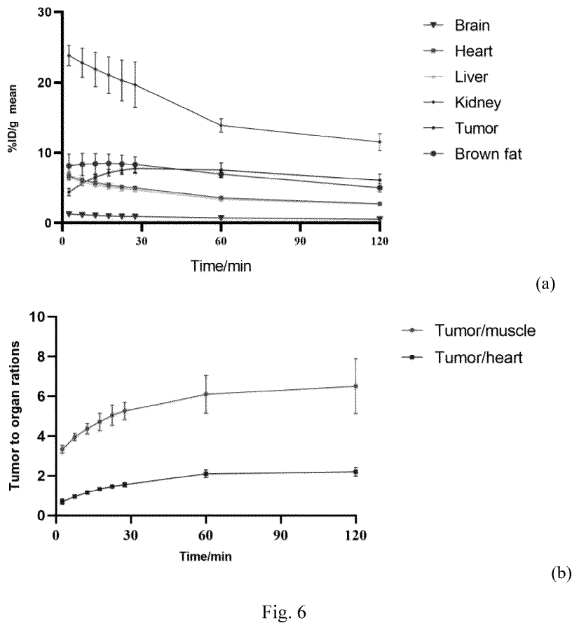

The results are shown in FIG. 5 (the arrow indicates the tumor site). ⁶⁸Ga-compound 1 can be rapidly accumulated in the tumor site, while the uptake in non-target organs is low. ⁶⁸Ga-compound 1 is mainly eliminated rapidly by metabolism through the renal pathway. According to the uptake ratio of tumor to non-target tissue (see FIG. 6 and Table 7 for details), 1 hour after administration, the uptake ratio of tumor to muscle and heart was approximately 6:1 and 2:1, respectively, and remained at a high level 2 hours after administration. The compound has significant target enrichment ability and a high target-to-sample ratio, which helps to improve the clarity and contrast of tumor imaging, showing good imaging characteristics and potential clinical application prospects.

TABLE 7

| | % ID/g | % ID/g | % ID/g |
|---|---|---|---|
| | average | average | average |
| | value | value | value |
| Tissues | (27.5 min) | (60 min) | (120 min) |
| Brain | 0.97 | 0.76 | 0.57 |
| Heart | 5.01 | 3.56 | 2.73 |
| Lung | 5.99 | 4.11 | 3.42 |
| Liver | 4.70 | 3.36 | 2.70 |
| Stomach | 3.15 | 1.69 | 1.29 |
| Kidney | 19.69 | 13.88 | 11.51 |
| Muscle | 1.48 | 1.26 | 0.98 |
| Tumor | 7.75 | 7.57 | 6.09 |
| Fat | 8.33 | 6.98 | 5.02 |
| Intestinal | 4.23 | 2.71 | 2.29 |
| Tumor/Muscle | 5.24 | 6.01 | 6.21 |

### Experimental Example 3 PET/CT Scanning Tissue Distribution and Targeting Experiment of ⁶⁸Ga-Compound 1 in OS-RC-2 Model

### Experimental Steps:

Six experimental mice in OS-RC-2 model were selected. Each animal was given 80 μCi of ⁶⁸Ga-Compound 1, and the pre-anesthetized animals were placed in a MicroPET/CT imaging cabin (SNPC-303 Super Nova, PINGSENG Healthcare (KunShan) Inc.). MicroPET/CT scanning was performed 1 hour after drug administration to obtain scanned images and the images were analyzed.

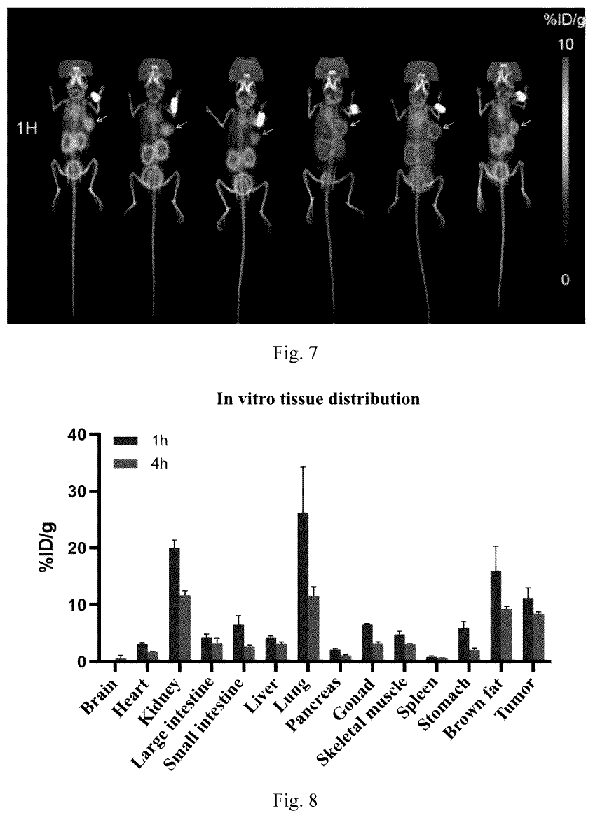

The results are shown in FIG. 7 (the arrow indicates the tumor site). ⁶⁸Ga-compound 1 can be rapidly accumulated in the tumor site 1 hour after administration, and the image of the tumor was obvious and clear, which was of guiding significance for clinical tumor diagnosis.

### Experimental Example 4 In Vitro Tissue Distribution Experiment of ⁶⁸Ga-Compound 1 in OS-RC-2 Model

### Experimental Steps:

Six experimental mice in OS-RC-2 model were selected. Each animal was given 100 μCi of ⁶⁸Ga-compound 1. Three mice were dissected at 1 h and 4 h after administration, respectively. 14 tissues and organs including brain, heart, kidney, large intestine, small intestine, liver, lung, pancreas, gonad, skeletal muscle, spleen, stomach, fat, and tumor were obtained to detect the γ-ray radioactivity count.

Table 8 lists the distribution of radioactivity in each tissue at each time point. As shown in FIG. 8 and Table 8, ⁶⁸Ga-compound 1 was significantly enriched in tumor tissue 1 hour after administration, and maintained a high uptake level 4 hours later. At the same time, the radioactivity uptake of kidneys, lungs, and brown fat (brown fat at the scapula) decreased rapidly over time, and the radioactivity distribution of other non-target organs was low. It can be seen that ⁶⁸Ga-compound 1 can quickly target tumor tissues and maintain high tumor uptake in vivo. At the same time, ⁶⁸Ga-compound 1 can be rapidly eliminated by metabolism through the renal pathway, reducing nonspecific background signals, which is beneficial to improving tumor imaging contrast and diagnostic accuracy, and having good in vivo safety.

TABLE 8

| | % ID/g | % ID/g |
|---|---|---|
| | Average | Average |
| Tissues | value (1 h) | value (4 h) |
| Brain | 0.22 | 0.65 |
| Heart | 3.08 | 1.75 |
| Kidney | 20.01 | 11.67 |
| Large Intestine | 4.21 | 3.24 |
| Small Intestine | 6.61 | 2.61 |
| Liver | 4.19 | 3.18 |
| Lung | 26.25 | 11.58 |
| Pancreas | 2.16 | 1.14 |
| Gonad | 6.61 | 3.22 |
| Skeletal muscle | 4.83 | 3.1 |
| Spleen | 0.93 | 0.71 |
| Stomach | 6.04 | 2.09 |
| Brown fat | 16.02 | 9.29 |
| Tumor | 11.19 | 8.41 |

### Experimental Example 5 Tissue Distribution and Targeting Experiment of ¹⁷⁷Lu-Compound 1 in OS-RC-2 Model

### Experimental Steps:

One experimental mouse in OS-RC-2 model was selected. The animal was intravenously injected with ¹⁷⁷Lu-compound 1 with a radioactive dose of 300 μCi. Small animal SPECT/CT scans were performed at 4, 24, 48, 96, 120, and 168 hours after administration. The animals were pre-anesthetized with an appropriate concentration of isoflurane/air mixture before scanning, and the anesthesia state was maintained during the scanning process. The acquired image data was reconstructed by the equipment software, and then processed and analyzed by PMOD software to generate tissue distribution maps and the radioactive uptake values of each tissue and organ were quantitatively determined (expressed as % ID/g).

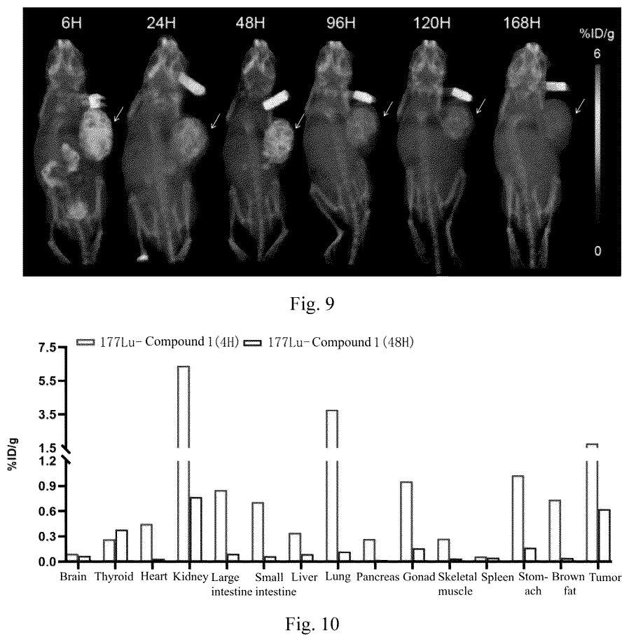

The results are shown in FIG. 9 and Table 9. ¹⁷⁷Lu-compound 1 was significantly enriched in tumor tissues 6 hours after administration (the area indicated by the arrow in the Fig.), and the uptake in non-target organs decreased significantly after 48 hours, and at 168 hours, the tumor still maintained obvious radioactive uptake. This result shows that ¹⁷⁷Lu-compound 1 has good accumulation ability and long retention time in tumor tissues, and is suitable for radiotherapy of tumors. At the same time, its uptake in non-target organs is low and its clearance is fast, indicating that the potential toxic side effects on non-target tissues are small, and it has good targeting and safety.

TABLE 9

| | % ID/g | % ID/g | % ID/g | % ID/g | % ID/g | % ID/g |
|---|---|---|---|---|---|---|
| Tissues | 6 h | 24 h | 48 h | 96 h | 120 h | 168 h |
| Tumor | 8.62 | 9.28 | 11.9 | 29.71 | 43.92 | 72.02 |

### Experimental Example 6 In Vitro Tissue Distribution Experiment of ¹⁷⁷Lu-Compound 1 in OS-RC-2 Model

### Experimental Steps:

Nine experimental mice in OS-RC-2 model were selected. Each animal was given 300 μCi of ¹⁷⁷Lu-compound 1. Three mice were dissected at 4 h, 48 h, and 168 h after administration, respectively. 15 tissues and organs including brain, thyroid, heart, kidney, large intestine, small intestine, liver, lung, pancreas, gonad, skeletal muscle, spleen, stomach, fat, and tumor were obtained to detect the γ-radioactivity count.

Table 10 lists the distribution of radioactivity in each tissue at each time point. As shown in FIG. 10, ¹⁷⁷Lu-compound 1 was significantly enriched in tumor tissue 4 hours after administration. Except for the kidney and lung, the radioactivity uptake in other normal tissues was not high.

48 hours after administration, the uptake in the kidney, lung, brown fat (brown fat in the scapula) and other normal tissues decreased rapidly, while there was still high uptake in the tumor. This shows that ¹⁷⁷Lu-compound 1 can specifically target tumors and can be quickly cleared from non-target organs. ¹⁷⁷Lu-compound 1 has good safety and clinical therapeutic potential.

TABLE 10

| | % ID/g | % ID/g |
|---|---|---|
| | Average | Average |
| Tissues | value (4 h) | value (48 h) |
| Brain | 0.09 | 0.07 |
| Thyroid | 0.27 | 0.38 |
| Heart | 0.45 | 0.04 |
| Kidney | 6.42 | 0.77 |
| Large intestine | 0.85 | 0.09 |
| Small Intestine | 0.71 | 0.07 |
| Liver | 0.34 | 0.09 |
| Lung | 3.78 | 0.12 |
| Pancreas | 0.27 | 0.02 |
| Gonad | 0.96 | 0.16 |
| Skeletal muscle | 0.27 | 0.04 |
| Spleen | 0.06 | 0.05 |
| Stomach | 1.03 | 0.17 |
| Brown fat | 0.74 | 0.04 |
| Tumor | 1.77 | 0.62 |

### Experimental Example 7 Pharmacokinetic Study of ⁶⁸Ga-Compound 1 in ICR Mouse Blood

Six ICR mice were randomly selected and each was given 100 μCi of the ⁶⁸Ga-compound 1. Blood was collected from the submandibular cavity at 5 min, 15 min, 30 min, 1 h, 1.5 h, 2 h, 3 h, and 4 h after administration. The gamma radioactivity count in the blood was determined, and the blood drug concentration at different time points was calculated. The pharmacokinetic software Mas was used to fit the data based on the non-compartmental model to calculate the pharmacokinetic parameters (see the table below for details).

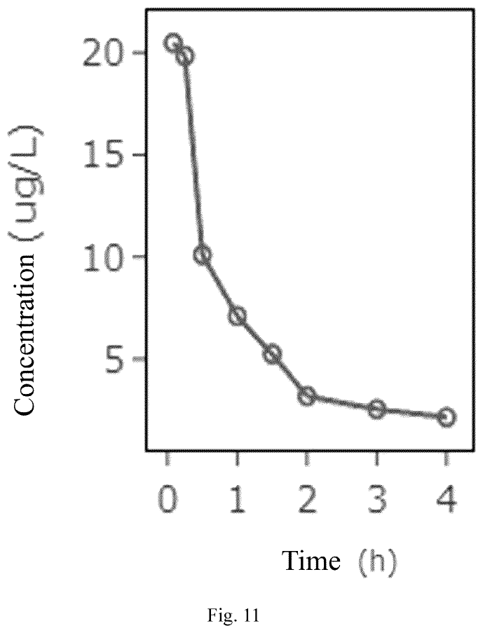

The results are shown in Table 11 and FIG. 11. The pharmacokinetic calculation results of ⁶⁸Ga-Compound 1 are as follows: elimination half-life T_{1/2} is 3.51 h, and peak time Tmax is about 0.08 h, indicating that the drug has good pharmacokinetic properties.

TABLE 11
Non-compartmental pharmacokinetic parameters calculated
based on the pharmacokinetic counting software MAS

| Parameter | Dose | Unit | Mean |
|---|---|---|---|
| λ | 30.59 ug/kg | 1/h | 0.20 |
| T_{1/2} | 30.59 ug/kg | h | 3.51 |
| Tₘₐₓ | 30.59 ug/kg | h | 0.08 |
| Cₘₐₓ | 30.59 ug/kg | ug/L | 20.47 |
| Cₗₐₛₜ | 30.59 ug/kg | ug/L | 2.14 |
| AUC₍₀₋ₜ₎ | 30.59 ug/kg | h*(ug/L) | 23.50 |
| AUC_{(0-inf)}_{_{—}}_{obs} | 30.59 ug/kg | h*(ug/L) | 34.34 |
| R_AUC₍ₑₓₜᵣₐₚ₎_{_{—}}_{obs} | 30.59 ug/kg | % | 31.55 |
| V_obs | 30.59 ug/kg | L/kg | 4.51 |
| CL_obs | 30.59 ug/kg | (L/h)/kg | 0.89 |
| AUMC₍₀₋ₜ₎_{_{—}}_{obs} | 30.59 ug/kg | (h*h)*(ug/L) | 27.28 |
| MRT₍₀₋ₜ₎ | 30.59 ug/kg | h | 1.16 |
| MRT_{(0-inf)}_{_{—}}_{obs} | 30.59 ug/kg | h | 3.65 |
| Vss_obs | 30.59 ug/kg | L/kg | 3.25 |

### Experimental Example 8 Tumor Efficacy Experiment of ¹⁷Lu-Compound 1

Eighteen experimental mice in OS-RC-2 model were randomly selected and divided into three groups, with 6 mice in each group, which were saline group, single dose group, and multiple dose groups, respectively. In the single dose group, each animal was given 2mCi of ¹⁷⁷Lu-Compound 1, and each mouse was given once, by tail vein injection; in the multiple dose group, each animal was given ¹⁷⁷Lu-Compound 1 twice, 2mCi each time, with an interval of four days between the two administrations, by tail vein injection; the body weight and tumor volume of the mice were measured before the experiment, and the body weight and tumor volume were measured every two or three days after administration. The long and short diameters of the tumor were measured to calculate the tumor volume, and the calculation formula is as follows: tumor volume (TV)=a×b²/2 (a is the long diameter, and b is the short diameter). The tumor growth inhibition rate TGI was also calculated.

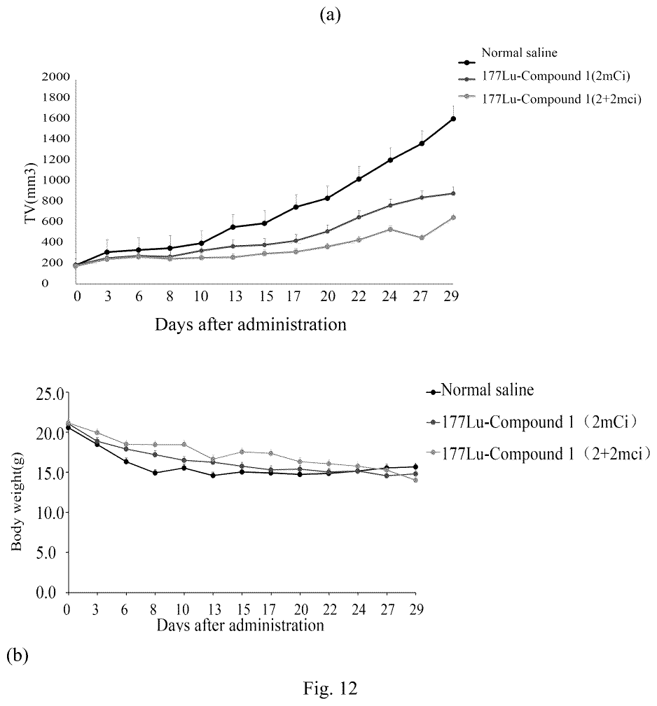

Twenty days after administration, the TGI values of the single dose group and the multiple dose group were 41.7% and 57.2%, respectively. As shown in FIG. 12, compared with the saline group, the single dose group and the multiple dose group showed obvious anti-tumor activity, and effectively inhibited the growth of tumors. Moreover, the tumor growth of the multiple dose group was slower, showing drug dose dependence. In the three groups of experiments, there was no significant difference in the weight change trend of mice. It can be seen that this drug has good anti-tumor efficacy and safety.

### Experimental Example 9 PET/CT Scanning Tissue Distribution and Targeting Verification of ⁶⁸Ga-Compound 2 in OS-RC-2 Model

### Experimental Steps:

Four experimental mice in OS-RC-2 model were selected. Each animal was given 80 μCi of ⁶⁸Ga-compound 2. After pre-anesthesia with an appropriate concentration of isoflurane/air mixed gas before scanning, the animals were placed in a MicroPET/CT imaging cabin (SNPC-303 Super Nova, PINGSENG Healthcare (KunShan) Inc.), and anesthesia was maintained with isoflurane/air mixed gas. Dynamic MicroPET/CT scanning was performed for 1 hour after drug administration and static MicroPET/CT scanning was performed 2 hours after administration. The acquired image data was reconstructed by the equipment software, and then processed and analyzed by PMOD software to generate tissue distribution maps and the radioactive uptake values of each tissue and organ were quantitatively determined (expressed as % ID/g).

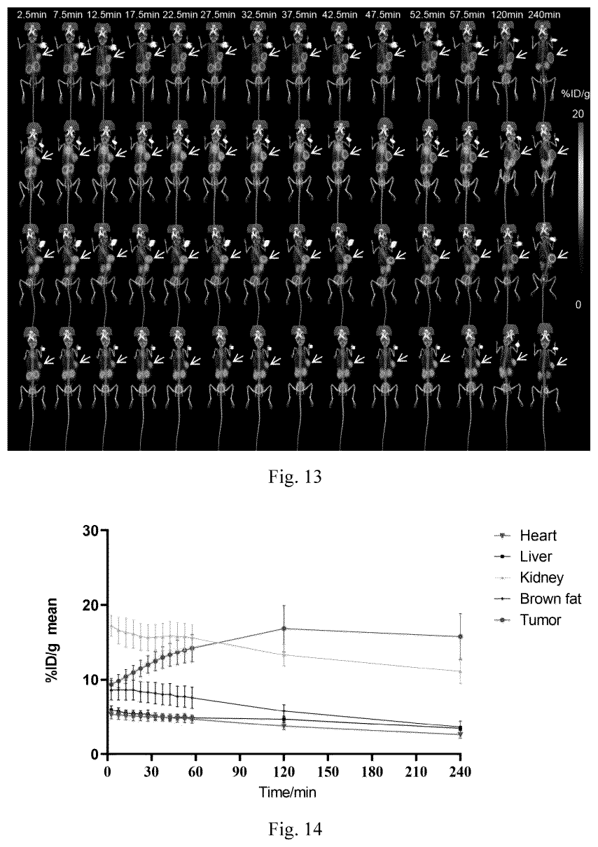

The results showed that, as shown in FIG. 13 (the arrow indicates the tumor site), ⁶⁸Ga-compound 2 was rapidly enriched in the tumor after administration, with little uptake and rapid metabolism through the kidneys in other non-target organs. Good imaging effects could be achieved in the tumor half an hour after administration. As can be seen from FIG. 14 and Table 12, one hour after administration, kidney uptake was low and tumor imaging was more obvious, which has guiding significance for clinical tumor diagnosis.

TABLE 12

| | % ID/g | % ID/g | % ID/g | % ID/g |
|---|---|---|---|---|
| | average | average | average | average |
| | value | value | value | value |
| Tissues | (27.5 min) | (57.5 min) | (120 min) | (240 min) |
| Brain | 0.95 | 0.84 | 0.77 | 0.61 |
| Heart | 4.92 | 4.67 | 3.73 | 2.60 |
| Liver | 5.33 | 4.84 | 4.66 | 3.43 |
| Lung | 6.05 | 6.22 | 5.00 | 3.18 |
| Kidney | 15.65 | 15.58 | 13.29 | 11.10 |
| Stomach | 8.25 | 9.29 | 11.24 | 9.74 |
| Fat | 8.28 | 7.54 | 5.72 | 3.62 |
| Muscle | 1.80 | 1.65 | 1.29 | 1.02 |
| Tumor | 11.99 | 14.20 | 16.81 | 15.75 |

### Experimental Example 10 In Vitro Tissue Distribution Experiment of ¹⁷⁷Lu-Compound 2 in OS-RC-2 Model

### Experimental Steps:

Six experimental mice in OS-RC-2 model were selected. Each animal was given 300 μCi of ¹⁷⁷Lu-compound 2. Three mice were dissected at 4 h and 48 h after administration, respectively. 15 tissues and organs including brain, thyroid, heart, kidney, large intestine, small intestine, liver, lung, pancreas, gonad, skeletal muscle, spleen, stomach, fat, and tumor were obtained to detect the γ-radioactivity count.

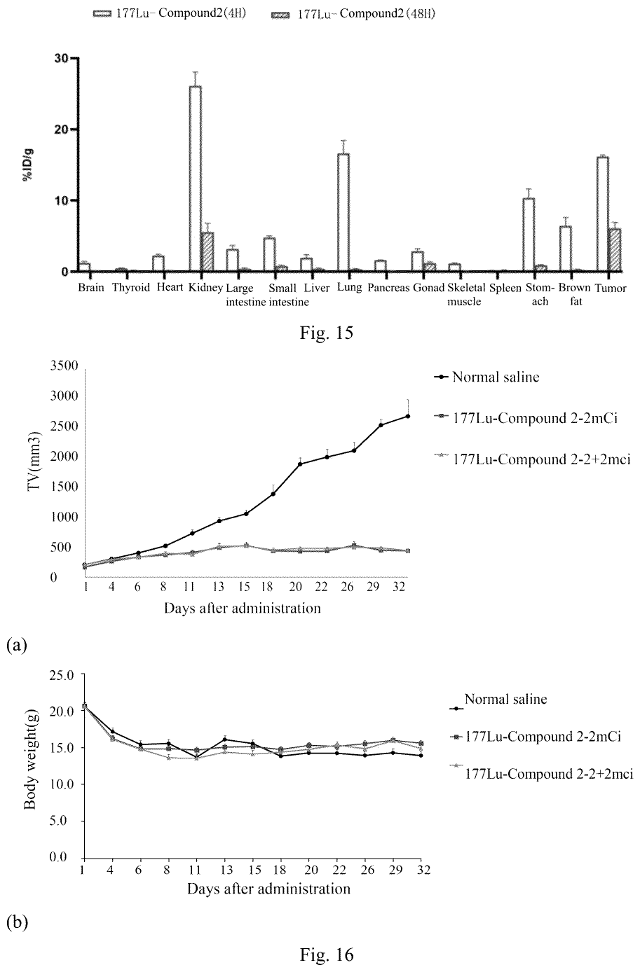

Table 13 lists the distribution of radioactivity in each tissue at each time point. As shown in FIG. 15, ¹⁷⁷Lu-compound 2 was enriched in tumor tissue 4 hours after administration. Except for the kidney, lung, stomach, and brown fat (brown fat at the scapula), the radioactivity uptake level in other normal tissues was low.

48 hours after administration, the radioactive uptake in the kidney, lung, stomach, brown fat (brown fat at the scapula) and other normal tissues decreased significantly, while t there was still high uptake in the tumor. This shows that ¹⁷⁷Lu-compound 2 can specifically target tumor tissue and maintain high radioactive retention at the tumor site for a long time, while achieving rapid clearance in non-target organs, suggesting that it has good safety and potential clinical therapeutic value.

TABLE 13

| | % ID/g | % ID/g |
|---|---|---|
| | Average | Average |
| Tissues | value (4 h) | value (48 h) |
| Thyroid | 1.24 | 0.14 |
| Brain | 0.47 | 0.22 |
| Heart | 2.29 | 0.16 |
| Kidney | 26.10 | 5.56 |
| Large intestine | 3.20 | 0.41 |
| Small Intestine | 4.79 | 0.82 |
| Liver | 1.95 | 0.41 |
| Lung | 16.61 | 0.37 |
| Pancreas | 1.63 | 0.08 |
| Gonad | 2.88 | 1.18 |
| Skeletal muscle | 1.18 | 0.09 |
| Spleen | 0.20 | 0.22 |
| Stomach | 10.38 | 0.90 |
| Brown fat | 6.43 | 0.28 |
| Tumor | 16.16 | 6.07 |

### Experimental Example 11 Efficacy Experiment of ¹⁷⁷Lu-Compound 2 in OS-RC-2 Model

### Experimental Steps:

Eighteen experimental mice in OS-RC-2 model were randomly selected and divided into three groups, with 6 mice in each group, which were saline group, single dose group, and multiple dose groups, respectively. In the single dose group, each animal was given 2mCi of ¹⁷⁷Lu-Compound 2, and each mouse was given once, by tail vein injection; in the multiple dose group, each animal was given ¹⁷⁷Lu-Compound 2 twice, 2mCi each time, with an interval of seven days between the two administrations, by tail vein injection; the body weight and tumor volume of the mice were measured before the experiment, and the body weight and tumor volume were measured every three days after administration. The long and short diameters of the tumor were measured to calculate the tumor volume, and the calculation formula is as follows: tumor volume (TV)=a×b²/2 (a is the long diameter, and b is the short diameter). The tumor growth inhibition rate TGI was also calculated.

32 days after administration, the TGI values of the single dose group and the multiple dose group were 81.0% and 85.6%, respectively. As shown in FIG. 16, compared with the saline group, the single dose group and the multiple dose group showed obvious anti-tumor activity, and effectively inhibited the growth of tumors. In the three groups of experiments, there was no significant difference in the weight change trend of mice. It can be seen that this drug has good anti-tumor efficacy and safety.

### Experimental Example 12 Safety Evaluation Experiment of ⁶⁸Ga/¹⁷⁷Lu-Compound 2

The experimental mice were female ICR mice purchased from Hengjia Biotechnology (Suzhou) Co., Ltd.

18 mice in the animal model were randomly selected and divided into 3 groups, with 6 mice in each group, which were Group A, Group B, and Group C, respectively. In Group A, each animal was given 300 μCi of ⁶⁸Ga-Compound 2; in Group B, each animal was given 1.5 mCi of ¹⁷⁷Lu-compound 2; in Group C, each animal was given an equal volume of saline; all three groups of mice were injected through the tail vein; the body weight and food intake of the animals were observed 3 times a week for 2 weeks. After the observation, the animals were dissected and the main organs were observed.

The results showed that there was no death or other obvious abnormality in the three groups of mice during the whole experiment, and there were no significant differences in body weight and food intake. No obvious abnormal changes were found in major organs during the autopsy.

### Example 13 In Vitro Tissue Distribution Experiment of 161Tb-Compound 2 in OS-RC-2 Model

### Experimental Steps:

Twelve experimental mice in OS-RC-2 model were selected. Each animal was given about 300 μCi of ¹⁶¹Tb-compound 2. Three mice were dissected at 4 h, 24 h, 48 h, and 120 h after administration, respectively. 14 tissues and organs including brain, thyroid, heart, kidney, large intestine, small intestine, liver, lung, pancreas, gonad, skeletal muscle, spleen, stomach, and fat, as well as tumor were obtained to detect the γ-radioactivity count.

Table 14 lists the distribution of radioactivity in each tissue at each time point. As shown in FIG. 22, ¹⁶¹Tb-Compound 2 showed higher radioactivity uptake in tumor tissue 24 hours after administration, while the radioactivity uptake in other normal tissues was lower. 48 hours after administration, the tumor site still maintained a high radioactivity accumulation.

TABLE 14

| | % ID/g | % ID/g | % ID/g | % ID/g |
|---|---|---|---|---|
| | average | average | average | average |
| | value | value | value | value |
| Tissues | (4 h) | (24 h) | (48 h) | (120 h) |
| Brain | 0.18 | 0.18 | 0.13 | 0.09 |
| Thyroid | 2.18 | 3.25 | 3.31 | 4.79 |
| Heart | 1.48 | 1.21 | 0.92 | 0.73 |
| Kidney | 10.71 | 6.63 | 4.72 | 3.14 |
| Large intestine | 1.07 | 5.00 | 0.70 | 0.21 |
| Small intestine | 1.32 | 0.98 | 0.55 | 0.19 |
| Liver | 3.09 | 5.47 | 2.83 | 1.85 |
| Lung | 7.73 | 5.23 | 3.91 | 3.26 |
| Pancreas | 0.52 | 0.63 | 0.38 | 0.34 |
| Gonad | 0.83 | 1.10 | 1.16 | 1.22 |
| Skeletal muscle | 0.51 | 0.37 | 0.30 | 0.25 |
| Spleen | 0.17 | 0.58 | 0.46 | 0.56 |
| Stomach | 2.57 | 1.59 | 0.76 | 0.57 |
| Brown fat | 1.85 | 1.75 | 1.89 | 1.78 |
| Tumor | 2.93 | 6.06 | 2.76 | 1.61 |

### Example 14 the Efficacy Experiment of 161Tb-Compound 2 in OS-RC-2 Model Mice

### Experimental Steps:

Sixteen experimental mice in OS-RC-2 model were randomly selected and divided into two groups, with 8 mice in each group, which were G1 and G2, respectively. In the G2 group, each animal was given 0.67 mCi of ¹⁶¹Tb-Compound 2, and each mouse was given once, by tail vein injection; in the G1 group, each animal was given glucose solution. The body weight and tumor volume of the mice were measured before the experiment, and the body weight and tumor volume were measured twice a week after administration. The long and short diameters of the tumor were measured to calculate the tumor volume, and the calculation formula is as follows: tumor volume (TV)=a×b²/2 (a is the long diameter, and b is the short diameter). The tumor growth inhibition rate TGI was also calculated.

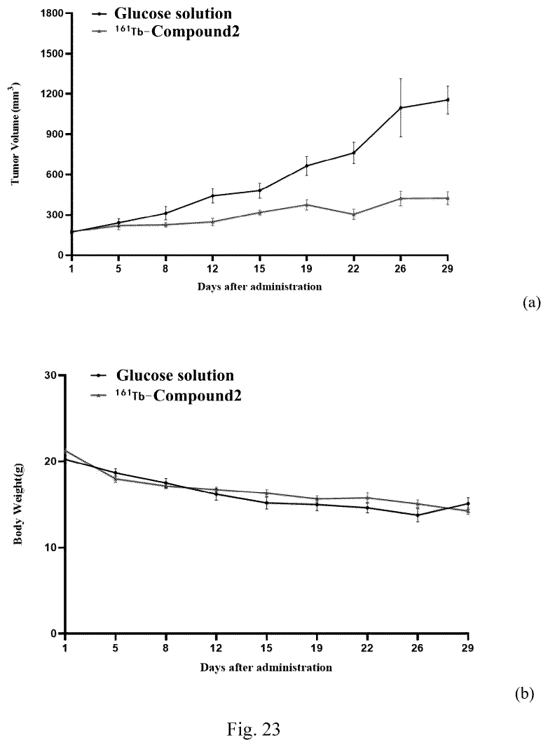

29 days after administration, the TGI value of the G2 group was 60.0%. As shown in FIG. 23, the G2 group showed significant anti-tumor activity and could effectively inhibit tumor growth, indicating that this drug has good anti-tumor efficacy and safety.

### Example 15 Tissue Distribution and Targeting Experiment of ⁶⁸Ga-Compound 3 in OS-RC-2 Model Mice

### Experimental Steps:

Four experimental mice in OS-RC-2 model were selected. Each animal was given 80 μCi of ⁶⁸Ga-Compound 3, and the pre-anesthetized animals were placed in a MicroPET/CT imaging cabin (SNPC-303 Super Nova, PINGSENG Healthcare (KunShan) Inc.). Dynamic MicroPET/CT scanning was performed after drug administration for 1 h, and static MicroPET/CT scanning was performed 2 hours after drug administration. The acquired image data was reconstructed by the equipment software, and then processed and analyzed by PMOD software to generate tissue distribution maps and the radioactive uptake values of each tissue and organ were quantitatively determined (expressed as % ID/g).

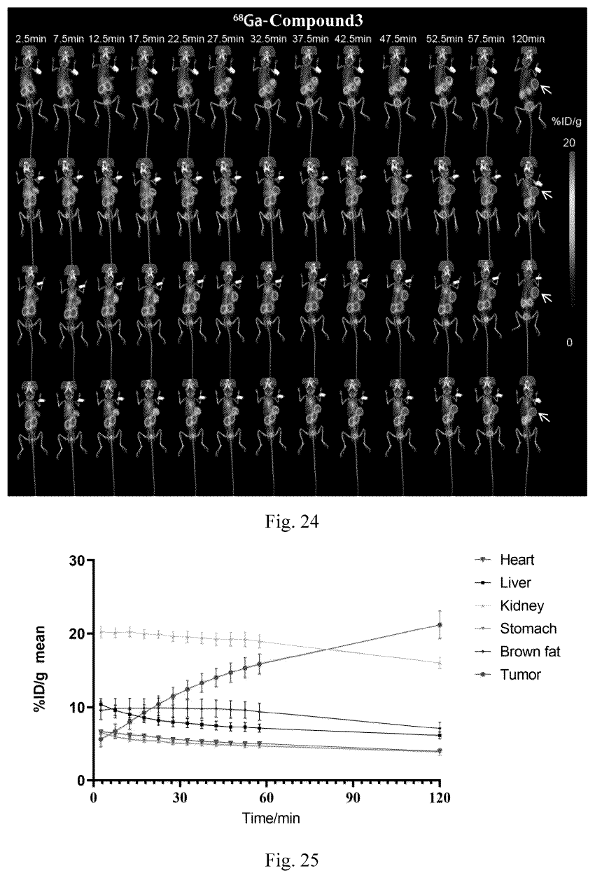

The results are shown in FIG. 24 (the arrow indicates the tumor site) and Table 15. ⁶⁸Ga-compound 3 can be rapidly accumulated in the tumor site after administration, while the uptake in non-target organs is low, and eliminated mainly by metabolism through the renal pathway. Good imaging effects can be obtained at the tumor site half an hour after administration. It can be seen from FIGS. 24 and 25 that 2 hours after administration, the radioactive uptake in the kidney is further reduced and the contrast of tumor imaging is significantly enhanced.

TABLE 15

| | % ID/g | % ID/g | % ID/g |
|---|---|---|---|
| | average | average | average |
| | value | value | value |
| Tissues | (27.5 min) | (57.5 min) | (120 min) |
| Brain | 1.00 | 0.94 | 0.81 |
| Heart | 5.61 | 5.03 | 3.98 |
| Liver | 7.98 | 7.14 | 6.14 |
| Lung | 6.84 | 7.08 | 6.38 |
| Kidney | 19.62 | 18.98 | 16.05 |
| Stomach | 5.09 | 4.71 | 3.91 |
| Intestinal | 4.97 | 4.80 | 4.43 |
| Fat | 9.88 | 9.40 | 7.11 |
| Muscle | 1.83 | 1.65 | 1.43 |
| Tumor | 11.49 | 15.86 | 21.23 |
| Tumor/Muscle | 6.28 | 9.61 | 14.85 |

### Example 16 Tissue Distribution and Targeting Experiments of ¹⁸F-Compound 4 in OS-RC-2 Model Mice

### Experimental Steps:

Four experimental mice in OS-RC-2 model were selected. Each animal was given 80 μCi of ¹⁸F-Compound 4, and the pre-anesthetized animals were placed in a MicroPET/CT imaging cabin (SNPC-303 Super Nova, PINGSENG Healthcare (KunShan) Inc.). Dynamic MicroPET/CT scanning was performed after drug administration for 1 hour, and static MicroPET/CT scanning was performed 2 hours after drug administration. A scanned image was obtained after reconstruction by the equipment software, and the scanned image was analyzed using the software.

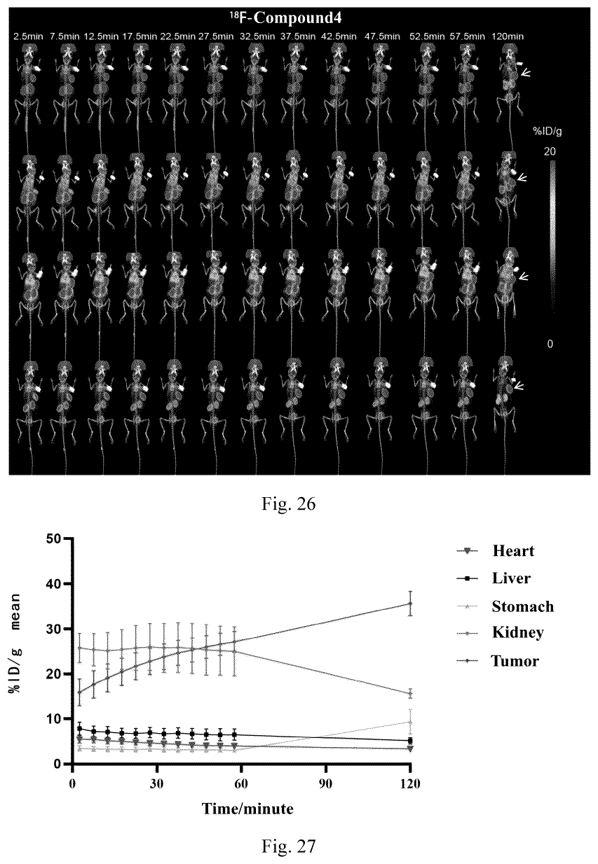

The results showed that, as shown in FIG. 26 (the arrow indicates the tumor site), ¹⁸F-compound 4 was rapidly enriched in the tumor site after administration, with less uptake in other non-target organs. Good imaging effects could be obtained in the tumor site half an hour after administration. It can be seen from FIGS. 26 and 27 that 2 hours after administration, the radioactive uptake in the kidney was further reduced, and the contrast of tumor imaging was significantly enhanced.

TABLE 16

| | % ID/g | % ID/g | % ID/g |
|---|---|---|---|
| | average | average | average |
| | value | value | value |
| Tissues | (27.5 min) | (57.5 min) | (120 min) |
| Brain | 0.85 | 0.81 | 0.63 |
| Heart | 4.58 | 3.95 | 3.30 |
| Liver | 6.94 | 6.48 | 5.10 |
| Lung | 11.33 | 11.62 | 8.53 |
| Kidney | 26.01 | 24.98 | 15.61 |
| Stomach | 3.30 | 2.96 | 9.37 |
| Intestinal | 5.98 | 6.27 | 6.33 |
| Fat | 17.46 | 16.57 | 12.56 |
| Muscle | 2.27 | 2.50 | 2.02 |
| Tumor | 22.75 | 27.08 | 35.59 |
| Tumor/Muscle | 10.0 | 10.9 | 17.6 |

### Example 17 Tissue Distribution and Targeting Experiments of ⁶⁸Ga-Compound 5, ⁶⁸Ga-Compound 6 in OS-RC-2 Model Mice

### Experimental Steps:

Three experimental mice in OS-RC-2 model were selected. Two of them were given 80 μCi of ⁶⁸Ga-Compound 5, and the other one was given 80 μCi of ⁶⁸Ga-Compound 6. Dynamic MicroPET/CT scanning was performed after drug administration for 1 hour, and static MicroPET/CT scanning was performed 2 hours and 4 hours (Compound 5 drug group) after drug administration. The tissue distribution map was obtained and the radioactive uptake values of each tissue and organ were quantitatively determined (expressed as % ID/g).

The results showed that, as shown in FIG. 33 (the arrow indicates the tumor site), ⁶⁸Ga-compound 5 and ⁶⁸Ga-compound 6 were rapidly enriched in the tumor site after administration, with little uptake and rapid metabolism through the kidneys in other non-target organs, achieving good imaging results at the tumor site within half an hour after administration. As can be seen from FIG. 33, two hours after administration, kidney uptake was low and tumor imaging was more obvious, which has guiding significance for clinical tumor diagnosis.

TABLE 17

| | % ID/g | % ID/g | % ID/g | % ID/g |
|---|---|---|---|---|
| | average | average | average | average |
| Tissues | value | value | value | value |
| ⁶⁸Ga-compound 5 | (27.5 min) | (57.5 min) | (120 min) | (240 min) |
| Brain | 0.92 | 0.84 | 0.48 | 0.36 |
| Heart | 4.36 | 3.65 | 1.92 | 1.40 |
| Liver | 4.80 | 4.32 | 2.65 | 1.95 |
| Lung | 3.99 | 3.56 | 1.71 | 1.36 |
| Kidney | 10.01 | 10.20 | 9.17 | 8.11 |
| Fat | 5.38 | 4.96 | 2.25 | 1.42 |
| Muscle | 1.83 | 1.52 | 0.83 | 0.61 |
| Tumor | 10.62 | 10.14 | 7.31 | 5.62 |
| Stomach | 0.70 | 0.62 | 0.45 | 0.31 |

TABLE 18

| Tissues | % ID/g | % ID/g | % ID/g |
|---|---|---|---|
| ⁶⁸Ga-compound 6 | (27.5 min) | (57.5 min) | (120 min) |
| Brain | 0.44 | 0.20 | 0.11 |
| Heart | 2.00 | 0.91 | 0.43 |
| Liver | 2.62 | 1.73 | 1.30 |
| Lung | 1.72 | 0.91 | 0.45 |
| Kidney | 7.03 | 4.17 | 2.60 |
| Fat | 3.16 | 1.59 | 0.36 |
| Muscle | 0.29 | 0.20 | 0.12 |
| Tumor | 4.29 | 3.09 | 1.69 |
| Stomach | 0.71 | 0.45 | 0.46 |

### Example 18 Clinical Trial of ⁶⁸Ga-Compound 2

### Clinical Trials:

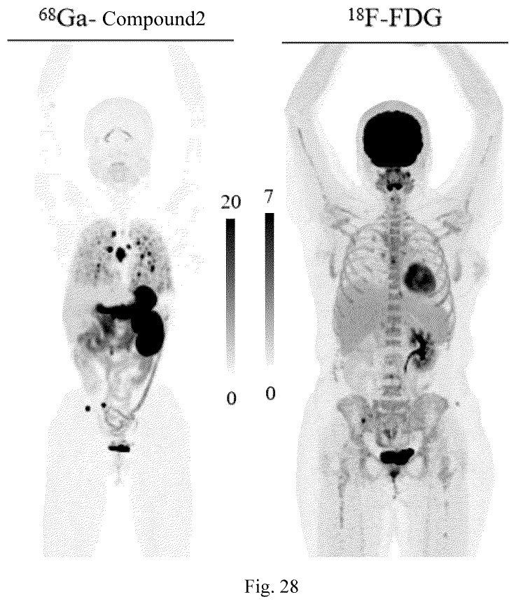

The patient was a 37-year-old female, weighing 80 kg. She had undergone radical resection for right renal mass in the past, and postoperative pathology showed clear cell carcinoma with sarcomatoid differentiation. Multiple lung metastases were found after surgery (1.3 cm in the right upper lobe and 1.4×0.7 cm in the left lower lobe). She received pepapanib combined with immunotherapy, and the efficacy was evaluated as partial remission (PR). A later bone scan showed lumbar bone metastasis. Denosumab anti-bone treatment was performed, and immunotherapy was discontinued. In order to clarify the distribution of systemic lesions, the patient underwent ¹⁸F-FDG and ⁶⁸Ga-Compound 2 PET/CT imaging. First, 1.96 mCi of ⁶⁸Ga-compound 2 was injected intravenously and scanned at 56 minutes after injection. One day later, 5.99 mCi of ¹⁸F-FDG was injected intravenously, and scanned at 156 minutes after injection. The original image was obtained by scanning, and further reconstructed to obtain the maximum intensity projection (MIP) image, as shown in FIG. 28, which was used to evaluate the distribution of radioactive tracers in the whole body and the metabolic characteristics of the lesion site. The radioactive uptake values of the lesions and tissues are shown in Table 19. It can be seen that the CA9 imaging results are as follows: high radioactive uptake was observed in multiple parts of the vertebral lesions, sacroiliac joints, iliac bones and digestive system, indicating that there was obvious CA9 expression activity in the tumor site. Compared with the ¹⁸F-FDG imaging results, the ¹⁸F-FDG uptake in the same site was less. Compared with ¹⁸F-FDG imaging, ⁶⁸Ga-compound 2 imaging showed higher targeted uptake and contrast at the site of tumor metastasis, indicating that CA9 PET/CT has high sensitivity and specificity, and can be used for the detection and evaluation of active lesions of clear cell carcinoma.

TABLE 19

Tracer

| | |
|---|---|
| ⁶⁸Ga-compound | ¹⁸F-FDG |

Time

| | |
|---|---|
| 1 h | 3 h |

Ingestion value

| | SUV mean | SUV mean |
|---|---|---|
| Lymph node 1 | 25.49 | 3.11 |
| Lymph node 2 | 36.43 | 3.22 |
| Lymph node 3 | 30.79 | 4.5 |
| Pulmonary nodules 1 | 9.3 | 1.36 |
| Pulmonary nodules 2 | 8.62 | 1.59 |
| Pulmonary nodules 3 | 8.89 | 2.1 |
| Pulmonary nodules 4 | 13 | 2.87 |
| Pulmonary nodules 5 | 14.96 | 1.71 |
| Pancreatic head lesions | 23.57 | 2.95 |
| Sacral lesions | 31.53 | 2.78 |
| Iliac bone lesions | 35.76 | 3.57 |
| Vertebral lesions | 9.12 | 2.57 |
| Intrahepatic lesions | 12.66 | 3.04 |

Although examples of the present disclosure have been shown and described, it will be appreciated by those skilled in the art that various changes, modifications, substitutions and variations can be made to the examples without departing from the principles and spirit of the present disclosure, and that the scope of the present disclosure is defined by the appended claims and their equivalents.

What is claimed is:

1. A compound of formula (I), or an isotopic variant, a hydrate, an ester or solvate, a tautomer or a stereoisomer thereof, or a pharmaceutically acceptable salt thereof: 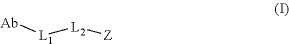
wherein,
Ab is a ligand targeting carbonic anhydrase IX, for example 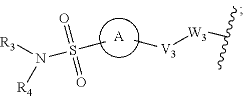
Ring A is selected from C₆₋₁₀ arylene and C₅₋₁₀ heteroarylene; wherein the C₆₋₁₀ arylene and C₅₋₁₀ heteroarylene are optionally substituted by 1, 2, 3 or 4 R_{A};
R_{A} is independently selected from H, D, halogen, CN, —OR, —SRₐ, —NR_{b}R_{c}, C₁₋₆ alkyl, C₁₋₆ haloalkyl, C₃₋₁₀ cycloalkyl, 3-10 membered heterocyclyl, C₆₋₁₀ aryl and 5-10 membered heteroaryl;
R₃ and R₄ are independently selected from H, halogen, C₁₋₆ alkyl, C₁₋₆ haloalkyl, C₃₋₇ cycloalkyl and 3-7 membered heterocyclyl;
or R₄ and R_{A} are connected to form a C₁₋₄ alkylene, a C₂₋₄ alkenylene and a C₂₋₄ alkynylene, wherein one or more methylene units are optionally and independently replaced by —CR*₂—, —NR*—, —NR*C(O)—, —C(O)NR*—, —NR*S(O)₂—, —S(O)₂NR*—, —O—, —C(O)—, —OC(O)—, —C(O)O—, —S—, —S(O)— or —S(O)₂—; and the ring formed by conneting R₄ with R_{A} is a ring structure fused with ring A;
V₃ is selected from a chemical bond, a C₁₋₁₀ alkylene group, a C₂₋₁₀ alkenylene group, a C₂₋₁₀ alkynylene group, and 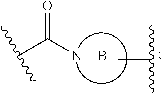
Ring B is a 3-7 membered heterocyclylene group;
W₃ is selected from a chemical bond, —NR—, —O—, —S— and —C(O)—;
R* is independently selected from H, D, halogen, C₁₋₆ alkyl and C₁₋₆ haloalkyl;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-10 membered heterocyclyl group;
L₁ is a linker;
L₂ is an amino acid chain formed by connecting 2-8 amino acid residues, wherein each amino acid residue can be optionally further substituted by one or more amino acid residues, and the amino acid residues are optionally substituted by 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent.

2. The compound of claim 1, having the following structure: 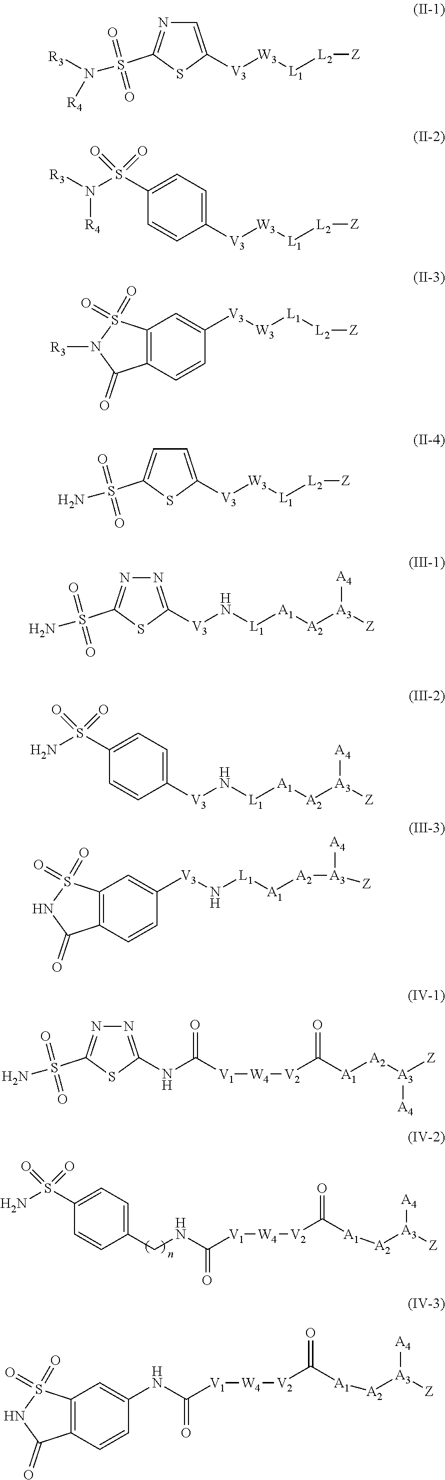
wherein,
A₁, A₂, and A₃ are amino acid residues;
A₄ is H or an amino acid residue;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted with 1, 2, 3, 4, 5 or 6 R₁ₛ;
R₁ₛ is independently selected from H, D, —OR, —SRₐ, —NR_{b}R_{c}, halogen, C₁₋₆ alkyl, C₁₋₆ haloalkyl, C₃₋₇ cycloalkyl and 3-7 membered heterocyclyl;
V₂ is a chemical bond or —(CR′R″)₁₋₆—;
R′ and R″ are independently selected from H, D, halogen, C₁₋₆ alkyl, C₁₋₆ haloalkyl and —C₁₋₆ alkylene-Ar;
Ar is selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
n is 0, 1, 2, 3, 4, 5 or 6;
the remaining variables are as defined in claim 1.

3. The compound of claim 1, wherein the compound has one or more of the following definitions:
i) wherein
L₁ is —W₁—V₁—W₄—V₂—W₂—;
W₁ is selected from a chemical bond, —NR—, —C(O)—, —C(O)O—, and —C(O)NR—;
W₂ is selected from —C(O)—, —OC(O)—, —NRC(O)—, —NR—, —C(O)NR—, and —O—;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted by 1, 2, 3, 4, 5 or 6 R₁ₛ; wherein one or more methylene units in the alkylene, alkenylene and alkynylene are optionally and independently replaced by —NR—, —NRC(O)—, —C(O)NR—, —NRS(O)₂—, —S(O)₂NR—, —O—, —C(O)—, —OC(O)—, —C(O)O—, —S—, —S(O)— or —S(O)₂—;
R is independently selected from H, C₁₋₆ alkyl or C₁₋₆ haloalkyl;
R₁ₛ is independently selected from H, D, —OR, —SRₐ, —NR_{b}R_{c}, halogen, C₁₋₆ alkyl, C₁₋₆ haloalkyl, C₃₋₇ cycloalkyl or 3-7 membered heterocyclyl; alternatively, R₁ₛ is independently in (S) or (R) configuration, or is in racemic form;
V₂ is a chemical bond or —(CR′R″)₁₋₆—;
R′ and R″ are independently selected from H, D, halogen, C₁₋₆ alkyl, C₁₋₆ haloalkyl and —C₁₋₆ alkylene-Ar;
alternatively, R′ is independently in (S) or (R) configuration, or is in racemic form;
Ar is independently selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
alternatively,
L₁ is —W₁—V₁—W₄—V₂—W₂—;
W₁ is selected from —C(O)—, —C(O)O— and —C(O)NR—; alternatively —C(O)—;
W₂ is selected from —C(O)—, —OC(O)— and —NRC(O)—; alternatively —C(O)—;
V₁ is selected from C₁₋₈ alkylene, C₂₋₈ alkenylene, C₂₋₈ alkynylene, phenylene and C₅₋₆ heteroarylene, alternatively C₁₋₈ alkylene (e.g. —(CH₂)ₘ—); which is optionally substituted by 1, 2, 3, 4, 5 or 6 R₁ₛ; wherein 1, 2 or 3 methylene units in the alkylene, alkenylene and alkynylene are optionally and independently replaced by —NR—, —NRC(O)—, —C(O)NR—, —C(O)—, —OC(O)— or —C(O)O—;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —ORₐ, —NR_{b}R_{c}, halogen, C₁₋₆ alkyl and C₁₋₆ haloalkyl; alternatively H, D, —ORₐ and —NR_{b}R_{c};
V₂ is a chemical bond or —(CR′R″)₁₋₄—;
R′ is independently selected from H, D or —C₁₋₄ alkylene-Ar; alternatively, R′ is independently in (S) or (R) configuration, or in racemic form; alternatively in (S) configuration; alternatively in (R) configuration;
R″ is independently selected from H, D, halogen, C₁₋₆ alkyl and C₁₋₆ haloalkyl;
Ar is independently selected from C₆₋₁₄ aryl and 5-14 membered heteroaryl, alternatively C₆₋₁₄ aryl;
W₄ is selected from a chemical bond, —OC(O)—, —NRC(O)—, —C(O)O— and —C(O)NR—;
alternatively,
L₁ is —C(O)—V₁—W₄—V₂—C(O)—;
wherein V₁ is selected from C₁₋₆ alkylene (e.g. —(CH₂)ₘ—), phenylene and C₅₋₆ heteroarylene; alternatively selected from C₁₋₆ alkylene (e.g. —(CH₂)ₘ—), phenylene, pyrrolylene, furylene or thienylene; alternatively C₁₋₆ alkylene (e.g. —(CH₂)ₘ—); alternatively C₁₋₄ alkylene (e.g. —(CH₂)ₘ—); which is optionally substituted by 1, 2, 3, 4, 5 or 6 R₁ₛ; wherein 1, 2 or 3 methylene units in the alkylene are optionally and independently replaced by —NR—, —NRC(O)—, —C(O)NR—, —C(O)—, —OC(O)— or —C(O)O—;
m is independently selected from 1, 2, 3, 4, 5 or 6, alternatively 1, 2, 3 or 4;
R is independently selected from H, C₁₋₆ alkyl and C₁₋₆ haloalkyl;
R₁ₛ is independently selected from H, D, —ORₐ, C₁₋₆ alkyl and C₁₋₆ haloalkyl; alternatively selected from H and —ORₐ;
V₂ is a chemical bond or —(CR′R″)₁₋₂— (alternatively —CHR′—);
R′ is independently selected from H or —C₁₋₄ alkylene-Ar, alternatively H or —CH₂—Ar; alternatively, R′ is independently in (S) or (R) configuration, or in racemic form; alternatively in (S) configuration; alternatively in (R) configuration;
R″ is independently selected from H, D, C₁₋₆ alkyl and C₁₋₆ haloalkyl; alternatively H or D;
Ar is independently C₆₋₁₄ aryl, such as phenyl, naphthyl, anthracenyl or phenanthryl; alternatively C₆₋₁₀ aryl, such as phenyl or naphthyl, alternatively naphthyl, alternatively 2-naphthyl;
W₄ is selected from a chemical bond, —OC(O)—, —NRC(O)—, —C(O)O— and —C(O)NR—; alternatively a chemical bond, —C(O)O— and —C(O)NR—; alternatively a chemical bond or —C(O)NR—;
for example,
L₁ is selected from: 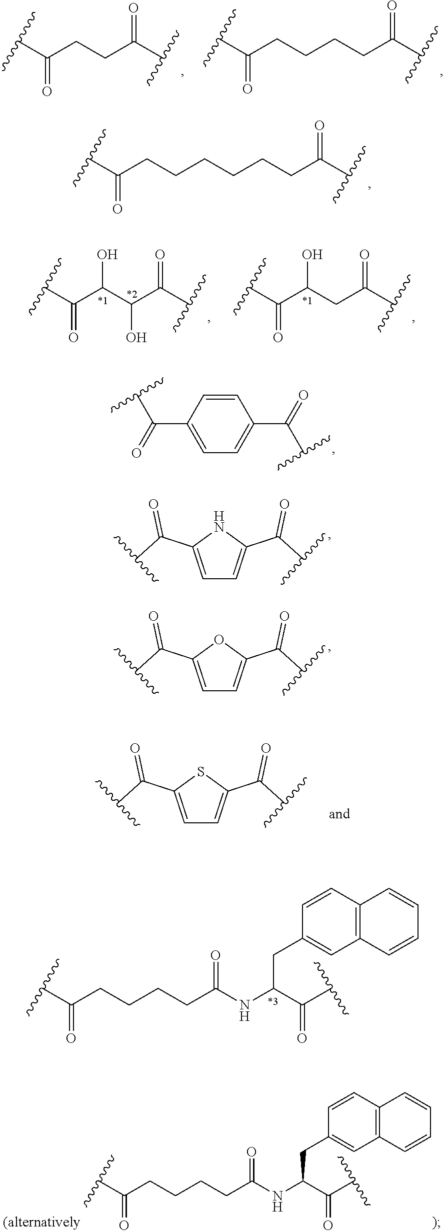
wherein, *1, *2 and *3 are chiral centers, independently selected from in (S) or (R) configuration, or in racemic form; alternatively in (S) configuration; alternatively in (R) configuration;
ii) wherein
L₂ is an amino acid chain formed by connecting 2-5 amino acid residues, wherein each amino acid residue can be optionally further substituted with one or more (alternatively 1, 2 or 3, alternatively 1) amino acid residues, and the amino acid residues can be optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
alternatively,
L₂ is 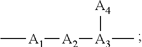
A₁, A₂, A₃ and A₄ are amino acid residues, and the amino acid residues are optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
alternatively,
L₂ is 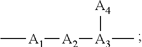
A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
still alternatively,
L₂ is 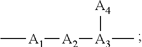
A₁ is a lysine residue; 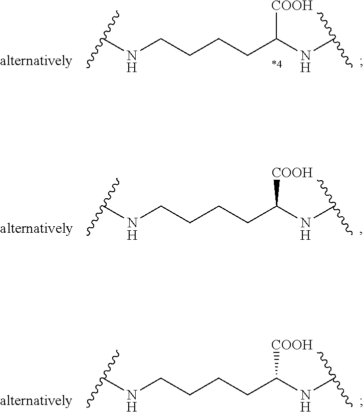 [SMILES: *NCCCCC(N*)C(=O)O ; *NCCCC[C@H](N*)C(=O)O ; *NCCCC[C@@H](N*)C(=O)O]
A₂ is selected from a tyrosine residue, an aspartic acid residue, a serine residue, a glutamic acid residue, a phenylalanine residue or an arginine residue, which is optionally substituted by 1, 2, 3 or 4 R_{L};
R_{L} is independently selected from halogen, —OR, —NR_{b}R_{c}, C₁₋₆ alkyl, C₁₋₆ haloalkyl and urea group;
alternatively halogen, C₁₋₆ alkyl, C₁₋₆ haloalkyl and urea group;
alternatively A₂ is selected from 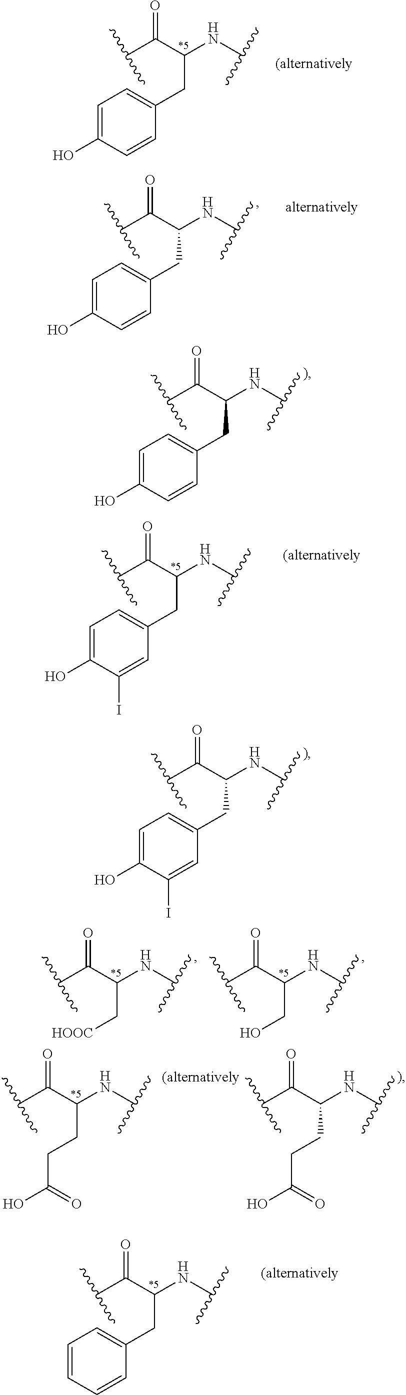 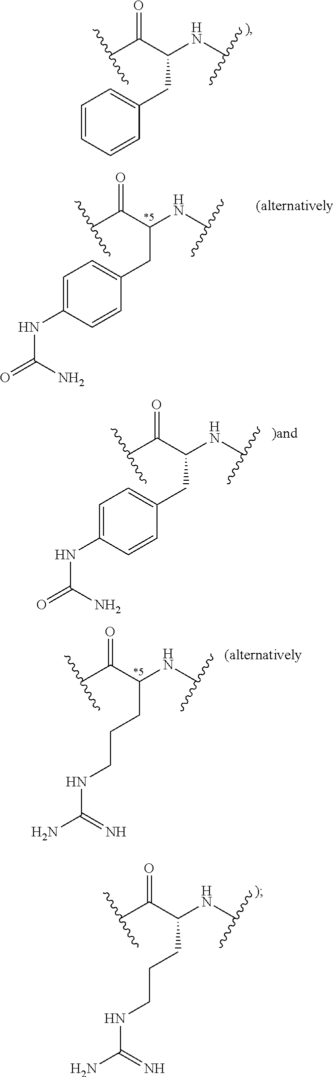
A₃ is a lysine residue; alternatively 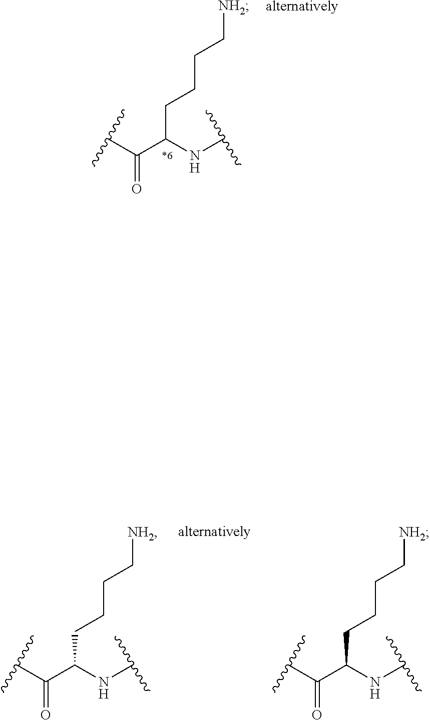
A₄ is selected from the group consisting of N-acetylglutamate residue, N-(4-carboxybutyryl)glutamate, glutamic acid residue, pyroglutamic acid residue, citrulline residue and aspartic acid residue; alternatively 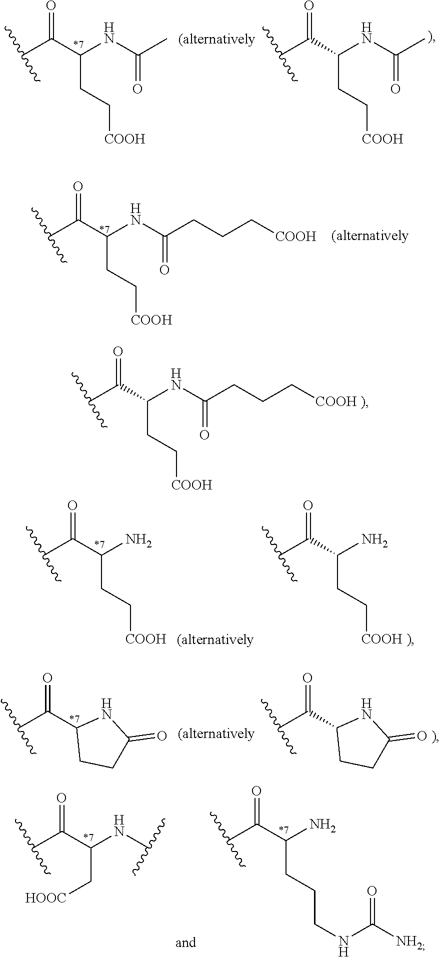
wherein, *4, *5, *6 and *7 are chiral centers, independently selected from in (S) or (R) configuration, or in racemic form; alternatively in (S) configuration; alternatively in (R) configuration;
for example,
L₂ is selected from: 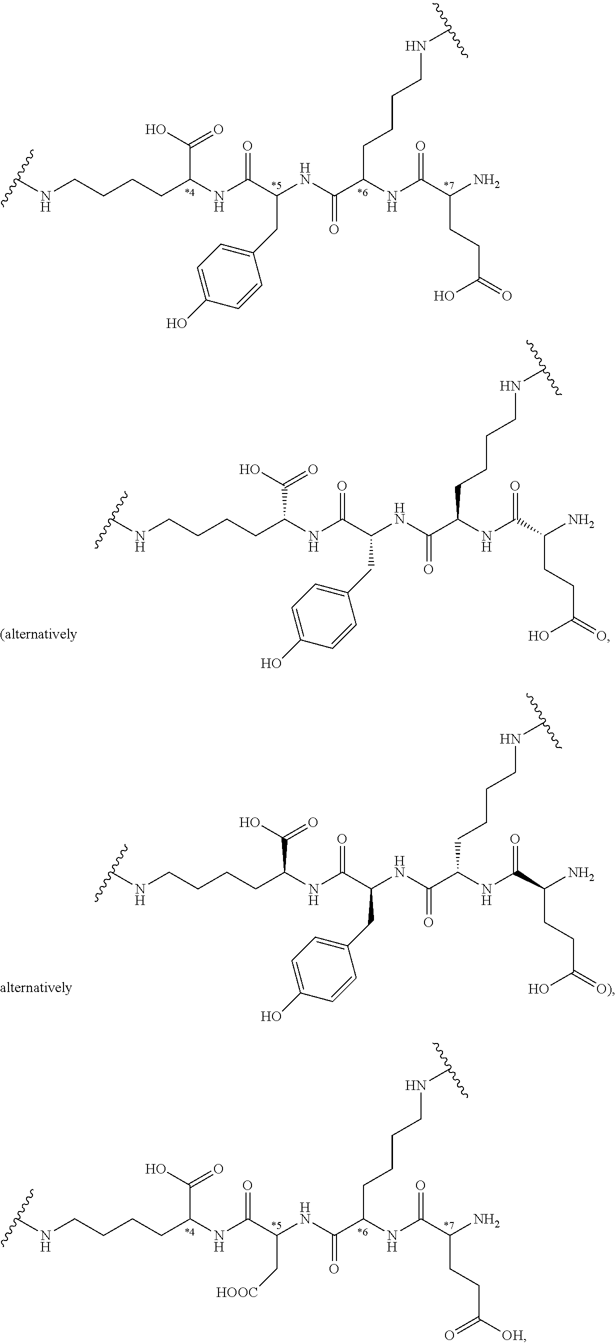 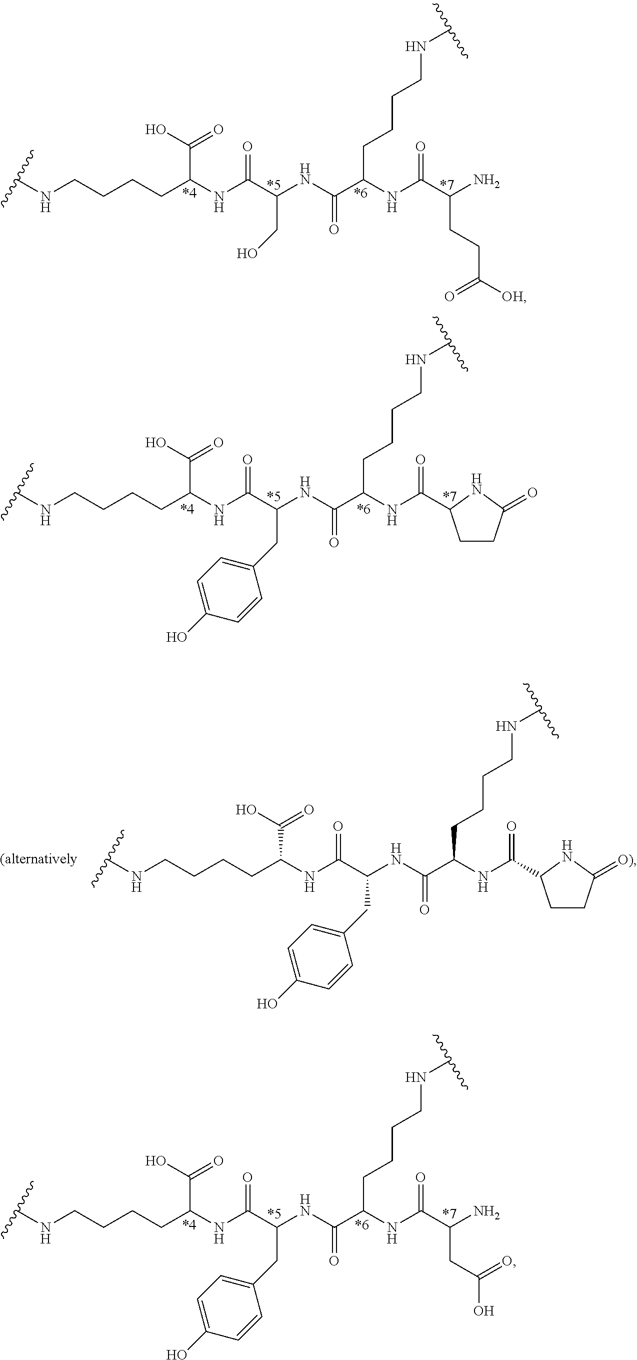 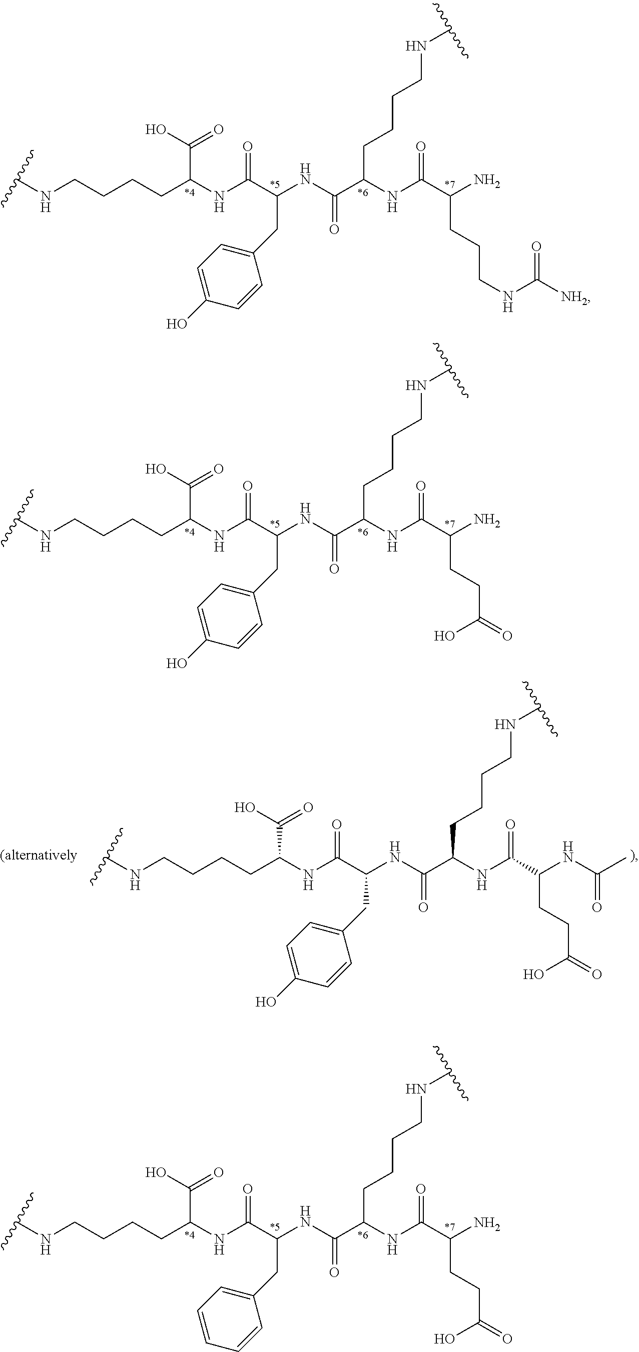 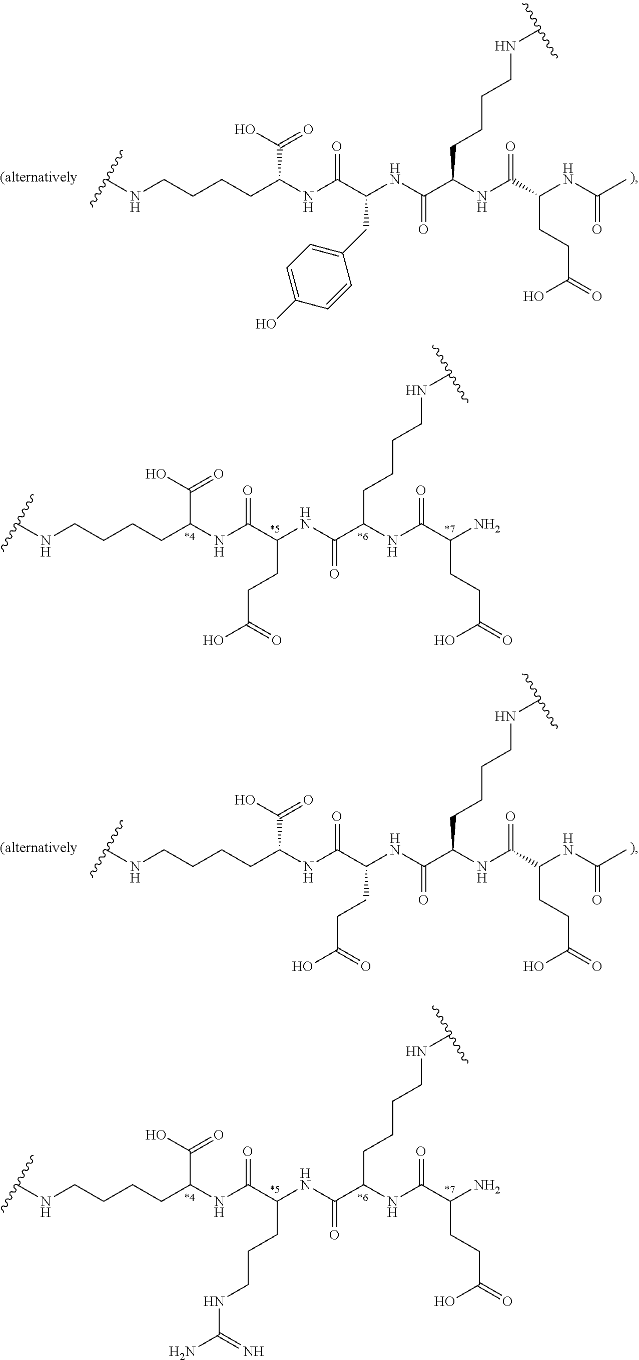 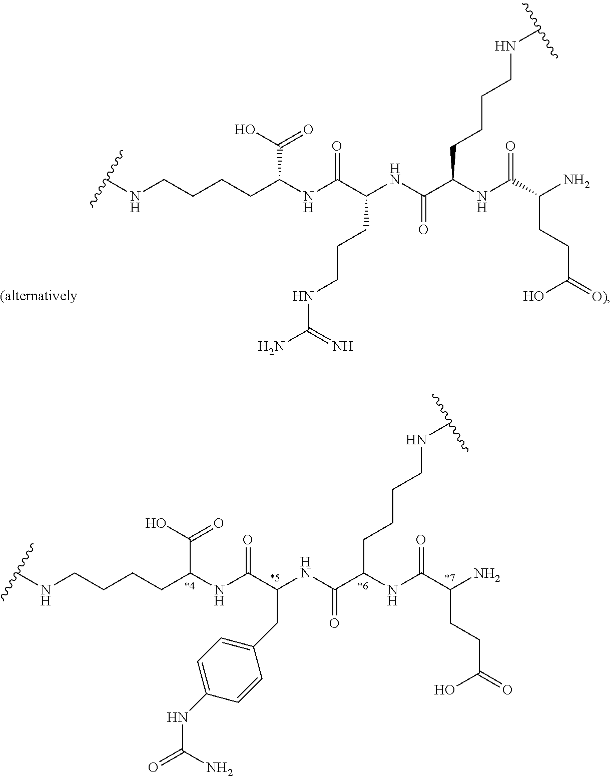 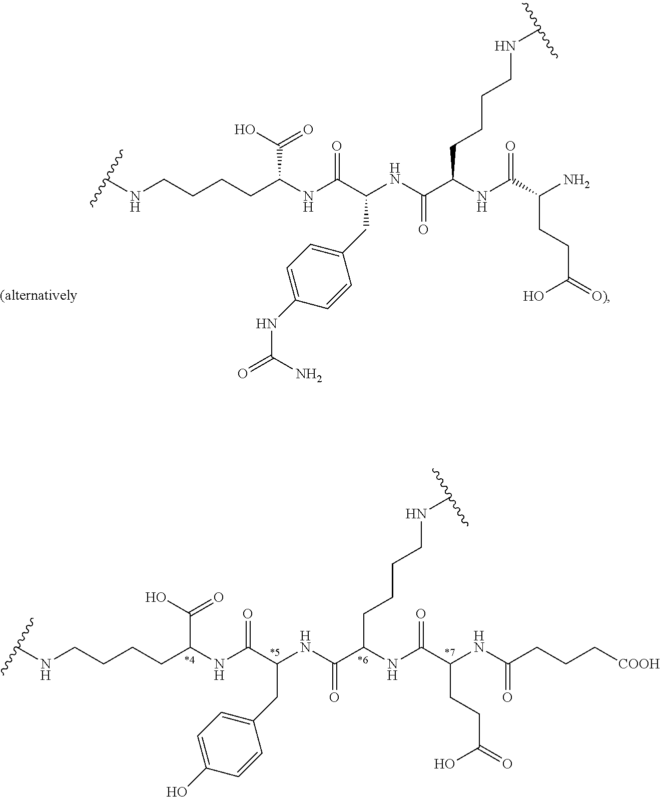 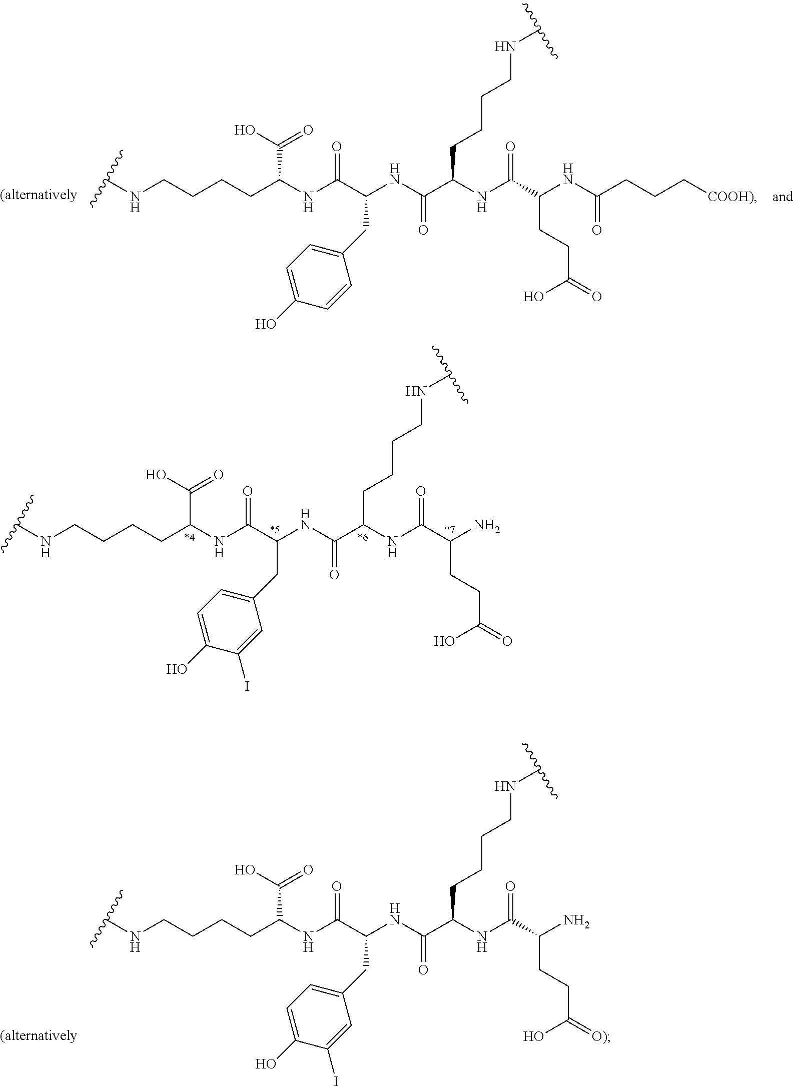
iii) wherein
Z is a chelating group derived from a chelating agent, wherein the chelating agent selected from:
1,4,7,10-tetraazacyclododecane-N,N′,N″,N′″-tetraacetic acid (DOTA),
N,N″-bis[2-hydroxy-5-(carboxyethyl)benzyl]ethylenediamine-N,N″-diacetic acid (HBED-CC),
1,4,7-triazacyclononane-1,4,7-triacetic acid (NOTA),
2-(4,7-bis(carboxymethyl)-1,4,7-triazononan-1-yl)glutaric acid (NODAGA),
2-(4,7,10-tris(carboxymethyl)-1,4,7,10-tetraazacyclododecane-1-yl)glutaric acid (DOTAGA),
1,4,7-triazacyclononanephosphinic acid (TRAP),
1,4,7-triazacyclononane-1-[methyl(2-carboxyethyl)phosphinic acid]-4,7-bis[methyl(2-hydroxymethyl)phosphinic acid](NOPO),
3,6,9,15-tetraazabicyclo[9.3.1]pentadeca-1(15),11,13-triene-3,6,9-triacetic acid (PCTA),
N′-{5-[acetyl(hydroxy)amino]pentyl}-N-[5-({4-[(5-aminopentyl)(hydroxy)amino]-4-oxobutanoyl}amino)pentyl]-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (oxy-Do3A),
p-isocyanatobenzyl-DTPA (SCN-Bz-DTPA),
1-(p-isocyanatobenzyl)-3-methyl-DTPA (1B3M),
2-(p-isocyanatobenzyl)-4-methyl-DTPA (1M3B),
1-(2)-methyl-4-isocyanatobenzyl-DTPA (MX-DTPA),
(R)-2-amino-3-(4-isothiocyanatophenyl)propyl]-trans-(S,S)-cyclohexane-1,2-diaminepentaacetic acid (p-SCN-Bn-CHX-A″-DTPA),
6-hydrazinopyridine-3-carboxylic acid (HYNIC),
2-(4-isothiocyanatophenyl)-1,4,7-triazacyclononane-1,4,7-triacetic acid (p-SCN-Bn-NOTA) or
2-[(4-isothiocyanatophenyl)methyl]-1,4,7,10-tetraazacyclododecane-1,4,7,10-tetraacetic acid (p-SCN-Bn-DOTA); 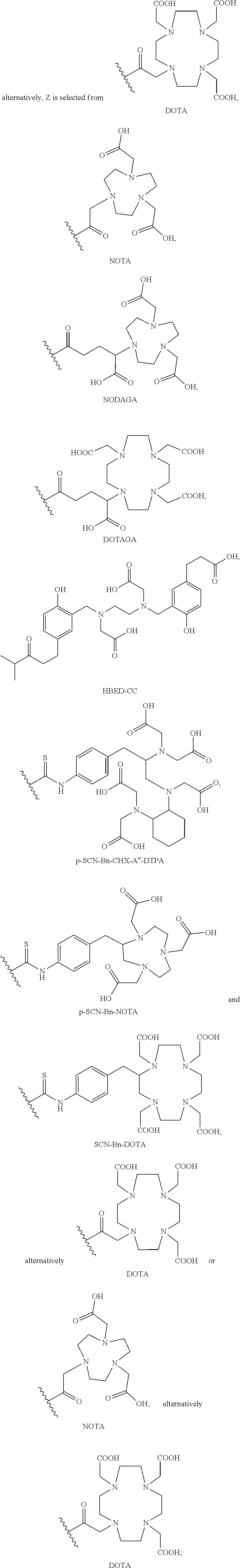
and
iv) wherein
Ab is 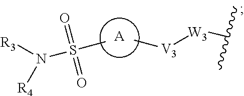
Ring A is selected from phenylene and C₅₋₆ heteroarylene; alternatively selected from phenylene and 1,3,4-thiadiazolylene; which is optionally substituted by 1, 2, 3 or 4 R_{A};
R_{A} is independently selected from H, D, halogen, CN, —OR, —SRₐ, —NR_{b}R_{c}, C₁₋₆ alkyl, C₁₋₆ haloalkyl, C₃₋₇ cycloalkyl and 3-7 membered heterocyclyl; alternatively selected from H, D, halogen, C₁₋₆ alkyl and C₁₋₆ haloalkyl;
R₃ and R₄ are independently selected from H, halogen, C₁₋₆ alkyl and C₁₋₆ haloalkyl;
or R₄ and R_{A} are connected to form a C₁₋₄ alkylene group, alternatively a C₁₋₂ alkylene group, wherein 1, 2 or 3 (alternatively 1) methylene units are optionally and independently replaced by —CR*₂—, —NR*—, —O—, —C(O)—, —OC(O)—, —C(O)O—, —S—, —S(O)— or —S(O)₂—, alternatively replaced by —CR*₂—, —C(O)—, —OC(O)— or —C(O)O—, alternatively replaced by —C(O)—; and the ring formed by connecting R₄ with R_{A} is a ring structure fused with ring A;
V₃ is selected from a chemical bond, a C₁₋₆ alkylene group, a C₂₋₆ alkenylene group, and a C₂₋₆ alkynylene group;
W₃ is selected from —NR— and —O—;
R* is independently selected from H, D, halogen, C₁₋₆ alkyl and C₁₋₆ haloalkyl;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
alternatively,
Ab is selected from: 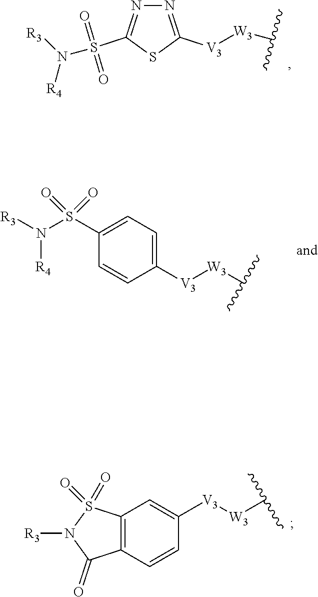
R₃ and R₄ are independently selected from H, halogen, C₁₋₆ alkyl and C₁₋₆ haloalkyl;
V₃ is selected from a chemical bond, a C₁₋₆ alkylene group, a C₂₋₆ alkenylene group, and a C₂₋₆ alkynylene group;
W₃ is selected from —NR— and —O—;
R is independently selected from H, C₁₋₆ alkyl and C₁₋₆ haloalkyl;
alternatively,
Ab is selected from: 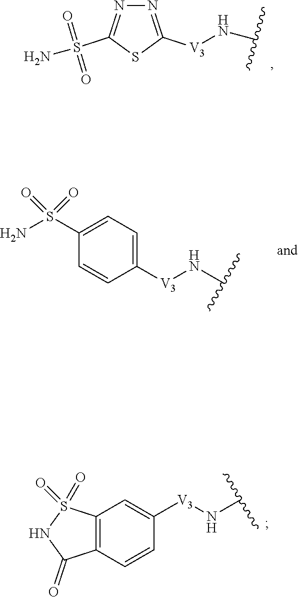
V₃ is selected from a chemical bond and a C₁₋₆ alkylene group (alternatively a C₁₋₄ alkylene group, alternatively a C₁₋₂ alkylene group);
still alternatively,
Ab is selected from: 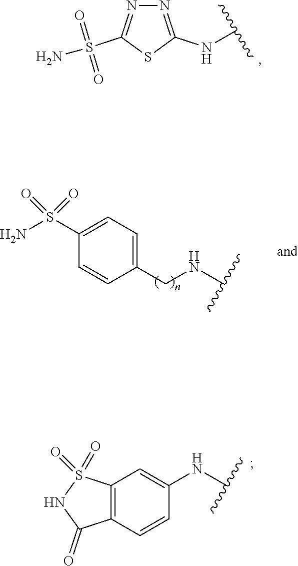
n is 0, 1, 2, 3, 4, 5 or 6, alternatively 0, 1, 2, 3 or 4, alternatively 0 or 2.

4. The compound according to claim 1, wherein the compound has one or more of the following definitions:
i) wherein
Ab is 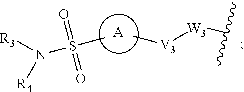
Ring A is selected from phenylene and C₅₋₆ heteroarylene; alternatively selected from phenylene and 1,3,4-thiadiazolylene; which is optionally substituted by 1, 2, 3 or 4 R_{A};
R_{A} is independently selected from H, D, halogen, CN, —OR, —SR, —NR_{b}R_{c}, C₁₋₆ alkyl, C₁₋₆ haloalkyl, C₃₋₇ cycloalkyl and 3-7 membered heterocyclyl; alternatively selected from H, D, halogen, C₁₋₆ alkyl and C₁₋₆ haloalkyl;
R₃ and R₄ are independently selected from H, halogen, C₁₋₆ alkyl and C₁₋₆ haloalkyl;
or R₄ and R_{A} are connected to form a C₁₋₄ alkylene group, alternatively a C₁₋₂ alkylene group, wherein 1, 2 or 3 (alternatively 1) methylene units are optionally and independently replaced by —CR*₂—, —NR*—, —O—, —C(O)—, —OC(O)—, —C(O)O—, —S—, —S(O)— or —S(O)₂—, alternatively replaced by —CR*₂—, —C(O)—, —OC(O)— or —C(O)O—, alternatively replaced by —C(O)—; and the ring formed by connecting R₄ with R_{A} is a ring structure fused with ring A;
V₃ is selected from a chemical bond, a C₁₋₆ alkylene group, a C₂₋₆ alkenylene group, and a C₂₋₆ alkynylene group;
W₃ is selected from —NR— and —O—;
R* is independently selected from H, D, halogen, C₁₋₆ alkyl and C₁₋₆ haloalkyl;
L₁ is —W₁—V₁—W₄—V₂—W₂—;
W₁ is selected from a chemical bond, —NR—, —C(O)—, —C(O)O—, and —C(O)NR—;
W₂ is selected from —C(O)—, —OC(O)—, —NRC(O)—, —NR—, —C(O)NR—, and —O—;
V₁ is selected from C₁₋₁₀ alkylene, C₂₋₁₀ alkenylene, C₂₋₁₀ alkynylene, C₆₋₁₀ arylene and C₅₋₁₀ heteroarylene, wherein the C₁₋₁₀ alkylene, C₂₋₁₀ alkenylene, C₂₋₁₀ alkynylene, C₆₋₁₀ arylene and C₅₋₁₀ heteroarylene are optionally substituted by 1, 2, 3, 4, 5 or 6 R₁ₛ; wherein one or more methylene units in the alkylene, alkenylene and alkynylene are optionally and independently replaced by —NR—, —NRC(O)—, —C(O)NR—, —NRS(O)₂—, —S(O)₂NR—, —O—, —C(O)—, —OC(O)—, —C(O)O—, —S—, —S(O)— or —S(O)₂—;
R₁ₛ is independently selected from H, D, —OR, —SRₐ, —NR_{b}R_{c}, halogen, C₁₋₆ alkyl, C₁₋₆ haloalkyl, C₃₋₇ cycloalkyl or 3-7 membered heterocyclyl; alternatively, R₁ₛ is independently in (S) or (R) configuration, or is in racemic form;
V₂ is a chemical bond or —(CR′R″)₁₋₆—;
R′ and R″ are independently selected from H, halogen, C₁₋₆ alkyl, C₁₋₆ haloalkyl, —C₁₋₆ alkylene-Ar;
alternatively, R′ is independently in (S) or (R) configuration, or is in racemic form;
Ar is independently selected from C₆₋₁₄ aryl and 5-14 membered heteroaryl;
W₄ is selected from a chemical bond, —C(O)—, —OC(O)—, —NRC(O)—, —NR—, —C(O)O—, —C(O)NR—, and —O—;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
L₂ is an amino acid chain formed by connecting 2-5 amino acid residues, wherein each amino acid residue can be optionally further substituted with one or more (alternatively 1, 2 or 3, alternatively 1) amino acid residues, and the amino acid residues can be optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent;
ii) wherein
Ab is selected from: 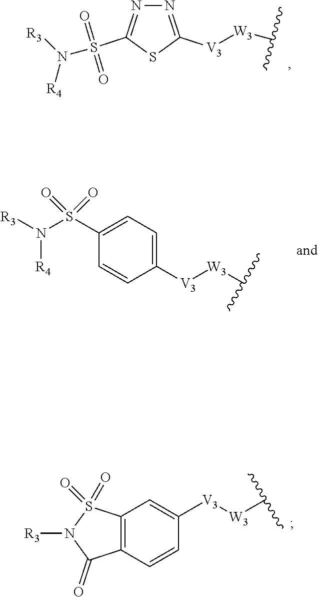
R₃ and R₄ are independently selected from H, halogen, C₁₋₆ alkyl and C₁₋₆ haloalkyl;
V₃ is selected from a chemical bond, a C₁₋₆ alkylene group, a C₂₋₆ alkenylene group, and a C₂₋₆ alkynylene group;
W₃ is selected from —NR— and —O—;
L₁ is —W₁—V₁—W₄—V₂—W₂—;
W₁ is selected from —C(O)—, —C(O)O— and —C(O)NR—; alternatively —C(O)—;
W₂ is selected from —C(O)—, —OC(O)— and —NRC(O)—; alternatively —C(O)—;
V₁ is selected from C₁₋₈ alkylene, C₂₋₈ alkenylene, C₂₋₈ alkynylene, phenylene and C₅₋₆ heteroarylene, alternatively C₁₋₈ alkylene (e.g. —(CH₂)ₘ—); which is optionally substituted by 1, 2, 3, 4, 5 or 6 R₁ₛ; wherein 1, 2 or 3 methylene units in the alkylene, alkenylene and alkynylene are optionally and independently replaced by —NR—, —NRC(O)—, —C(O)NR—, —C(O)—, —OC(O)— or —C(O)O—;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —ORₐ, —NR_{b}R_{c}, halogen, C₁₋₆ alkyl and C₁₋₆ haloalkyl; alternatively H, D, —ORₐ and —NR_{b}R_{c};
V₂ is a chemical bond or —(CR′R″)₁₋₄—;
R′ is independently selected from H, D or —C₁₋₄ alkylene-Ar; alternatively, R′ is independently in (S) or (R) configuration, or in racemic form; alternatively in (S) configuration; alternatively in (R) configuration;
R″ is independently selected from H, D, halogen, C₁₋₆ alkyl and C₁₋₆ haloalkyl;
Ar is independently selected from C₆₋₁₄ aryl and 5-14 membered heteroaryl, alternatively C₆₋₁₄ aryl;
W₄ is selected from a chemical bond, —OC(O)—, —NRC(O)—, —C(O)O— and —C(O)NR—;
L₂ is 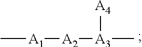
A₁, A₂, A₃ and A₄ are amino acid residues, and the amino acid residues are optionally substituted with 1, 2, 3, 4, 5 or 6 R_{L};
R_{L} is independently selected from halogen, NO₂, —OR, —NR_{b}R_{c}, C₁₋₆ alkyl, C₁₋₆ haloalkyl, urea group, C₁₋₆ acyl, —C(O)—C₁₋₆ alkylene-COOH, sulfonic acid group, methylsulfonyl, phosphate group and phosphite group;
Z is a chelating group derived from a chelating agent;
iii) wherein
Ab is selected from: 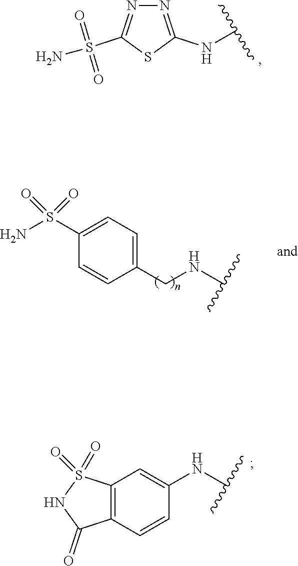
n is 0, 1, 2, 3, 4, 5 or 6, alternatively 0, 1, 2, 3 or 4, alternatively 0 or 2;
L₁ is —C(O)—V₁—W₄—V₂—C(O)—;
wherein V₁ is selected from C₁₋₆ alkylene (e.g. —(CH₂)ₘ—), phenylene and C₅₋₆ heteroarylene; alternatively selected from C₁₋₆ alkylene (e.g. —(CH₂)ₘ—), phenylene, pyrrolylene, furylene or thienylene; alternatively C₁₋₆ alkylene (e.g. —(CH₂)ₘ—); alternatively C₁₋₄ alkylene (e.g. —(CH₂)ₘ—); which is optionally substituted by 1, 2, 3, 4, 5 or 6 R₁ₛ; wherein 1, 2 or 3 methylene units in the alkylene are optionally and independently replaced by —NR—, —NRC(O)—, —C(O)NR—, —C(O)—, —OC(O)— or —C(O)O—;
m is independently selected from 1, 2, 3, 4, 5 or 6, alternatively 1, 2, 3 or 4;
R is independently selected from H, C₁₋₆ alkyl and C₁₋₆ haloalkyl;
R₁ₛ is independently selected from H, D, —ORₐ, C₁₋₆ alkyl and C₁₋₆ haloalkyl; alternatively selected from H and —ORₐ;
V₂ is a chemical bond or —(CR′R″)₁₋₂— (alternatively —CHR′—);
R′ is independently selected from H or —C₁₋₄ alkylene-Ar; alternatively H or —CH₂—Ar; alternatively, R′ is independently in (S) or (R) configuration, or in racemic form; alternatively in (S) configuration; alternatively in (R) configuration;
R″ is independently selected from H, D, C₁₋₆ alkyl and C₁₋₆ haloalkyl; alternatively H or D;
Ar is independently C₆₋₁₄ aryl, such as phenyl, naphthyl, anthracenyl or phenanthryl; alternatively C₆₋₁₀ aryl, such as phenyl or naphthyl, alternatively naphthyl, alternatively 2-naphthyl;
W₄ is selected from a chemical bond, —OC(O)—, —NRC(O)—, —C(O)O— and —C(O)NR—; alternatively a chemical bond, —C(O)O— and —C(O)NR—; alternatively a chemical bond or —C(O)NR—;
L₂ is 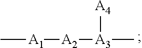
A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —ORₐ, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
Z is a chelating group derived from a chelating agent, wherein the chelating agent is selected from:
1,4,7,10-tetraazacyclododecane-N,N′,N″,N′″-tetraacetic acid (DOTA),
N,N″-bis[2-hydroxy-5-(carboxyethyl)benzyl]ethylenediamine-N,N″-diacetic acid (HBED-CC),
1,4,7-triazacyclononane-1,4,7-triacetic acid (NOTA),
2-(4,7-bis(carboxymethyl)-1,4,7-triazononan-1-yl)glutaric acid (NODAGA),
2-(4,7,10-tris(carboxymethyl)-1,4,7,10-tetraazacyclododecane-1-yl)glutaric acid (DOTAGA),
1,4,7-triazacyclononanephosphinic acid (TRAP),
1,4,7-triazacyclononane-1-[methyl(2-carboxyethyl)phosphinic acid]-4,7-bis[methyl(2-hydroxymethyl)phosphinic acid](NOPO),
3,6,9,15-tetraazabicyclo[9.3.1]pentadeca-1(15),11,13-triene-3,6,9-triacetic acid (PCTA),
N′-{5-[acetyl(hydroxy)amino]pentyl}-N-[5-({4-[(5-aminopentyl)(hydroxy)amino]-4-oxobutanoyl}amino)pentyl]-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (oxy-Do3A),
p-isocyanatobenzyl-DTPA (SCN-Bz-DTPA),
1-(p-isocyanatobenzyl)-3-methyl-DTPA (1B3M),
2-(p-isocyanatobenzyl)-4-methyl-DTPA (1M3B),
1-(2)-methyl-4-isocyanatobenzyl-DTPA (MX-DTPA),
(R)-2-amino-3-(4-isothiocyanatophenyl)propyl]-trans-(S,S)-cyclohexane-1,2-diaminepentaacetic acid (p-SCN-Bn-CHX-A″-DTPA),
6-hydrazinopyridine-3-carboxylic acid (HYNIC),
2-(4-isothiocyanatophenyl)-1,4,7-triazacyclononane-1,4,7-triacetic acid (p-SCN-Bn-NOTA) or
2-[(4-isothiocyanatophenyl)methyl]-1,4,7,10-tetraazacyclododecane-1,4,7,10-tetraacetic acid (p-SCN-Bn-DOTA); and
iv) wherein
Ab is selected from: 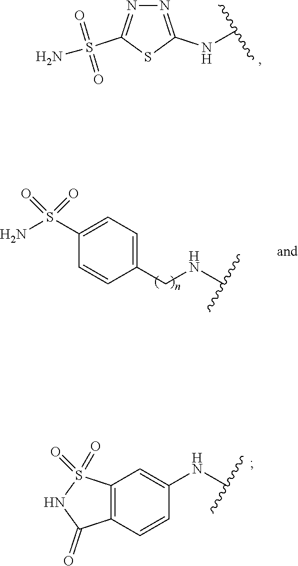
n is 0, 1, 2, 3, 4, 5 or 6, alternatively 0, 1, 2, 3 or 4, alternatively 0 or 2;
L₁ is selected from: 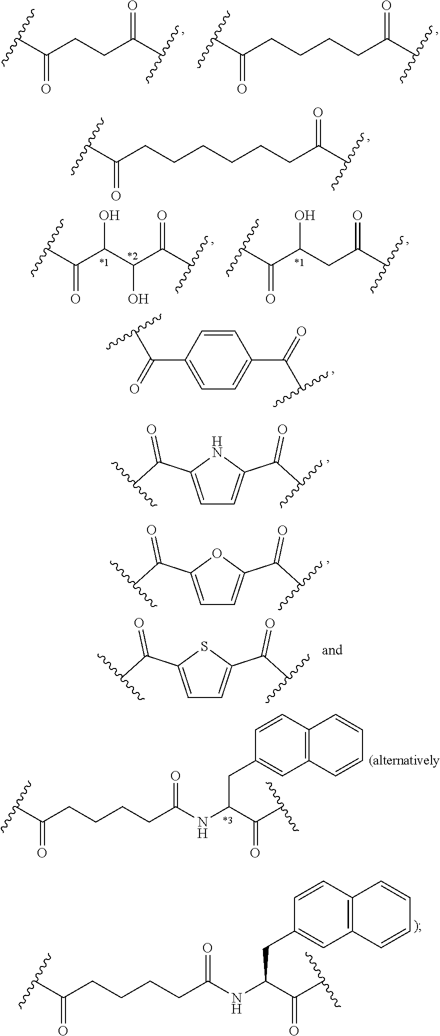
wherein, *1, *2 and *3 are chiral centers, independently selected from in (S) or (R) configuration, or in racemic form; alternatively in (S) configuration; alternatively in (R) configuration;
L₂ is 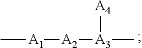
A₁ is a lysine residue; 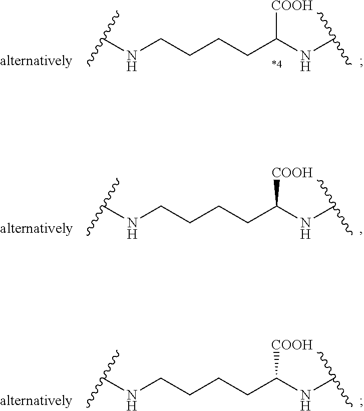 [SMILES: *NCCCCC(N*)C(=O)O ; *NCCCC[C@H](N*)C(=O)O ; *NCCCC[C@@H](N*)C(=O)O]
A₂ is selected from a tyrosine residue, an aspartic acid residue, a serine residue, a glutamic acid residue, a phenylalanine residue or an arginine residue, which is optionally substituted by 1, 2, 3 or 4 R_{L};
R_{L} is independently selected from halogen, —OR, —NR_{b}R_{c}, C₁₋₆ alkyl, C₁₋₆ haloalkyl and urea group;
alternatively halogen, C₁₋₆ alkyl, C₁₋₆ haloalkyl and urea group;
alternatively A₂ is selected from 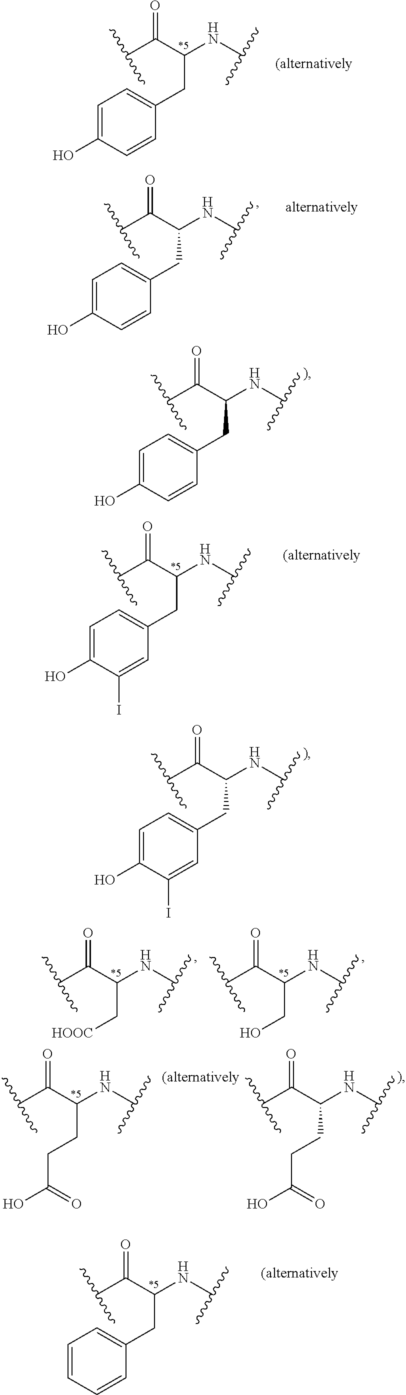 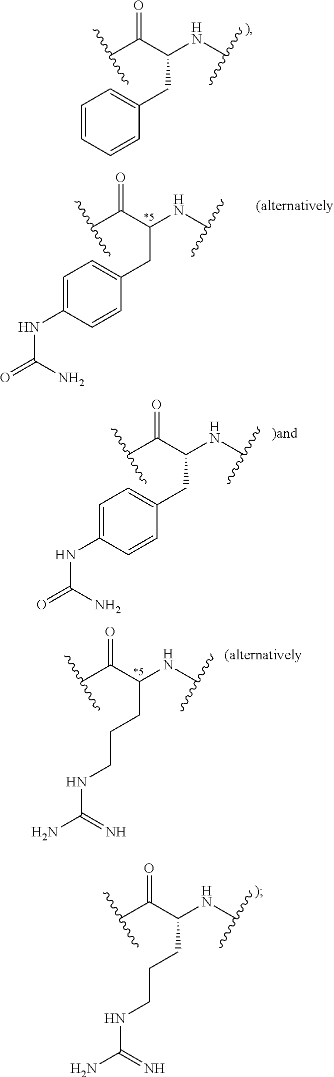
A₃ is a lysine residue; alternatively 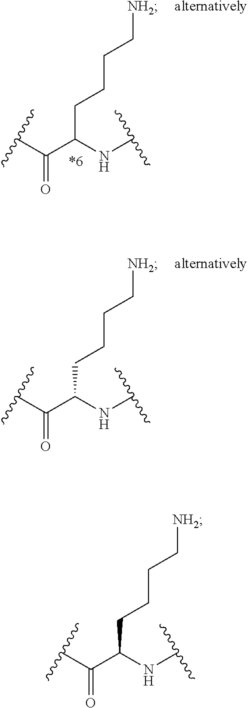
A₄ is selected from N-acetylglutamate residue, N-(4-carboxybutyryl)glutamate, glutamic acid residue, pyroglutamic acid residue, citrulline residue or aspartic acid residue; alternatively 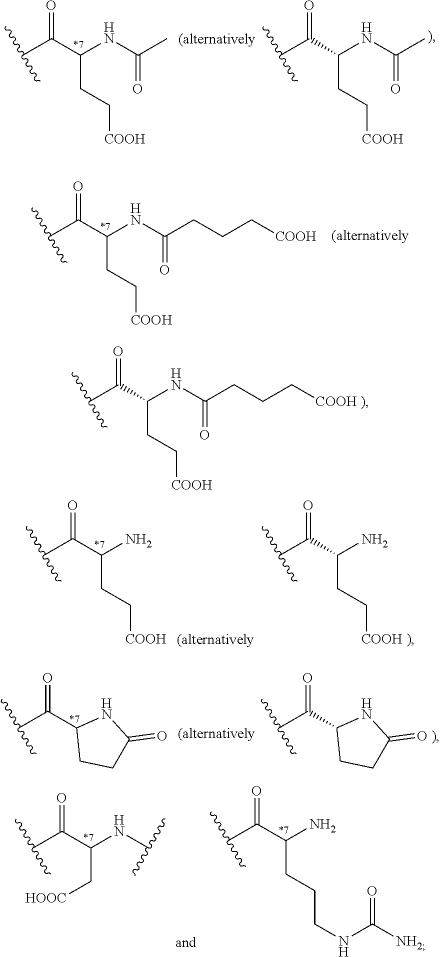
wherein, *4, *5, *6 and *7 are chiral centers, independently selected from in (S) or (R) configuration, or in racemic form; alternatively in (S) configuration; alternatively in (R) configuration;
Z is selected from 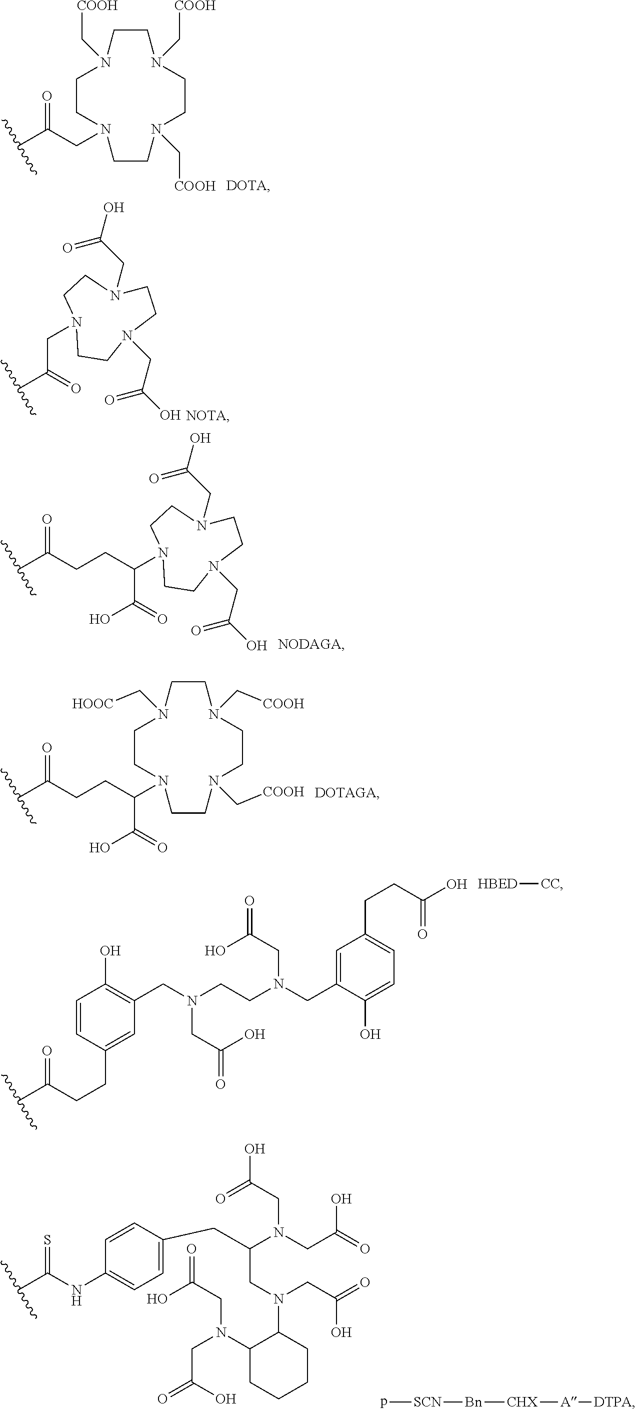 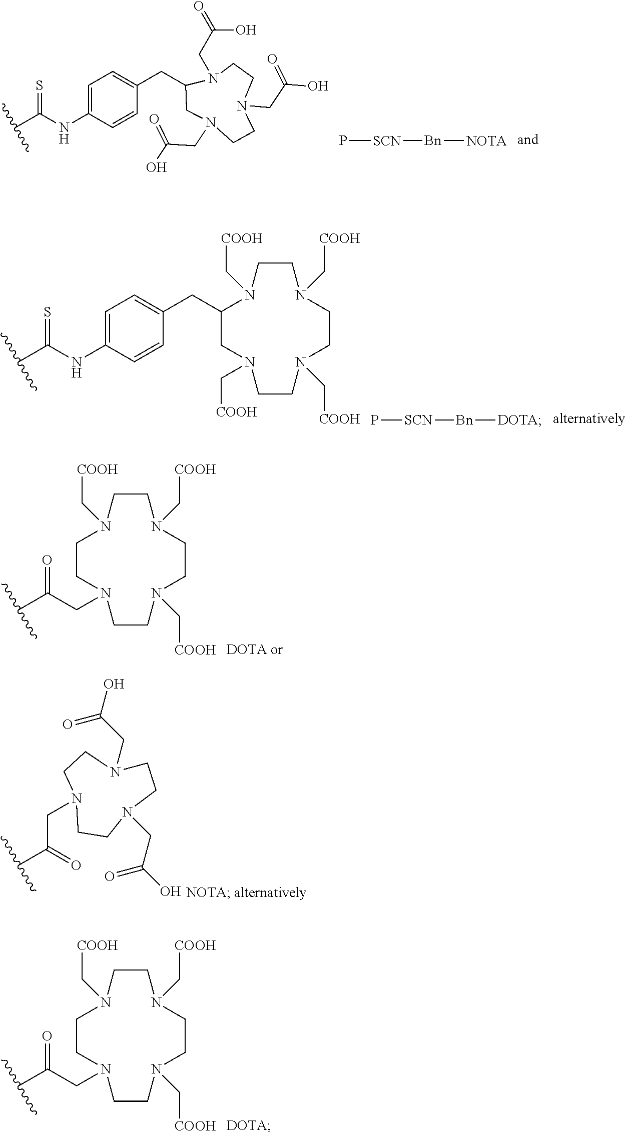
alternatively,
L₂ is selected from:

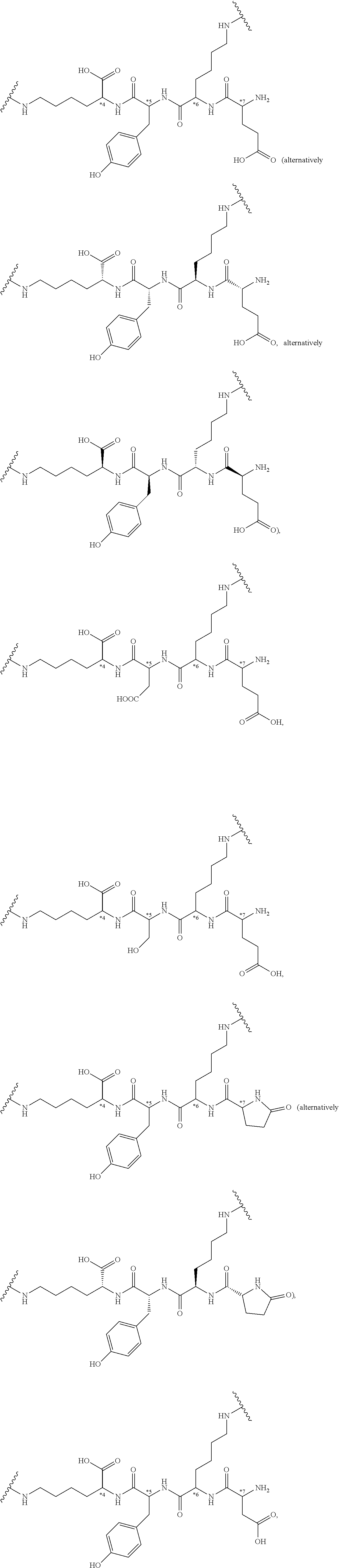

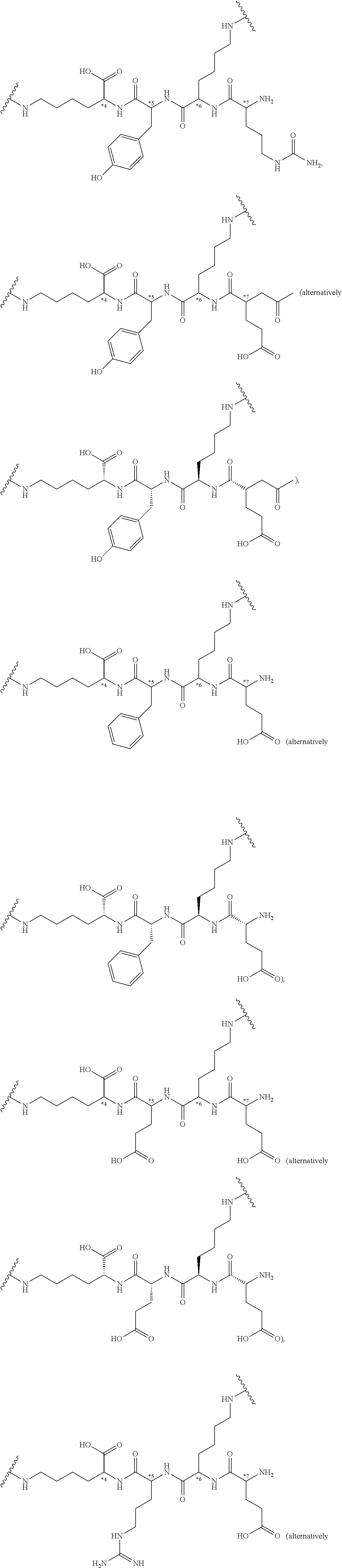

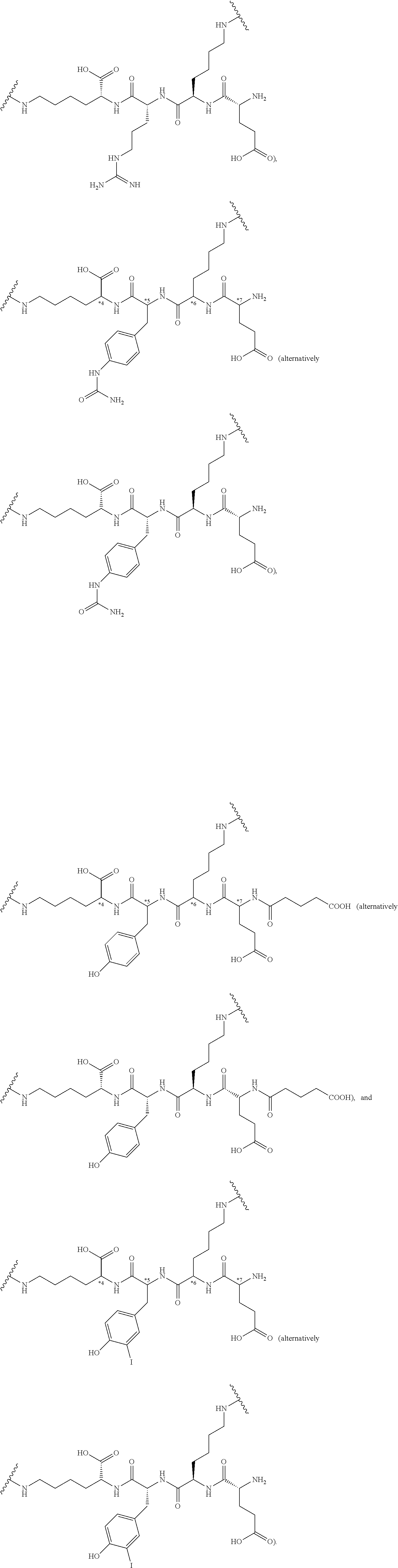

5. The compound according to claim 1, wherein the compound has one or more of the following definitions: i) wherein V₃ is selected from a chemical bond, a C₁₋₆ alkylene group, a C₂₋₆ alkenylene group, a C₂₋₆ alkynylene group, and 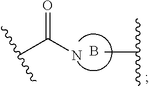
alternatively a chemical bonds, a C₁₋₆ alkylene group (alternatively, a C₁₋₄ alkylene groups, alternatively C₁₋₂ alkylene groups) and 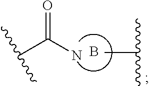
alternatively a chemical bond and 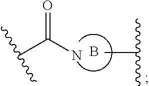
alternatively,
Ring B is a 3-7 membered heterocyclylene group; alternatively, a 4-6 membered heterocyclylene group; alternatively, a 5-membered heterocyclylene group, for example 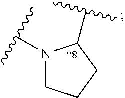 [SMILES: *C1CCCN1*]
wherein *8 is a chiral center, which is independently selected from in (S) or (R) configuration, or is in racemic form; alternatively in (S) configuration; alternatively in (R) configuration;
the remaining variables are as defined in claim 1; and
ii) wherein V₃ is selected from —NR—, —O— and —C(O)—, alternatively —NR— and —C(O)—; the remaining variables are as defined in claim 1.

6. The compound according to claim 1, wherein Ab is selected from: 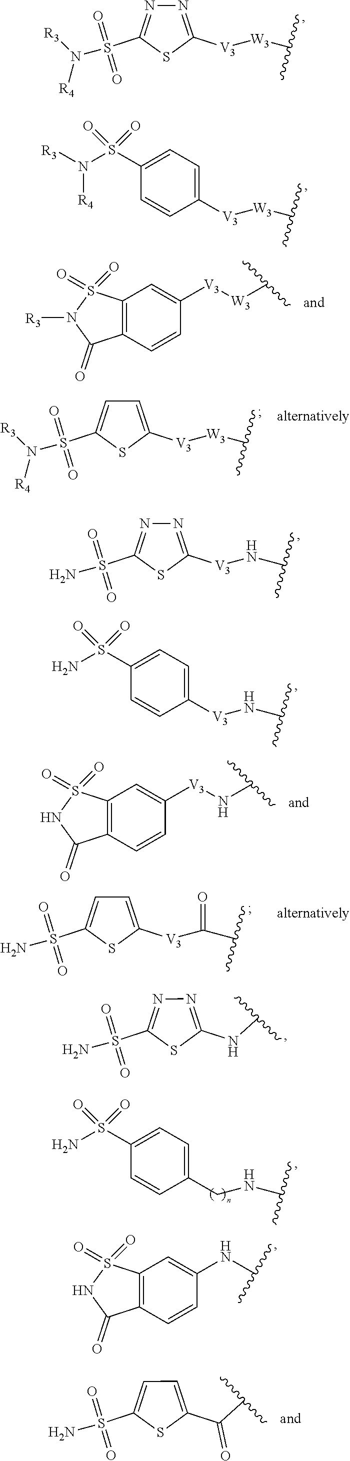 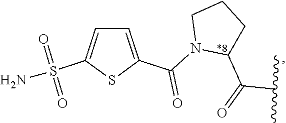 [SMILES: *C(=O)C1CCCN1C(=O)c1ccc(S(N)(=O)=O)s1]
wherein *8 is a chiral center, which is independently selected from in (S) or (R) configuration, or in racemic form;
alternatively in (S) configuration; alternatively in (R) configuration;
the remaining variables are defined in claim 1.

7. The compound according to claim 3, wherein L₁ is —W₁—V₁—W₄—V₂—W₂—;
W₁ is selected from —NR—, —C(O)—, —C(O)O— and —C(O)NR—; alternatively —NR— or —C(O)—;
alternatively, R₁ₛ is independently selected from H, —ORₐ, —NR_{b}R_{c}, C₁₋₆ alkyl and C₁₋₆ haloalkyl; alternatively H, —ORₐ and —NR_{b}R_{c}; alternatively H and —NR_{b}R_{c};
the remaining variables are described in claim 3;
alternatively,
L₁ is selected from: 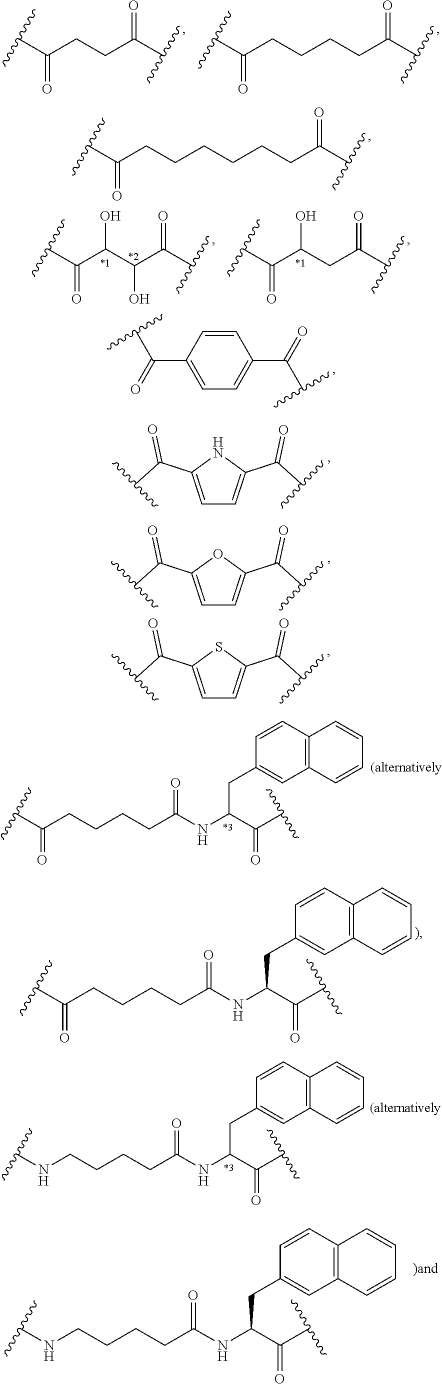 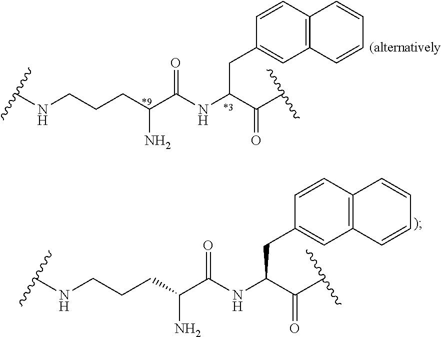 [SMILES: *NCCCC(N)C(=O)NC(Cc1ccc2ccccc2c1)C(*)=O ; *NCCC[C@@H](N)C(=O)N[C@@H](Cc1ccc2ccccc2c1)C(*)=O]
wherein *1, *2, *3 and *9 are chiral centers, which are independently selected from in (S) or (R) configurations, or in racemic form; alternatively in (S) configuration; alternatively in (R) configuration; the remaining variables are described in claim 3.

8. The compound according to claim 1, which has a structure represented by formula (III-1) or formula (IV-1), 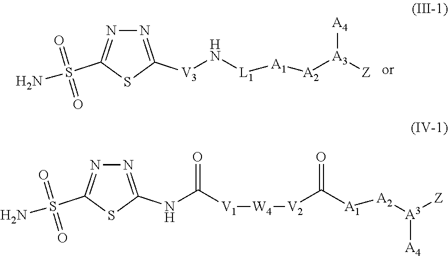
wherein,
V₃ is selected from a chemical bond, a C₁₋₆ alkylene group, a C₂₋₆ alkenylene group, and a C₂₋₆ alkynylene group;
L₁ is —W₁—V₁—W₄—V₂—W₂—;
W₁ is selected from —C(O)—, —C(O)O— and —C(O)NR—; alternatively —C(O)—;
W₂ is selected from —C(O)—, —OC(O)— and —NRC(O)—; alternatively —C(O)—;
V₁ is selected from C₁₋₈ alkylene, C₂₋₆ alkenylene and C₂₋₆ alkynylene, alternatively C₁₋₈ alkylene (e.g. —(CH₂)ₘ—), which is optionally substituted by 1, 2, 3, 4, 5 or 6 R₁ₛ;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —ORₐ, —NR_{b}R_{c}, halogen, C₁₋₆ alkyl and C₁₋₆ haloalkyl; alternatively H, D, —ORₐ and —NR_{b}R_{c}; alternatively, R₁ₛ is independently in (S) or (R) configuration, or is in racemic form;
V₂ is a chemical bond or —(CR′R″)₁₋₆—; alternatively —(CR′R″)₁₋₆—;
R′ is independently selected from H, D or —C₁₋₄ alkylene-Ar; alternatively, R′ is independently in (S) or (R) configuration, or is in racemic form;
R″ is independently selected from H, D, halogen, C₁₋₆ alkyl and C₁₋₆ haloalkyl;
Ar is independently selected from C₆₋₁₄ aryl and 5-14 membered heteroaryl; alternatively C₆₋₁₄ aryl;
W₄ is selected from a chemical bond, —OC(O)—, —NRC(O)—, —C(O)O— and —C(O)NR—;
Rₐ, R_{b} and R_{c} are independently selected from H, C₁₋₆ alkyl and C₁₋₆ haloalkyl; or R_{b}, R_{c} and the atom to which they are attached are taken together to form a 3-7 membered heterocyclyl group;
A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
Z is as defined in claim 1.

9. The compound of claim 8, wherein
V₃ is selected from a chemical bond and a C₁₋₆ alkylene group (alternatively a C₁₋₄ alkylene group, alternatively a C₁₋₂ alkylene group); alternatively a chemical bond;
L₁ is —W₁—V₁—W₄—V₂—W₂—;
W₁ is —C(O)—;
W₂ is —C(O)—;
V₁ is selected from C₁₋₆ alkylene, C₂₋₆ alkenylene and C₂₋₆ alkynylene, alternatively C₁₋₆ alkylene (e.g. —(CH₂)ₘ—), alternatively C₁₋₄ alkylene (e.g. —(CH₂)ₘ—), which is optionally substituted by 1, 2, 3, 4, 5 or 6 R₁ₛ;
m is independently selected from 1, 2, 3, 4, 5 or 6, alternatively 1, 2, 3 or 4;
R₁ₛ is independently selected from H, D, —ORₐ, C₁₋₆ alkyl and C₁₋₆ haloalkyl; alternatively selected from H and —ORₐ; alternatively selected from H and D;
V₂ is a chemical bond or —(CR′R″)₁₋₄—; alternatively a chemical bond or —(CR′R″)₁₋₂— (alternatively —CHR′—);
alternatively —(CR′R″)₁₋₂— (alternatively —CHR′—);
R′ is independently selected from H or —C₁₋₄ alkylene-Ar, alternatively H or —CH₂—Ar; alternatively, R′ is independently in (S) or (R) configuration, or in racemic form; alternatively in (S) configuration;
R″ is independently selected from H, D, C₁₋₆ alkyl and C₁₋₆ haloalkyl; alternatively H or D;
Ar is independently C₆₋₁₄ aryl, such as phenyl, naphthyl, anthracenyl or phenanthryl; alternatively C₆₋₁₀ aryl, such as phenyl or naphthyl, alternatively naphthyl, alternatively 2-naphthyl;
W₄ is selected from a chemical bond or —C(O)NR—;
R is independently selected from H, C₁₋₆ alkyl and C₁₋₆ haloalkyl;
Rₐ is independently selected from H, C₁₋₆ alkyl and C₁₋₆ haloalkyl;
A₁ is a lysine residue;
A₂ is a tyrosine residue;
A₃ is a lysine residue;
A₄ is a glutamic acid residue or an N-acetylglutamic acid residue, alternatively a glutamic acid residue;
Z is selected from 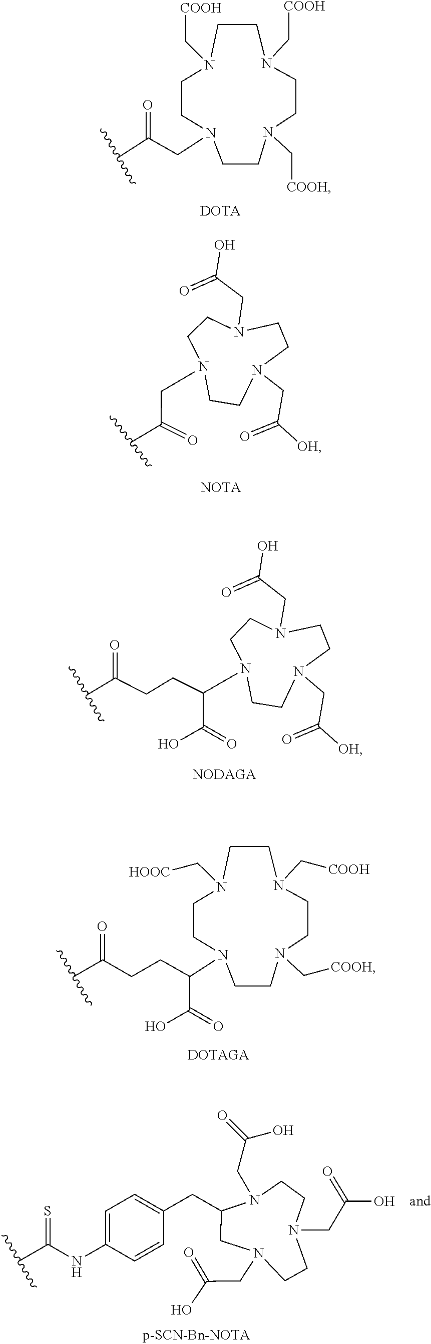 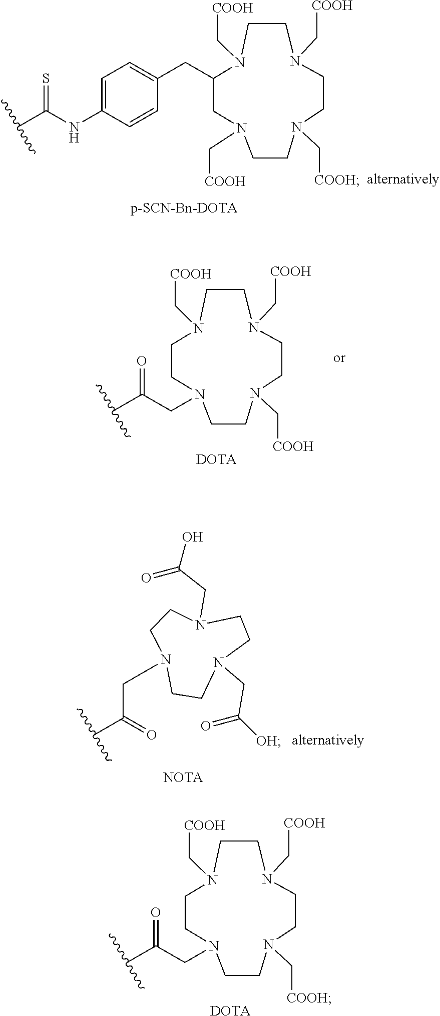
alternatively, 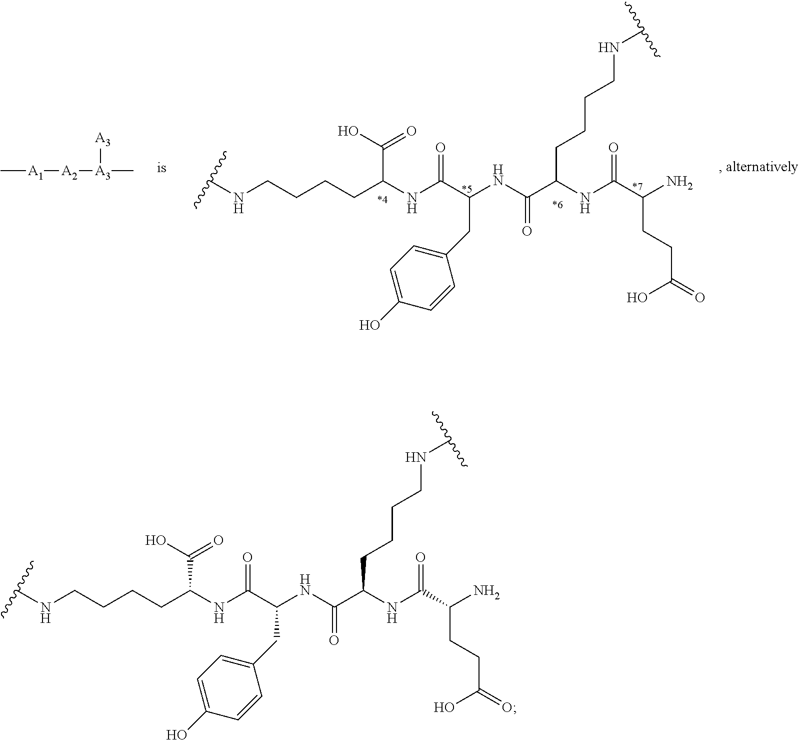
wherein, *4, *5, *6 and *7 are chiral centers, independently selected from in (S) or (R) configuration, or in racemic form; alternatively in (R) configuration;
alternatively, L₁ is 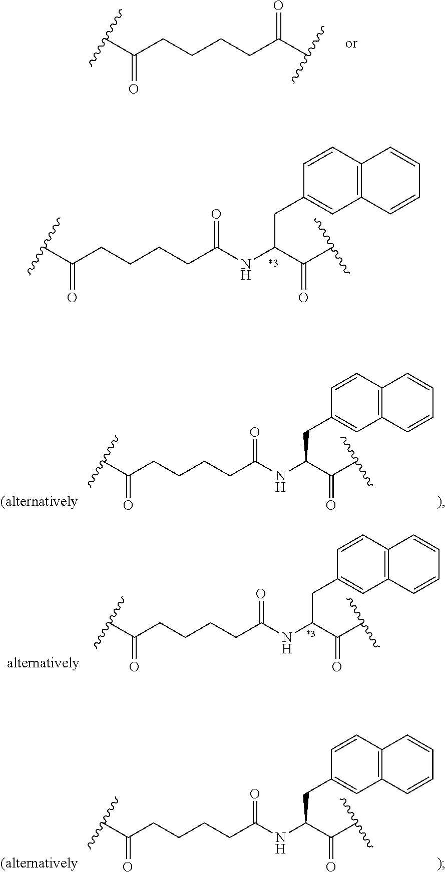
wherein, *3 is a chiral center, independently selected from in (S) or (R) configuration, or in racemic form;
alternatively in (S) configuration.

10. The compound according to claim 1, which has a structure represented by formula (II-4), 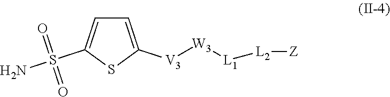
wherein,
V₃ is selected from a chemical bond, a C₁₋₆ alkylene group, a C₂₋₆ alkenylene group and a C₂₋₆ alkynylene group, and 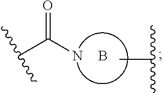
Ring B is a 3-7 membered heterocyclyl group;
W₃ is selected from —NR— and —C(O)—; alternatively —C(O)—;
L₁ is —W₁—V₁—W₄—V₂—W₂—;
W₁ is selected from —NR—, —C(O)—, —C(O)O— and —C(O)NR—; alternatively —NR— or —C(O)—;
W₂ is selected from —C(O)—, —OC(O)— and —NRC(O)—; alternatively —C(O)—;
V₁ is selected from C₁₋₈ alkylene, C₂₋₆ alkenylene and C₂₋₆ alkynylene, alternatively C₁₋₈ alkylene (e.g. —(CH₂)ₘ—), which is optionally substituted by 1, 2, 3, 4, 5 or 6 R₁ₛ;
m is independently selected from 1, 2, 3, 4, 5, 6, 7 or 8;
R is independently selected from H, C₁₋₆ alkyl and C₁₋₆ haloalkyl;
R₁ₛ is independently selected from H, D, —ORₐ, —NR_{b}R_{c}, halogen, C₁₋₆ alkyl and C₁₋₆ haloalkyl; alternatively selected from H, D, —ORₐ and —NR_{b}R_{c}; alternatively, R₁ₛ is independently in (S) or (R) configuration, or in racemic form;
V₂ is a chemical bond or —(CR′R″)₁₋₆—; alternatively —(CR′R″)₁₋₆—;
R′ is independently selected from H, D or —C₁₋₄ alkylene-Ar, alternatively, R′ is independently in (S) or (R) configuration, or in racemic form;
R″ is independently selected from H, D, halogen, C₁₋₆ alkyl and C₁₋₆ haloalkyl;
Ar is independently selected from C₆₋₁₄ aryl and 5-14 membered heteroaryl; alternatively C₆₋₁₄ aryl;
W₄ is selected from a chemical bond, —OC(O)—, —NRC(O)—, —C(O)O— and —C(O)NR—;
Rₐ, R_{b} and R_{c} are independently selected from H, C₁₋₆ alkyl and C₁₋₆ haloalkyl; or R_{b}, R_{c} and the atom to which they are attached together form a 3-7 membered heterocyclyl group;
L₂ is 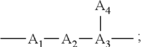
A₁, A₂, A₃ and A₄ are selected from glycine residues, alanine residues, phenylalanine residues, lysine residues, tyrosine residues, aspartic acid residues, serine residues, glutamic acid residues, pyroglutamic acid residues, citrulline residues or arginine residues; alternatively selected from lysine residues, tyrosine residues, aspartic acid residues, serine residues, glutamic acid residues, pyroglutamic acid residues, citrulline residues, phenylalanine residues or arginine residues, which are optionally substituted with 1, 2, 3 or 4 R_{L};
R_{L} is independently selected from halogen, —OR, —NR_{b}R_{c}, C₁₋₆ alkyl, C₁₋₆ haloalkyl, urea group, C₁₋₆ acyl and —C(O)—C₁₋₆ alkylene-COOH; alternatively halogen, C₁₋₆ alkyl, C₁₋₆ haloalkyl, urea group, C₁₋₆ acyl and —C(O)—C₁₋₆ alkylene-COOH; alternatively C₁₋₆ acyl;
Z is as defined in claim 1.

11. The compound of claim 10, wherein,
V₃ is selected from a chemical bond, a C₁₋₆ alkylene group (alternatively a C₁₋₄ alkylene group, alternatively a C₁₋₂ alkylene group), and 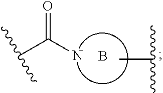
alternatively a chemical bond and 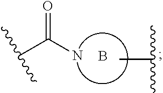
Ring B is a 4-6 membered heterocyclyl group; alternatively a 5-membered heterocyclyl group; for example 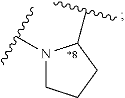 [SMILES: *C1CCCN1*]
W₃ is selected from —NR— and —C(O)—; alternatively —C(O)—;
L₁ is —W₁—V₁—W₄—V₂—W₂—;
W₁ is —NR—; alternatively —NH—;
W₂ is —C(O)—;
V₁ is selected from C₁₋₆ alkylene, C₂₋₆ alkenylene and C₂₋₆ alkynylene, alternatively C₁₋₆ alkylene (e.g. —(CH₂)ₘ—), alternatively C₁₋₄ alkylene (e.g. —(CH₂)ₘ—), which is optionally substituted by 1, 2, 3, 4, 5 or 6 R₁ₛ;
m is independently selected from 1, 2, 3, 4, 5, and 6, alternatively 1, 2, 3, and 4;
R₁ₛ is independently selected from H, D, —OR, —NR_{b}R_{c}, C₁₋₆ alkyl and C₁₋₆ haloalkyl; alternatively selected from H, —ORₐ and —NR_{b}R_{c}; alternatively H and —NR_{b}R_{c};
V₂ is a chemical bond or —(CR′R″)₁₋₄—; alternatively a chemical bond or —(CR′R″)₁₋₂— (alternatively —CHR′—);
alternatively —(CR′R″)₁₋₂— (alternatively —CHR′—);
R′ is independently selected from H or —C₁₋₄ alkylene-Ar, alternatively, H or —CH₂—Ar; alternatively, R′ is independently in (S) or (R) configuration, or in racemic form; alternatively in (S) configuration;
R″ is independently selected from H, D, C₁₋₆ alkyl and C₁₋₆ haloalkyl; alternatively H, or D;
Ar is independently C₆₋₁₄ aryl, such as phenyl, naphthyl, anthracyl or phenanthryl; alternatively C₆₋₁₀ aryl, such as phenyl, or naphthyl, alternatively naphthyl, alternatively 2-naphthyl;
W₄ is selected from a chemical bond and —C(O)NR—; alternatively —C(O)NR—;
R is independently selected from H, C₁₋₆ alkyl and C₁₋₆ haloalkyl;
Rₐ, R_{b} and R_{c} are independently selected from H, C₁₋₆ alkyl and C₁₋₆ haloalkyl; alternatively H;
L₂ is 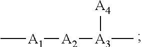
A₁ is a lysine residue;
A₂ is a tyrosine residue;
A₃ is a lysine residue;
A₄ is a glutamic acid residue or an N-acetylglutamic acid residue, alternatively a glutamic acid residue;
Z is selected from 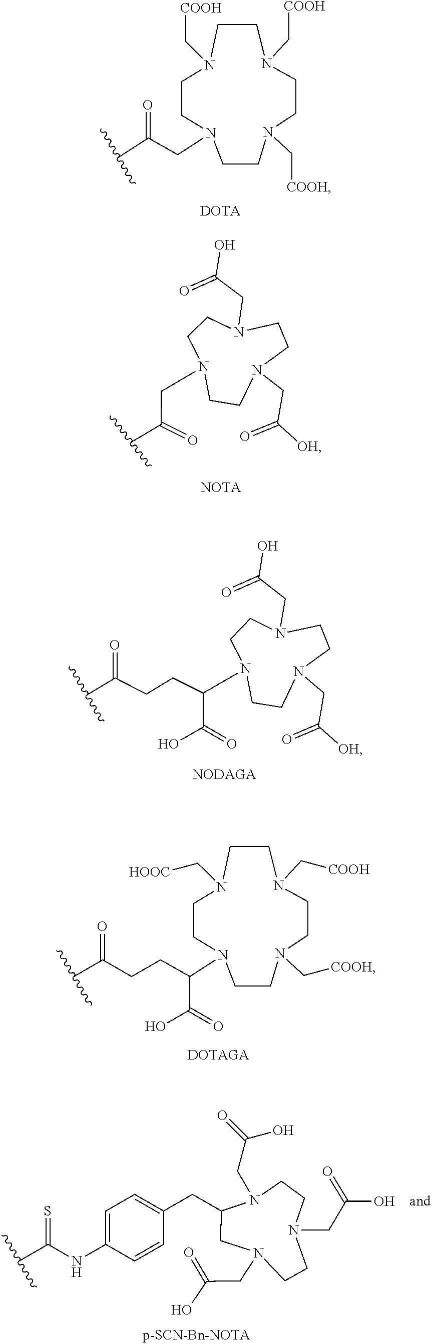 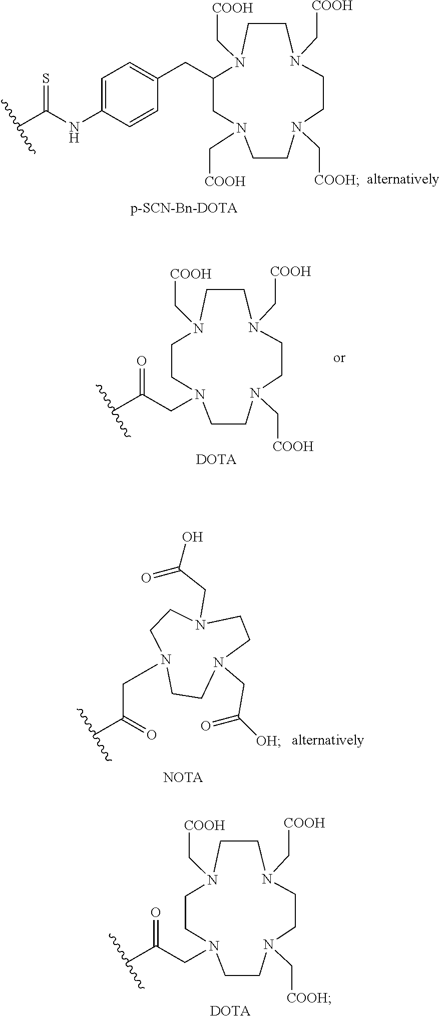
alternatively, 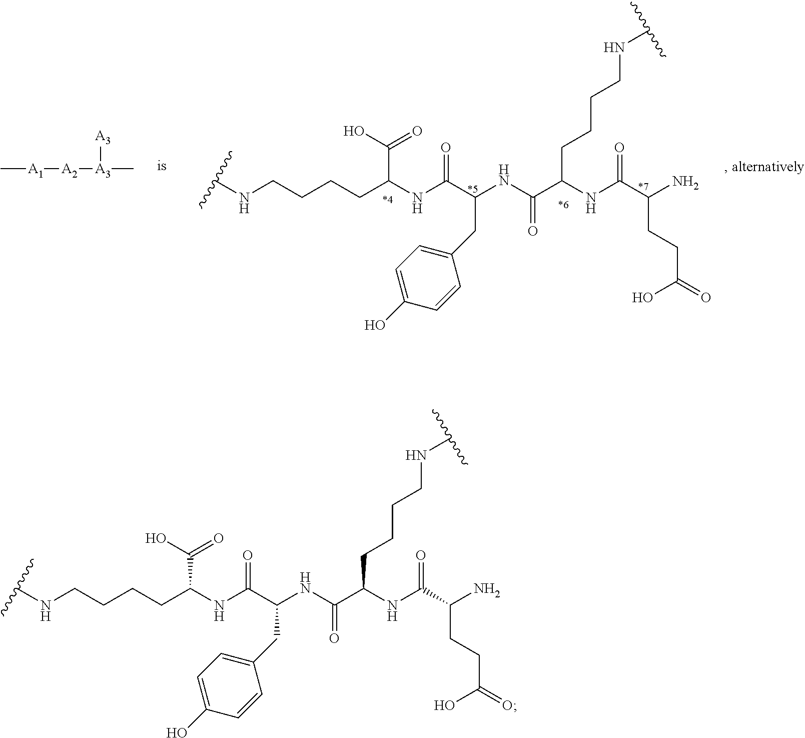
wherein, *4, *5, *6, *7, and *8 are chiral centers, independently selected from in (S) or (R) configuration, or in racemic form; alternatively in (R) configuration;
alternatively, L₁ is 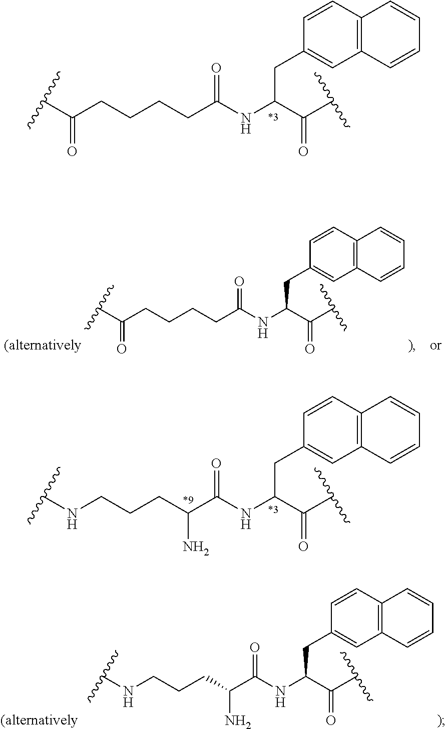
wherein, *3 and *9 are chiral centers, independently selected from in (S) or (R) configuration, or in racemic form; alternatively in (S) configuration.

12. The compound of claim 1, wherein the compound is selected from:

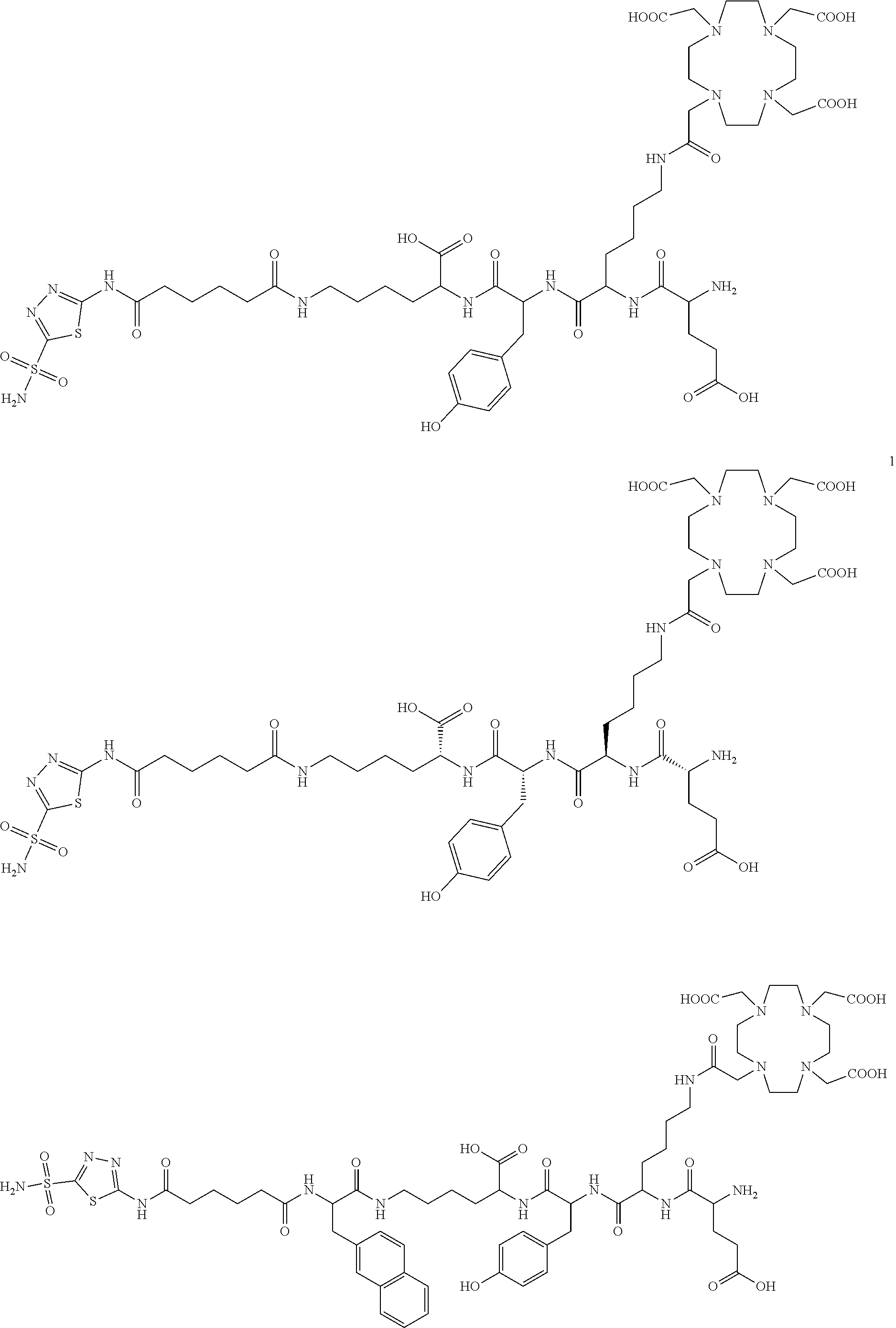 [SMILES: NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCCCC(=O)Nc1nnc(S(N)(=O)=O)s1)C(=O)O ; N[C@H](CCC(=O)O)C(=O)N[C@H](CCCCNC(=O)CN1CCN(CC(=O)O)CCN(CC(=O)O)CCN(CC(=O)O)CC1)C(=O)N[C@H](Cc1ccc(O)cc1)C(=O)N[C@H](CCCCNC(=O)CCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)C(Cc1ccc2ccccc2c1)NC(=O)CCCCC(=O)Nc1nnc(S(N)(=O)=O)s1)C(=O)O]

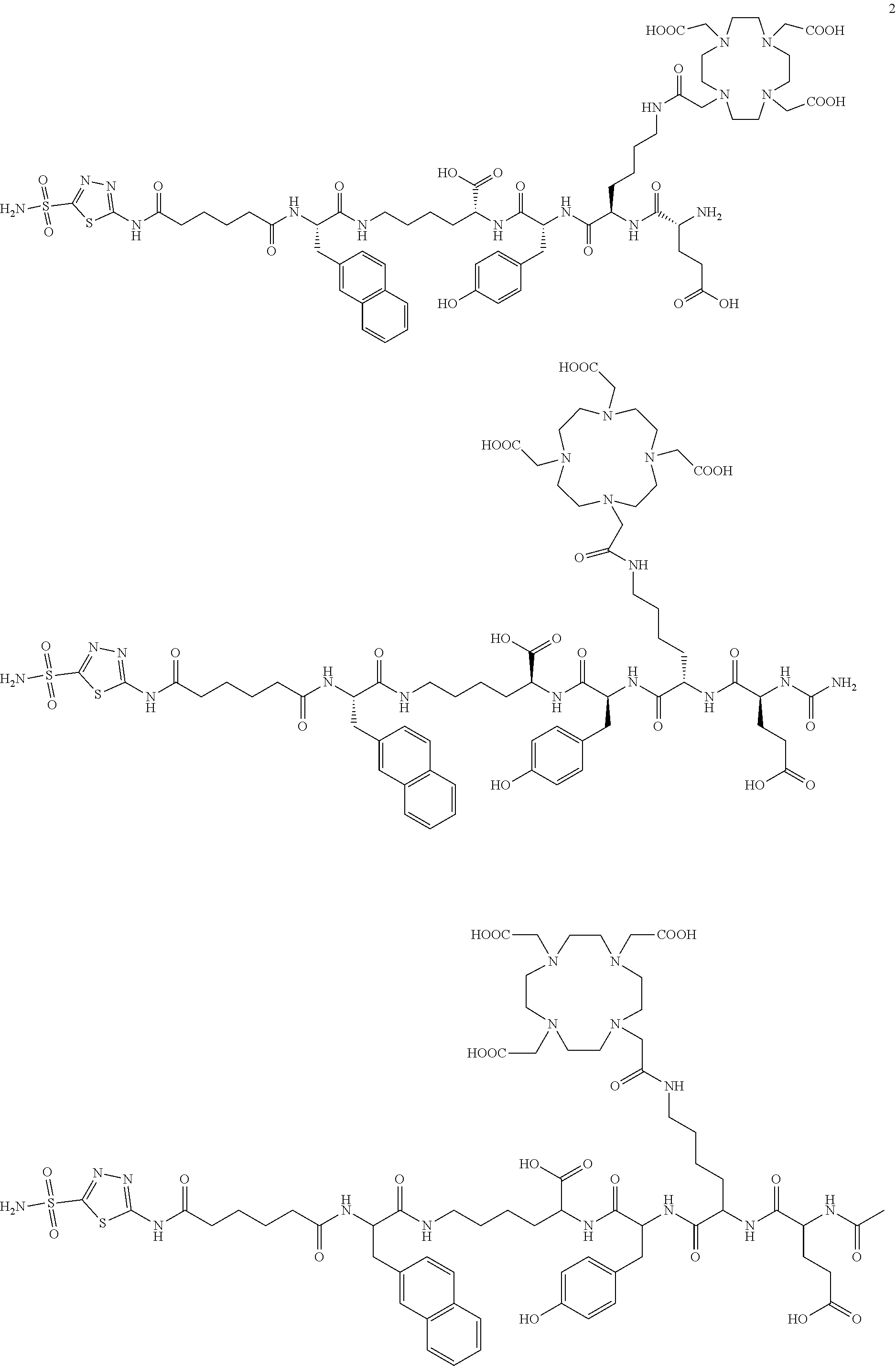

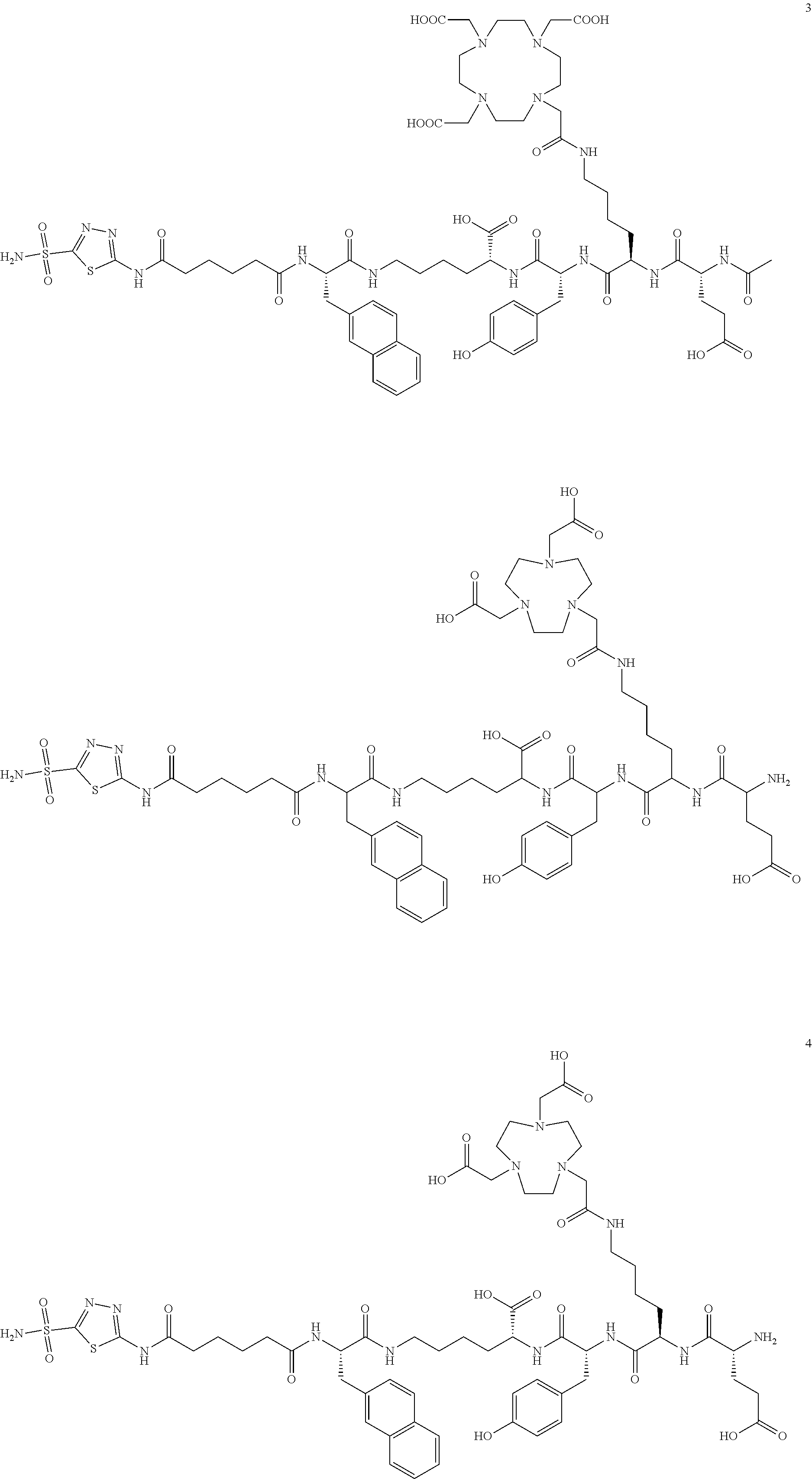

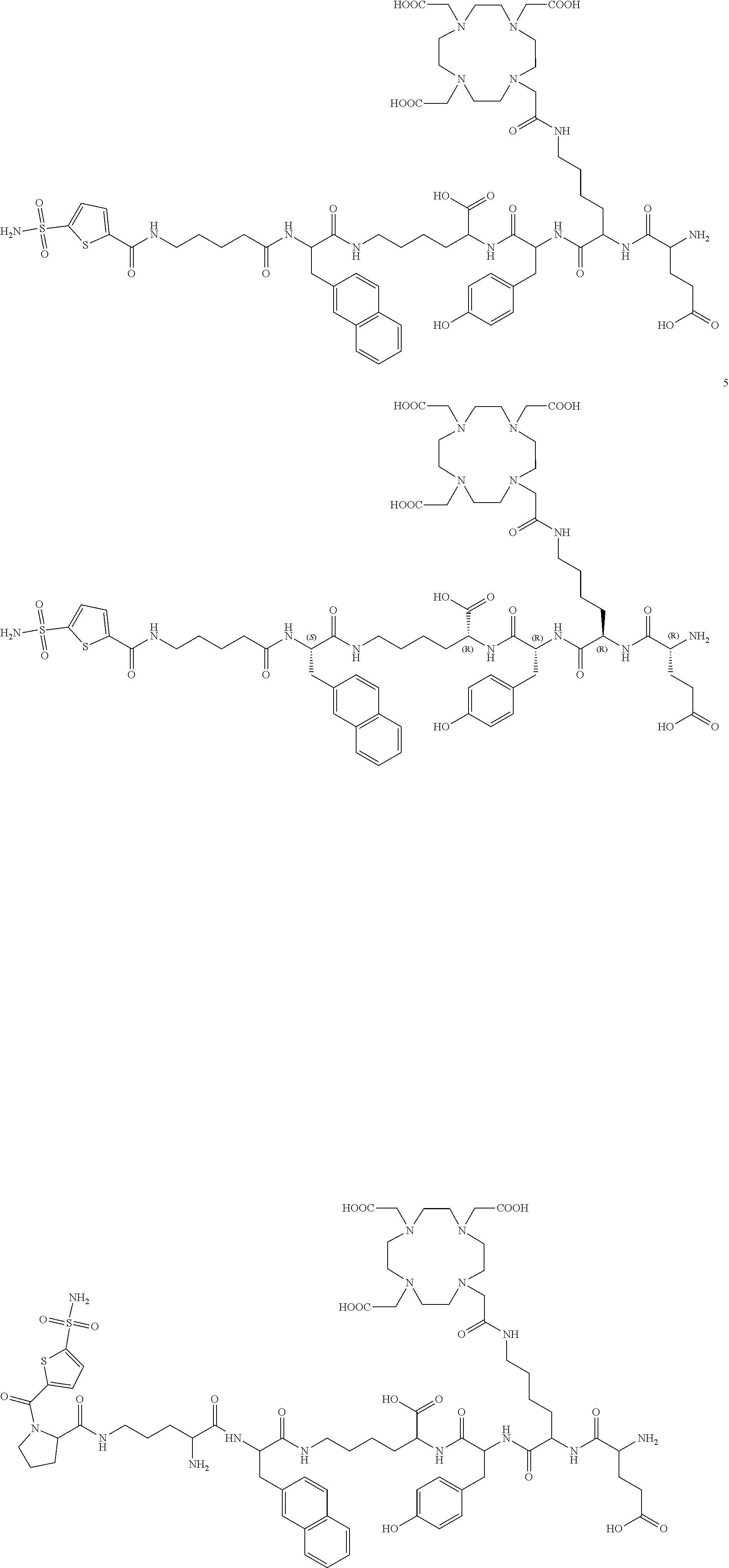

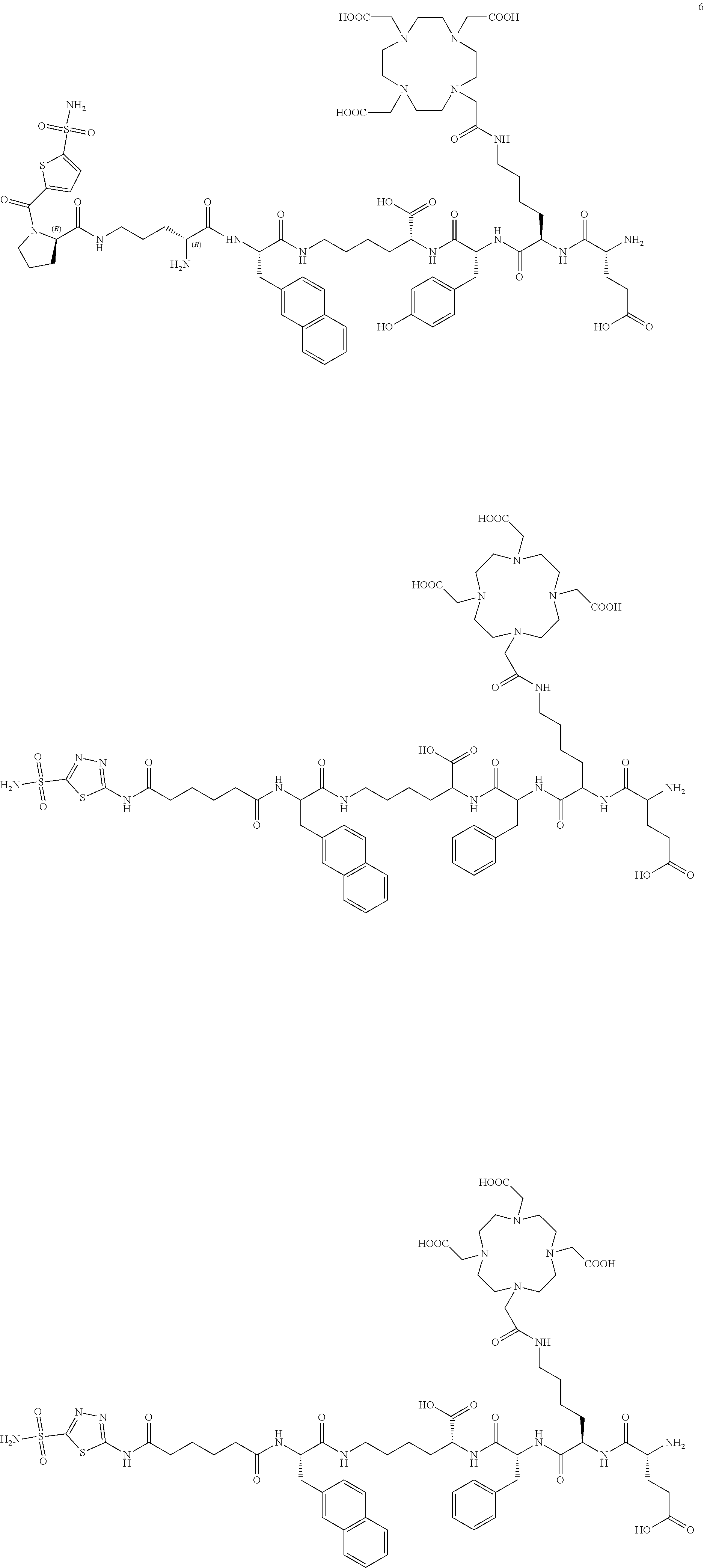

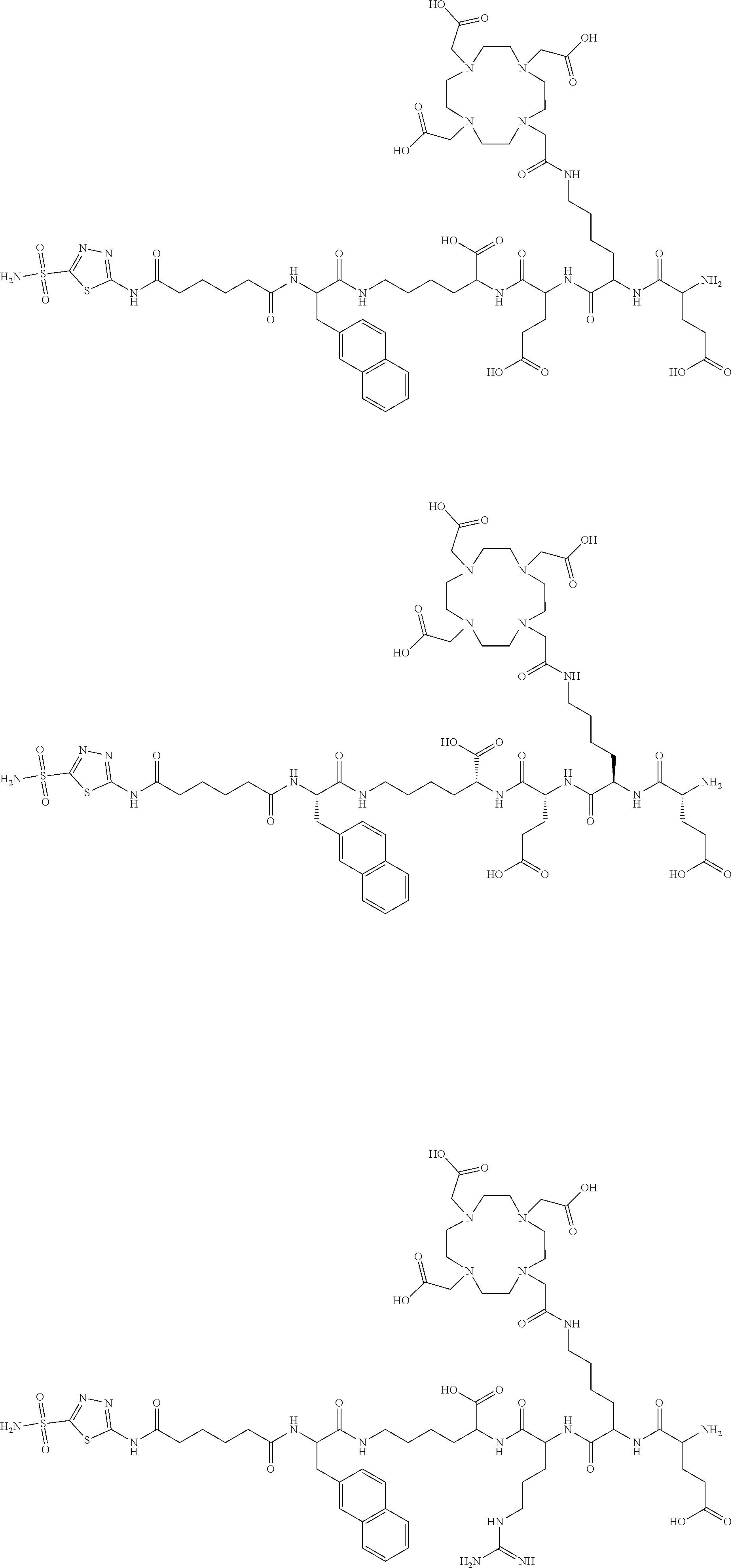

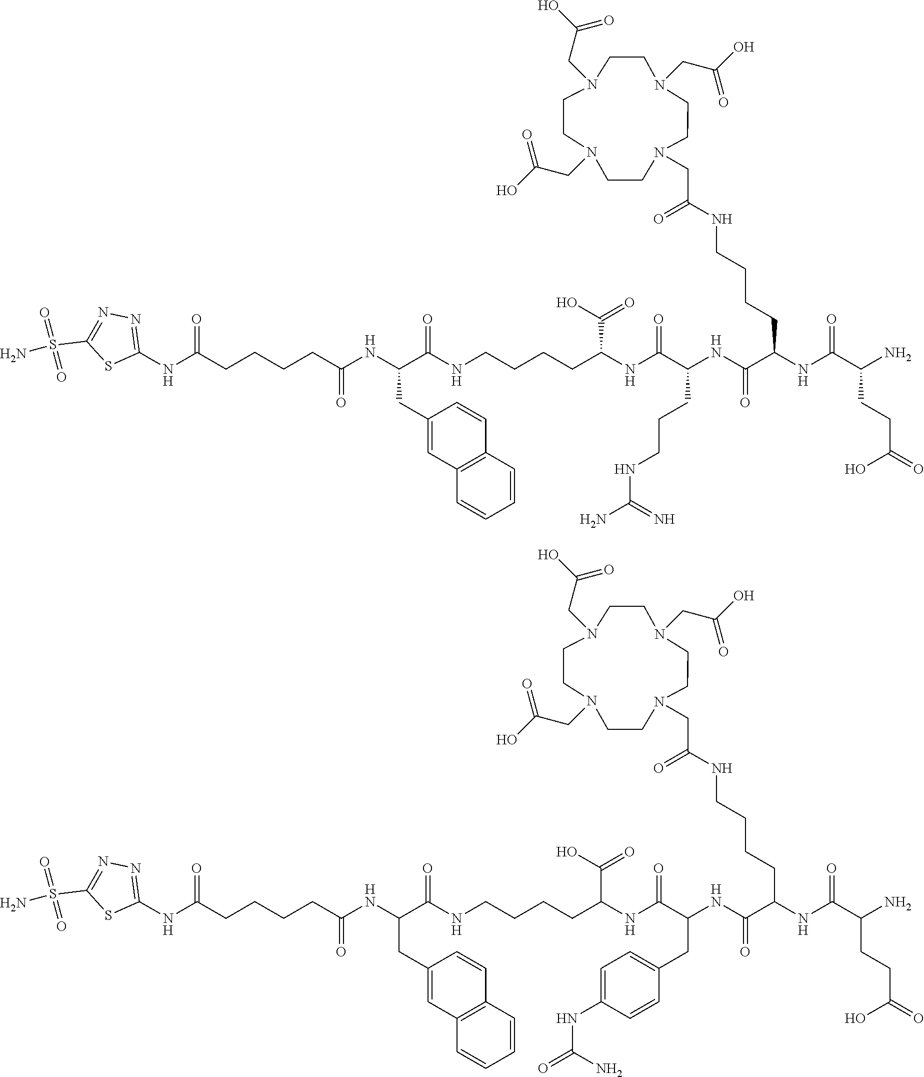 [SMILES: N=C(N)NCCC[C@@H](NC(=O)[C@@H](CCCCNC(=O)CN1CCN(CC(=O)O)CCN(CC(=O)O)CCN(CC(=O)O)CC1)NC(=O)[C@H](N)CCC(=O)O)C(=O)N[C@H](CCCCNC(=O)[C@H](Cc1ccc2ccccc2c1)NC(=O)CCCCC(=O)Nc1nnc(S(N)(=O)=O)s1)C(=O)O ; NC(=O)Nc1ccc(CC(NC(=O)C(CCCCNC(=O)CN2CCN(CC(=O)O)CCN(CC(=O)O)CCN(CC(=O)O)CC2)NC(=O)C(N)CCC(=O)O)C(=O)NC(CCCCNC(=O)C(Cc2ccc3ccccc3c2)NC(=O)CCCCC(=O)Nc2nnc(S(N)(=O)=O)s2)C(=O)O)cc1]

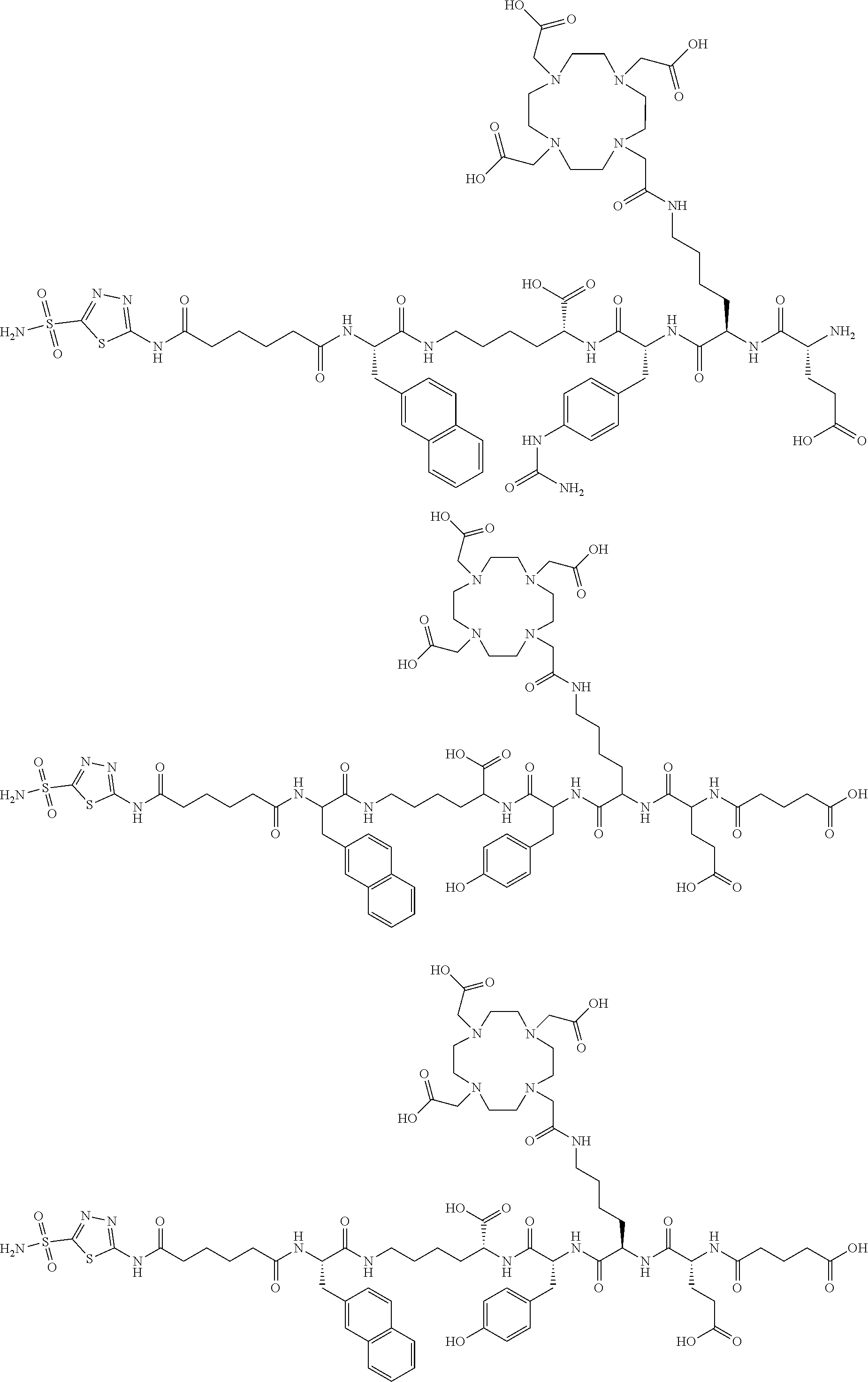

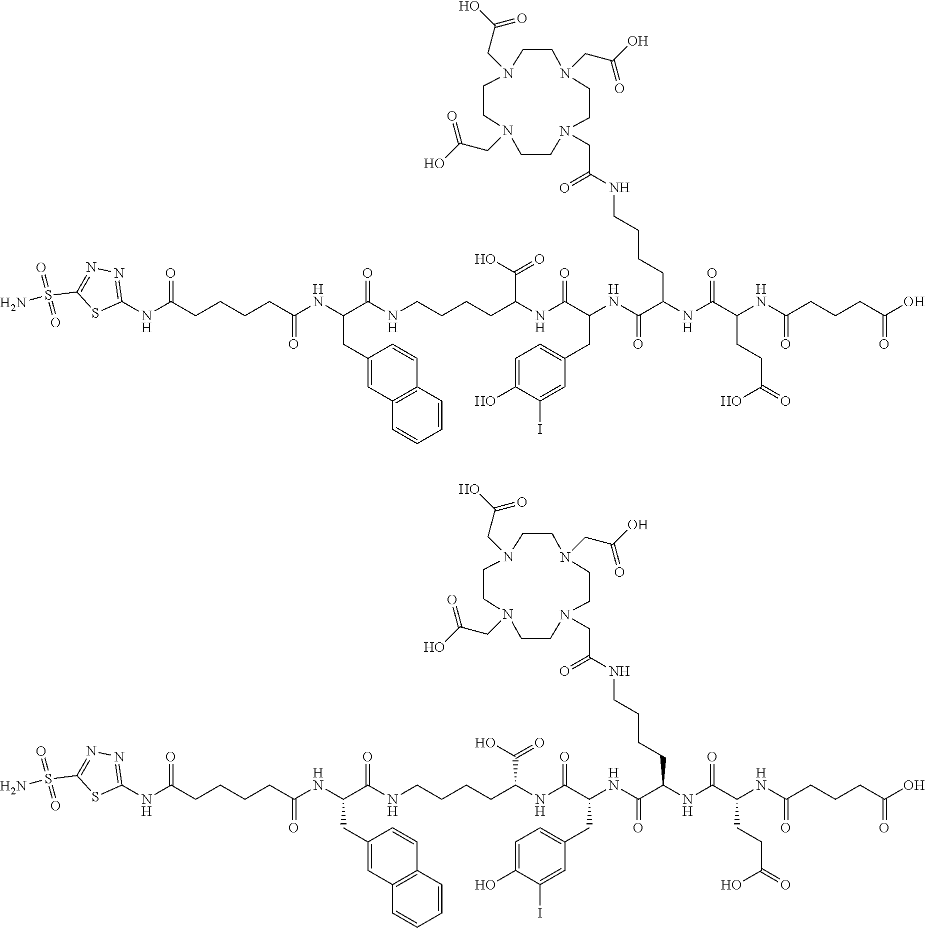 [SMILES: NS(=O)(=O)c1nnc(NC(=O)CCCCC(=O)NC(Cc2ccc3ccccc3c2)C(=O)NCCCCC(NC(=O)C(Cc2ccc(O)c(I)c2)NC(=O)C(CCCCNC(=O)CN2CCN(CC(=O)O)CCN(CC(=O)O)CCN(CC(=O)O)CC2)NC(=O)C(CCC(=O)O)NC(=O)CCCC(=O)O)C(=O)O)s1 ; NS(=O)(=O)c1nnc(NC(=O)CCCCC(=O)N[C@@H](Cc2ccc3ccccc3c2)C(=O)NCCCC[C@@H](NC(=O)[C@@H](Cc2ccc(O)c(I)c2)NC(=O)[C@@H](CCCCNC(=O)CN2CCN(CC(=O)O)CCN(CC(=O)O)CCN(CC(=O)O)CC2)NC(=O)[C@@H](CCC(=O)O)NC(=O)CCCC(=O)O)C(=O)O)s1]

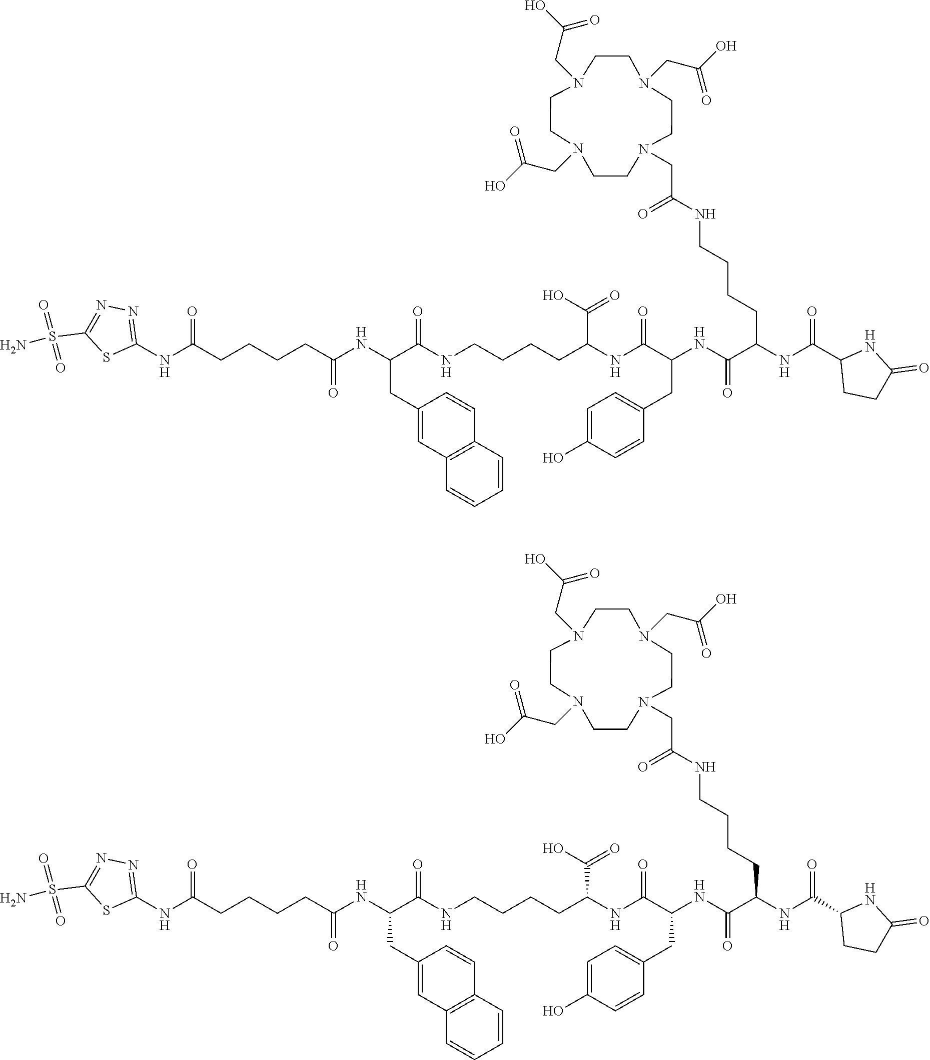 [SMILES: NS(=O)(=O)c1nnc(NC(=O)CCCCC(=O)NC(Cc2ccc3ccccc3c2)C(=O)NCCCCC(NC(=O)C(Cc2ccc(O)cc2)NC(=O)C(CCCCNC(=O)CN2CCN(CC(=O)O)CCN(CC(=O)O)CCN(CC(=O)O)CC2)NC(=O)C2CCC(=O)N2)C(=O)O)s1 ; NS(=O)(=O)c1nnc(NC(=O)CCCCC(=O)N[C@@H](Cc2ccc3ccccc3c2)C(=O)NCCCC[C@@H](NC(=O)[C@@H](Cc2ccc(O)cc2)NC(=O)[C@@H](CCCCNC(=O)CN2CCN(CC(=O)O)CCN(CC(=O)O)CCN(CC(=O)O)CC2)NC(=O)[C@H]2CCC(=O)N2)C(=O)O)s1]

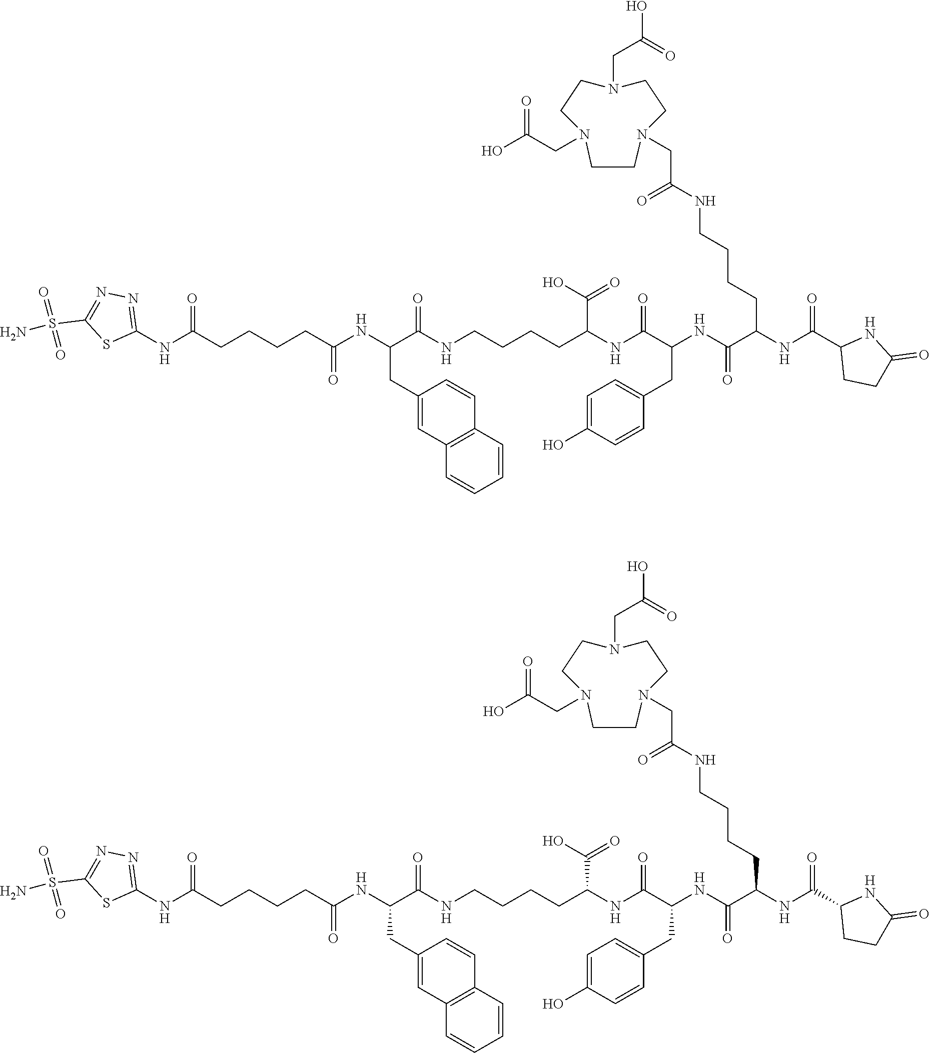 [SMILES: NS(=O)(=O)c1nnc(NC(=O)CCCCC(=O)NC(Cc2ccc3ccccc3c2)C(=O)NCCCCC(NC(=O)C(Cc2ccc(O)cc2)NC(=O)C(CCCCNC(=O)CN2CCN(CC(=O)O)CCN(CC(=O)O)CC2)NC(=O)C2CCC(=O)N2)C(=O)O)s1 ; NS(=O)(=O)c1nnc(NC(=O)CCCCC(=O)N[C@@H](Cc2ccc3ccccc3c2)C(=O)NCCCC[C@@H](NC(=O)[C@@H](Cc2ccc(O)cc2)NC(=O)[C@@H](CCCCNC(=O)CN2CCN(CC(=O)O)CCN(CC(=O)O)CC2)NC(=O)[C@H]2CCC(=O)N2)C(=O)O)s1]

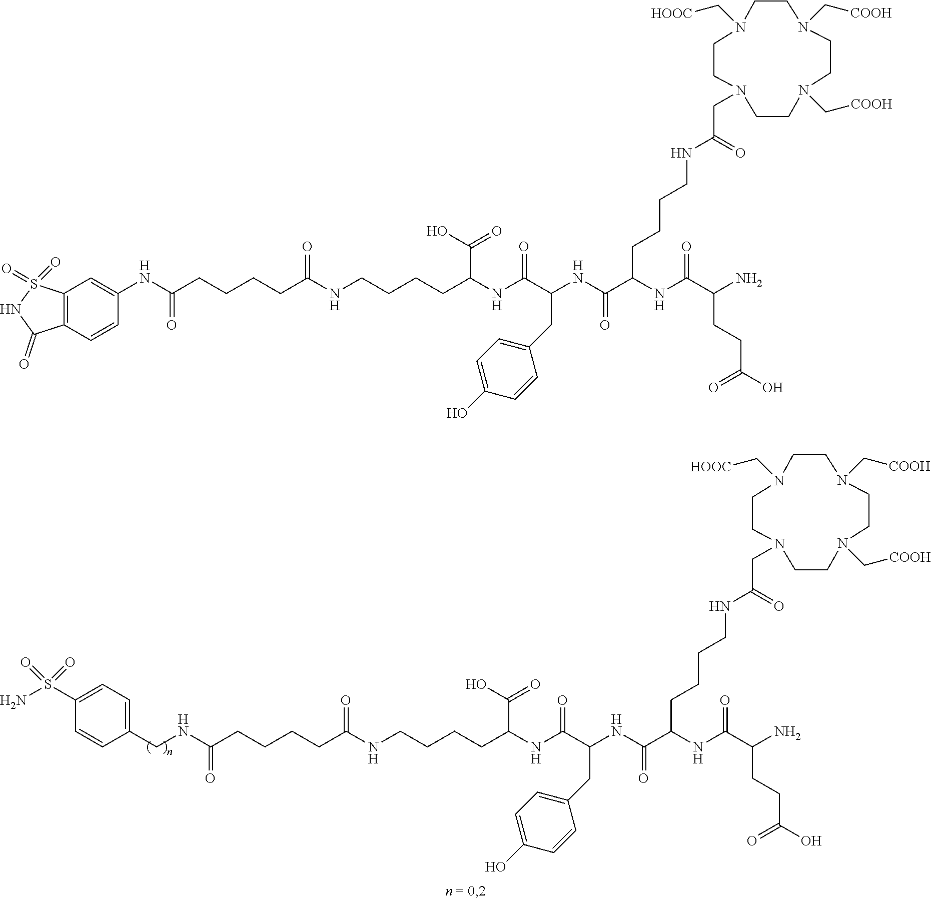

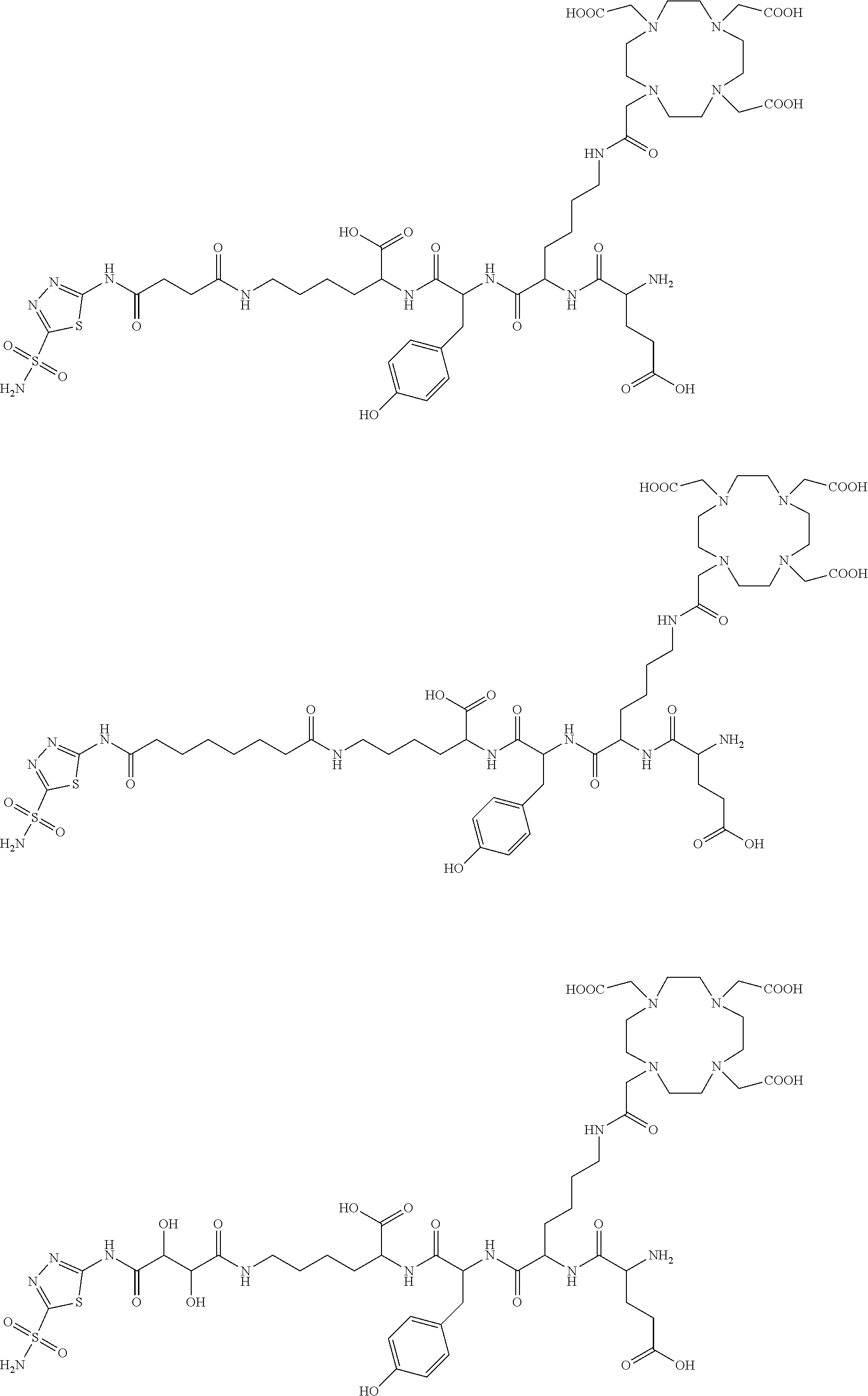 [SMILES: NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CCCCCCC(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)C(O)C(O)C(=O)Nc1nnc(S(N)(=O)=O)s1)C(=O)O]

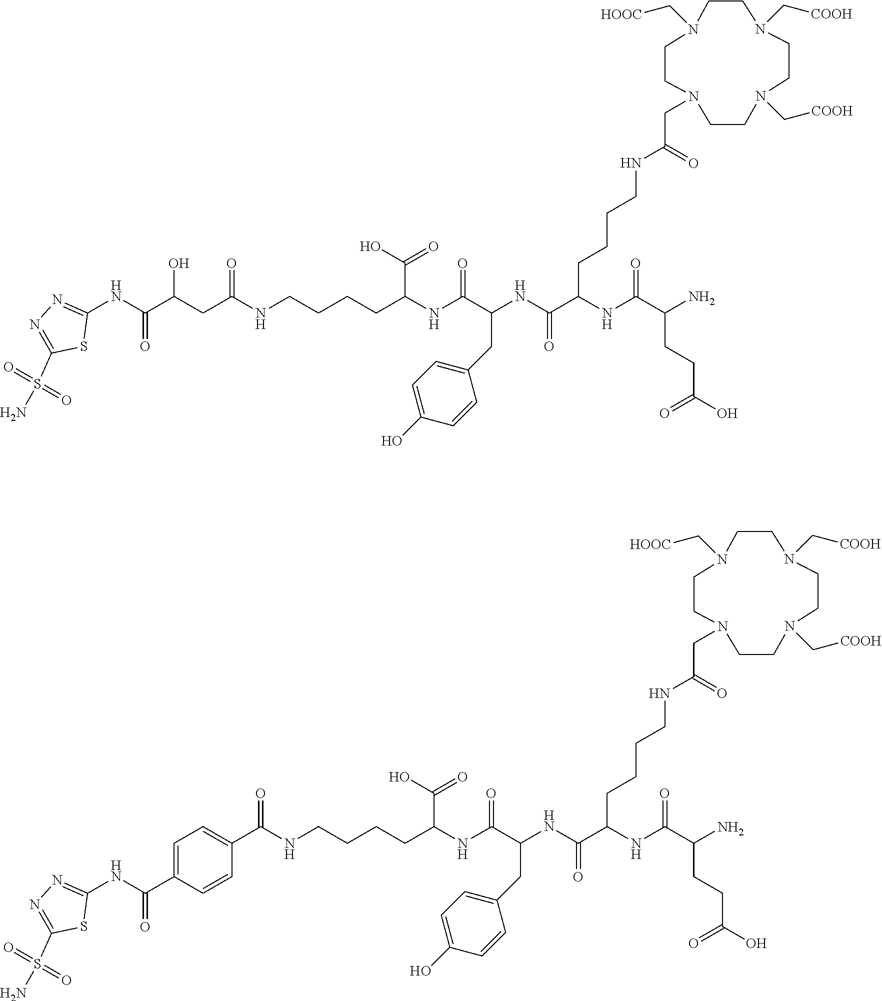 [SMILES: NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)CC(O)C(=O)Nc1nnc(S(N)(=O)=O)s1)C(=O)O ; NC(CCC(=O)O)C(=O)NC(CCCCNC(=O)CN1CCN(CC(=O)O)CCN(CC(=O)O)CCN(CC(=O)O)CC1)C(=O)NC(Cc1ccc(O)cc1)C(=O)NC(CCCCNC(=O)c1ccc(C(=O)Nc2nnc(S(N)(=O)=O)s2)cc1)C(=O)O]

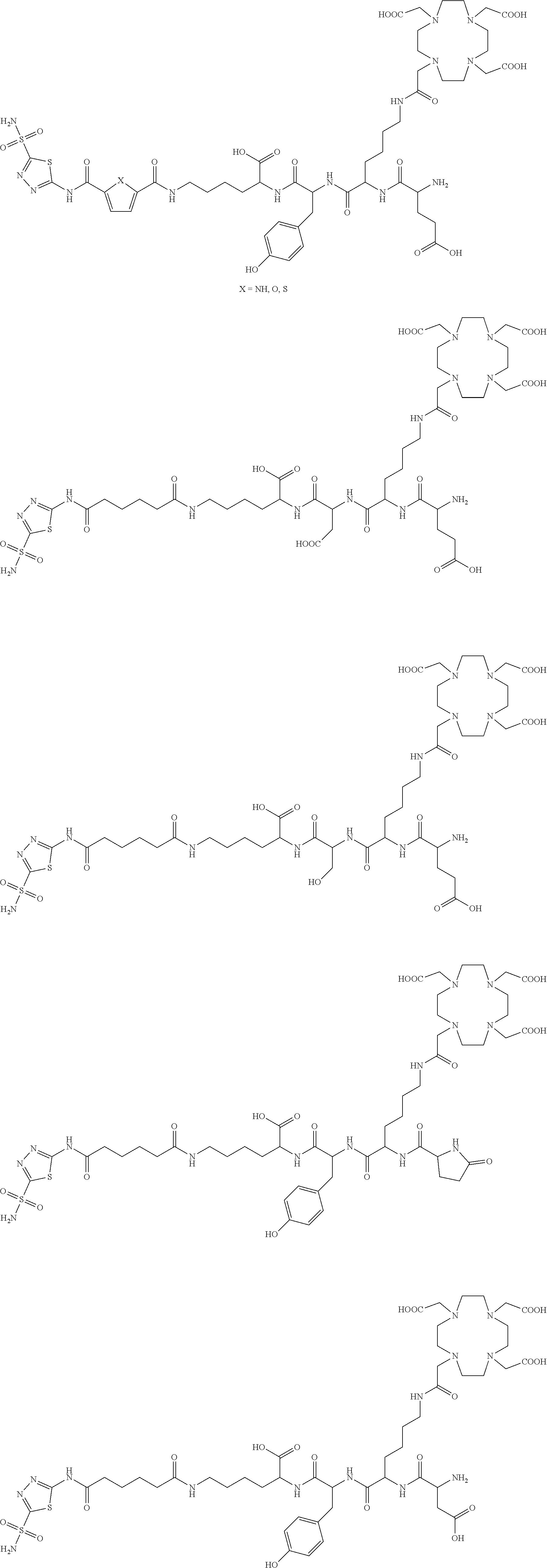

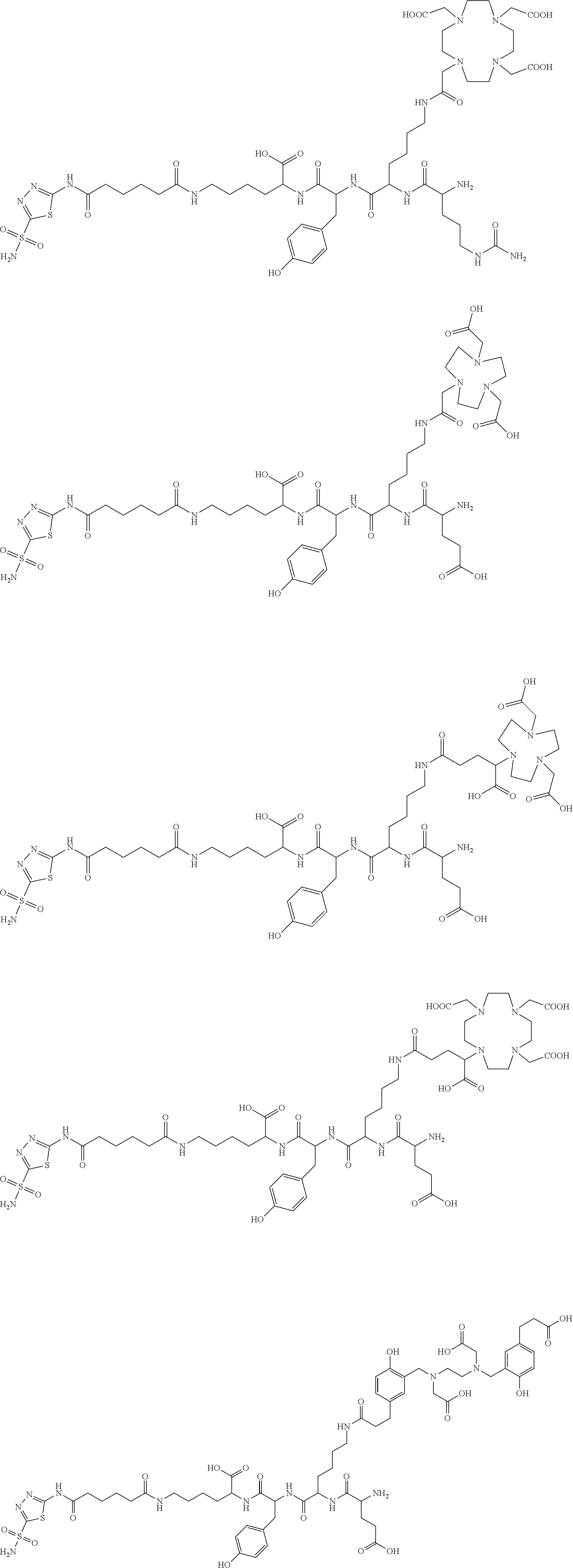

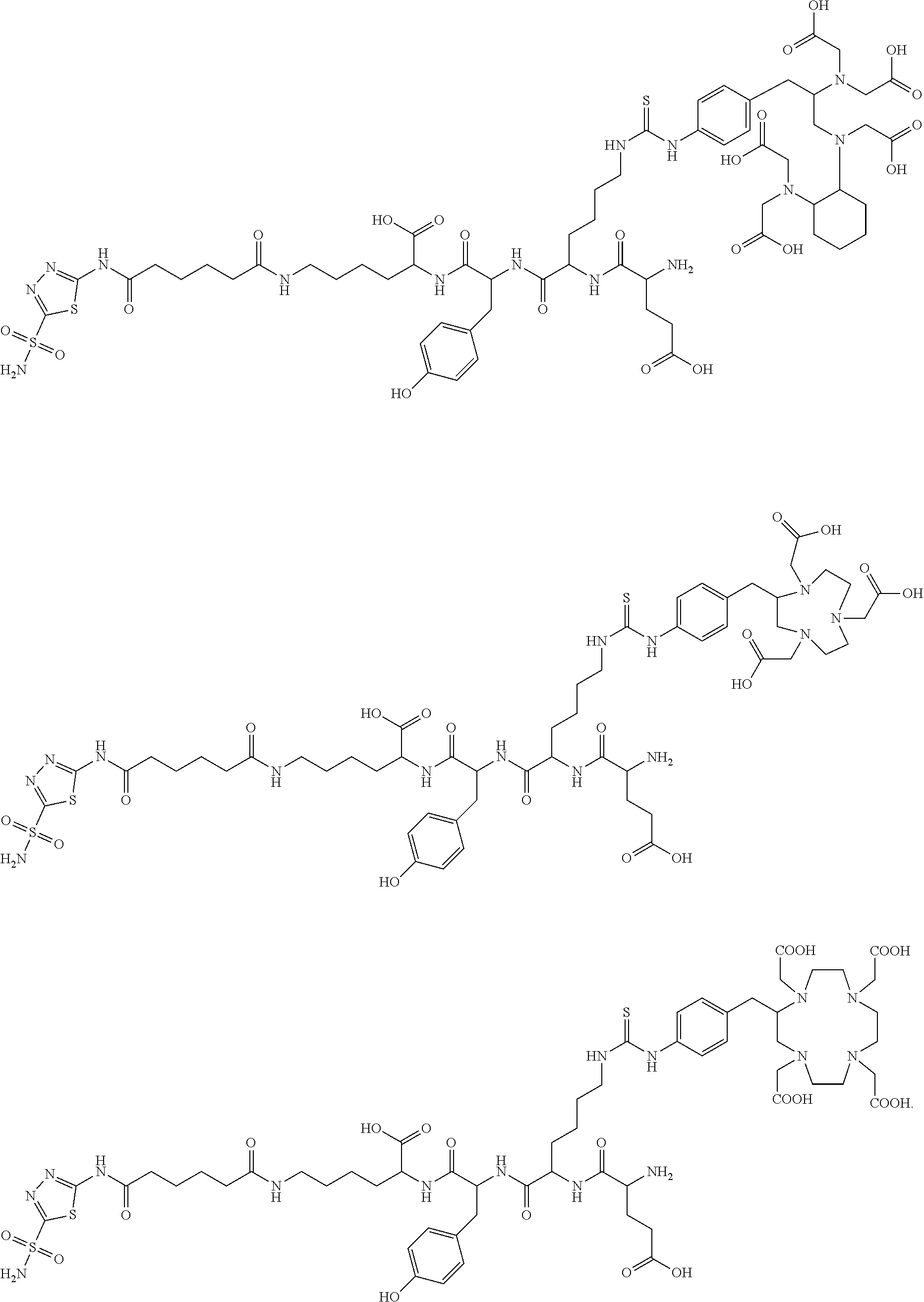 [SMILES: NC(CCC(=O)O)C(=O)NC(CCCCNC(=S)Nc1ccc(CC(CN(CC(=O)O)C2CCCCC2N(CC(=O)O)CC(=O)O)N(CC(=O)O)CC(=O)O)cc1)C(=O)NC(Cc1ccc(O)cc1)C(=O)NC(CCCCNC(=O)CCCCC(=O)Nc1nnc(S(N)(=O)=O)s1)C(=O)O ; NC(CCC(=O)O)C(=O)NC(CCCCNC(=S)Nc1ccc(CC2CN(CC(=O)O)CCN(CC(=O)O)CCN2CC(=O)O)cc1)C(=O)NC(Cc1ccc(O)cc1)C(=O)NC(CCCCNC(=O)CCCCC(=O)Nc1nnc(S(N)(=O)=O)s1)C(=O)O ; NC(CCC(=O)O)C(=O)NC(CCCCNC(=S)Nc1ccc(CC2CN(CC(=O)O)CCN(CC(=O)O)CCN(CC(=O)O)CCN2CC(=O)O)cc1)C(=O)NC(Cc1ccc(O)cc1)C(=O)NC(CCCCNC(=O)CCCCC(=O)Nc1nnc(S(N)(=O)=O)s1)C(=O)O]

13. A compound comprising a compound of formula (I), or an isotopic variant, hydrate, ester or solvate, tautomer, stereoisomer, or pharmaceutically acceptable salt thereof, and M complexed therewith,
wherein,
the compound of formula (I) is as defined in claim 1;
M is selected from at least one of radionuclides or non-radioactive elements (alternatively radionuclides); alternatively, the radionuclide is selected from ⁶⁸Ga, ¹⁸F, ⁹⁹mTc, ⁸⁹Zr, ¹²⁴I, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or ⁸⁶Y, or is selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb, ⁴⁷Sc, ^{186/188}Re, ^{212/213}Bi, ¹⁴⁹Pm, ²¹²Pb, ²¹¹At, ²²³Ra, ¹⁶¹Tb, ²²⁵Ac or ²²⁷Th;
alternatively, the radionuclide is selected from ⁶⁸Ga, ¹⁸F, ⁶⁴Cu, ¹⁶¹Tb or ¹⁷⁷Lu;
alternatively, the radionuclide ¹⁸F is formed by complexing ¹⁸FA1 with the compound of formula (I);
alternatively, the M is selected from ⁶⁸Ga, ¹⁸F, ¹⁶¹Tb or ¹⁷⁷Lu; alternatively ⁶⁸Ga or ¹⁷⁷Lu.

14. A compound of formula (V), or an isotopic variant, a hydrate, an ester or solvate, a tautomer or a stereoisomer thereof, or a pharmaceutically acceptable salt thereof: 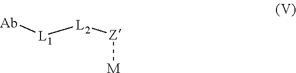
wherein,
Ab, L₁ and L₂ are as defined in claim 1;
Z′ is a coordination group formed by complexing a chelating group Z derived from a chelating agent with M, wherein Z is as defined in claim 1;
M is selected from at least one of radionuclides or non-radioactive elements (alternatively radionuclides);
alternatively, the radionuclide is selected from ⁶⁸Ga, ¹⁸F, ⁹⁹mTc, ⁸⁹Zr, ¹²⁴, ⁷⁶Br, ⁴³Sc, ¹¹¹In, ⁴⁵Ti, ⁵²Mn, ⁵⁹Fe, ⁶⁴Cu, ⁹⁴mTc, ⁶⁷Ga, ^{71/72/74}As, ^{82m}Rb or 16Y, or is selected from ¹⁷⁷Lu, ⁹⁰Y, ¹³¹I, ¹⁵³Sm, ⁶⁷Cu, ⁸⁹Sr, ¹⁶⁶Ho, ¹⁷⁷Yb ⁴⁷Sc, ^{186/188}Re, ^{212/213}Bi, ¹⁴⁹Pm, ²¹²Pb, ²¹¹At, ²²³Ra, ¹⁶¹Tb, ²²⁵Ac or ²²⁷Th;
alternatively, the radionuclide is selected from ⁶⁸Ga, ¹⁸F, ⁶⁴Cu, ¹⁶¹Tb or ¹⁷⁷Lu;
alternatively, the radionuclide ¹⁸F is formed by ¹⁸FA1 complexation;
alternatively, the M is selected from ⁶⁸Ga, ¹⁸F, ¹⁶¹Tb or ¹⁷⁷Lu; alternatively ⁶⁸Ga or ¹⁷⁷Lu.

15. A pharmaceutical composition comprising the compound of claim 1, or an isotopic variant, a hydrate, an ester or solvate, a tautomer or a stereoisomer thereof, or a pharmaceutically acceptable salt thereof; optionally, the pharmaceutical composition further comprises a pharmaceutically acceptable carrier or excipient.

16. A method for inhibiting the expression of carbonic anhydrase IX, comprising administering to a subject the compound of claim 1, or an isotopic variant, hydrate, ester or solvate, tautomer or stereoisomer thereof, or a pharmaceutically acceptable salt thereof.

17. A method for diagnosing and/or treating one or more tumors, cancers or cells expressing carbonic anhydrase IX, comprising administering to a subject a compound of claim 1, or an isotopic variant, hydrate, ester or solvate, tautomer or stereoisomer thereof, or a pharmaceutically acceptable salt thereof.

18. The method of claim 17, wherein the diagnostic modality is selected from optical imaging and/or nuclear imaging; alternatively, the nuclear imaging is selected from PET imaging and/or SPECT imaging;
alternatively, the treatment is selected from radiotherapy and/or assisted surgery with fluorescent surgical navigation.

19. The method of claim 18, wherein the disease associated with carbonic anhydrase IX expression is selected from tumors;
alternatively, the disease associated with carbonic anhydrase IX expression is selected from renal cancer, lung cancer, colorectal cancer, gastric cancer, pancreatic cancer, melanoma, breast cancer, cervical cancer, bladder cancer, ovarian cancer, brain cancer, head and neck cancer, astrocytoma and oral cancer.

20. A method for imaging a tissue expressing carbonic anhydrase IX, comprising administering to the tissue the compound of claim 1, or an isotopic variant, a hydrate, an ester or solvate, a tautomer or a stereoisomer thereof, or a pharmaceutically acceptable salt thereof, and imaging the tissue after administration;
alternatively, the imaging is emission computed tomography, performed by positron emission tomography or single photon emission computed tomography.
